(19) United States
(12) Patent Application Publication
(10) Pub. No.: US 2026/0256568 A1
(43) Pub. Date: Sep. 3, 2026

(54) TISSUE REPAIR IMPLANT DEVICE

(71) Applicants: Smith & Nephew, Inc., Memphis, TN (US); Smith & Nephew Orthopaedics AG, Zug (CH); Smith & Nephew Asia Pacific Pte. Limited, Singapore (SG)

(72) Inventors: Timothy Young, Upton, MA (US); Dennis P. Colleran, North Attleboro, MA (US); Nehal N. Patel, Boston, MA (US); Collin W. Shale, Medford, MA (US); Marc J. Balboa, Hopkinton, MA (US); William Middleton, Middleboro, MA (US); Helawe Betre, Round Rock, TX (US); Mark E. Housman, North Attleboro, MA (US); Robert Bearden, Aledo, TX (US); Hadi El Heneidi, Watertown, MA (US); Jon-Paul Rogers, North Smithfield, RI (US); Benjamin M. Hall, Roslindale, MA (US); Rick Fu, Randolph, MA (US); Christopher D. MacCready, Medfield, MA (US); Matthew D. Cunningham, Lakeville, MA (US); Ali Hosseini, Quincy, MA (US); Kieran Lenehan, Cambridge, MA (US); David A. Yeung, Andover, MA (US); Brendan Jablonski, Boston, MA (US); Nicholas J. Cotton, Westborough, MA (US); Haley Schierwagen, Melrose, MA (US)

(73) Assignees: Smith & Nephew, Inc., Memphis, TN (US); Smith & Nephew Orthopaedics AG, Zug (CH); Smith & Nephew Asia Pacific Pte. Limited, Singapore (SG)

(21) Appl. No.: 19/650,681

(22) Filed: Apr. 17, 2026

Related U.S. Application Data
(63) Continuation of application No. PCT/US2024/054937, filed on Nov. 7, 2024.
(60) Provisional application No. 63/597,113, filed on Nov. 8, 2023.
(60) Provisional application No. 63/598,573, filed on Nov. 14, 2023.
(60) Provisional application No. 63/598,633, filed on Nov. 14, 2023.
(60) Provisional application No. 63/598,676, filed on Nov. 14, 2023.
(60) Provisional application No. 63/598,692, filed on Nov. 14, 2023.
(60) Provisional application No. 63/598,771, filed on Nov. 14, 2023.
(60) Provisional application No. 63/598,798, filed on Nov. 14, 2023.
(60) Provisional application No. 63/598,969, filed on Nov. 15, 2023.
(60) Provisional application No. 63/598,976, filed on Nov. 15, 2023.
(60) Provisional application No. 63/598,988, filed on Nov. 15, 2023.
(60) Provisional application No. 63/602,713, filed on Nov. 27, 2023.
(60) Provisional application No. 63/602,721, filed on Nov. 27, 2023.

(51) Int. Cl. A61F 2/08; A61L 27/24; A61L 27/36

(52) U.S. Cl. CPC A61F 2/0811; A61L 27/24; A61L 27/3662; A61F 2210/0076; A61F 2220/0016; A61F 2250/0036

(57) ABSTRACT

Medical implants are disclosed. A medical implant includes a first base material having a first end region, a second end region and a feltable material adjacent to the first base material. Further, the feltable material includes a plurality of feltable fibers configured to attach the implant to a target tissue.

### CROSS REFERENCE TO RELATED APPLICATIONS

This application is continuation of International Application No. PCT/US2024/054937, filed on Nov. 7, 2024, which claims the benefit of and priority to related to U.S. Patent Application Ser. Nos. 63/597,113 filed on Nov. 8, 2023, 63/598,573 filed on Nov. 14, 2023, 63/598,633 filed on Nov. 14, 2023, 63/598,676 filed on Nov. 14, 2023, 63/598,692 filed on Nov. 14, 2023, 63/598,771 filed on Nov. 14, 2023, 63/598,798 filed on Nov. 14, 2023, 63/598,969 filed on Nov. 15, 2023, 63/598,976 filed on Nov. 15, 2023, 63/598,988 filed on Nov. 15, 2023, 63/602,713 filed on Nov. 27, 2023, and 63/602,721 filed on Nov. 27, 2023, the entire disclosures of which are incorporated herein by reference.

### TECHNICAL FIELD

The present disclosure pertains generally, but not by way of limitation, to orthopedic implants, compositions, and methods of treatment. More particularly, the present invention relates to a soft tissue repair implant, such as one that is configured for placement in the area of a tear or lesion of a tendon or ligament, and compositions and methods for promoting and enhancing healing at a soft tissue-to-bone interface in a surgical repair.

### BACKGROUND

Overuse, injury, aging and health conditions can damage soft tissues of the body. For example, a common soft tissue injury is damage to the rotator cuff or rotator cuff tendons. Damage to the rotator cuff is a potentially serious medical condition that may occur during hyperextension, from an acute traumatic tear or from overuse of the joint. Other common soft tissues injuries include injuries to the anterior cruciate ligament (ACL), the lateral collateral ligament (LCL), meniscus of the knee, tendons/ligaments of the hip capsule, Achilles tendon and other various ligaments of the shoulder, knee, ankle and hip, for example. Current procedures may attempt to alleviate impingement or make room for movement of the injured soft tissue to prevent further damage and relieve discomfort but may not repair or strengthen the soft tissue. In some instances, biologic tissue scaffolds (e.g., biologic implants, decellularized implants, dermal grafts, synthetic grafts, etc.) may be used to reinforce degenerated tendons, ligaments or other injured soft tissues. Further, it can be appreciated that various attachment techniques may be utilized to attach biologic tissue scaffolds to soft tissue and/or bone at one or more connection points, whereby the robustness of the mechanical connection of the implant to the soft tissue and/or bone may significantly influence the overall healing of the injury. Example attachment techniques disclosed herein may utilize felting techniques to achieve high fixation connections when attaching a tissue repair implant to soft tissue and/or bone.

### BRIEF SUMMARY

This disclosure provides design, material, manufacturing method, and use alternatives for medical devices. A medical implant includes a first base material having a first end region, a second end region and a feltable material adjacent to the first base material. Further, the feltable material includes a plurality of feltable fibers configured to attach the implant to a target tissue.

Alternatively or additionally to any of the embodiments above, wherein at least a portion of the first base material is formed from collagen.

Alternatively or additionally to any of the embodiments above, wherein at least a portion of the first base material is formed from a polymer.

Alternatively or additionally to any of the embodiments above, wherein the feltable material is dispersed within the first base material.

Alternatively or additionally to any of the embodiments above, wherein the feltable material is chemically bonded to the first base material.

Alternatively or additionally to any of the embodiments above, wherein the feltable material is positioned on top of the first base material.

Alternatively or additionally to any of the embodiments above, wherein the feltable material is positioned along the perimeter of the first base material, and wherein a central portion of the first base material is free of feltable material.

Alternatively or additionally to any of the embodiments above, further comprising a second base material, wherein the feltable material is positioned between the first base material and the second base material.

Alternatively or additionally to any of the embodiments above, wherein at least a portion of the first base material and the second base material are formed from collagen.

Alternatively or additionally to any of the embodiments above, further comprising a reinforcing material, wherein the reinforcing material is positioned between the feltable material and the second base material, and wherein the feltable material is positioned between the first base material and the reinforcing material.

Alternatively or additionally to any of the embodiments above, wherein the medical implant includes a length extending from the first end region of the base material to the second end region of the base material, and wherein the feltable material extends along only a portion of the length.

Alternatively or additionally to any of the embodiments above, wherein the first end region is devoid of the feltable material.

Alternatively or additionally to any of the embodiments above, wherein the first end region has a first thickness and the second end region has a second thickness greater than the first thickness.

Alternatively or additionally to any of the embodiments above, further comprising a reinforcing material, wherein the reinforcing material is positioned between the first base material and the second base material, and wherein the first base material is positioned between the feltable material and the reinforcing material.

Alternatively or additionally to any of the embodiments above, wherein the feltable material is positioned along the second end region of the medical implant and wherein the first end region is devoid of the feltable material.

Alternatively or additionally to any of the embodiments above, further comprising a reinforcing material.

Alternatively or additionally to any of the embodiments above, wherein the first base material is positioned between the reinforcing material and the feltable material.

Alternatively or additionally to any of the embodiments above, wherein the feltable material is positioned along the perimeter of the first base material, and wherein a central portion of the first base material is free of the feltable material.

Another medical implant includes a first base material having a first end region, a second end region, a feltable material coupled to the first base material and a suture coupled to the first end region. Further, the feltable material is configured to anchor the second end region to a first target tissue and the suture is configured to anchor the first end region to a bone anchor.

Another medical implant includes a base component including a first end region, a first base material and a feltable material. The medical implant further includes a first suture coupled to a first end region, a second suture coupled to a first end region, a first bone anchor coupled to the first suture and a second bone anchor coupled to the second suture.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a perspective view of a repair implant;

FIG. 2 is a cross-sectional view of a shoulder showing a tendon tear and an exemplary repair implant positioned thereon;

FIG. 3 is a cross-sectional view of a shoulder showing a felting needle being used to attached the exemplary repair implant of FIG. 2 to the tendon;

FIGS. 4A-4I are top views of a various repair implants;

FIG. 5 is a perspective view of a repair implant;

FIG. 6 is a perspective view of the repair implant shown in FIG. 5;

FIG. 7 is a perspective view of a repair implant;

FIG. 8 is an exploded view of the repair implant shown in FIG. 7;

FIG. 9 is a perspective view of a repair implant;

FIG. 10 is an exploded view of the repair implant shown in FIG. 9;

FIG. 11 is a perspective view of a repair implant;

FIG. 12 is an exploded view of the repair implant shown in FIG. 11;

FIG. 13 is a perspective view of a repair implant;

FIG. 14 is a perspective view of a repair implant;

FIG. 15 is a perspective view of a repair implant;

FIG. 16 is a perspective view of a repair implant;

FIG. 17 is a perspective view of a repair implant;

FIG. 18 is a perspective view of a repair implant;

FIG. 19 is a perspective view of a repair implant;

FIG. 20 illustrates a manufacturing technique used to construct a repair implant;

FIG. 21 is a perspective view of a repair implant constructed using the manufacturing technique of FIG. 20;

FIG. 22 is a perspective view of a repair implant;

FIG. 23 is a perspective view of a repair implant;

FIG. 24 is an exploded view of the repair implant shown in FIG. 23;

FIG. 25 is a cross-sectional view of a shoulder showing a tendon tear and the repair implant of FIG. 24 positioned thereon;

FIG. 26 is a cross-sectional view of a shoulder showing a felting needle being used to attached the repair implant of FIG. 24 to the tendon;

FIG. 27 is a perspective view of a repair implant;

FIG. 28 is a perspective view of a repair implant;

FIGS. 29A-29E are top views of various repair implants;

FIG. 30 is a perspective view of a repair implant;

FIG. 31 is a perspective view of a repair implant;

FIG. 32 illustrates a repair implant positioned along a tendon;

FIGS. 33A-33E are perspective views of various repair implants;

FIG. 34 illustrates a repair implant positioned along a tendon;

FIG. 35 is a perspective view of a repair implant;

FIG. 36 is a perspective view of a repair implant;

FIG. 37 is a perspective view of a knee joint including a torn meniscus;

FIG. 38 is a perspective view of a repair implant positioned along the torn meniscus shown in FIG. 37;

FIG. 39 is a perspective view of a repair implant;

FIG. 40 illustrates a knee joint including a torn tendon;

FIG. 41 illustrates a repair implant positioned along the torn tendon shown in FIG. 40;

FIG. 42 is a side view of the repair implant positioned along the torn tendon shown in FIG. 40;

FIG. 43 is a perspective view of a repair implant;

FIG. 44 is a perspective view of a repair implant;

FIG. 45 illustrates the repair implant of FIG. 44 implanted along a torn tendon;

FIG. 46 is a perspective view of a repair implant;

FIG. 47 is an exploded view of a portion of the repair implant shown in FIG. 46;

FIG. 48 illustrates a portion of the repair implant shown in FIG. 46;

FIG. 49 is an exploded view of a portion of the repair implant shown in FIG. 46;

FIG. 50 illustrates a knee joint including a torn anterior cruciate ligament;

FIG. 51 illustrates the knee joint shown in FIG. 50 including tibial and femoral tunnels formed therein;

FIG. 52 illustrates a repair implant positioned along the torn anterior cruciate ligament shown in FIG. 50;

FIG. 53 is a perspective view of a repair implant;

FIG. 54 illustrates a repair implant positioned along the torn anterior cruciate ligament shown in FIG. 50;

FIG. 55 is a cross-sectional view of the repair implant of FIG. 53 taken along line 55 -55 of FIG. 54;

FIG. 56 is a perspective view of a repair implant;

FIG. 57 illustrates a knee joint including an iliotibial band and a lateral collateral ligament;

FIG. 58 illustrates a repair implant positioned along the iliotibial band shown in FIG. 57;

FIG. 59 illustrates a repair implant positioned along the iliotibial band shown in FIG. 57;

FIG. 60 is a perspective view of a repair implant;

FIG. 61 is a side view of the repair implant shown in FIG. 60;

FIG. 62 illustrates the repair implant of FIG. 60 implanted along a torn tendon;

FIG. 63 illustrates a repair implant implanted along a torn tendon;

FIG. 64 is a side view of the repair implant of FIG. 63 implanted along a torn tendon;

FIG. 65 illustrates a repair implant implanted at along a torn tendon;

FIG. 66 is a side view of the repair implant of FIG. 65 positioned along a torn tendon;

FIG. 67 is a perspective view of a repair implant;

FIG. 68 is a side view of the repair implant of FIG. 67 implanted at a target site in a first configuration;

FIG. 69 is a side view of the repair implant of FIG. 67 implanted at a target site in a second configuration;

FIG. 70 is a perspective view of a repair implant;

FIG. 71 is a perspective view of a repair implant;

FIG. 72 illustrates a knee joint including a torn anterior cruciate ligament;

FIG. 73 illustrates the knee joint shown in FIG. 72 including tibial and femoral tunnels formed therein;

FIG. 74 illustrates a repair implant positioned along the torn anterior cruciate ligament shown in FIG. 73 in a first configuration;

FIG. 75 illustrates a repair implant positioned along the torn anterior cruciate ligament shown in FIG. 73 in a first configuration;

FIG. 76 is a side view of a portion of a medical device positioned adjacent to a repair implant which has been positioned at a target site;

FIG. 77 is a side view of the medical device of FIG. 76 felting the repair implant to the target site;

FIG. 78 is a side view of a portion of a medical device positioned adjacent to a repair implant which has been positioned at a target site;

FIG. 79 is a side view of the medical device of FIG. 78 felting the repair implant to the target site;

FIG. 80 is a side view of the medical device of FIGS. 78-79 after felting the repair implant to the target site;

FIG. 81 illustrates a felting needle delivery device in a first configuration;

FIG. 82 illustrates the felting needle delivery device of FIG. 81 in a second configuration;

FIG. 83 illustrates the felting needle delivery device of FIG. 81 in a third configuration;

FIG. 84 is a perspective view of a portion of a felting needle delivery device;

FIG. 85 is a perspective view of a repair implant;

FIG. 86 illustrates the repair implant of FIG. 85 implanted along a torn tendon;

FIG. 87 is a perspective view of a repair implant;

FIG. 88 is a perspective view of a repair implant;

FIG. 89 is a perspective view of a repair implant;

FIG. 90 is a perspective view of a repair implant;

FIG. 91 illustrates the repair implant of FIG. 88 implanted along a torn tendon;

FIG. 92 is a perspective view of a repair implant;

FIG. 93 illustrates the repair implant of FIG. 92 implanted along a torn tendon;

FIG. 94 is a perspective view of a repair implant;

FIG. 95 is a perspective view of a repair implant;

FIG. 96 is a perspective view of a repair implant;

FIG. 97 is a perspective view of a repair implant in a first configuration;

FIG. 98 is a perspective view of the repair implant of FIG. 97 in a second configuration;

FIG. 99 is a perspective view of a repair implant;

FIG. 100 illustrates a repair implant implanted along a torn tendon;

FIG. 101 illustrates a repair implant implanted along a torn tendon;

FIG. 102 is a perspective view of a repair implant;

FIG. 103 is a perspective view of a repair implant;

FIG. 104 is a perspective view of a shoulder showing a tendon tear;

FIG. 105 illustrates the repair implant of FIG. 103 implanted along a torn tendon;

FIG. 106 is a perspective view of a repair implant;

FIG. 107 illustrates the repair implant of FIG. 106 implanted along a torn tendon;

FIG. 108 is a perspective view of a repair implant;

FIG. 109 is a perspective view of a repair implant;

FIG. 110 is a perspective view of a repair implant;

FIG. 111 is a perspective view of a repair implant;

FIG. 112 is a perspective view of a repair implant;

FIG. 113 is a perspective view of a repair implant;

FIG. 114 is a perspective view of a repair implant;

FIG. 115 is a perspective view of a repair implant;

FIG. 116 is a perspective view of a repair implant;

FIG. 117 is a perspective view of a repair implant;

FIG. 118 is a perspective view of a repair implant;

FIG. 119 is a perspective view of a repair implant;

FIG. 120 is a perspective view of a repair implant;

FIG. 121 is a perspective view of a repair implant;

FIG. 122 is a perspective view of a delivery device couple to a repair implant;

FIG. 123 is a perspective view of a portion of the delivery device of FIG. 122 coupled to a repair implant;

FIG. 124 is a perspective view of a repair implant;

FIG. 125 illustrates the repair device of FIG. 124 positioning a repair implant along a torn tendon;

FIG. 126 illustrates a repair implant implanted along a torn tendon;

FIG. 127 illustrates a repair implant implanted along a torn tendon;

FIG. 128 illustrates a repair implant implanted along a torn tendon;

FIG. 129 is a perspective view of a repair implant;

FIG. 130 is a perspective view of a repair implant;

FIG. 131 illustrates the repair implant of FIG. 130 implanted along a torn tendon;

FIG. 132 illustrates a repair implant delivery device.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

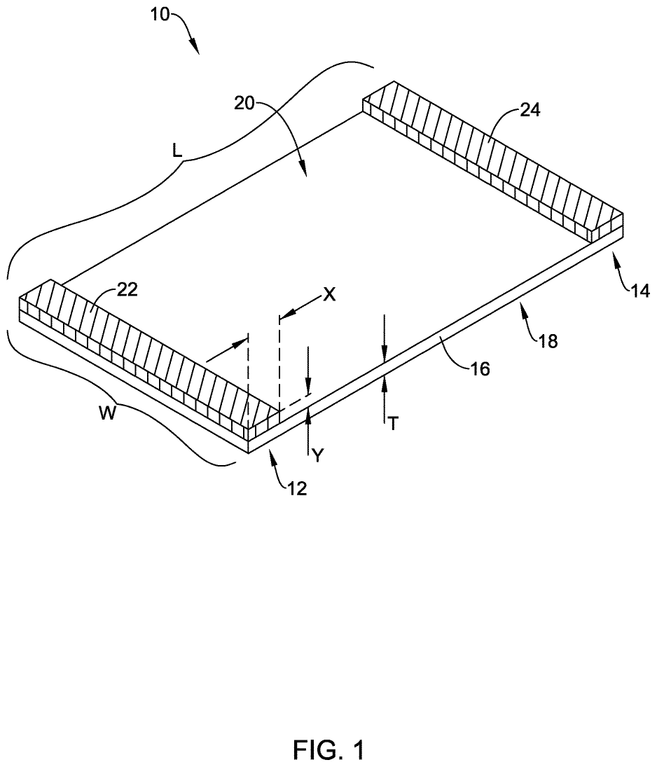

FIG. 1 illustrates a repair implant 10. The repair implant 10 may include a first end region 12 and a second end region 14 positioned opposite the first end region 12. Additionally, the repair implant 10 may include a base 16. The base 16 may include a bottom surface 18 and an upper surface 20.

In some embodiments, the repair implant 10 may be configured to provide a combination of structural features, properties and functions that may be used to treat partial or full thickness tears of soft tissues, including partial or full thickness tears of the rotator cuff tendons, ACL repair, LCL repair, the gluteal tendon, meniscus tears, tendons/ligaments of the hip joint capsule, Achilles tendon, etc. While the repair implant 10 may be described with respect to specific conditions and scenarios of the shoulder, knee and hip, it should be understood that the repair implants described herein may be used in other conditions or scenarios where it is desirable to encourage tissue ingrowth while also providing additional mechanical properties such as, but not limited to strength and stiffness. These features, properties, and functions may include: rapid deployment and fixation by arthroscopic means that complement current procedures, tensile properties that result in desired sharing of anatomical load between the implant and native tendon during rehabilitation, selected porosity and longitudinal pathways for tissue in-growth, sufficient cyclic straining of the implant, having new tissue in-growth, induction of a healing response, and, in some cases, the repair implant may be bioabsorbable or otherwise absorbable to provide transfer of additional load to native soft tissue (e.g., tendon) over time. In some instances, the implant 10 may be described as a bioinductive implant, which may include a bioabsorbable implant device that provides a coating of collagen over injured tissue to facilitate healing.

FIG. 1 illustrates that the repair implant 10 may include a generally rectangular configuration, other shapes can be used, as desired. Further, the base 16 may include a single layer. However, while the repair implant 10 is illustrated as having a single layer base 16, it is contemplated that the repair implant 10 may include any number of layers desired, such as, but not limited to, one, two, three, four, or more layers. The base 16 may include both mechanical properties (e.g., strength, stiffness, etc.) to support the load on the repair implant 10 upon initial implantation in addition to biological properties (e.g., collagen) that may facilitate rapid tissue ingrowth.

Further, the sheet-like base 16 may include a longitudinal dimension “L”, a lateral dimension “W” and a thickness “T”. In some embodiments, the lateral dimension W may be about 20 millimeters (mm) to about 50 mm, the longitudinal dimension L may be about 25 mm to about 50 mm and the thickness T may be about 0.5 mm to about 5 mm. It is contemplated that the thickness T of the implant 16 may be thicker when hydrated. Upon implantation, the longitudinal dimension L may extend generally in, or parallel to, the load bearing direction of the soft tissue (e.g., tendon) for which the implant 10 is supporting.

In some examples, the base 16 may include a bioinductive material including, but not limited to collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers. For example, the base 16 may include reconstituted collagen manufactured from highly purified type 1 collagen from bovine tendons. However, other sources of collagen may be used as well. In some embodiments, the bioinductive base 16 may include a lattice structure that can be 3D printed in a flat 2-dimensional pattern or a 3-dimensional pattern. While 3D printing is one illustrative example, other suitable manufacturing techniques may be utilized to manufacture the base 16. For example, a lattice structure and/or other structures described herein may be formed by molding which may be punched, woven, etc. to form the desired arrangement. The bioinductive base 16 may be porous or included pathways to encourage tissue in-growth. In some embodiments, the sheet-like structure of the bioinductive base 16 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding. As will be discussed in greater detail herein, additional (e.g., multiple) layers may be added to reinforce and/or strength the base 16.

FIG. 1 further illustrates that the repair implant 10 may further include one or more portions of feltable material positioned on the upper surface 20 of the base 16. For example, FIG. 1 illustrates the implant 10 may include a first strip of feltable material 22 positioned along the first end region 12 of the implant 10 and a second strip of feltable material 24 positioned along the second end region 14 of the implant 10. In the example illustrated in FIG. 1, each of the first strip of feltable material 22 and the second strip of feltable material 24 may extend across the width W of the base 16. However, it can be appreciated that the first strip of feltable material 22, the second strip of feltable material 24 or both the first strip of feltable material 22 and the second strip of feltable material 24 may extend partially across the width of the base 16. Additionally, it can be appreciated that each of the first strip of feltable material 22 and the second strip of feltable material 24 may include a width “X”, which may be about 0.1 mm to about 20 mm, or about 0.5 mm to about 18 mm, or about 1 mm to about 16 mm, or about 4 mm to about 12 mm, or about 6 mm to about 10 mm. Additionally, it can be appreciated that each of the first strip of feltable material 22 and the second strip of feltable material 24 may include a thickness “Y”, which may be about 0.1 mm to about 6 mm, or about 0.2 mm to about 2 mm, or about 0.3 mm to about 2 mm, or about 0.4 mm to about 1 mm or about 0.5 mm to about 1.5 mm, or about 0.75 mm.

In some examples, the feltable material 22, 24 may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material 22, 24 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material 22, 24 may include combinations of any of the materials disclosed. For example, the feltable material 22, 24 may be formed from a combination of any of natural fibers, synthetic fibers, biodegradable fibers, collagen fibers, or the like. It can be further appreciated that the individual fibers used to construct the feltable material 22, 24 may be randomly aligned with one another to form the non-woven felted structure. In other examples, however, the individual fibers used to construct the feltable material 22, 24 may be aligned with one another to achieve unidirectional stiffness properties with the non-woven felted structure.

In some examples, the thickness of the individual fibers used to construct the feltable material 22, 24 may be about 0.05 mm to about 6 mm, or about 0.1 mm to about 5 mm, or about 0.5 mm to about 2 mm, or about 0.4 mm to about 1 mm or about 0.5 mm to about 1.5 mm. In some examples, the density of the individual fibers used to construct the feltable material 22, 24 may be about 0.025 g/cc to 2 g/cc, or about 0.05 g/cc to 1 g/cc. In some examples, the porosity (void volume) of the individual fibers used to construct the feltable material 22, 24 may be about 40% to 90%.

As discussed herein, an accepted treatment for rotator cuff tears may include reattaching the torn tendon to the footprint of the greater tuberosity. For example, in treating rotator cuff tears, an accepted practice may include the placement of an implant over the repaired tendon to mechanically reinforce the repaired tendon. Therefore, there is an ongoing need to deliver and adequately position medical implants during an arthroscopic procedure in order to treat injuries to the rotator cuff, rotator cuff tendons, or other soft tissue or tendon injuries throughout a body.

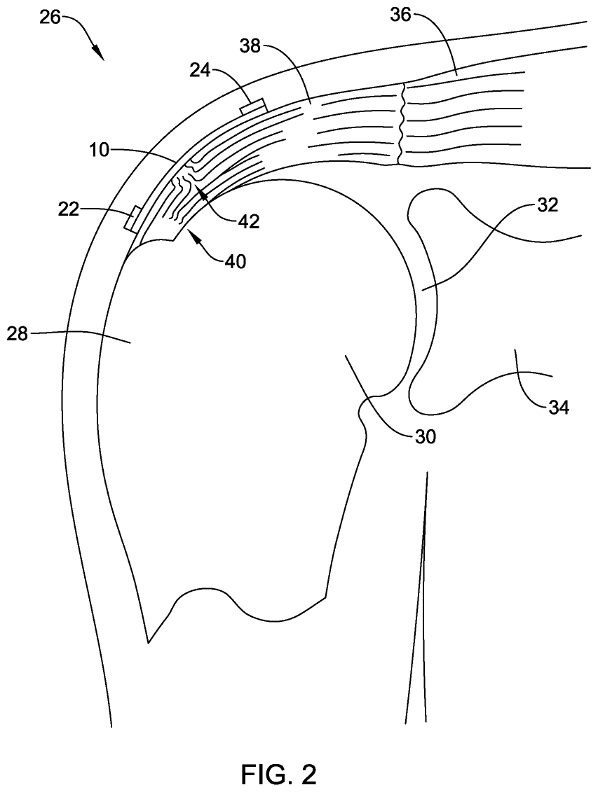

FIG. 2 shows a cross-sectional view of a shoulder 26 including the placement of the example implant 10 described herein. In some examples, delivery of the implant 10 (e.g., bioinductive implant 10) to a target site of a patient may require a user to create an incision in the patient sufficient to access the target implant site. After creating this access site, the user may insert an implant delivery system through the access site and position the distal end of the implant delivery system adjacent the target implant site. The user may then manipulate the implant delivery system to deploy an implant out of a delivery sheath adjacent the target implant site.

FIG. 2 further illustrates the humerus 30 mating with the glenoid fossa 32 of the scapula 34. The glenoid fossa 32 comprises a shallow depression in the scapula 34. A supraspinatus tendon 36 is also shown. These muscles (along with others) control the movement of humerus 30 relative to scapula 34. A distal tendon 38 of the supraspinatus tendon 36 meets the humerus 30 at an insertion point 40.

In FIG. 2, the tendon 38 includes a tear 42 extending partially through the tendon 38. The tear 42 may be described as a partial thickness tear. The depicted partial thickness tear 42 is on the bursal side of the tendon, however, the tear may also be on the opposite or articular side of the tendon 38 and/or may include internal tears to the tendon 38 not visible on either surface. Further, the tendon repair implant 10 may overlay multiple tears. Additionally, while the repair implant 10 has been described as being placed over a tendon, it should be understood that the repair implant 10 may also be used to connect bone to bone. Further, while the tear 42 is illustrated as a partial thickness tear, it should be understood the implant 10 can also be used to repair a full thickness tear. For example, the repair implant 10 may be used to reinforce a repair that is fully approximated or to bridge between the tendon and the bone when the tendon cannot be approximated back to the bone. The repair implants disclosed herein may provide additional tensile strength while maintaining the ability of the implant 10, or portions of the implant 10, to be completely absorbed and remodeled by the body. Further, as discussed herein, in addition to the rotator cuff, the repair implant 10 may be used with other soft tissue repairs, such as, but not limited to the anterior cruciate ligament (ACL), the lateral collateral ligament (LCL), meniscus of the knee, tendons/ligaments of the hip capsule, Achilles tendon and other various ligaments of the shoulder, knee, ankle and hip, for example.

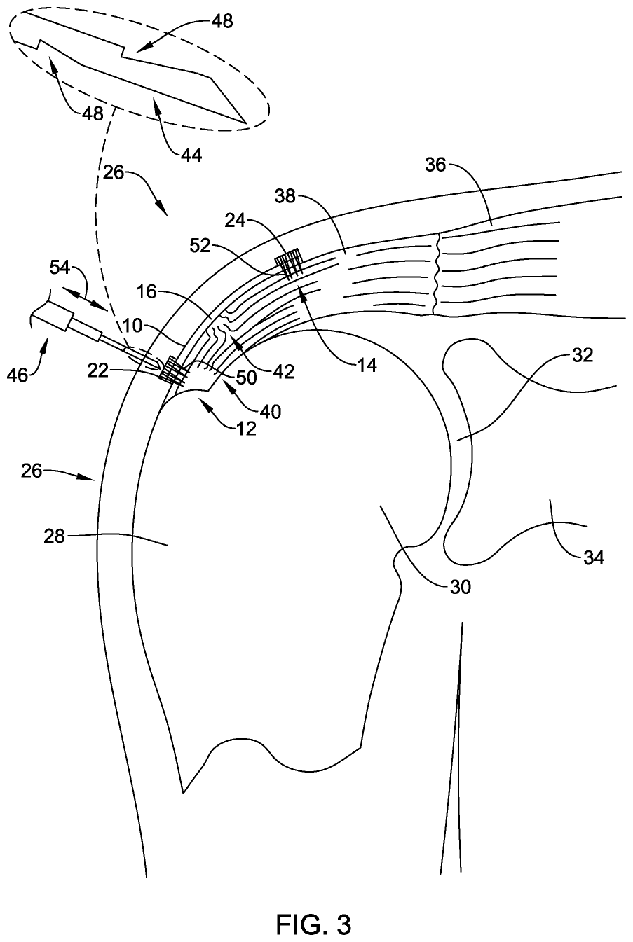

FIG. 3 illustrates the positioning and attachment of the tendon repair implant 10 relative to the partial tear 42 described herein. As illustrated in FIG. 3, the first end region 12 of the repair implant 10 may be configured to be positioned on a portion of the torn tendon which remains fixed to the footprint of the greater tuberosity 28 of the humerus 30 while the second end region 14 of the repair implant 10 may be configured to be positioned on the distal tendon 38 of the supraspinatus tendon 36. It can be further appreciated from FIG. 3 that the base 16 may positioned over the partial thickness tear 42 on the bursal side of the tendon 38.

FIG. 3 further illustrates that attachment of the repair implant 10 to the footprint of the greater tuberosity 28 and the tendon 38 may be achieved via a felting process. For example, FIG. 3 illustrates that a user may introduce a felting needle 44 into the patient's shoulder 26. The felting needle 44 may be attached to a felting drive mechanism 46, whereby the felting drive mechanism 46 is configured to rapidly displace (e.g., oscillate, drive) the needle 44 back and forth along the longitudinal axis of the needle 44 (the longitudinal displacement of the needle 44 is illustrated by the double arrow 54 in FIG. 3). It can be appreciated that the drive mechanism 46 may be driven by a motor (e.g., the drive mechanism may be coupled to a motorized system) or, alternatively, via pneumatic, magnetic, hydraulic or similar systems. FIG. 3 shows the felting drive mechanism 46 including a single needle 44, however, it may include a plurality of needles 44.

It can be appreciated that felting the first end region 12 of the repair implant 10 to a portion of the torn tendon which remains fixed to the footprint of the greater tuberosity 28 of the humerus 30 and the second end region 14 of the repair implant 10 to the distal tendon 38 of the supraspinatus tendon 36 may include a user activating the felting drive mechanism 46 to repeatedly advance the needle 44 through the felted material 22 (to attach the first end region 12 of the repair implant 10 to a portion of the torn tendon which remains fixed to the footprint of the greater tuberosity 28 of the humerus 30) and through the felted material 24 (to attach the second end region 14 of the repair implant 10 to the distal tendon 38 of the supraspinatus tendon 36) such that some of the fibers 50 of the felted material 22 are pushed and/or pulled into a portion of the torn tendon which remains fixed to the footprint of the greater tuberosity 28 and the fibers 52 of the felted material 24 are pushed and/or pulled into the tendon 38 via the needle 44 to produce a strong, robust attachment region between the felted material 22 and a portion of the torn tendon which remains fixed to the footprint of the greater tuberosity 28 and the felted material 24 and the tendon 38. The felting techniques described herein may be configured to provide attachment regions which more uniformly distribute forces which may be imparted to the repair implant 10 (and variations thereof disclosed herein) during the healing and injury rehabilitating process. More uniform distribution of imparted forces may reduce the chance that the repair implant will be dislodged, disengaged, or prematurely removed from the target implantation site.

The detailed view of FIG. 3 illustrates that the needle 44 may include one or more barbs 48 spaced apart from one another along the longitudinal axis of the needle 44. The barbs 48 may be configured such that they engage the fibers 50, 52 of the feltable material 22, 24, when the needle 44 is being displaced along its longitudinal axis. Accordingly, the barbs 48 may push and/or pull the fibers 50, 52 in the longitudinal “stitching” direction of the needle 44. For example, when the needle 44 penetrates the feltable material 22, 24, the needle 44 pushes and/or pulls a number of fibers 50, 52 into a portion of the torn tendon which remains fixed to the footprint of the greater tuberosity 28 and the tendon 38, respectively. It can be appreciated that increasing both the total number of fibers 50, 52 and the depth to which the needle 44 pushes/pulls those fibers 50, 52 into a portion of the torn tendon which remains fixed to the footprint of the greater tuberosity 28 and the tendon 38, respectively, will strengthen the mechanical connection between the first end region 12 of the implant 10 and a portion of the torn tendon which remains fixed to the footprint of the greater tuberosity 28 and the second end region 14 of the implant 10 and the tendon 38. Accordingly, it can be appreciated that the felting drive mechanism 46 may be configured to allow a user to adjust the depth of the back and forth movement of the needle 44 so that, depending on the specific medical procedure being performed, the depth and oscillation speed of the needle 44 may be adjusted by the user.

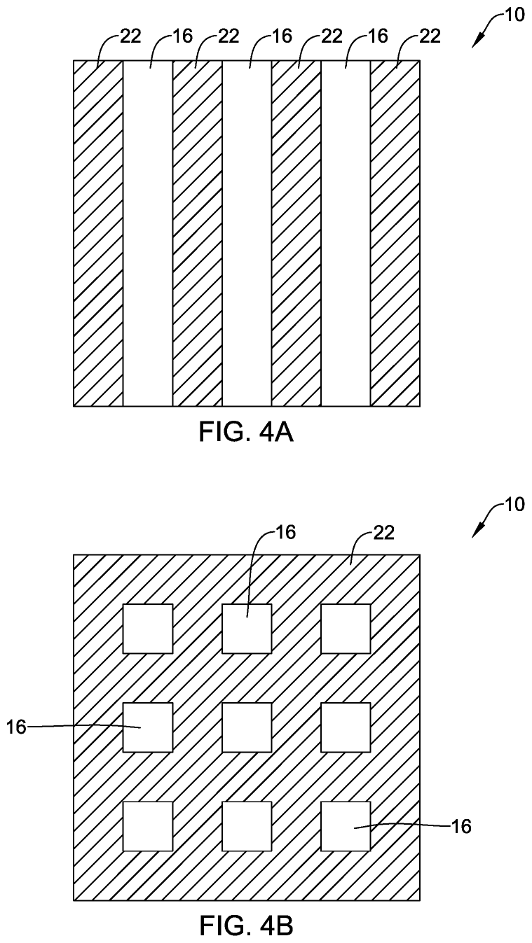

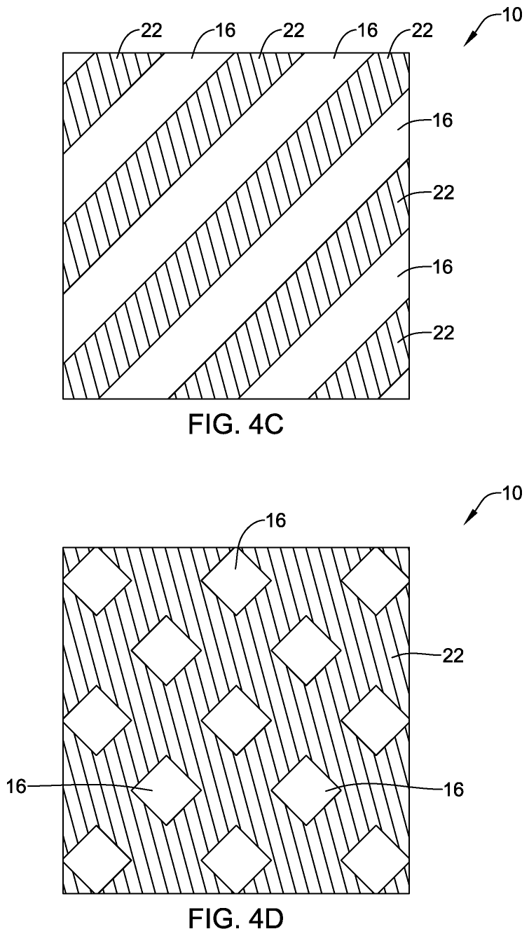

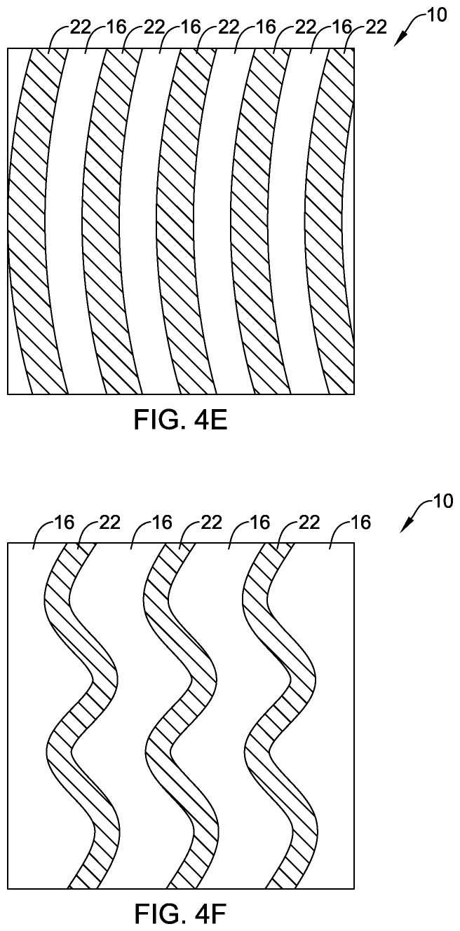

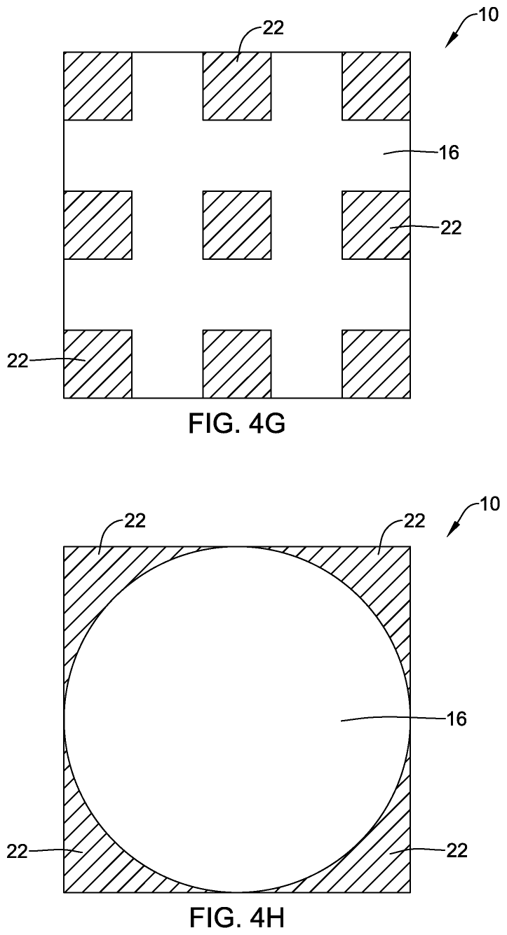

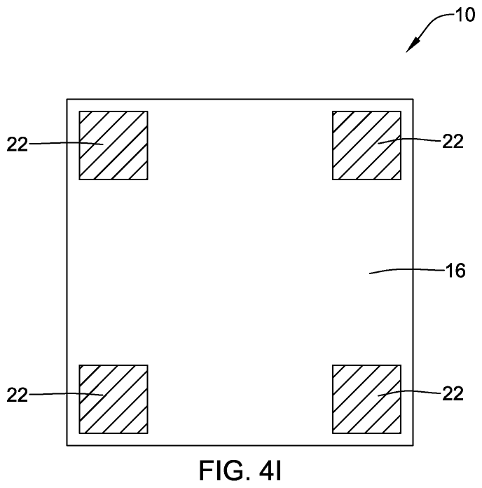

FIGS. 4A-4I schematically depict various example arrangements of the base 16 and the top layer of feltable material 22 of the implant 10. These examples are in addition to the example arrangement of the top layer of feltable materials 22, 24 and the base 16 described with respect to FIG. 1. The example arrangements of the base 16 and feltable layer 22 shown in FIGS. 4A-4I are schematic representations that depict alternative arrangements. The edges/boundaries shown in FIGS. 4A-4I are intended to mimic the edge/boundaries of the pattern/arrangement shown in FIG. 1, or a variation thereof, and can be utilized in any of the devices disclosed as alternatives of the base/top layer.

FIGS. 4A-4I describe an implant which includes feltable material on a top surface of a base 16. It can be appreciated that the feltable material described with respect to the examples disclosed in FIGS. 4A-4I may include feltable material on the bottom surface of a base 16, or on both the top surface and the bottom surface of a base 16.

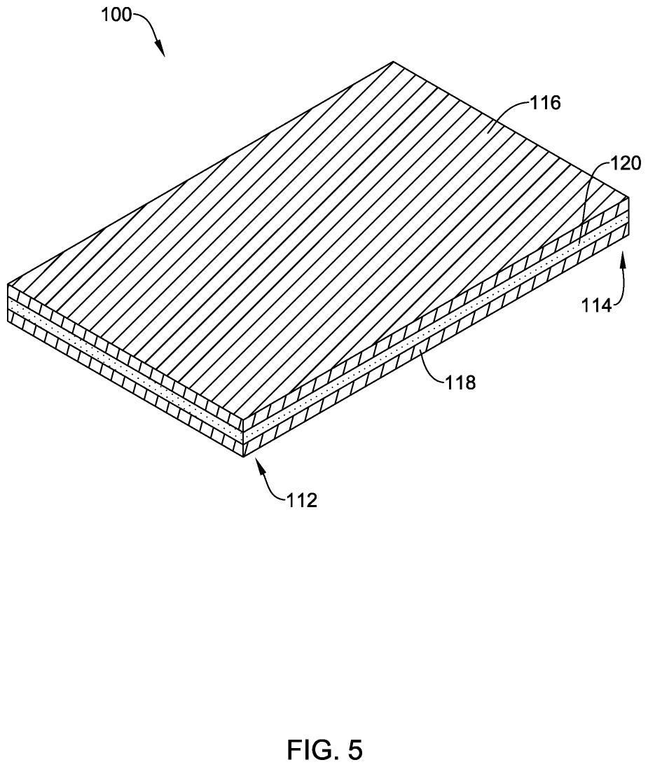

FIG. 5 illustrates a repair implant 100. The repair implant 100 may include a first end region 112 and a second end region 114 opposite the first end region 112. FIG. 5 further illustrates that the repair implant 100 may include a top layer 116, a bottom layer 118 and a middle layer 120 positioned between (e.g., sandwiched between) the top layer 116 and the bottom layer 118. One or more of the top layer 116, the middle layer 120 and/or the bottom layer 118 may have a thickness of about 0.5 mm to about 5 mm. Additionally, in some examples, the middle layer 120 may include a biologic material. The biologic material may include collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers.

Additionally, it can be appreciated that each of the top layer 116 and the bottom layer 118 may include a feltable material. Like that described herein with respect to FIG. 1, the individual fibers forming the top layer 116 and the bottom layer 118 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the top layer 116 and the bottom layer 118 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material forming the top layer 116 and the bottom layer 118 may include combinations of any of the materials disclosed. For example, the feltable material forming the top layer 116 and the bottom layer 118 may be formed from a combination of any of natural fibers, synthetic fibers, biodegradable fibers, collagen fibers, or the like.

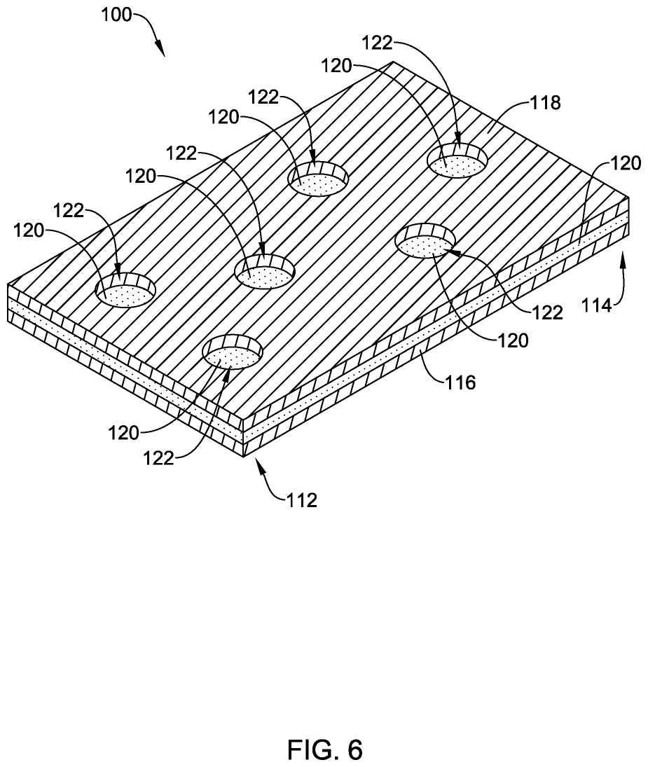

FIG. 6 is a perspective view of the repair implant 100 showing the bottom layer 118. FIG. 6 illustrates that the bottom layer 118 may include one or more apertures 122 (e.g., openings, spaces, channels, etc.) extending through the wall of the feltable material forming the bottom layer 118. FIG. 6 illustrates the apertures 122 having a circular shape. However, it is contemplated that the apertures 122 may include a variety of shapes such as square, rectangular, ovular, crescent-shaped, triangular, polygonal, etc. Additionally, the apertures 122 may include a diameter of about 1 mm to about 10 mm, or about 2 mm to about 8 mm, or about 3 mm to about 7 mm, or about 5 mm.

FIG. 6 further illustrates that apertures 122 in the bottom layer 118 extend through the bottom layer 118 to the middle layer 120 such that the middle layer 120 (which may include collagen or other biologic material) is exposed. It can be appreciated that when the implant 100 is positioned at a target site (e.g., on the supraspinatus tendon during a rotator cuff repair), the top layer 116 and the bottom layer 118 may be attached to the target tissue (e.g., tendon, bone) using the felting techniques described herein. It can be further appreciated that when the implant 100 is positioned at a target site (e.g., on the supraspinatus tendon during a rotator cuff repair) the apertures 122 may be positioned on the bursal side of the tendon, whereby the collagen or other biologic material forming the middle layer 120 may pass through the apertures 122 and contact the tissue forming the tendon after the repair implant 100 has been attached to the target tissue using any of the felting techniques described herein.

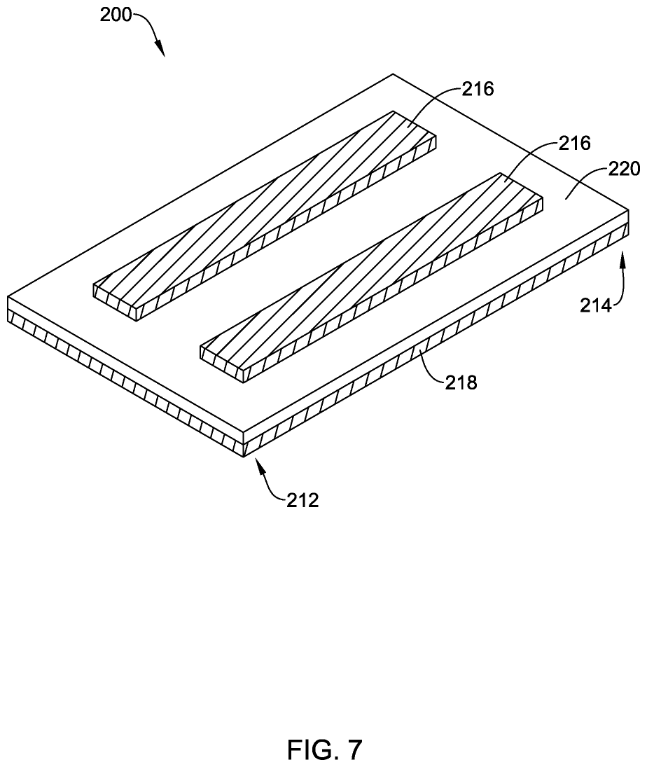

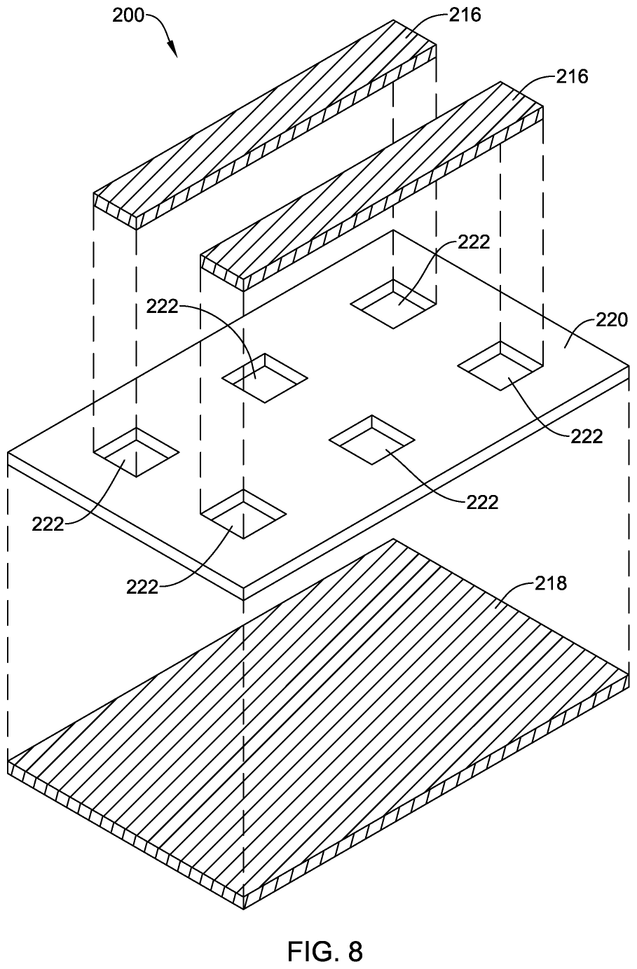

FIG. 7 illustrates a repair implant 200. The repair implant 200 may include a first end region 212 and a second end region 214 opposite the first end region 212. FIG. 7 further illustrates that the repair implant 200 may include two top layer feltable elements 216, a bottom layer 218 and a middle layer 220 positioned between the bottom layer 218 and the feltable elements 216. One or more of the feltable elements 216, the middle layer 220 and/or the bottom layer 218 may have a thickness of about 0.5 mm to about 5 mm. Additionally, in some examples, the middle layer 220 may include a biologic material. The biologic material may include collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers.

Additionally, it can be appreciated that each of the feltable elements 216 and the bottom layer 218 may include a feltable material. Like that described herein with respect to FIG. 1, the individual fibers forming the feltable elements 216 and the bottom layer 218 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the feltable elements 216 and the bottom layer 218 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material forming the feltable elements 216 and the bottom layer 218 may include combinations of any of the materials disclosed. For example, the feltable material forming the feltable elements 216 and the bottom layer 218 may be formed from a combination of any of natural fibers, synthetic fibers, biodegradable fibers, collagen fibers, or the like.

FIG. 8 is an exploded view of the repair implant 200. FIG. 8 illustrates that the middle layer 218 may include one or more apertures 222 (e.g., openings, spaces, channels, etc.) extending through the middle layer 218. FIG. 8 illustrates the apertures 222 having a square shape. However, it is contemplated that the apertures 222 may include a variety of shapes such as circular, rectangular, ovular, crescent-shaped, triangular, polygonal, etc. Additionally, the apertures 222 may include a length and width of about 1 mm to about 10 mm, or about 2 mm to about 8 mm, or about 3 mm to about 7 mm, or about 5 mm.

Further, FIG. 8 illustrates that the feltable elements 216 may be aligned with the apertures 222 such that feltable elements 216 overlay the two rows (e.g., longitudinal rows) of apertures 222 when the feltable elements 216 are positioned along the middle layer 220. It can be appreciated that attaching the feltable elements 216, the middle layer 220 and the bottom layer to one another (e.g., to form the complete repair implant 200), the feltable elements 216 and the bottom layer 218 of feltable material may be ultrasonically welded together through the apertures 222. In other words, the middle layer 220 may be sandwiched and held in place via the feltable elements 216 and the bottom layer 218 of feltable material being ultrasonically welded together. In other examples, the feltable elements 216 and the bottom layer 218 of feltable material may be “pre-felted” together through the apertures 222. In other words, the middle layer 220 may be sandwiched and held in place via the feltable elements 216 and the bottom layer 218 of feltable material being pre-felted together. Pre-felting may include using the felting techniques described herein to felt together the feltable elements 216 and the bottom layer 218 of feltable material.

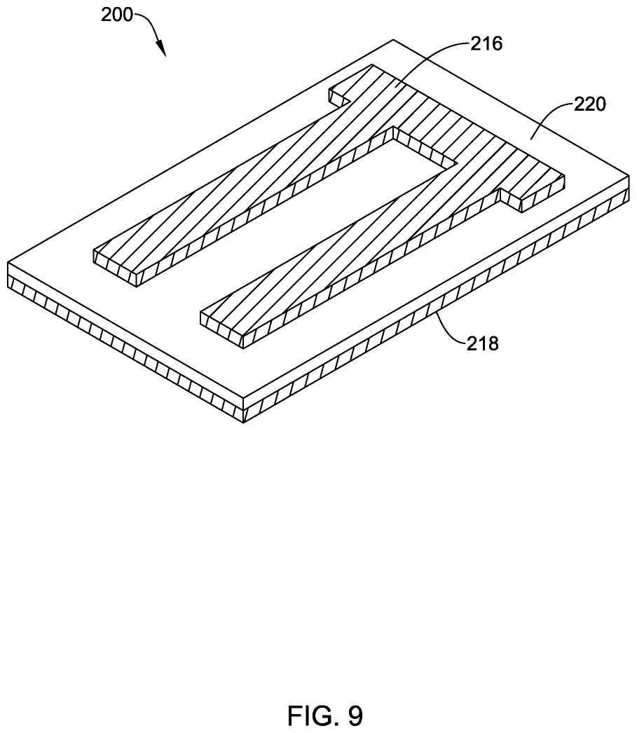

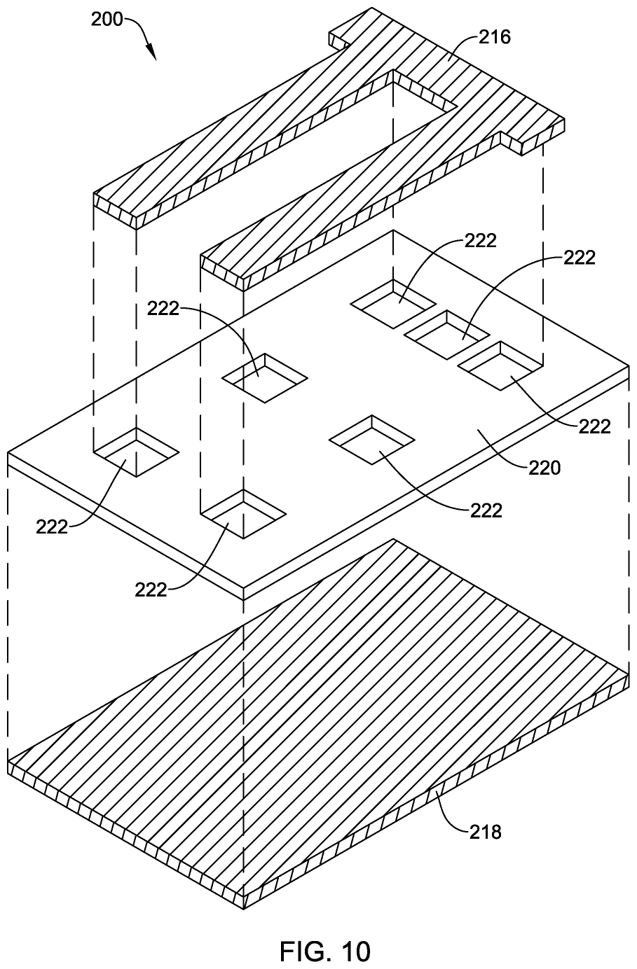

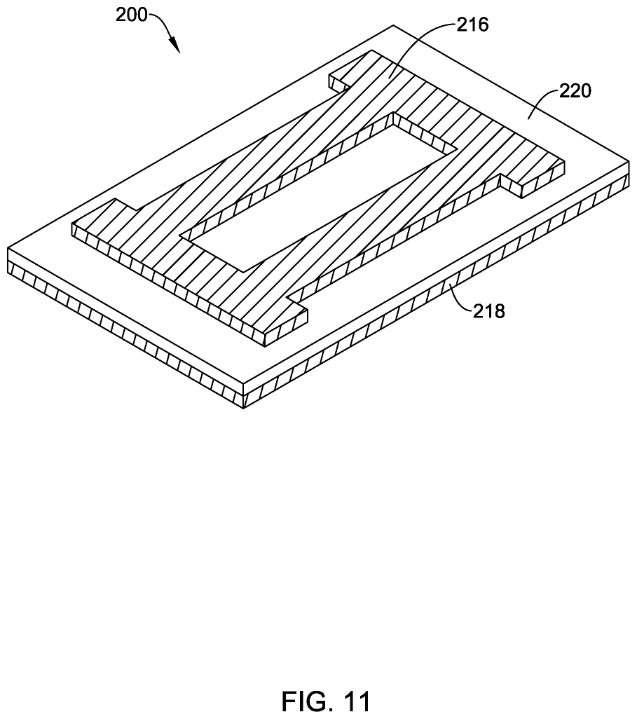

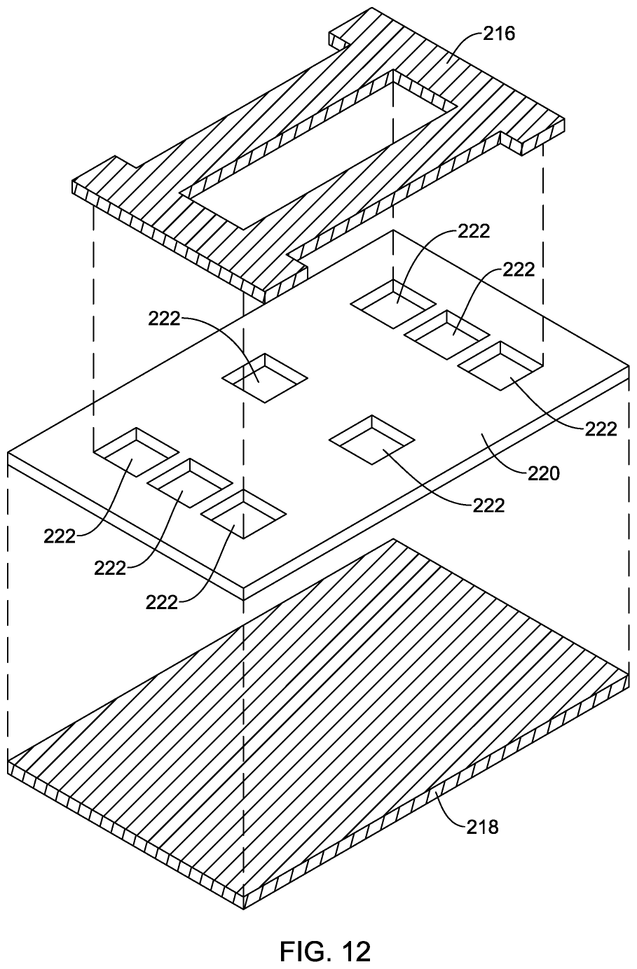

FIGS. 9-12 schematically depict various example arrangements of the feltable element(s) 216, the middle biologic layer 220 (including various arrangements of the apertures 222) and the bottom layer of feltable material 218. These examples are in addition to the example arrangement of the feltable elements 216, the middle biologic layer 220 (including various arrangements of the apertures 222) and the bottom layer of feltable material 218 described with respect to FIGS. 7-8. The example arrangements of the feltable elements 216, the middle biologic layer 220 (including various arrangements of the apertures 222) and the bottom layer of feltable material 218 shown in FIGS. 9-12 are schematic representations that depict alternative arrangements. The edges/boundaries shown in FIGS. 9-12 are intended to mimic the edge/boundaries of the pattern/arrangement shown in FIGS. 7-8, or a variation thereof, can be utilized in any of the devices disclosed as alternatives of the feltable elements 216, the middle biologic layer 220 (including various arrangements of the apertures 222) and the bottom layer of feltable material 218.

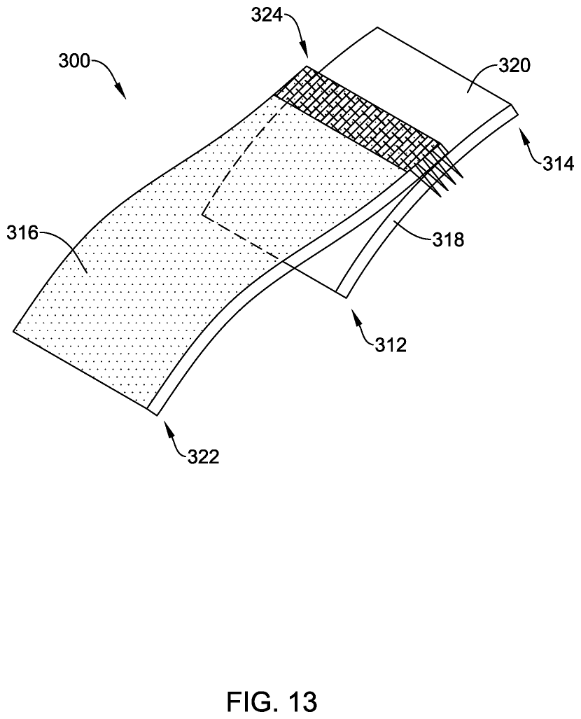

FIG. 13 illustrates a repair implant 300. The repair implant 300 may include a biologic component 320 having a first end region 312 and a second end region 314. The biologic component 320 may include collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers.

Additionally, FIG. 13 illustrates that the repair implant 300 may also include feltable element 316 (e.g., a feltable strip of material) having a first end region 324 and a second end region 322. Like that described herein with respect to FIG. 1, the individual fibers forming the feltable element 316 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the feltable element 316 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material forming the feltable element 316 may include combinations of any of the materials disclosed. For example, the feltable material forming the feltable element 316 may be formed from natural fibers, synthetic fibers, biodegradable fibers, collagen fibers, absorbable polymers, non-absorbable polymers, combinations thereof or the like.

FIG. 13 illustrates that a portion of the first end region 324 of the feltable element 316 may be attached to the biologic component 320 using felting techniques described herein. For example, FIG. 13 illustrates a plurality of individual fibers of the feltable element 316 after the fibers been passed through the biologic using needle-driven felting techniques as described herein. It can be appreciated that utilizing a felting attachment technique may create a strong attachment region that spans the width of both the feltable element 316 and the biologic implant 320. In other examples, feltable element 316 may be attached to the biologic implant 320 via one or more sutures.

In some examples, it can be appreciated that the feltable element 316 may be pre-felted to the biologic component 320 prior to the repair implant 300 being inserted and implanted within a patient. It can be further appreciated that when being utilized in a rotator cuff repair, for example, the biologic component 320 may be attached to the supraspinatus tendon and the unattached portion of the feltable element 316 (including the second end region 322) may be attached (e.g., felted, sutured, anchored or combinations of felting, suturing, anchoring, etc.) to the footprint of the greater tuberosity.

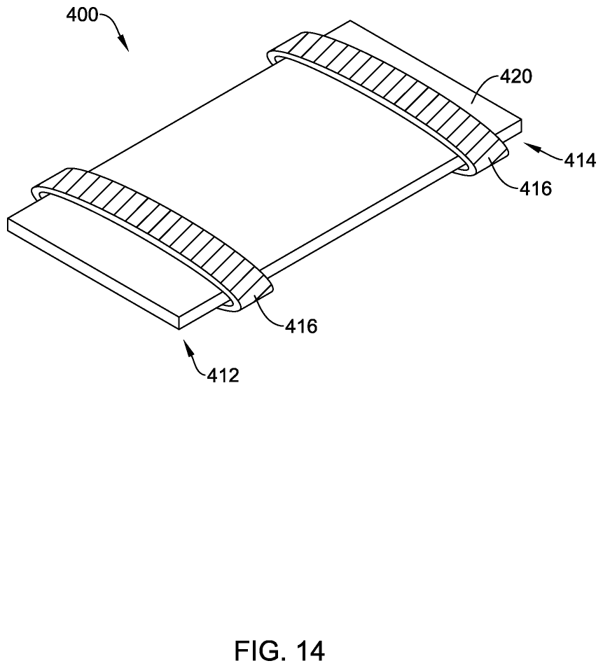

FIG. 14 illustrates a repair implant 400. The repair implant 400 may include a biologic component 420 having a first end region 412 and a second end region 414. In some instances, the biologic component 420 may be described as a collagen implant. The biologic component 420 may include collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers.

Additionally, FIG. 14 illustrates that the repair implant 400 may also include feltable elements 416. FIG. 14 illustrates the repair implant 400 including two feltable elements 416, however, it can be appreciated that the repair implant 400 may include 1, 2, 3, 4, 5, 6 or more feltable elements 416. As illustrated in FIG. 14, each of the feltable elements 416 may extend circumferentially around the biologic component 420 along an axis transverse to the longitudinal axis of the biologic component 420. In other examples, the feltable elements 416 may extend circumferentially around the biologic component 420 along an axis parallel to the longitudinal axis of the biologic component 420. In yet other examples, feltable elements 416 may extend circumferentially around the biologic component 420 along an axis transverse to the longitudinal axis and an axis parallel to the longitudinal axis of the biologic component 420. Additionally, in some examples, the feltable elements 416 may be positioned along the first end region 412, the second end region 414, or both the first end region 412 and the second end region 414 of the biologic component 420. It is also contemplated that the feltable elements 416 may be disposed at any location along the longitudinal axis of the biologic component 420.

Like that described herein with respect to FIG. 1, the individual fibers forming the feltable elements 416 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the elements 416 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material forming the elements 416 may include combinations of any of the materials disclosed. For example, the feltable material forming the elements 416 may be formed from a combination of any of natural fibers, synthetic fibers, biodegradable fibers, collagen fibers, or the like.

It can be appreciated that the feltable elements 416 may be attached to the biologic component 420 using felting techniques described herein. For example, the individual fibers of the feltable elements 416 may be passed through the biologic component 420 using needle-driven felting techniques described herein. In other examples, the feltable elements 416 may be attached to the biologic component 420 via one or more sutures.

In some examples, it can be appreciated that the feltable elements 416 may be pre-felted to the biologic component 420 prior to the repair implant 400 being inserted and implanted within a patient. It can be further appreciated that when being utilized in a rotator cuff repair, for example, the biologic component 420 may be implanted at the target site via felting one of the feltable bands 416 to the supraspinatus tendon and felting the other of the feltable bands 416 to the to the footprint of the greater tuberosity.

In some examples, a collagen-based repair implant (e.g., the bioinductive collagen implants discussed herein) may include a biologic component (e.g., collagen or a collagen-like material) combined with a plurality of non-woven feltable fibers. It can be appreciated that the feltable fibers may be integrated within a collagen-based, biologic component. The feltable fibers may permit the collagen-based, biologic component to be attached at a target tissue site via felting techniques described herein. Additionally, it can be appreciated that the feltable fibers may strengthen the biologic component without reducing its effectiveness when implanted. For example, the feltable fibers may improve suture retention strength (e.g., enhanced resistance to suture pull-out). The following examples illustrate various repair implants which include a collagen-based, biologic component combined with feltable fibers.

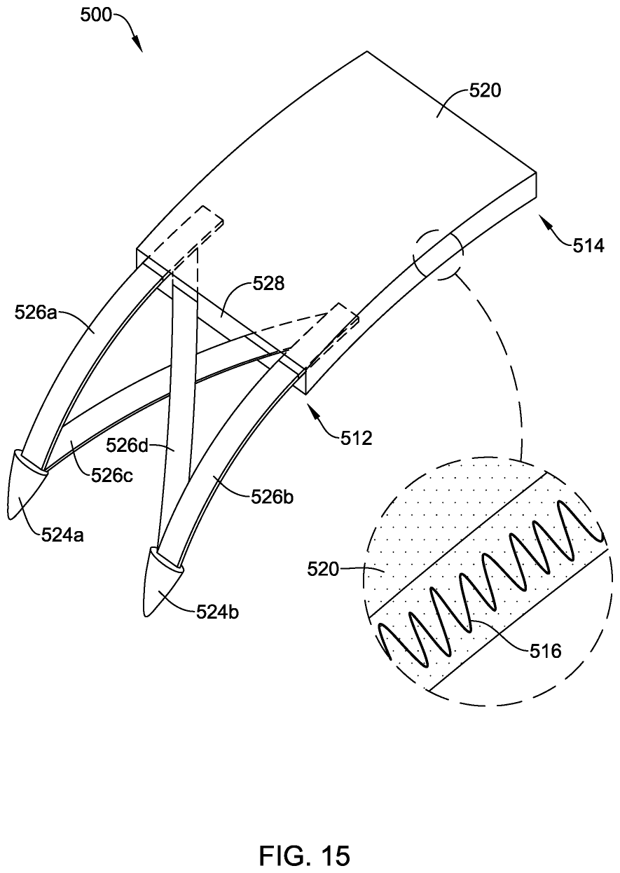

FIG. 15 illustrates a repair implant 500. The repair implant 500 may include a biologic component 520 having a first end region 512 and a second end region 514. The biologic component 520 may include collagen, a collagen element and/or a collagen-like material similar to other bioinductive collagen constructs described herein. In some instances, the biologic component 520 may be described as a collagen implant.

Additionally, the detailed view of FIG. 15 illustrates that the repair implant 500 may also include a plurality of feltable fibers 516 integrated with the biologic component 520. In some examples, the feltable fibers 516 may be uniformly dispersed within the biologic component 520 from the first end region 512 to the second end region 514. In other examples, the feltable fibers 516 may be non-uniformly dispersed within the biologic component 520. In yet other examples, the feltable fibers 516 may be described as a “felting mesh” that is integrated within the biologic component 520.

The feltable fibers 516 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 516 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 516 may include combinations of any of the materials disclosed. For example, the feltable fibers 516 may be formed from a combination of any of natural fibers, synthetic fibers, biodegradable fibers, collagen fibers, or the like.

FIG. 15 further illustrates that the repair implant 500 may also include one or more sutures attached to the biologic component 520. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 15 illustrates that the repair implant 500 may include sutures 526a, 526b, 526c, 526d each of which has a first end which may pass through the face 528 of the biologic component 520 and extend into the biologic component 520. It can be appreciated that the first ends of the sutures 526a, 526b, 526c, 526d may be anchored within a portion of the biologic component 520. Further, in some examples, the individual fibers used to construct the sutures 526a, 526b, 526c, 526d may engage the feltable fibers 516, thereby anchoring the sutures 526a, 526b, 526c, 526d within the feltable mesh formed by the feltable fibers 516. In some examples, the sutures 526a, 526b, 526c, 526d may be slidable relative to the biologic component 520. Other examples of slidable sutures will be described in greater detail herein.

FIG. 15 further illustrates that a second end of the sutures 526a, 526c may be attached to a first anchor 524a (e.g., bone anchor) and that a second end of the sutures 526b, 526d may be attached to a second anchor 524b (e.g., bone anchor). In other examples, the second ends of the sutures 526a, 526c may be attached to a first bone staple and the second ends of the sutures 526b, 526d may be attached to a second bone staple. It can be appreciated that when being utilized in a rotator cuff repair, for example, the first end region 514 of the biologic component 520 may be attached (e.g., felted) to the supraspinatus tendon using felting techniques described herein and the bone anchors 524a, 524b may be attached to the footprint of the greater tuberosity. In other examples, the first end region 514 of the biologic component 520 may be attached to the supraspinatus tendon with anchors, staples, combinations thereof or other suitable attachment technique.

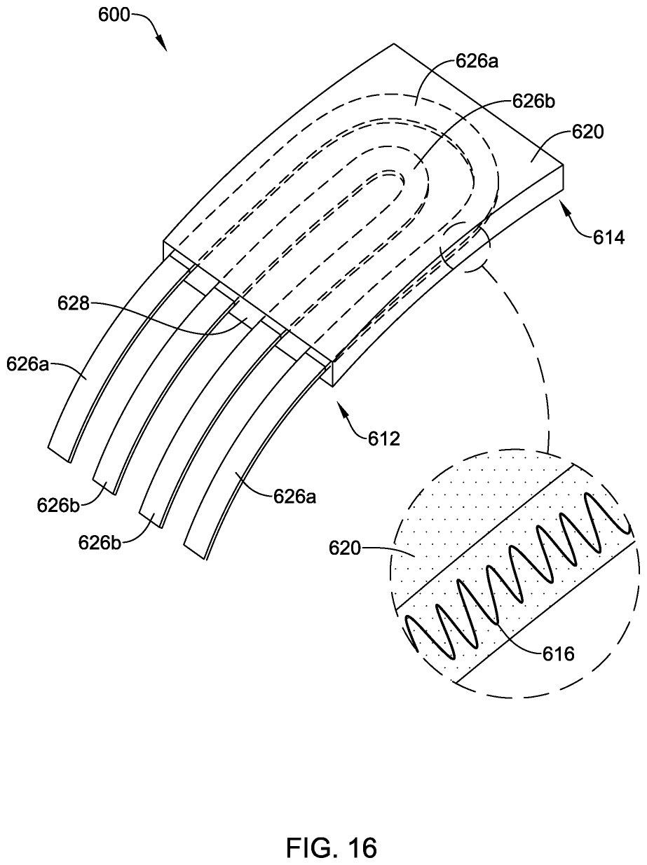

FIG. 16 illustrates a repair implant 600. The repair implant 600 may include a biologic component 620 having a first end region 612 and a second end region 614. The biologic component 620 may include collagen, a collagen element and/or a collagen-like material similar to other bioinductive collagen constructs described herein. In some instances, the biologic component 620 may be described as a collagen implant.

Additionally, the detailed view of FIG. 16 illustrates that the repair implant 600 may also include a plurality of feltable fibers 616 integrated into the biologic component 620. In some examples, the feltable fibers 616 may be uniformly dispersed within the biologic component 620 from the first end region 612 to the second end region 614. In other examples, the feltable fibers 616 may be non-uniformly dispersed within the biologic component 620. In yet other examples, the feltable fibers 616 may be described as a “felting mesh” that is integrated within the biologic component 620.

The feltable fibers 616 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 616 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 616 may include combinations of any of the materials disclosed. For example, the feltable fibers 616 may be formed from a combination of any of natural fibers, synthetic fibers, biodegradable fibers, collagen fibers, or the like.

FIG. 16 further illustrates that the repair implant 600 may also include one or more sutures 626a, 626b attached to the biologic component 620. Each of the sutures 626a, 626b may include a first end, a second end and a medial region extending between the first end and the second end. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

Further, FIG. 16 illustrates that a portion of the medial region of each of the sutures 626a, 626b may pass through the face 628 of the biologic component 620 and extend into the biologic component 620. In some examples, each of the sutures 626a, 626b may form a “U-shape” within the biologic component 620, whereby the medial portion the sutures 626a, 626b forming the U-shape may be anchored within a portion of the biologic component 620. Further, in some examples, the sutures 626a, 626b may engage the feltable fibers 616, thereby anchoring the sutures 626a, 626b within the feltable mesh formed by the feltable fibers 616. It can be appreciated that when being utilized in a rotator cuff repair, for example, the first end region 614 of the biologic component 620 may be attached (e.g., felted) to the supraspinatus tendon using felting techniques described herein and the first and second ends of each of the sutures 626a, 626b may be attached to the footprint of the greater tuberosity using anchors, staples, combinations thereof or other suitable attachment technique. In other examples, the first end region 614 of the biologic component 620 may be attached to the supraspinatus tendon with anchors, staples, combinations thereof or other suitable attachment technique.

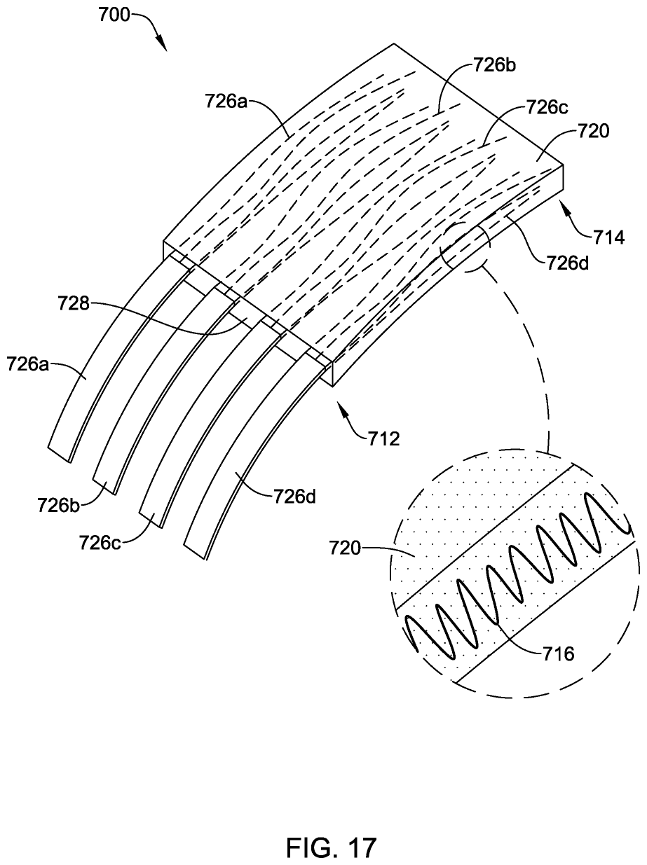

FIG. 17 illustrates a repair implant 700. The repair implant 700 may include a biologic component 720 having a first end region 712 and a second end region 714. The biologic component 720 may include collagen, a collagen element and/or a collagen-like material similar to the bioinductive collagen construct described herein. In some instances, the biologic component 720 may be described as a collagen implant.

Additionally, the detailed view of FIG. 17 illustrates that the repair implant 700 may also include a plurality of feltable fibers 716 integrated into the biologic component 720. In some examples, the feltable fibers 716 may be uniformly dispersed within the biologic component 720 from the first end region 712 to the second end region 714. In other examples, the feltable fibers 716 may be non-uniformly dispersed within the biologic component 720. In yet other examples, the feltable fibers 716 may be described as a “felting mesh” that is integrated within the biologic component 720.

The feltable fibers 716 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 716 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 716 may include combinations of any of the materials disclosed. For example, the feltable fibers 716 may be formed from a combination of any of natural fibers, synthetic fibers, biodegradable fibers, collagen fibers, or the like.

FIG. 17 further illustrates that the repair implant 700 may also include one or more sutures attached to the biologic component 720. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 17 illustrates that the repair implant 700 may include sutures 726a, 726b, 726c, 726d each of which have a first end and a second end opposite the first end, whereby the first end of each suture 726a, 726b, 726c, 726d may pass through the face 728 of the biologic component 720 and extend into the biologic component 720.

It can be further appreciated that each of the sutures 726a, 726b, 726c, 726d may each be formed from a plurality of fibers, whereby the plurality of fibers of the first end of each of the sutures 726a, 726b, 726c, 726d may spread out and anchor within a portion of the biologic component 720. Further, in some examples, the fibers of each of the sutures 726a, 726b, 726c, 726d may engage the feltable fibers 716, thereby anchoring the sutures 726a, 726b, 726c, 726d within the feltable mesh formed by the feltable fibers 716. It can be appreciated that when being utilized in a rotator cuff repair, for example, the first end region 714 of the biologic component 720 may be attached (e.g., felted) to the supraspinatus tendon using felting techniques described herein and the second ends of each of the sutures 726a, 726b, 726c, 726d may be attached to the footprint of the greater tuberosity using anchors, staples, combinations thereof or other suitable attachment technique. In other examples, the first end region 714 of the biologic component 720 may be attached to the supraspinatus tendon with anchors, staples, combinations thereof or other suitable attachment technique.

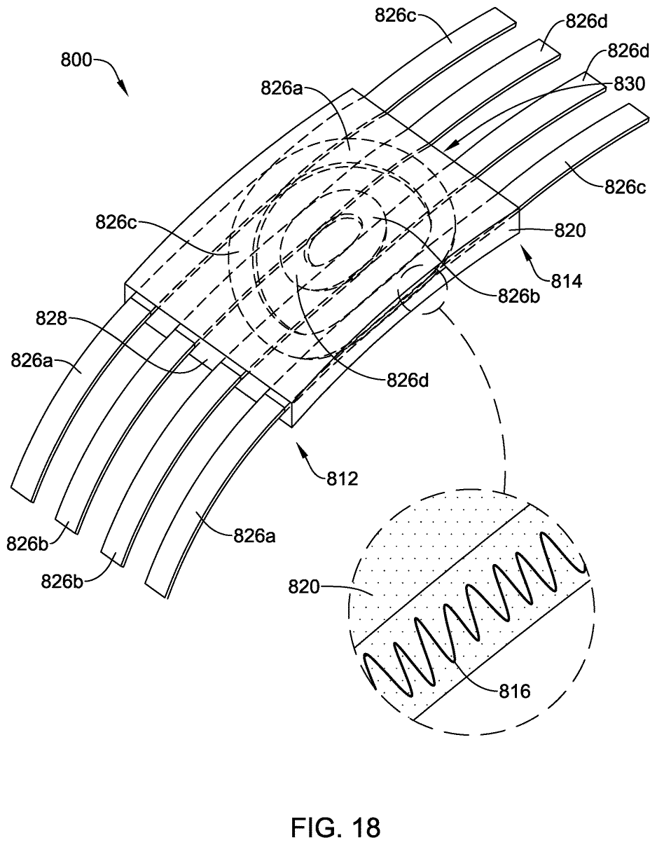

FIG. 18 illustrates a repair implant 800. The repair implant 800 may include a biologic component 820 having a first end region 812 and a second end region 814. The biologic component 820 may include collagen, a collagen element and/or a collagen-like material similar to the bioinductive collagen construct described herein. In some instances, the biologic component 820 may be described as a collagen implant.

Additionally, the detailed view of FIG. 18 illustrates that the repair implant 800 may also include a plurality of feltable fibers 816 integrated into the biologic component 820. In some examples, the feltable fibers 816 may be uniformly dispersed within the biologic component 820 from the first end region 812 to the second end region 814. In other examples, the feltable fibers 816 may be non-uniformly dispersed within the biologic component 820. In yet other examples, the feltable fibers 816 may be described as a “felting mesh” that is integrated within the biologic component 820.

The feltable fibers 816 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 816 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 816 may include combinations of any of the materials disclosed. For example, the feltable fibers 816 may be formed from a combination of any of natural fibers, synthetic fibers, biodegradable fibers, collagen fibers, or the like.

FIG. 18 further illustrates that the repair implant 800 may also include one or more sutures 826a, 826b, 826c, 826d attached to the biologic component 820. Each of the sutures 826a, 826b, 826, 826d may include a first end, a second end and a medial region extending between the first end and the second end. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

Further, FIG. 18 illustrates that a portion of the medial region of each of the sutures 826a, 826b may pass through a first face 828 of the biologic component 820 and extend into the biologic component 820. FIG. 18 further illustrates that a portion of the medial region of each of the sutures 826c, 826d may pass through a second face 830 (opposite the first face 828) of the biologic component 820 and extend into the biologic component 820.

Additionally, in some examples, each of the sutures 826a, 826b, 826c, 826d may form a “U-shape” within the biologic component 820, whereby the medial portion of the sutures 826a, 826b, 826c, 826d forming the U-shape may be anchored within a portion of the biologic component 820. FIG. 18 illustrates that the direction of the U-shape of the sutures 826a, 826b may be opposite the direction of the U-shape of the sutures 826c, 826d. Further, in some examples, the sutures 826a, 826b, 826c, 826d may engage the feltable fibers 816, thereby anchoring the sutures 826a, 826b, 826c, 826d within the feltable mesh formed by the feltable fibers 816. It can be appreciated that when being utilized in a rotator cuff repair, for example, a portion of the biologic component 820 may be attached (e.g., felted) to the supraspinatus tendon using felting techniques described herein, the first and second ends of each of the sutures 826a, 826b may be attached to the footprint of the greater tuberosity and the first and second ends of each of the sutures 826a, 826b may be attached to tendon, a medial row knotless anchor, tied off to one another, not stitched into tissue, or cut off and not used as applicable, for example.

An advantage of the examples illustrated in FIGS. 16-18 may include that the sutures/fibers shown in the examples of FIGS. 16-18 may more evenly distribute the load across the implants thereby decreasing stress concentrations placed on the implant under loading conditions.

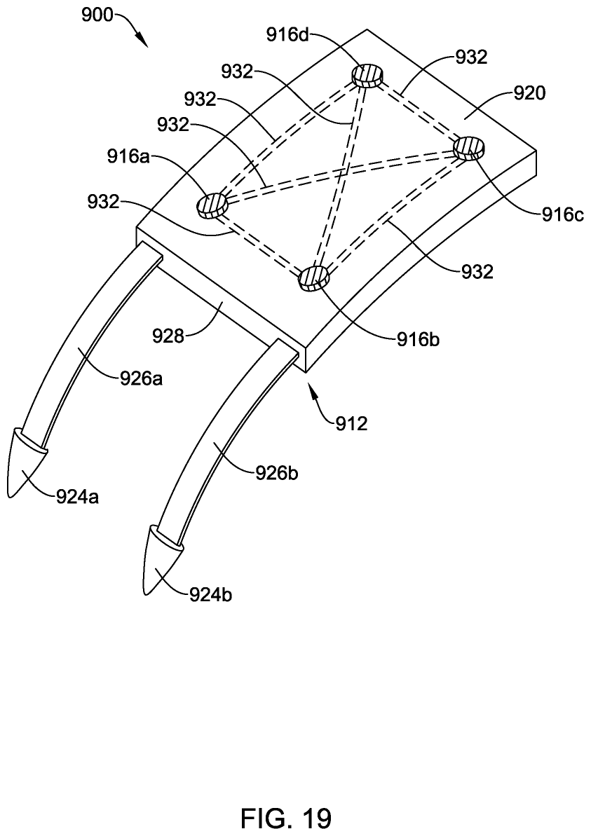

FIG. 19 illustrates a repair implant 900. The repair implant 900 may include a biologic component 920 having a first end region 912 and a second end region 914. The biologic component 920 may include collagen, a collagen element and/or a collagen-like material similar to the bioinductive collagen construct described herein. In some instances, the biologic component 920 may be described as a collagen implant.

Additionally, FIG. 19 illustrates that the repair implant 900 may also include a plurality of feltable anchors 916a, 916b, 916c, 916d positioned on the upper surface of the biologic component 920. FIG. 19 illustrates that the repair implant 900 may include four feltable anchors 916a, 916b, 916c, 916d arranged a substantially rectangular pattern. However, while FIG. 19 illustrates the repair implant 900 having four feltable anchors 916a, 916b, 916c, 916d, it is contemplated that the repair implant 900 may include 1, 2, 3, 4, 5, 6 or more feltable anchors. Additionally, it is contemplated the that the feltable anchors 916a, 916b, 916c, 916d may be arranged along the biologic component 920 in a variety of patterns (e.g., circular, ovular, triangular, etc.).

The feltable anchors 916a, 916b, 916c, 916d may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable anchors 916a, 916b, 916c, 916d may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable anchors 916a, 916b, 916c, 916d may include combinations of any of the materials disclosed. For example, the feltable anchors 916a, 916b, 916c, 916d may be formed from a combination of any of natural fibers, synthetic fibers, biodegradable fibers, collagen fibers, or the like.

FIG. 19 further illustrates that the repair implant 900 may also include one or more sutures attached to the biologic component 920. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 19 illustrates that the repair implant 900 may include sutures 926a, 926b each of which have a first end which may pass through the face 928 of the biologic component 920 and extend into the biologic component 920. It can be appreciated that the first ends of the sutures 926a, 926b may be anchored within a portion of the biologic component 920. Further, in some examples, the individual fibers used to construct the sutures 926a may engage the feltable anchor 916a and the individual fibers used to construct the suture 926b may engage the feltable anchor 916b. In some examples, the sutures 926a, 926b may be sliceable relative to the biologic component 920. Other examples of slidable sutures will be described in greater detail herein.

FIG. 19 further illustrates that repair implant 900 may further include a plurality of reinforcing sutures 932 extending between the feltable anchors 916a, 916b, 916c, 916d. It can be appreciated that the reinforcing sutures 932 may be integrated into the biologic component 920, thereby strengthening the biologic component 920 when implanted at a target tissue site.

FIG. 19 further illustrates that a second end of the suture 926a may be attached to a first bone anchor 924a and a second end of the sutures 926b may be attached to a second bone anchor 924b. In other examples, the second end of the suture 926a may be attached to a first bone staple and the second end of the suture 926b may be attached to a second bone staple. It can be appreciated that when being utilized in a rotator cuff repair, for example, the biologic component 920 may be attached (e.g., felted) to the supraspinatus tendon by felting the feltable anchors 916a, 916b, 916c, 916d to the tendon using any of the felting techniques described herein. Additionally, sutures 926a, 926b may be attached to the footprint of the greater tuberosity via the bone anchors 924a, 924b.

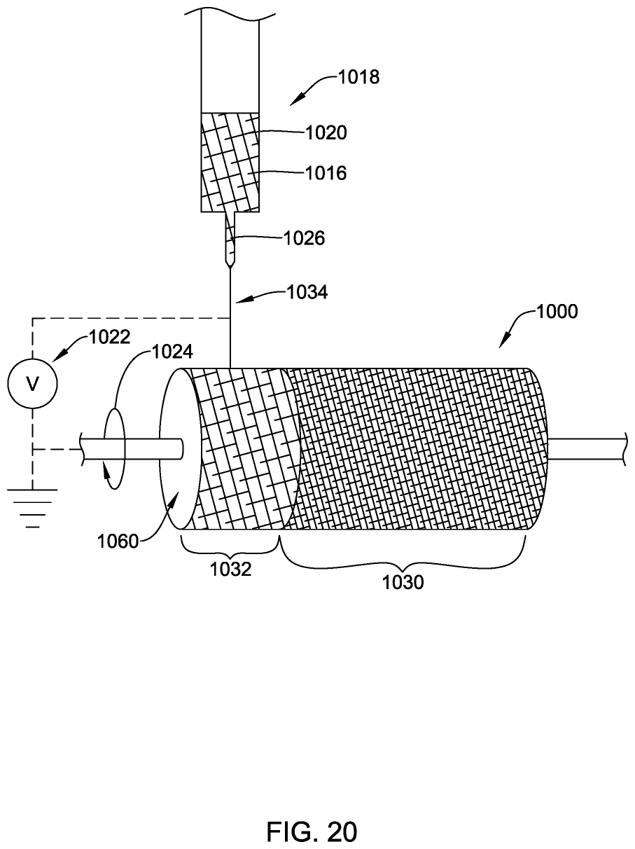

FIG. 20 illustrates a method for constructing a collagen-based repair implant such as the bioinductive collagen implants discussed herein. The collagen-based repair implant shown in FIG. 20 may include a biologic component (e.g., collagen or a collagen-like material) combined with a plurality of non-woven feltable fibers. For example, FIG. 20 illustrates a method for constructing a feltable, electrospun, collagen implant. Electrospinning is a manufacturing technique that produces a fine fiber scaffolding by injecting a polymeric-fiber material (e.g., solution, slurry, blended slurry, viscous collagen-fiber solution, etc.) over a high-voltage field. It can be appreciated that electrospinning feltable fibers with collagen may produce a biologic scaffold whereby the feltable fibers are integrated within the collagen. Further, the feltable fibers may permit the collagen-based, biologic component to be attached at a target tissue site via felting techniques described herein. Additionally, it can be appreciated that the feltable fibers may strengthen the biologic component without reducing its biologic effectiveness when implanted. For example, the feltable fibers may improve suture retention strength (e.g., enhanced resistance to suture pull-out).

FIG. 20 illustrates a feltable, electrospun, collagen implant 1000 being constructed via an electrospinning manufacturing technique. FIG. 20 illustrates a collagen-fiber solution 1034 being ejected from a nozzle 1026 onto a rotating mandrel 1060 (or, alternatively, on a plate) over a high-voltage field 1022. Rotation of the mandrel 1060 (or, alternatively, a plate) by the directional arrow 1024 in FIG. 20. The collagen-fiber solution may be stored in a reservoir 1018 prior to being ejected from the nozzle 1026 onto the mandrel 1060. While FIG. 20 illustrates the collagen-fiber solution being ejected from a single nozzle 1026, it can be appreciated that the process may include more than one nozzle. For example, multiple nozzles may permit a blend of collagen and another biodegradable polymer (or similar material) to be combined with the feltable fibers 1016 to construct the feltable, electrospun, implant 1000.

It can be appreciated that the collagen-fiber solution 1034 may be formed from a blend of collagen 1020 (or a similar, collagen-like material) and feltable fibers 1016. The feltable fibers 1016 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 1016 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 1016 may include combinations of any of the materials disclosed. For example, the feltable fibers 1016 may be formed from a combination of any of natural fibers, synthetic fibers, biodegradable fibers, collagen fibers, or the like.

In some examples, the ratio of the collagen 1020 (or collagen-like material) to feltable fibers 1016 making up the collagen-fiber solution 1034 may be tailored to create a feltable collagen-based fiber structure having specific mechanical properties (e.g., strength, flexibility, etc.), feltability, bioinductivity and/or degradation profiles. For example, the ratio of the collagen 1020 (or collagen-like material) to feltable fibers 1016 making up the collagen-fiber solution 1034 may be about 1-5% collagen to about 95-100% feltable fibers, or about 5-10% collagen to about 90-95% feltable fibers, or about 10-15% collagen to about 85-90% feltable fibers, or about 15-20% collagen to about 80-85% feltable fibers, or about 20-25% collagen to about 75-80% feltable fibers, or about 25-30% collagen to about 70-75% feltable fibers, or about 30-35% collagen to about 65-70% feltable fibers, or about 35-40% collagen to about 60-65% feltable fibers, or about 40-45% collagen to about 55-60% feltable fibers, or about 45-50% collagen to about 45-50% feltable fibers, or about 50% collagen to about 50% feltable fibers. In other examples, the ratio of the ratio of the feltable fibers 1016 to collagen 1020 (or collagen-like material) making up the collagen-fiber solution 1034 may be about 1-5% feltable fibers to about 95-100% collagen, or about 5-10% feltable fibers to about 90-95% collagen, or about 10-15% feltable fibers to about 85-90% collagen, or about 15-20% feltable fibers to about 80-85% collagen, or about 20-25% feltable fibers to about 75-80% collagen, or about 25-30% feltable fibers to about 70-75% collagen, or about 30-35% feltable fibers to about 65-70% collagen, or about 35-40% feltable fibers to about 60-65% collagen, or about 40-45% feltable fibers to about 55-60% collagen. In addition to the ratio of the collagen 1020 (or collagen-like material) to feltable fibers 1016 making up the collagen-fiber solution 1034 the voltage, mandrel rotation speed and ejection rate of the solution may be adjusted to further tailor the specific mechanical properties (e.g., strength, flexibility, etc.), feltability, bioinductivity and/or degradation profiles of the feltable, electrospun, collagen implant 1000.

Additionally, the electrospinning manufacturing technique described herein may permit the construction of a feltable, electrospun, collagen implant 1000 having different density profiles (e.g., ratio of feltable fibers 1016 to collagen 1030) along the longitudinal axis of the implant 1000. For example, FIG. 20 illustrates that the electrospinning manufacturing technique may be utilized to construct an implant 1000 having a first region 1032 whereby the ratio of feltable fibers 1016 to collagen 1020 is less than the ratio of feltable fibers 1016 to collagen 1020 in a second region 1030. In other words, the electrospinning manufacturing technique may be utilized to construct an implant 1000 having first region 1032 whereby the density of feltable fibers 1016 within the feltable-collagen implant 1000 is less than the density of feltable fibers in a second region 1030. It can be appreciated that the electrospinning technique described herein may be utilized to create an implant 1000 having a variety of different densities of feltable fibers 1016, patterns of feltable fibers 1016, and/or regions of varying ratios of feltable fibers 1016 to collagen 1020.

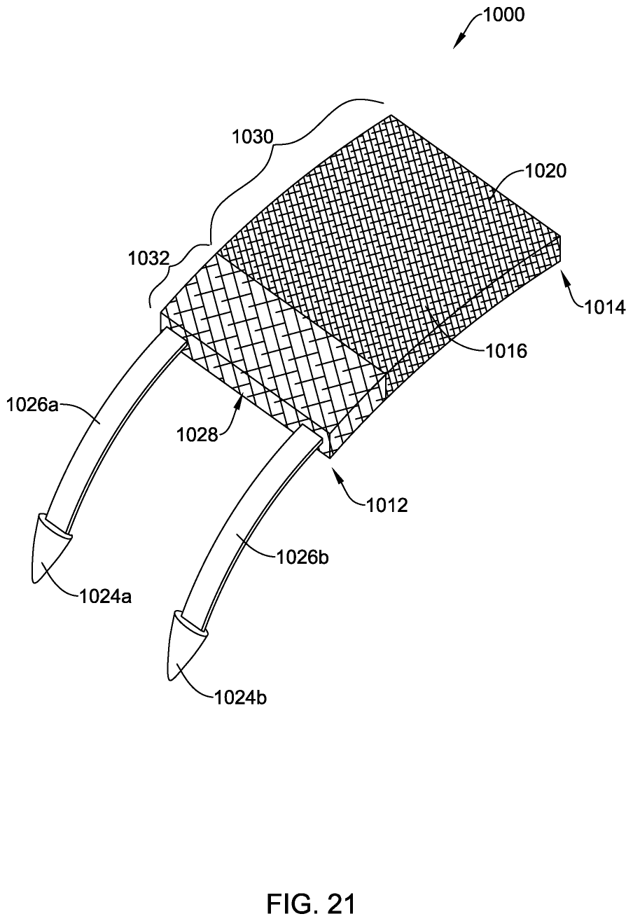

FIG. 21 illustrates a feltable, electrospun, collagen repair implant 1000 constructed using the electrospinning manufacturing technique described herein. The repair implant 1000 may include a biologic component 1020 having a first end region 1012 and a second end region 1014. The biologic component 1020 may be include a feltable fiber and collagen (or collagen element and/or a collagen-like material similar to the bioinductive collagen construct described herein) scaffold. Further, as described herein, the repair implant 1000 may include a first region 1032 whereby the ratio of feltable fibers 1016 to collagen 1020 is less than the ratio of feltable fibers 1016 to collagen 1020 in a second region 1030 (e.g., the density of feltable fibers 1016 is greater in the second region 1030 compared to the first region 1032). Additionally, it can be appreciated that, in some examples, the second end region 1014 of the implant 1000 may be attached to a tendon. Accordingly, configuring the second region 1030 to have a greater density of feltable fibers 1016 may be beneficial because the thicker, stronger, fiber-dense second region 1030 may provide a stronger, reinforced scaffold when felting the second end region 1014 to tendon, or alternatively, using a non-felting attachment technique (e.g., bone anchors, staples, etc.) or some combination of felting and non-felting attachment techniques to attach the second end region 1014 of the implant 1000 to a tendon.

FIG. 21 further illustrates that the repair implant 1000 may also include one or more sutures attached to the biologic component 1020. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 21 illustrates that the repair implant 1000 may include sutures 1026a, 1026b each of which have a first end which may pass through the face 1028 of the biologic component 1020 and extend into the biologic component 1020. It can be appreciated that the first ends of the sutures 1026a, 1026b may be anchored within a portion of the biologic component 1020. Further, in some examples, the individual fibers used to construct the sutures 1026a, 1026b may engage the feltable fibers 1016 of the biologic component 1020.

FIG. 21 further illustrates that a second end of the suture 1026a may be attached to a first bone anchor 1024a and that a second end of the sutures 1026b may be attached to a second bone anchor 1024b. In other examples, the second end of the suture 1026a may be attached to a first bone staple and the second end of the suture 1026b may be attached to a second bone staple. It can be appreciated that when being utilized in a rotator cuff repair, for example, the first end region 1014 of the biologic component 1020 may be attached (e.g., felted) to the supraspinatus tendon using any of the felting techniques described herein and the bone anchors 1024a, 1024b may be attached to the footprint of the greater tuberosity.

Further, it can be appreciated that, in general, the electrospinning manufacturing techniques described herein may integrate collagen (or similar material) onto an existing feltable scaffold. Additionally, the electrospinning manufacturing techniques described herein may integrate feltable fibers onto an existing collagen (or similar material) scaffold. Additionally, the electrospinning manufacturing techniques described herein may integrate feltable fibers onto a slurry of viscous existing collagen (or similar material) scaffold that is able to entrap the feltable fibers and form a collagen construct. Additionally, the electrospinning manufacturing techniques described herein may be utilized to form a feltable fiber scaffold which does not include a collagen component.

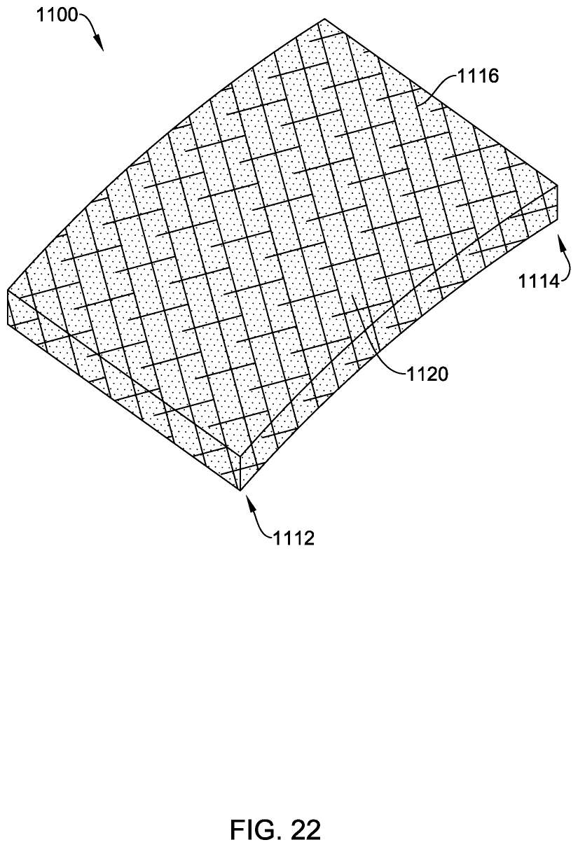

FIG. 22 illustrates a repair implant 1100. The repair implant 1100 may include a first end region 1112 and a second end region 1114 opposite the first end region 1112. FIG. 22 further illustrates that the repair implant 1100 may be formed by adding a biologic material 1120 to a pre-formed, feltable fiber scaffold 1116. The biologic material 1120 may include collagen, a collagen element and/or a collagen-like material similar to other bioinductive collagen constructs described herein.

Additionally, it can be appreciated that individual fibers forming the feltable fiber scaffold 1116 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the feltable fiber scaffold 1116 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fiber scaffold 1116 may include combinations of any of the materials disclosed. For example, the feltable fiber scaffold 1116 may be formed from a combination of any of natural fibers, synthetic fibers, biodegradable fibers, collagen fibers, or the like. It can be appreciated that when the implant 1100 is positioned at a target site (e.g., on a supraspinatus tendon during a rotator cuff repair), the first end region 1112 and/or the second end region 1114 may be attached to a target tissue using the felting techniques described herein.

In some examples, adding a biologic material 1120 (e.g., collagen or a collagen-like material, Stravix®, Gravix®, etc.) to a pre-formed, feltable fiber scaffold 1116 may include soaking the fiber scaffold 1116 in a collagen slurry during a manufacturing process, potting collagen to the fiber scaffold 1116, spraying collagen to the fiber scaffold 1116, adhering collagen to the fiber scaffold 1116, suturing collagen to the fiber scaffold 1116, pre-felting collagen to the fiber scaffold 1116, overlaying the fiber scaffold 1116 onto a collagen construct during a manufacturing process, or any combination thereof.

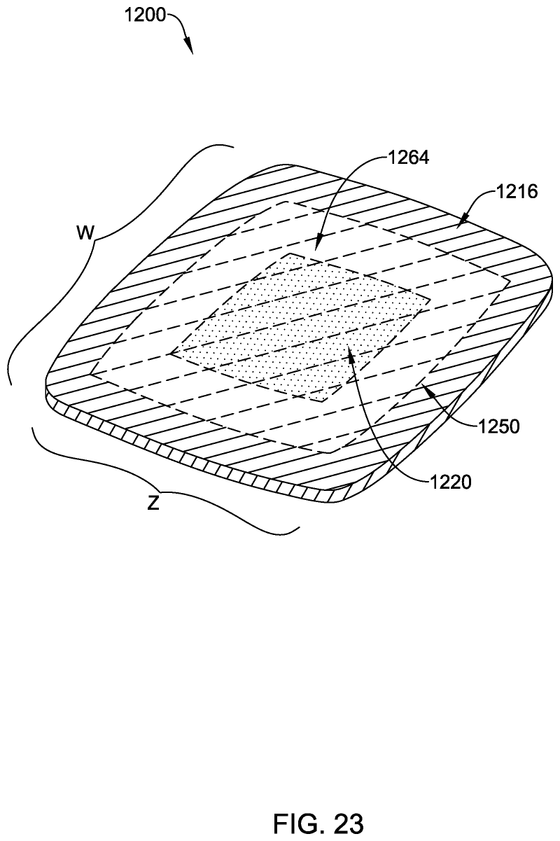

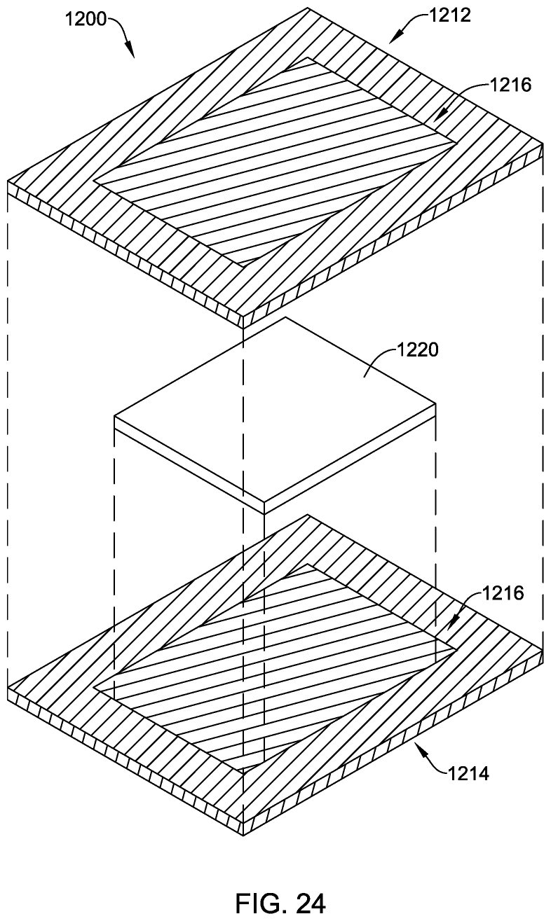

FIGS. 23-24 illustrate a repair implant 1200. The repair implant 1200 shown in FIG. 23 may include a feltable pouch (e.g., feltable patch, feltable scaffold, etc.) having a biologic material 1220 embedded within a cavity 1264 formed by sealing the edges of a top feltable fiber sheet 1212 (shown in FIG. 24) to a second feltable fiber sheet 1214 (shown in FIG. 24). The sealed seam defining the edges of the cavity 1264 in which the biologic material 1220 is disposed is shown by the dashed line 1250 in FIG. 23. It can be appreciated that just prior to completely forming the sealed seam (and thereby creating the enclosed cavity 1264) the biologic material 1220 may be injected through a yet-to-be-sealed opening between the top feltable fiber sheet 1212 (shown in FIG. 24) and the second feltable fiber sheet 1214 (shown in FIG. 24), whereby the opening is sealed after the biologic material 1220 is injected into the cavity 1264, thereby creating the sealed feltable pouch 1200 including the biologic material 1220 embedded therein.

It can be appreciated that sealing the edges of the top feltable fiber sheet 1212 (shown in FIG. 24) to the second feltable fiber sheet 1214 (shown in FIG. 24) may include felting the edges of the top feltable fiber sheet 1212 (shown in FIG. 24) to the second feltable fiber sheet 1214 (shown in FIG. 24) to form feltable “edges” of the implant 1200 which include feltable fibers 1216. Additionally, other techniques are contemplated to seal the edges of the top feltable fiber sheet 1212 (shown in FIG. 24) to the second feltable fiber sheet 1214 (shown in FIG. 24). These techniques may include gluing, suturing, melting the fibers together, chemical bonding, adhesive bonding, etc.

In some examples, the feltable pouch 1200 may include a lateral dimension “Z” and a longitudinal dimension “W”. In some embodiments, lateral dimension Z and longitudinal dimension W of the repair implant 1200 may range from about 20 millimeters (mm) to about 50 mm in the lateral direction Z and about 25 mm to about 50 mm in the longitudinal direction W.

The biologic material 1220 may include collagen, a collagen element, a collagen paste, a collagen slurry, and/or a collagen-like material similar to the bioinductive collagen construct described herein. Additionally, the biologic material 1220 may include (e.g., be impregnated with) adipose tissue, bursar, platelet-rich plasma, bone marrow aspirate concentrate, combinations thereof or other similar biologics. Additionally, the biologic material 1220 may be incorporated into the feltable pouch via casting the biologic (e.g., collagen), spraying the biologic (e.g., collagen), 3D printing the biologic (e.g., collagen), injecting the biologic (e.g.,, collagen, collagen slurry) into the pouch, combinations thereof or using similar methods. Additionally, the repair implant 1200 may be combined with Stravix®, Gravix®, fasica latta, nerve tubules, wound dressings, etc.

Additionally, it can be appreciated that the feltable material 1216 forming the feltable fiber scaffold may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material 1216 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fiber material 1216 may include combinations of any of the materials disclosed. For example, the feltable fiber material 1216 may be formed from a combination of any of natural fibers, synthetic fibers, biodegradable fibers, collagen fibers, or the like. It can be appreciated that when the implant 1200 is positioned at a target site (e.g., on the supraspinatus tendon during a rotator cuff repair), the first end region 1212 and the second end region 1214 may be attached to the target tissue (e.g., tendon, bone) using the felting techniques described herein.

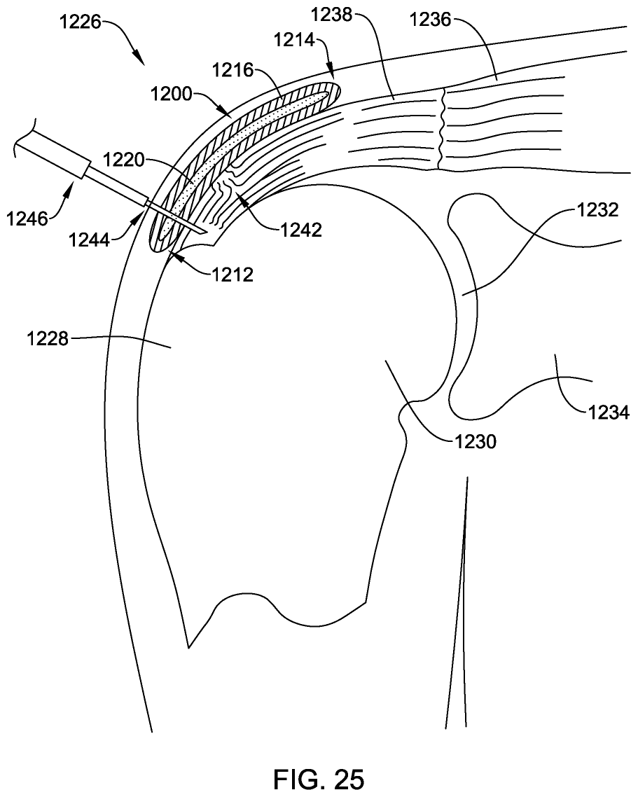

FIGS. 25-46 shows a cross-sectional view of a shoulder 1226 including the placement of the example implant 1200 described herein. In some examples, delivery of the implant 1200 (e.g., bioinductive implant 1200) to a target site of a patient may require a user to create an incision in the patient sufficient to access the target implant site. After creating this access site, the user may insert an implant delivery system through the access site and position the distal end of the implant delivery system adjacent the target implant site. The user may then manipulate the implant delivery system to deploy an implant out of a delivery sheath adjacent the target implant site.

FIG. 25 further illustrates the humerus 1230 mating with the glenoid fossa 1232 of the scapula 34. The glenoid fossa 1232 comprises a shallow depression in the scapula 1234. A supraspinatus tendon 1236 is also shown. These muscles (along with others) control the movement of humerus 1230 relative to scapula 1234.

In FIG. 25, the tendon 1238 includes a tear 1242 extending partially through the tendon 1238. The tear 1242 may be described as a partial thickness tear. The depicted partial thickness tear 1242 is on the bursal side of the tendon, however, the tear may also be on the opposite or articular side of the tendon 1238 and/or may include internal tears to the tendon 1238 not visible on either surface. Further, the tendon repair implant 1200 may overlay multiple tears. Additionally, while the repair implant 1200 has been described as being placed over a tendon, it should be understood that the repair implant 1200 may also be used to connect bone to bone. Further, while the tear 1242 is illustrated as a partial thickness tear, it should be understood the implant 1200 can also be used to repair a full thickness tear. For example, the repair implant 1200 may be used to reinforce a repair that is fully approximated or to bridge between the tendon and the bone when the tendon cannot be approximated back to the bone. The repair implants disclosed herein may provide additional tensile strength while maintaining the ability of the implant 1200, or portions of the implant 1200, to be completely absorbed and remodeled by the body. Further, as discussed herein, in addition to the rotator cuff, the repair implant 1200 may be used with other soft tissue repairs, such as, but not limited to the anterior cruciate ligament (ACL), the lateral collateral ligament (LCL), meniscus of the knee, tendons/ligaments of the hip capsule, Achilles tendon and other various ligaments of the shoulder, knee, ankle and hip, for example.

As illustrated in FIG. 25, the first end region 1212 of the repair implant 1200 may be configured to be positioned on a portion of the torn tendon which remains fixed to the footprint of the greater tuberosity 1228 while the second end region 1214 of the repair implant 1200 may be configured to be positioned on the distal tendon 1238 of the supraspinatus tendon 1236. It can be further appreciated from FIG. 25 that the biologic material 1220 in the feltable pouch may positioned over the partial thickness tear 1242.

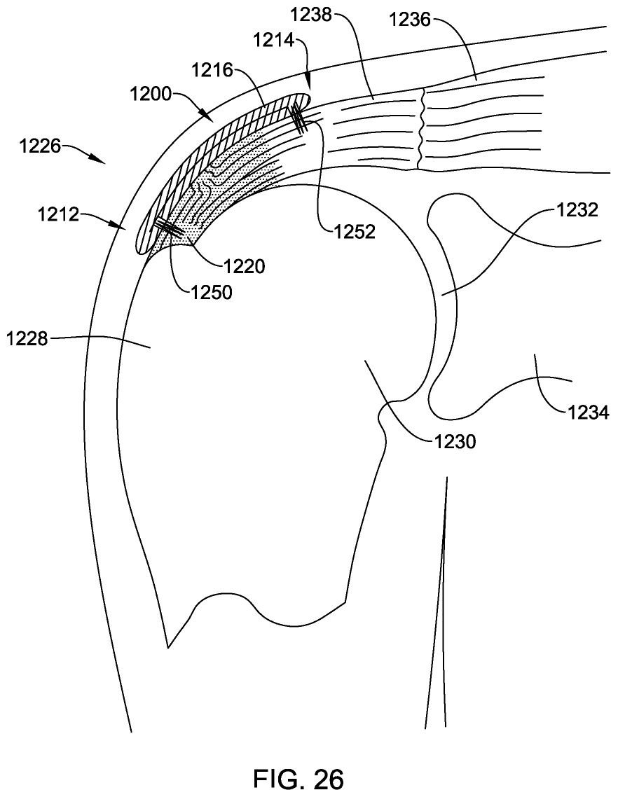

FIGS. 25-26 further illustrate that attachment of the repair implant 1200 to the portion of the torn tendon which remains fixed to the footprint of the greater tuberosity 1228 and the tendon 1238 may be achieved via a felting process. For example, FIG. 25 illustrates that a user may introduce a felting needle 1244 into the patient's shoulder 1226. The felting needle 1244 may be attached to a felting drive mechanism 1246 whereby the felting drive mechanism 1246 is configured to rapidly displace (e.g., oscillate, drive) the needle 1244 back and forth along the longitudinal axis of the needle 1244.

It can be appreciated that attaching the first end region 1212 of the repair implant 1200 to the portion of the torn tendon 1238 which remains fixed to the footprint of the greater tuberosity 1228 and the second end region 1214 of the repair implant 1200 to the distal tendon 1238 of the supraspinatus tendon 1236 may include a user activating the felting drive mechanism 1246 to repeatedly advance the needle 1244 through the feltable pouch to attach the first end region 1212 of the repair implant 1200 to the portion of the torn tendon 1238 which remains fixed to the footprint of the greater tuberosity 1228 and attach the second end region 1214 of the repair implant 1200 to the distal tendon 1238 of the supraspinatus tendon 1236. It can be appreciated that some of the fibers 1250 (shown in FIG. 26) of the feltable material 1216 may be pushed and/or pulled into the portion of the torn tendon 1238 which remains fixed to the footprint of the greater tuberosity 1228 and some of the fibers 1252 (shown in FIG. 26) of the feltable material 1216 may be pushed and/or pulled into the tendon 1238 via the needle 1244 to produce a robust connection between the felted material 1216 and the portion of the torn tendon which remains fixed to the footprint of the greater tuberosity 1228 and also between the felted material 1216 and the tendon 1238.

Additionally, FIGS. 25-26 illustrate that during the felting process to attach the first end region 1212 of the repair implant 1200 to the portion of the torn tendon 1238 which remains fixed to the footprint of the greater tuberosity 1228 and the second end region 1214 of the repair implant 1200 to the distal tendon 1238, the needle 1244 may pass into and through the cavity 1264 (shown in FIG. 23), thereby creating an opening in the feltable pouch (e.g., puncturing the feltable pouch) and releasing the biologic material 1220 into the tissue near the tear 1242. It can be appreciated that the first end region 1212 of the repair implant 1200 may remain attached to the portion of the torn tendon 1238 which remains fixed to the footprint of the greater tuberosity 1228 and the second end region 1214 of the repair implant 1200 may remain attached to the distal tendon 1238 as the biologic material 1220 is released into the tissue near the tear 1242.

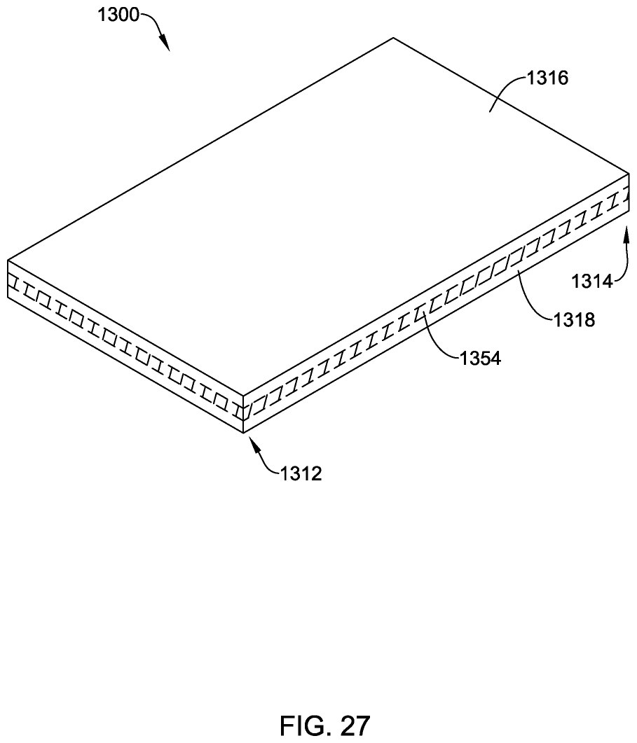

FIGS. 27-29E illustrate various repair plant configurations in which reinforcing material (e.g., reinforcing fibers) may be utilized to strengthen a repair implant.

FIG. 27 illustrates a repair implant 1300. The repair implant 1300 may include a first end region 1312 and a second end region 1314. FIG. 27 further illustrates that the repair implant 1300 may include a top layer 1316, a bottom layer 1318 and a middle layer 1354 positioned between the top layer 1316 and the bottom layer 1318. One or more of the top layer 1316, the middle layer 1354 and/or the bottom layer 1318 may have a thickness of about 0.5 mm to about 5 mm. Additionally, the middle layer 1354 may include plurality of reinforcing fibers extending from the first end region 1312 to the second end region 1314.

Additionally, it can be appreciated that each of the top layer 1316, the middle layer 1354 and/or the bottom layer 118 may include a feltable material or other type of fiber. The individual fibers forming the top layer 1316, the middle layer 1354 and/or the bottom layer 1318 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the top layer 1316, the middle layer 1354 and/or the bottom layer 1318 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material forming the top layer 1316, the middle layer 1354 and/or the bottom layer 1318 may include combinations of any of the materials disclosed. For example, the feltable material forming the top layer 1316, the middle layer 1354 and/or the bottom layer 1318 may be formed from a combination of any of natural fibers, synthetic fibers, biodegradable fibers, collagen fibers, or the like.

Further, in some examples, the reinforcing fibers forming the middle layer 1354 may include sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

It can be appreciated that when the implant 1300 is positioned at a target site (e.g., on the supraspinatus tendon during a rotator cuff repair), the first end region 1312 and the second end region 1314 may be attached to the target tissue (e.g., tendon, bone) using the felting techniques described herein.

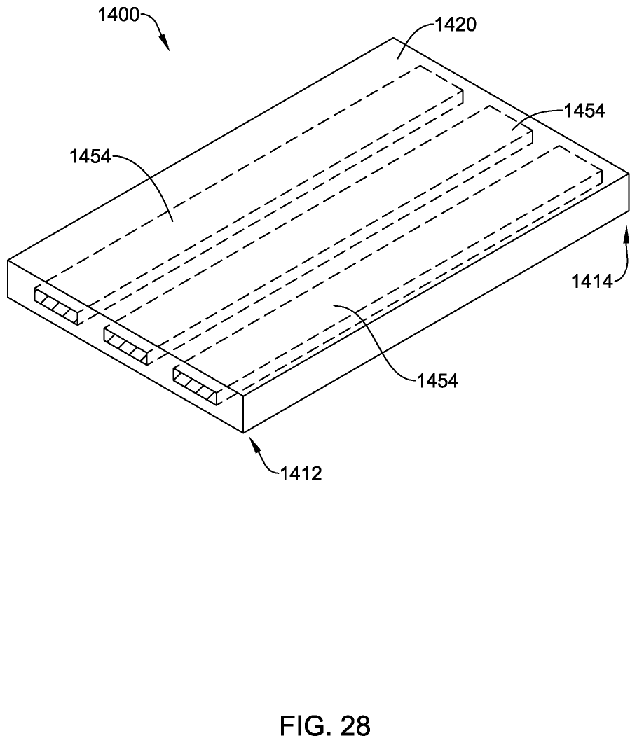

FIG. 28 illustrates a repair implant 1400. The repair implant 1400 may include a first end region 1412 and a second end region 1414 opposite the first end region 1412. The repair implant 1400 may include a first end region 1412 and a second end region 1414. Further, the repair implant may include a non-woven feltable scaffold 1420 formed from a plurality of feltable fibers. Additionally, the feltable scaffold 1420 may include plurality of reinforcing fibers 1454 integrated with (e.g., attached to, embedded within, etc.) the feltable scaffold 1420 and extending from the first end region 1412 to the second end region 1414. The reinforcing fibers 1454 may including longitudinal strips of fiber material that extend from the first end region 1412 to the second end region 1414. FIG. 28 illustrates three reinforcing fibers 1454 positioned within the feltable scaffold 1420. However, it is contemplated that the repair implant may include 1, 2, 3, 4, 5, 6 or more reinforcing fibers 1454.

The individual fibers forming the feltable fiber scaffold 1420 and/or the reinforcing fibers 1454 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the feltable fiber scaffold 1420 and/or the reinforcing fibers 1454 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fiber scaffold 1420 and/or the reinforcing fibers 1454 may include combinations of any of the materials disclosed. For example, the feltable fiber scaffold 1420 and/or the reinforcing fibers 1454 may be formed from a combination of any of natural fibers, synthetic fibers, biodegradable fibers, collagen fibers, or the like.

Further, in some examples, the reinforcing fibers 1454 may include sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

It can be appreciated that when the implant 1400 is positioned at a target site (e.g., on the supraspinatus tendon during a rotator cuff repair), the first end region 1412 and the second end region 1414 may be attached to the target tissue (e.g., tendon, bone) using the felting techniques described herein.

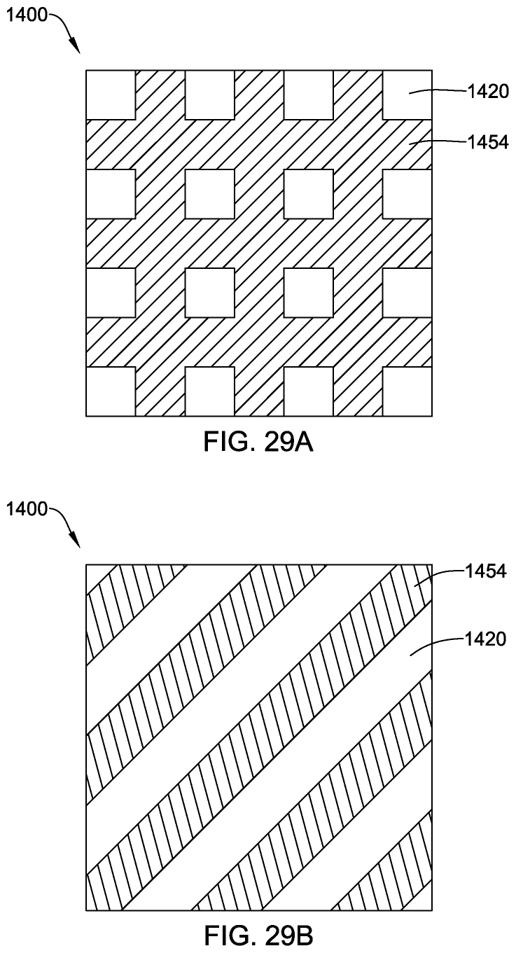

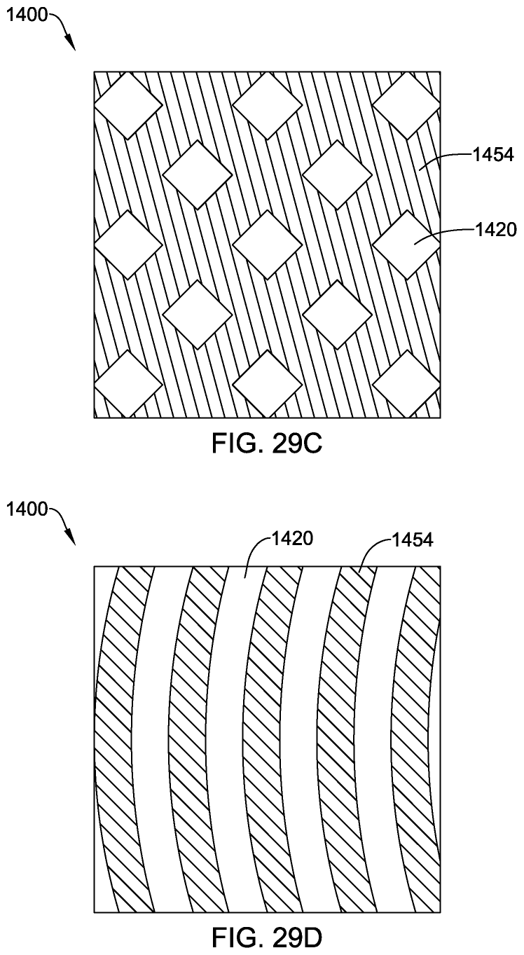

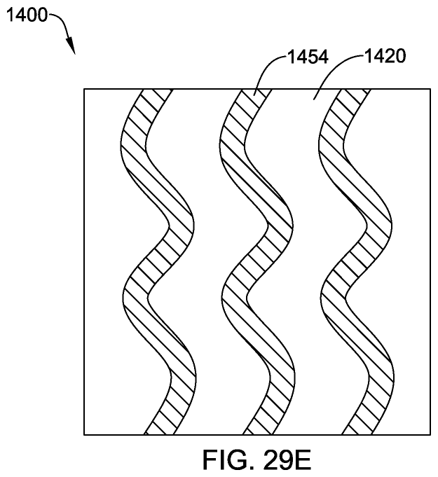

FIGS. 29A-29E schematically depict various example arrangements of the feltable scaffold 1420 and reinforcing fibers 1454 integrated with (e.g., attached to, embedded within, etc.) the feltable scaffold 1420. These examples are in addition to the example arrangement of the feltable scaffold 1420 and reinforcing fibers 1454 described with respect to FIG. 28. The example arrangements of the feltable scaffold 1420 and reinforcing fibers 1454 shown in FIGS. 29A-29E are schematic representations that depict alternative arrangements. The edges/boundaries shown in FIGS. 29A-29E are intended to mimic the edge/boundaries of the pattern/arrangement shown in FIG. 28, or a variation thereof, can be utilized in any of the devices disclosed as alternatives of the feltable scaffold 1420 and reinforcing fibers 1454.

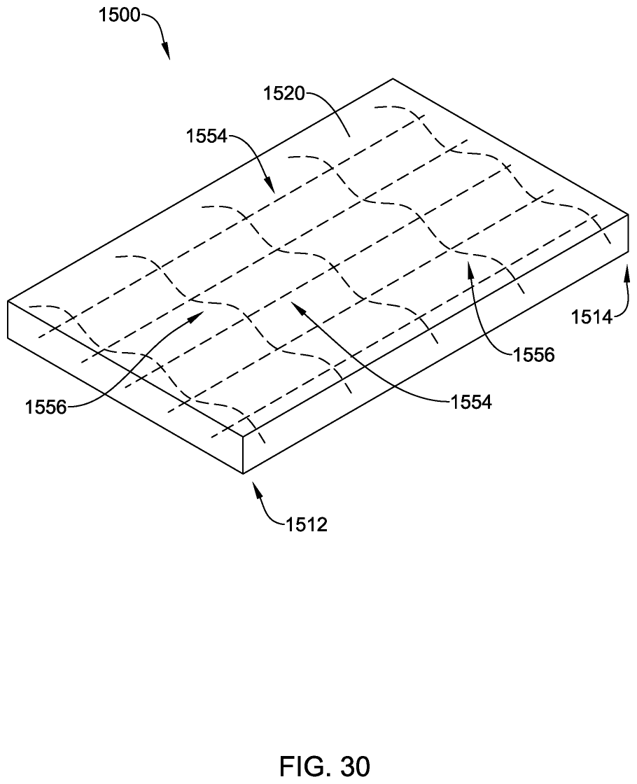

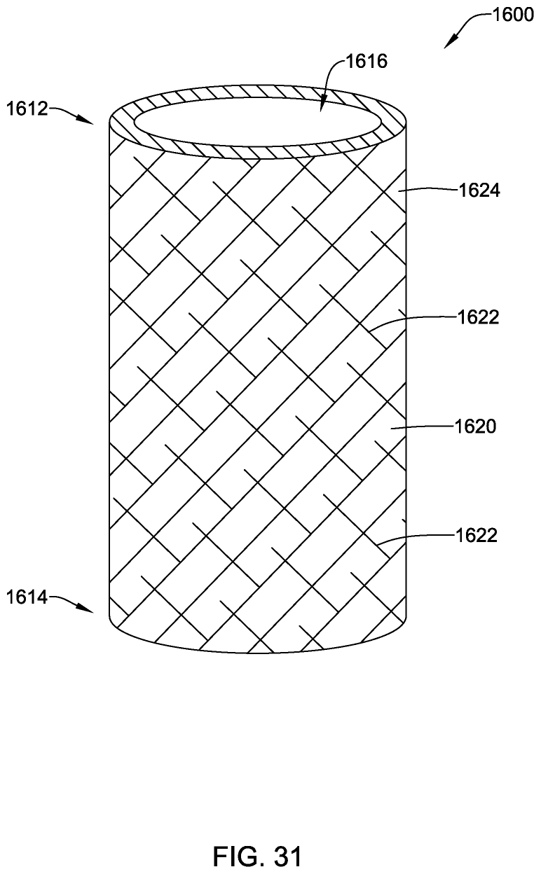

FIG. 30 illustrates a repair implant 1500. The repair implant 1500 may include a first end region 1512 and a second end region 1514 opposite the first end region 1512. Further, the repair implant may include a non-woven feltable scaffold 1520 formed from a plurality of feltable fibers. Further, the feltable scaffold 1520 may include a plurality of reinforcing fibers 1554 integrated with (e.g., attached to, embedded within, etc.) the feltable scaffold 1520 and extending generally along the longitudinal axis of the repair implant 1500 from the first end region 1512 to the second end region 1514. Further yet, the feltable scaffold 1520 may include plurality of reinforcing fibers 1556 integrated with (e.g., attached to, embedded within, etc.) the feltable scaffold 1520 and extending generally transverse to the longitudinal axis of the repair implant 1500.

Additionally, it can be appreciated that the reinforcing fibers 1554 and the reinforcing fibers 1556 may be interwoven in a configuration that allows the individual reinforcing fibers 1554, 1556 to constrict and tighten down on the target tissue (e.g., tendon tear, tendon injury site, etc.) when a longitudinal force is placed upon the feltable scaffold 1520.

The individual fibers forming the feltable fiber scaffold 1520 and/or the reinforcing fibers 1554, 1556 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material forming the feltable fiber scaffold 1520 and/or the reinforcing fibers 1554, 1556 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fiber scaffold 1520 and/or the reinforcing fibers 1554, 1556 may include combinations of any of the materials disclosed. For example, the feltable fiber scaffold 1520 and/or the reinforcing fibers 1554, 1556 may be formed from a combination of any of natural fibers, synthetic fibers, biodegradable fibers, collagen fibers, or the like.

Further, in some examples, the reinforcing fibers 1554, 1556 may include sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

It can be appreciated that when the implant 1500 is positioned at a target site (e.g., on the supraspinatus tendon during a rotator cuff repair), the first end region 1512 and the second end region 1514 may be attached to the target tissue (e.g., tendon, bone) using the felting techniques described herein.

It can be appreciated that any of the fiber-reinforced feltable fiber scaffolds or variations thereof described in FIGS. 27-30 may also include a biologic material. The biologic material may include collagen, a collagen element, a collagen paste, a collagen slurry, and/or a collagen-like material similar to other bioinductive collagen constructs described herein. Additionally, the biologic material may include adipose tissue, bursar, platelet-rich plasma, bone marrow aspirate concentrate, combinations thereof or other similar biologic materials.

It can be further appreciated that any of the example repair implants, variations of the repair implants, manufacturing techniques utilized to construct the repair implants and variations thereof described herein may also be applicable to any type of graft construct including, but not limited to, a decellularized graft, an autograft, an allograft, a xenograft, a synthetic graft, etc. For example, it can be appreciated that a graft construct (e.g., dermal graft, decellularized graft, an autograft, an allograft, a xenograft, a synthetic graft, or similar construct) may include a feltable material, feltable scaffold, feltable fibers and biologic material (including collagen, a collagen element, a collagen paste, a collagen slurry, and/or a collagen-like material similar to the bioinductive collagen constructs described herein) similar to the embodiments and variations thereof described herein. In one example, a feltable fiber could be integrated with a graft construct such that the feltable material overlays an entire graft construct. In other examples, a feltable fiber could be integrated with a graft construct such that it extends around only around the perimeter of the graft construct. In other examples, a feltable fiber could be integrated with a graft construct that is applied in patterned arrangement (such as the felted pattern arrangements disclosed in FIG. 1 and FIGS. 4A-4I) over the graft construct. In yet other examples, a feltable fiber could be integrated with a graft construct such that it is applied as an open mesh over the graft construct, or any combination thereof.

It can be further appreciated that the feltable materials and variations thereof described herein may be integrated with a biologic construct (e.g., collagen implant, collagen-like material implant, hydrogel implant or any similar biologic material implant) using a variety of attachment techniques including, but not limited to, pre-suturing feltable material to a biologic construct or other feltable element, suturing feltable material to a biologic construct or other feltable element, pre-suturing feltable material to a biologic construct or other feltable element, gluing feltable material to a biologic construct or other feltable element, welding feltable material to a biologic construct or other feltable element, localized heating and melting of feltable material to a biologic construct, potting of feltable material to a biologic construct, chemical bonding a feltable material to a biologic construct or other feltable element, adhesive bonding a feltable material to a biologic construct or other feltable element or any combinations thereof. Examples of feltable materials and variations thereof integrated with a biologic construct are disclosed herein.

FIG. 31 illustrates a repair implant 1600. The repair implant 1600 may include a first end region 1612 and a second end region 1614 positioned opposite the first end region 1612. FIG. 31 illustrates that the repair implant 1600 may include a generally tubular configuration extending from the first end region 1612 to the second end region 1614. Additionally, FIG. 31 illustrates that the repair implant 1600 may include a lumen 1616 extending from the first end region 1612 to the second end region 1614.

Further, the repair implant 1600 may include a base material 1624 formed from a biologic component. The base material 1624 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 1600 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 1600. For example, a lattice structure and/or other structures described herein may be formed by molding which may be punched, woven, etc. to form the desired arrangement. The repair implant 1600 may be porous or included pathways to encourage tissue in-growth. In some embodiments, the repair implant 1600 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 31 further illustrates that the repair implant 1600 may further include a feltable material 1622 integrated (e.g., interwoven, interlinked, interlaced, knitted, crisscrossed, netted, randomly-dispersed, uniformly-dispersed, arranged, etc.) within the base material 1624. For example, FIG. 31 illustrates the repair implant 1600 may include a plurality of feltable fibers 1622 integrated within a collagen base material 1624. It can be appreciated that the feltable fibers 1622 may be distributed within the collagen base material 1624 from the first end region 1612 to the second end region 1614 of the repair implant 1600.

In some examples, the plurality of individual feltable fibers 1622 may be matted, condensed, pressed, bonded, etc. together to form a non-woven structure or construct integrated with the collagen base material 1624. In some examples, the individual feltable fibers 1622 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 1622 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 1622 may include combinations of any of the materials disclosed. For example, the feltable fibers 1622 may be formed from a combination of any of natural fibers, synthetic fibers, biodegradable fibers, collagen fibers, or the like. It can be further appreciated that the feltable fibers 1622 may be randomly aligned with one another to form a non-woven felted structure. In other examples, however, the feltable fibers 1622 may be aligned with one another to achieve unidirectional stiffness properties with the non-woven felted structure.

In some embodiments, the repair implant 1600 may be configured to provide a combination of structural features, properties and functions that may be used to treat partial or full thickness tears of soft tissues, including partial or full thickness tears of the rotator cuff tendons, ACL repair, LCL repair, the gluteal tendon, meniscus tears, tendons/ligaments of the hip joint capsule, Achilles tendon, etc. The repair implant 1600 may encourage tissue ingrowth while also providing additional mechanical properties such as, but not limited to strength and stiffness. For example, the implant 1600 may provide a layer of collagen over injured tissue to facilitate healing.

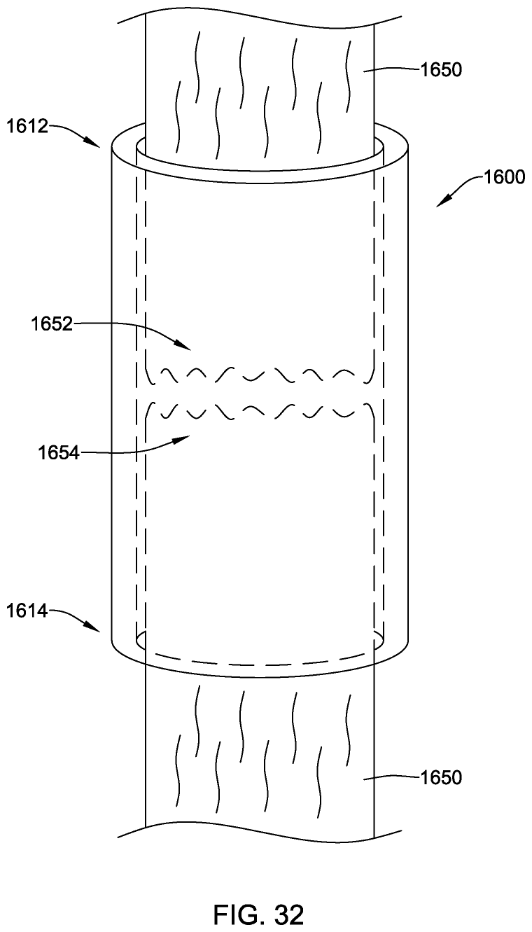

FIG. 32 illustrates the repair implant 1600 positioned along a torn tendon 1650. It can be appreciated from FIG. 32 that a first end 1652 of the torn tendon 1650 may be positioned within the lumen 1616 (shown in FIG. 31) of the first end region 1612 of the repair implant 1600 and that a second end 1654 of the torn tendon 1650 may be positioned within the lumen 1616 (shown in FIG. 31) of the second end region 1614 of the repair implant 1600. It can be further appreciated that after the first end 1652 and the second end 1654 of the torn tendon 1650 are positioned with the lumen 1616 of the repair implant 1600, the feltable fibers 1622 (shown in FIG. 31) of the repair implant 1600 may be felted to the first end 1652 and the second end 1654 of the torn tendon 1650, respectively. It can be appreciated that felting the feltable fibers 1622 of the repair implant 1600 to the first end 1652 and the second end 1654 of the torn tendon 1650 may secure the repair implant 1600 (including the collagen base material 1624) to the torn tendon 1650, thereby permitting the collagen biologic material to aid in the healing of the torn tendon. In some examples, a tendon repair using the repair implant 1600 may be performed using a medical device configured to hold the lumen of the repair implant 1600 open, while a suture pulls the torn tendon into the lumen of the repair implant 1600 and holds the tendon in place relative to the repair implant 1600 until felting the repair implant 1600 to the tendon is complete.

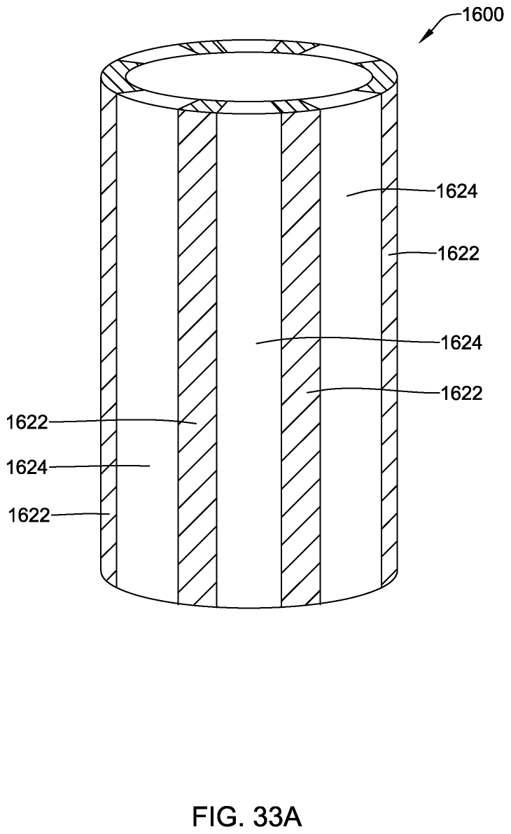

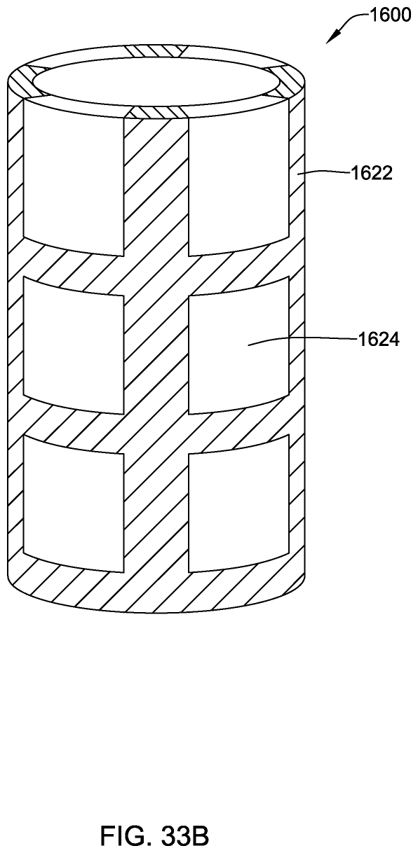

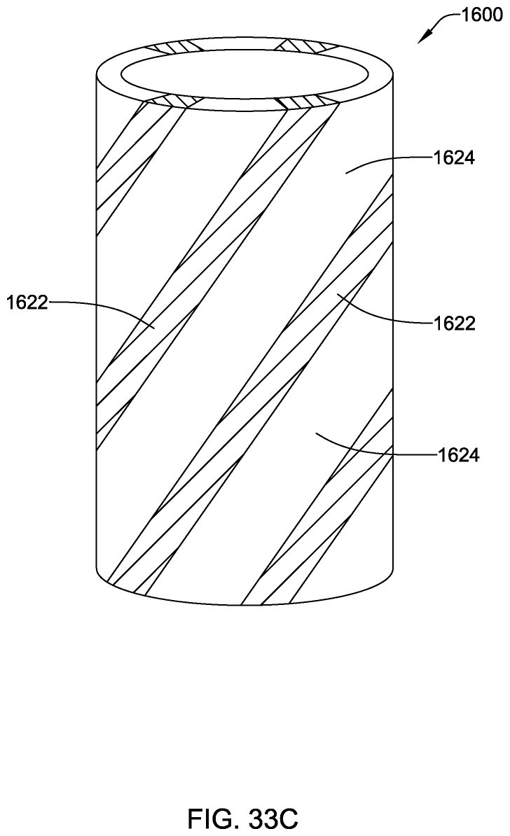

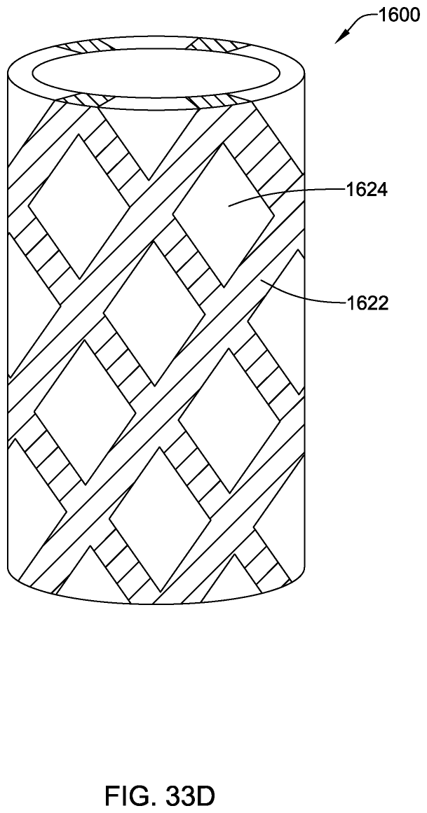

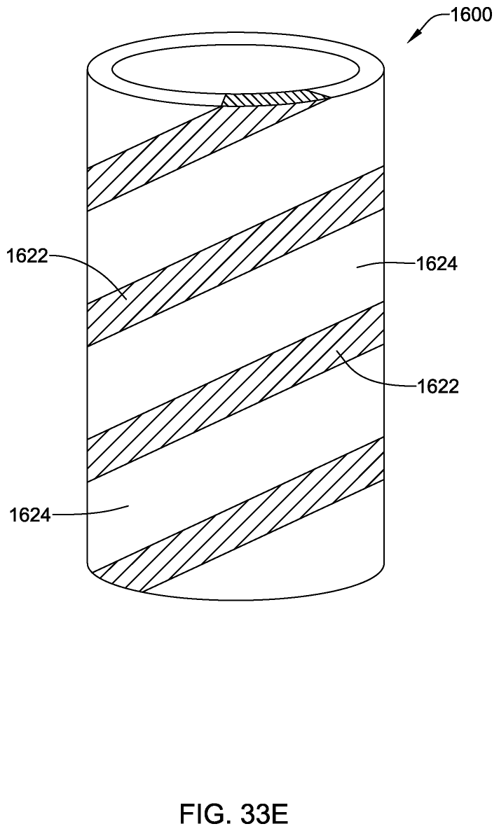

FIG. 33A-33E schematically depict various example arrangements of the feltable fibers 1622 integrated (e.g., interwoven, interlinked, interlaced, knitted, crisscrossed, netted, randomly-dispersed, uniformly-dispersed, arranged, etc.) within the base material 1624. These examples are in addition to the example arrangement of the feltable fibers 1622 within the base material 1624 described with respect to FIG. 31. The example arrangements of the feltable fibers 1622 within the base material 1624 shown in FIGS. 33A-33E are schematic representations that depict alternative arrangements. The edges/boundaries shown in FIGS. 33A-33E are intended to mimic the edge/boundaries of the pattern/arrangement shown in FIG. 31, or a variation thereof, can be utilized in any of the devices disclosed as alternatives of the feltable fibers and base material.

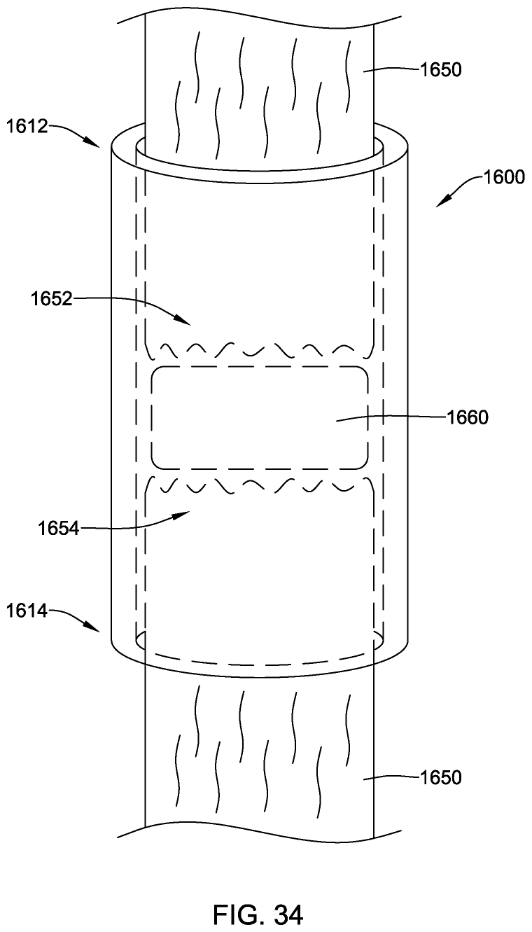

Like that described with respect to FIG. 31, FIG. 34 illustrates the repair implant 1600 positioned along a torn tendon 1650. It can be appreciated from FIG. 34 that a first end 1652 of the torn tendon 1650 may be positioned within the lumen 1616 (shown in FIG. 31) of the first end region 1612 of the repair implant 1600 and that a second end 1654 of the torn tendon 1650 may be positioned within the lumen 1616 (shown in FIG. 31) of the second end region 1614 of the repair implant 1600. Additionally, FIG. 34 illustrates that, in some examples, a biologic material 1660 may be positioned between the first end 1652 and the second end 1654 of the torn tendon 1650. The biologic material 1660 may include collagen, a collagen element and/or a collagen-like material similar to other bioinductive collagen constructs described herein, an absorbable material such as PGA, PLGA, HA, or other suitable absorbable polymers, non-absorbable materials, or any combination of collagen, absorbable polymers, and nonabsorbable polymers.

It can be further appreciated that after the first end 1652 of the torn tendon 1650, the second end 1654 of the torn tendon 1650 and the biologic material 1660 are positioned with the lumen 1616 of the repair implant 1600, the feltable fibers 1622 (shown in FIG. 31) of the repair implant 1600 may be felted to the first end 1652 and the second end 1654 of the torn tendon 1650, respectively, thereby positioning (e.g., trapping) the biologic material 1660 between the first end 1652 and the second end 1654 of the torn tendon 1650. It can be appreciated that felting the feltable fibers 1622 of the repair implant 1600 to the first end 1652 and the second end 1654 of the torn tendon 1650 may secure the repair implant 1600 (including the collagen base material 1624) to the torn tendon 1650, thereby permitting the collagen biologic material 1624 of the repair implant 1600 and the biologic material 1660 (positioned between the first end 1652 and the second end 1654 of the torn tendon 1650) to aid in the healing of the torn tendon.

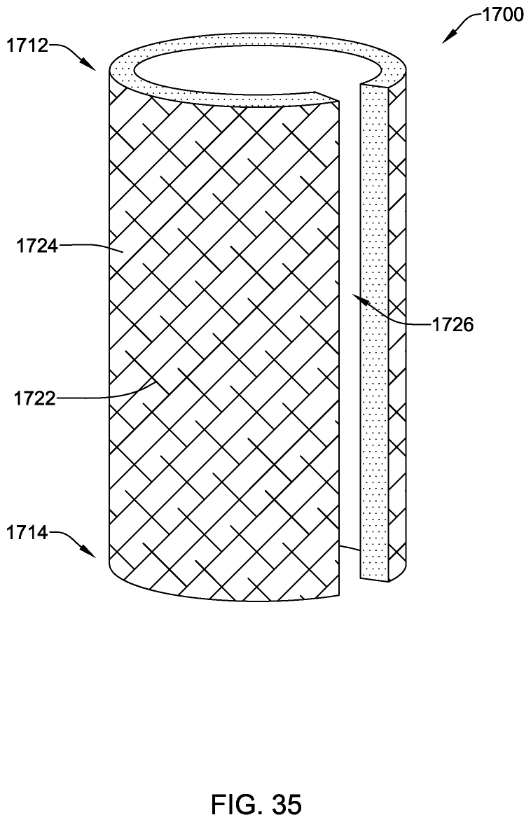

FIG. 35 illustrates a repair implant 1700. The repair implant 1700 may include a first end region 1712 and a second end region 1714 positioned opposite the first end region 1712. FIG. 35 illustrates that the repair implant 1700 may include a generally tubular configuration extending from the first end region 1712 to the second end region 1714. Additionally, FIG. 35 illustrates that the repair implant 1700 may include a slit 1726 extending from the first end region 1712 to the second end region 1714.

Further, the repair implant 1700 may include a base material 1724 formed from a biologic component. The base material 1724 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 1700 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 1700. For example, a lattice structure and/or other structures described herein may be formed by molding which may be punched, woven, etc. to form the desired arrangement. The repair implant 1700 may be porous or included pathways to encourage tissue in-growth. In some embodiments, the repair implant 1700 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 35 further illustrates that the repair implant 1700 may further include a feltable material 1722 integrated (e.g., interwoven, interlinked, interlaced, knitted, crisscrossed, netted, randomly-dispersed, uniformly-dispersed, arranged, etc.) within the base material 1724. For example, FIG. 35 illustrates the repair implant 1700 may include a plurality of feltable fibers 1722 integrated within a collagen base material 1724. It can be appreciated that the feltable fibers 1722 may be distributed within the collagen base material 1724 from the first end region 1712 to the second end region 1714 of the repair implant 1700.

In some examples, the plurality of individual feltable fibers 1722 may be matted, condensed, pressed, bonded, etc. together to form a non-woven structure or construct integrated with the collagen base material 1724. In some examples, the individual feltable fibers 1722 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 1722 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 1722 may include combinations of any of the materials disclosed. For example, the feltable fibers 1722 may be formed from a combination of any of natural fibers, synthetic fibers, biodegradable fibers, collagen fibers, or the like. It can be further appreciated that the feltable fibers 1722 may be randomly aligned with one another to form a non-woven felted structure. In other examples, however, the feltable fibers 1722 may be aligned with one another to achieve unidirectional stiffness properties with the non-woven felted structure.

It can be further appreciated that the slit 1726 may ease the positioning of the repair implant 1700 at a target tissue repair site. For example, it can be appreciated that a user may spread the repair implant 1700 open along the slit 1726, thereby permitting the user to wrap the repair implant 1700 around a target tissue repair site. It can be appreciated that the repair implant 1700 may be spread open and wrapped around a torn tendon (e.g., a torn tendon similar to the torn tendon illustrated in FIG. 32) or other partial or full thickness tear of a soft tissue. It can be further appreciated that once the repair implant 1700 has been wrapped around a target tissue site (e.g., a torn tendon), the feltable fibers 1722 of the repair implant 1700 may be felted to the target tissue site (e.g., a torn tendon). It can be appreciated that felting the feltable fibers 1722 of the repair implant 1700 to the target tissue site (e.g., a torn tendon similar to the torn tendon illustrated in FIG. 32), may secure the repair implant 1700 (including the collagen base material 1724) to the torn tendon or other soft tissue and permit the collagen biologic material of the repair implant 1700 to aid in the healing of the torn tendon or other soft tissue site.

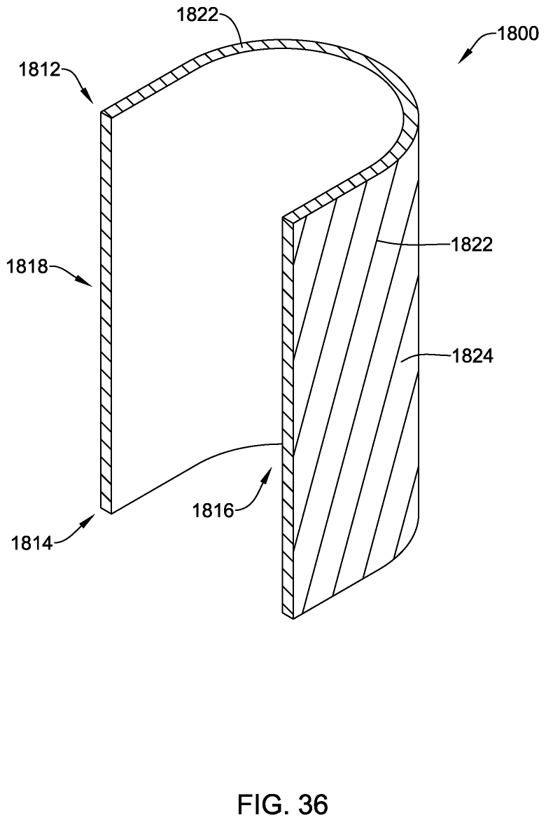

FIG. 36 illustrates a repair implant 1800. The repair implant 1800 may include a first end region 1812 and a second end region 1814 positioned opposite the first end region 1812. Further, the repair implant 1800 may include an edge 1816 and a lower edge 1818. Further, FIG. 36 illustrates that the repair implant 1800 may include a generally U-shaped configuration extending from the upper edge 1816 to the bottom edge 1818.

Further, the repair implant 1800 may include a base material 1824 formed from a biologic component. The base material 1824 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 1800 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 1800. For example, a lattice structure and/or other structures described herein may be formed by molding which may be punched, woven, etc. to form the desired arrangement. The repair implant 1800 may be porous or included pathways to encourage tissue in-growth. In some embodiments, the repair implant 1800 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 36 further illustrates that the repair implant 1800 may further include a feltable material 1822 integrated (e.g., interwoven, interlinked, interlaced, knitted, crisscrossed, netted, randomly-dispersed, uniformly-dispersed, arranged, etc.) within the base material 1824. For example, FIG. 36 illustrates the repair implant 1800 may include a plurality of feltable fibers 1822 integrated within a collagen base material 1824. It can be appreciated that the feltable fibers 1822 may be distributed within the collagen base material 1824 from the first end region 1812 to the second end region 1814 of the repair implant 1800.

In some examples, the plurality of individual feltable fibers 1822 may be matted, condensed, pressed, bonded, etc. together to form a non-woven structure or construct integrated with the collagen base material 1824. In some examples, the individual feltable fibers 1822 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 1822 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 1822 may include combinations of any of the materials disclosed. For example, the feltable fibers 1822 may be formed from a combination of any of natural fibers, synthetic fibers, biodegradable fibers, collagen fibers, or the like. It can be further appreciated that the feltable fibers 1822 may be randomly aligned with one another to form a non-woven felted structure. In other examples, however, the feltable fibers 1822 may be aligned with one another to achieve unidirectional stiffness properties with the non-woven felted structure.

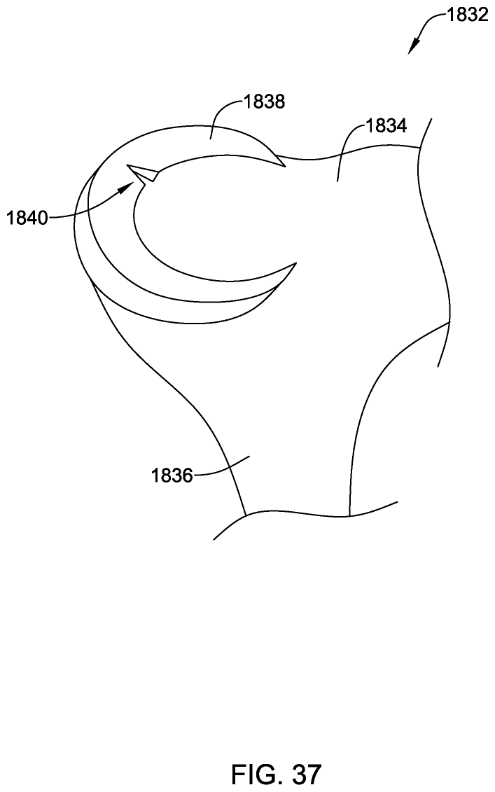

FIG. 37 illustrates a portion of a knee joint 1832 including the tibial plateau 1834 of the tibia 1836. Further, FIG. 37 illustrates a portion of the meniscus 1838 positioned along the tibial plateau 1834. Further yet, FIG. 37 illustrates a partial tear 1840 in the meniscus 1838.

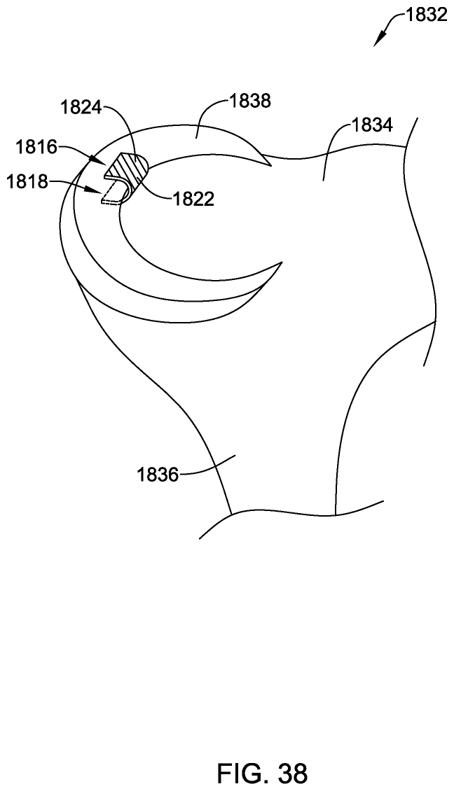

FIG. 38 illustrates the repair implant 1800 positioned along the tear 1840 (shown in FIG. 37) of the meniscus 1838. It can be appreciated from FIG. 38 that the upper edge 1816 of the repair implant 1800 may be positioned along the superior surface of the meniscus 1834 and the lower edge 1818 of the repair implant 1800 may be positioned along the inferior surface of the meniscus 1834 such the body of the repair implant 1800 sandwiches the tear 1840 in the meniscus 1834. In other words, the repair implant 1800 may extend partially around the tear 1840 in the meniscus 1834, whereby the upper edge 1816 is on top of the tear 1840 and the lower edge 1818 is underneath the tear 1840.

Further, it can be appreciated that after the repair implant 1800 is be positioned along the tear 1840 in the meniscus 1834, the feltable fibers 1822 of the repair implant 1800 may be felted to various portions of the meniscus 1834. It can be appreciated that felting the feltable fibers 1822 of the repair implant 1800 to the meniscus 1834 may secure the repair implant 1800 (including the collagen base material 1824) to the meniscus 1834, thereby permitting the feltable fibers 1822 to reinforce the meniscus 1834 and the collagen biologic material to aid in the healing of the tear 1840 in the meniscus 1834.

Additionally, it can be appreciated that in some examples, the repair implant 1800 may be configured such that the felted fibers 1822 of the repair implant 1800 may be positioned toward the outer surfaces (e.g., surfaces facing away from the target tissue) of the repair implant 1800 while the collagen biologic material may be positioned primarily along the inner surface (e.g., along the surfaces which contact the target tissue of the target tissue site) of the repair implant 1800. In other words, the repair implant 1800 may be configured such that the collagen biologic material may be in contact with the tear 1840 of the meniscus 1834 while the feltable fibers 1822 may be positioned away from the tear 1840 of the meniscus 1834. It can be appreciated that when configured such that the collagen biologic material is in contact with the tear 1840 of the meniscus 1834 while the feltable fibers 1822 may are positioned away from the tear 1840 of the meniscus 1834, felting the feltable fibers 1822 to the target tissue site (e.g., to the meniscus 1834 of FIG. 38) may include pushing the feltable fibers 1822 through the collagen biologic material and into the meniscus 1834.

While FIGS. 37-38 illustrate the repair implant 1800 being utilized in a meniscus repair, it can be appreciated that the repair implant 1800 may be utilized in other anatomical repairs, such as a rotator cuff repair, Achilles repair, or other suitable application.

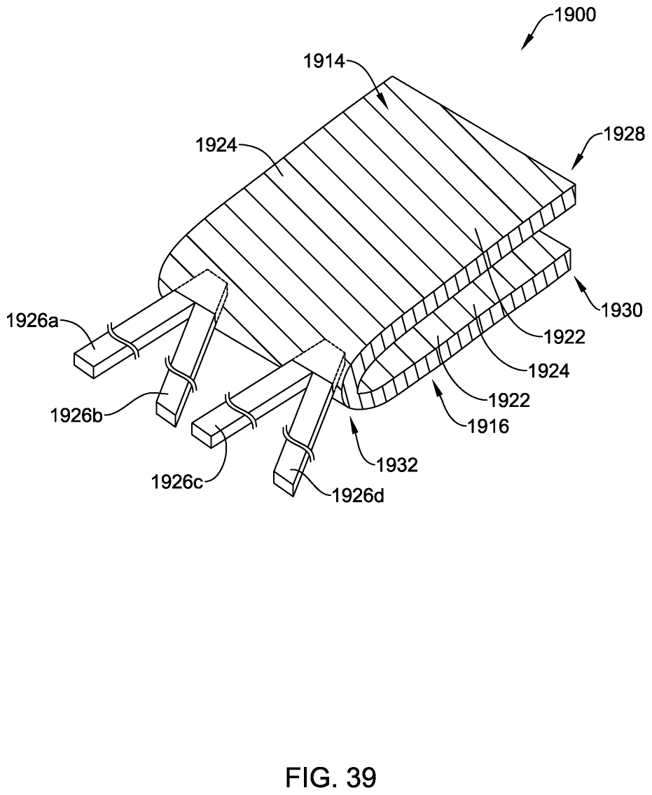

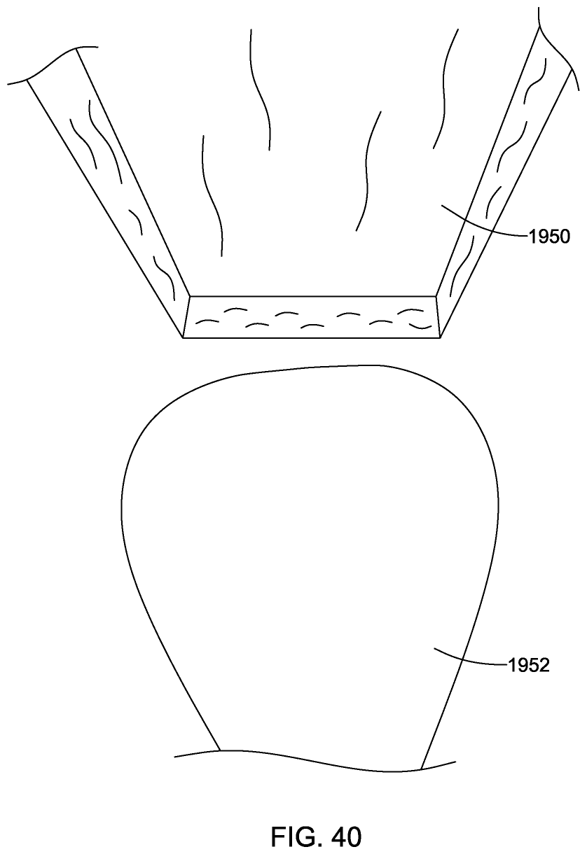

FIG. 39 illustrates a repair implant 1900. The repair implant 1900 may be folded upon itself along a folded region 1932 to define a first flap 1914 positioned adjacent a second flap 1916. FIG. 39 illustrates that the first flap 1914 may include a first free edge 1928 and the second flap 1916 may include a second free edge 1930. It some examples, the first flap 1914 and the second flap 1916 may be able to flex (e.g., spread) away from one another.

Further, the repair implant 1900 (including the first flap 1914, the second flap 1916 and the folded region 1932) may include a base material 1924 formed from a biologic component. The base material 1924 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 1900 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 1900. For example, a lattice structure and/or other structures described herein may be formed by molding which may be punched, woven, etc. to form the desired arrangement. The repair implant 1900 may be porous or included pathways to encourage tissue in-growth. In some embodiments, the repair implant 1900 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 39 further illustrates that the repair implant 1900 may further include a feltable material 1922 which is integrated (e.g., interwoven, interlinked, interlaced, knitted, crisscrossed, netted, randomly-dispersed, uniformly-dispersed, arranged, etc.) within the base material 1924. For example, FIG. 38 illustrates the repair implant 1900 may include a plurality of feltable fibers 1922 integrated within a collagen base material 1924. It can be appreciated that the feltable fibers 1922 may be distributed within the collagen base material 1924 forming the first flap 1914, a second flap 1916 and the folded region 1932 of the repair implant 1900.

In some examples, the plurality of individual feltable fibers 1922 may be matted, condensed, pressed, bonded, etc. together to form a non-woven structure or construct integrated with the collagen base material 1924. In some examples, the individual feltable fibers 1922 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 1922 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 1922 may include combinations of any of the materials disclosed. For example, the feltable fibers 1922 may be formed from a combination of any of natural fibers, synthetic fibers, biodegradable fibers, collagen fibers, or the like. It can be further appreciated that the feltable fibers 1922 may be randomly aligned with one another to form a non-woven felted structure. In other examples, however, the feltable fibers 1922 may be aligned with one another to achieve unidirectional stiffness properties with the non-woven felted structure.

FIG. 39 further illustrates that the repair implant 1900 may also include one or more sutures attached to the biologic component 1924. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 39 illustrates that the repair implant 1900 may include sutures 1926a, 1926b, 1926c, 1926d each of which have a first end which may pass through the folded region 1932 of the biologic component 1924 and extend into the biologic component 1924. It can be appreciated that the first ends of the sutures 1926a, 1926b, 1926c, 1926d may be anchored within a portion of the biologic component 1924. Further, in some examples, the first end of the sutures 1926a, 1926b, 1926c, 1926d may engage the feltable fibers 1922, thereby anchoring the sutures 1926a, 1926b, 1926c, 1926d within a feltable mesh formed by the feltable fibers 1922. As will be discussed in greater detail below, it can be appreciated that the sutures 1926a, 1926b, 1926c, 1926d may include a second end, opposite the first end, whereby the second end is configured to anchor into bone, etc.

FIG. 40 illustrates a perspective view of a portion of a shoulder joint including a rotator cuff tendon 1950 and the footprint of the greater tuberosity 1952 of a humerus.

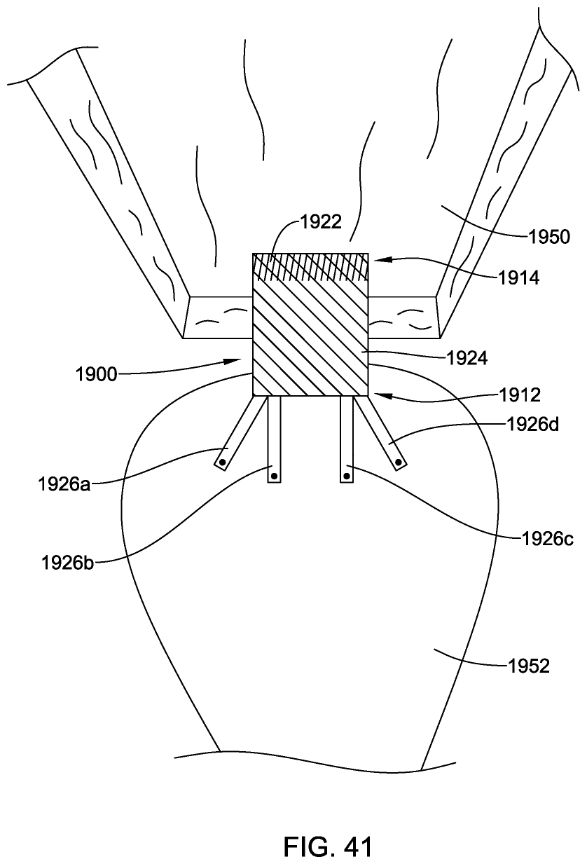

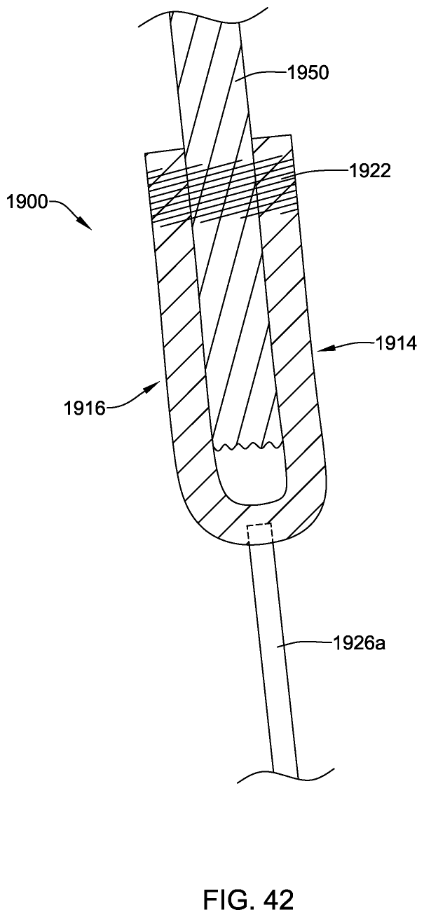

FIG. 41 illustrates a perspective view of the example implant 1900 positioned along the shoulder joint shown in FIG. 40. FIG. 41 illustrates that the first flap 1914 and the second flap 1916 (shown in FIG. 39 and FIG. 42) of the repair implant 1900 may be attached to the tendon 1950 via felting the feltable fibers 1922 to the tendon using any of the felting techniques described herein. Further, FIG. 41 illustrates that the sutures 1926a, 1926b, 1926c, 1926d may be attached to the footprint of the greater tuberosity 1952 using any suitable anchor (e.g., knotless bone anchor, bone anchor, etc.) described herein.

FIG. 42 illustrates the first flap 1914 and the second flap 1916 of the repair implant 1900 felted to the tendon 1950. It can be appreciated the a felting technique utilized to felt the first flap 1914 and the second flap 1916 of the repair implant 1900 felted to the tendon 1950 may include pushing the feltable fibers 1922 of the first flap into the tissue of the tendon 1950 while also pulling the feltable fibers 1922 of the second flap 1916 back into the tissue of the tendon 1950. In other words, felting of the first flap 1914 and the second flap 1916 to the tendon may occur without having to reposition the felting device to push the feltable fibers 1922 into the second flap 1916. In other examples, the feltable fibers 1922 of the first flap 1914 may be pushed into the tendon 1950, whereby the felting device may be repositioned such that the feltable fibers 1922 of the second flap 1916 may be pushed into the tendon 1950.

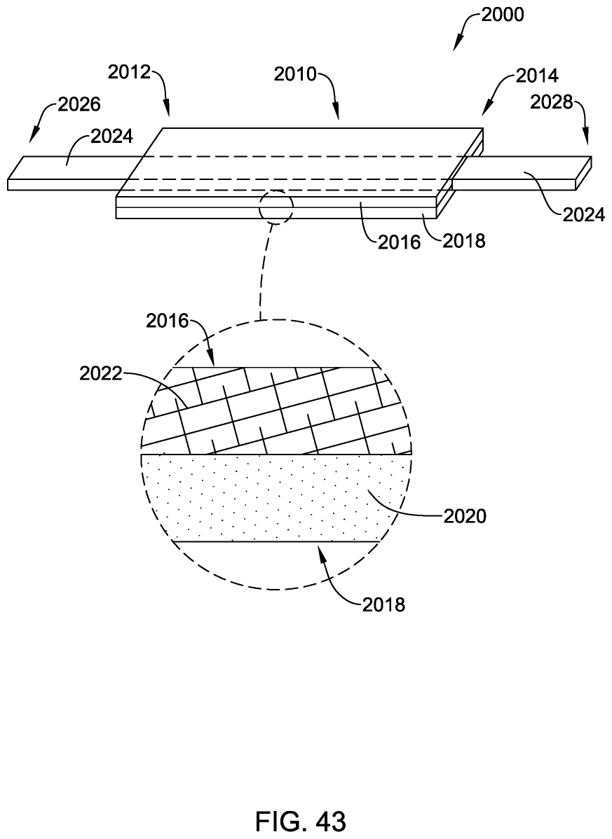

FIG. 43 illustrates a repair implant 2000. The repair implant 2000 may include a base component 2010 having a first end 2012 and a second end 2014. In some examples, the base component 2010 may include a rectangular shape. However, in other examples, the base component 2010 may include a circular, ovular, triangular, square, polygonal, or other geometric shape. Additionally, FIG. 43 further illustrates that the base component 2010 may include a first layer 2016 and a second layer 2018. For example, the detailed view of FIG. 43 illustrates the first layer 2016 positioned adjacent to (e.g., contacting, engaging, etc.) the second layer 2018.

Further, the detailed view of FIG. 43 illustrates that the first layer 2016 of the base component 2010 may include a feltable material 2022. For example, the detailed view of FIG. 43 illustrates the first layer 2016 may include a plurality of feltable fibers 2022 which extend from the first end 2012 to the second end 2014 of the base component 2010.

In some examples, the plurality of individual feltable fibers 2022 forming the first layer 2016 may be matted, condensed, pressed, bonded, etc. together to form a non-woven structure or construct. In some examples, the individual feltable fibers 2022 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2022 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2022 may include combinations of any of the materials disclosed. For example, the feltable fibers 2022 may be formed from a combination of any of natural fibers, synthetic fibers, biodegradable fibers, collagen fibers, or the like. It can be further appreciated that the feltable fibers 2022 may be randomly aligned with one another to form a non-woven felted structure. In other examples, however, the feltable fibers 2022 may be aligned with one another to achieve unidirectional stiffness properties with the non-woven felted structure.

Further, the detailed view of FIG. 43 illustrates that the second layer 2018 of the base component 2010 may be formed from a biologic component 2020. For example, the second layer 2018 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the second layer 2018 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the second layer 2018 of the base component 2010. For example, a lattice structure and/or other structures described herein may be formed by molding which may be punched, woven, etc. to form the desired arrangement. The second layer 2018 of the base component 2010 may be porous or included pathways to encourage tissue in-growth. In some embodiments, the second layer 2018 of the base component 2010 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 43 further illustrates that the repair implant 2000 may also include one or more sutures 2024 coupled to the base component 2010. For purposes of discussion herein, suture 2024 may include suture-wire, suture tape, fiber wire, braided sutures, woven sutures, non-absorbable sutures, absorbable sutures, synthetic sutures, natural sutures, multifilament sutures, monofilament sutures and any combinations thereof. As illustrated in FIG. 43, the suture 2024 may include a first end region 2026 which extends beyond the first end 2012 of the base component 2010, thereby defining a length of the suture 2024 which extends laterally away from the first end 2012 of the base component 2010. Additionally, the suture 2024 may include a second end region 2028 which extends beyond the second end 2014 of the base component 2010, thereby defining a length of the suture 2024 which extends laterally away from the second end 2012 of the base component 2010.

FIG. 43 illustrates that the suture 2024 may be integrated with the first layer 2016 and/or the second layer 2018 of the base component 2010. For example, FIG. 43 illustrates that the suture 2024 may be positioned between the first layer 2016 and the second layer 2018 of the base component 2010. In some examples, one or more of the individual fibers 2022 of the first layer 2016 may be engaged with the suture 2024. For example, one or more of the individual fibers 2022 of the first layer 2016 may be felted to the material used to form the suture 2024, thereby securing the suture 2024 to a portion of the plurality of fibers 2022 defining the first layer 2016.

It can be appreciated that the suture 2024 may be utilized to secure the repair implant 2000 at a target tissue site.

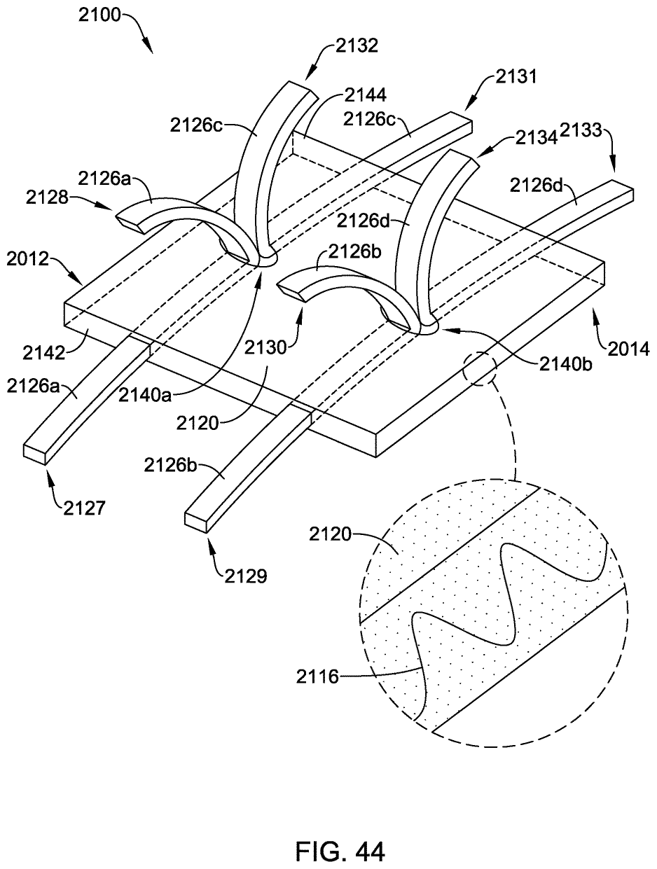

FIG. 44 illustrates a repair implant 2100. The repair implant 2100 may include a biologic component 2120 having a first end region 2112 and a second end region 2114. The biologic component 2120 may include collagen, a collagen element and/or a collagen-like material similar to other bioinductive collagen constructs described herein. In some instances, the biologic component 2120 may be described as a collagen implant.

Additionally, the detailed view of FIG. 44 illustrates that the repair implant 2100 may also include a plurality of feltable fibers 2116 integrated into the biologic component 2120. In some examples, the feltable fibers 2116 may be uniformly dispersed within the biologic component 2120 from the first end region 2112 to the second end region 2114. In other examples, the feltable fibers 2116 may be non-uniformly dispersed within the biologic component 2120. In yet other examples, the feltable fibers 2116 may be described as a “felting mesh” that is integrated within the biologic component 2120.

The feltable fibers 2116 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2116 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2116 may include combinations of any of the materials disclosed. For example, the feltable fibers 2116 may be formed from a combination of any of natural fibers, synthetic fibers, biodegradable fibers, collagen fibers, or the like.

FIG. 44 further illustrates that the repair implant 2100 may also include one or more sutures attached to the biologic component 2120. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 44 illustrates that the repair implant 2100 may include sutures 2126a, 2126b, 2126c, 2126d.

Further, FIG. 44 illustrates that each of the sutures 2126a, 2126b, 2126c, 2126d may pass through a face of the biologic component 2120, extend into a portion of the biologic component 2120 and exit the biologic component 2120 through an aperture 2140a, 2140b. For example, FIG. 44 illustrates the repair implant 2120 may include a first suture 2126a having a first end 2127 and a second end 2128. Additionally, the suture 2126a may extend through a face 2142 of the biologic component 2120, through a portion of the biologic component 2120 and exit the biologic component 2120 through the aperture 2140a. Similarly, FIG. 44 illustrates the repair implant 2120 may include a second suture 2126b having a first end 2129 and a second end 2130. Additionally, the suture 2126b may extend through the face 2142 of the biologic component 2120, through a portion of the biologic component 2120 and exit the biologic component 2120 through an aperture 2140b. Similarly, FIG. 44 illustrates the repair implant 2120 may include a third suture 2126c having a first end 2131 and a second end 2132. Additionally, the suture 2126c may extend through a face 2144 of the biologic component 2120, through a portion of the biologic component 2120 and exit the biologic component 2120 through the aperture 2140a. Similarly, FIG. 44 illustrates the repair implant 2120 may include a fourth suture 2126d having a first end 2133 and a second end 2134. Additionally, the suture 2126d may extend through a face 2144 of the biologic component 2120, through a portion of the biologic component 2120 and exit the biologic component 2120 through the aperture 2140b.

Further, in some examples, the individual fibers used to construct the sutures 2126a, 2126b, 2126c, 2126d may engage the feltable fibers 2116, thereby anchoring the sutures 2126a, 2126b, 2126c, 2126d within the feltable mesh formed by the feltable fibers 2116.

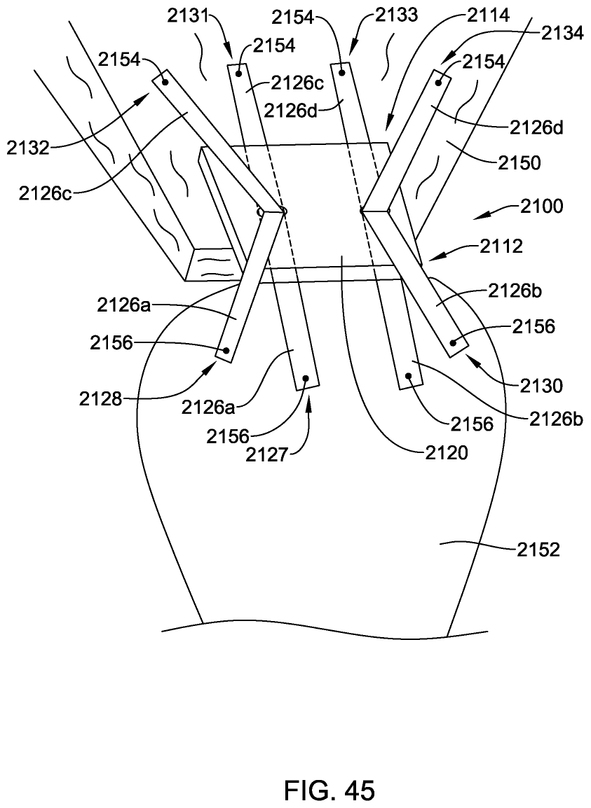

FIG. 45 illustrates a perspective view of the example tendon repair device 2100 implanted within a shoulder. It can be appreciated from FIG. 45 that the second end region 2114 of the tendon repair device 2100 may be positioned adjacent to a rotator cuff tendon 2150 and the first end region 2112 of the tendon repair device 2100 may be positioned adjacent to the footprint of the greater tuberosity 2152. Further, FIG. 45 illustrates that the first and second ends 2131, 2132, respectively, of the third suture 2126c and the first and second ends 2133, 2134, respectively, of the fourth suture 2126d may be attached to the rotator cuff tendon via one or more anchors 2154. Similarly, FIG. 45 illustrates that the first and second ends 2127, 2128, respectively, of the first suture 2126a and the first and second ends 2129, 2130, respectively, of the second suture 2126b may be attached to the footprint of the greater tuberosity 2152 via one or more anchors 2156. It can be further appreciated that each of the sutures 2126a, 2126b, 2126c, 2126d may be individually tensioned (via the placement and attachment of the anchors 2154, 2156) to tailor the placement and engagement of the biologic component 2120 relative to the torn portion of the rotator cuff tendon 2150.

Additionally, it can be appreciated that, in some examples, after the repair implant 2100 has been initially positioned and attached using the sutures 2126a, 2126b, 2126c, 2126d as described herein, the repair implant 2100 may be further secured via felting portions of the repair implant 2100 to the rotator cuff tendon 2150 and/or the footprint of the greater tuberosity 2152. For example, the first end region 2112 of the repair implant 2100 may be felted to the footprint of the greater tuberosity 2152 while the second end region 2114 of the repair implant 2100 may be felted to the rotator cuff tendon 2150.

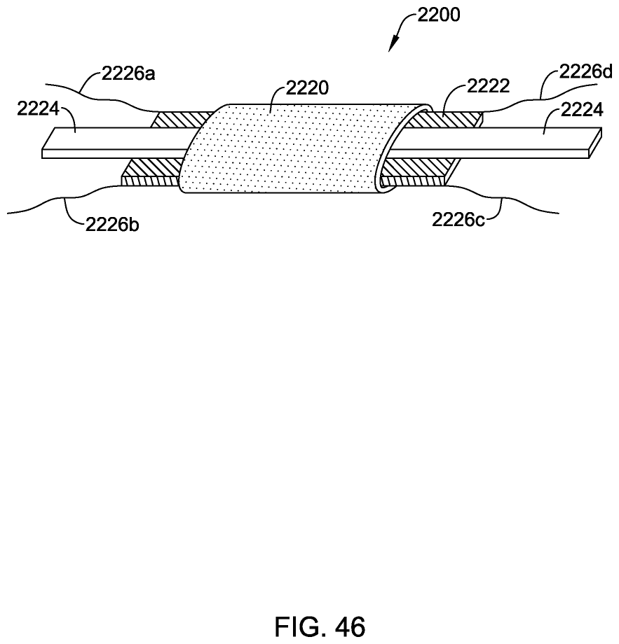

FIG. 46 illustrates another repair implant 2200. The repair implant 2200 may include a felting patch 2222. Additionally, the repair implant 2200 may include a first suture 2226a, a second suture 2226b, a third suture 2226c and a fourth suture 2226d attached thereto and extending away from the felting patch 2222. Further, FIG. 46 illustrates that the repair implant 2200 may further include a strip of suture tape 2224 coupled to the felting patch 2222. Further yet, the repair implant 2200 may also include a tubular biologic component 2220 coupled to the felting patch 2222 and/or the suture tape 2224.

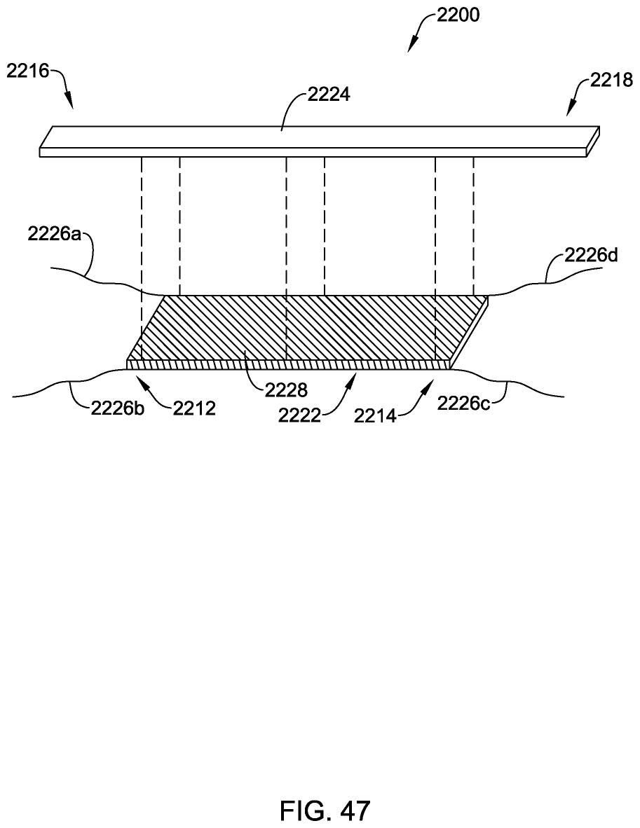

FIG. 47 illustrates an exploded view of a portion of the repair implant 2200 described herein. FIG. 47 illustrates the felting patch 2222 having a first end region 2212 and a second end region 2214. As discussed herein, FIG. 47 illustrates that the repair implant 2200 may include a first suture 2226a and a second suture 2226b extending from the first end region 2212 of the felting patch 2222 and a third suture 2226c and a fourth suture 2226d extending from the second end region 2214 of the felting patch 2222. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. Further, in some examples, each of the first suture 2226a, the second suture 2226b, the third suture 2226c, and the fourth suture 2226d may be coupled to the felting patch 2222 at each of the four corners of the felting patch 2222. However, it is also contemplated that the sutures 2226a-2226d may be coupled to any portion of the felting patch 2222.

It can be appreciated that the felting patch 2222 may be formed from a plurality of feltable fibers 2228. In some examples, the feltable fibers 2228 may be described as a “felting mesh.” The feltable fibers 2228 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2228 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2228 may include combinations of any of the materials disclosed. For example, the feltable fibers 2228 may be formed from a combination of any of natural fibers, synthetic fibers, biodegradable fibers, collagen fibers, or the like.

Additionally, FIG. 47 illustrates the suture tape 2224 described herein. The suture tape 2224 may include a first end 2216 and a second 2218. It can be appreciated that the first end 2216 of the suture tape 2224 may extend past the first end region 2212 of the felting patch 2222. Similarly, it can be appreciated that the second end 2218 of the suture tape 2224 may extend past the second end region 2214 of the felting patch 2222.

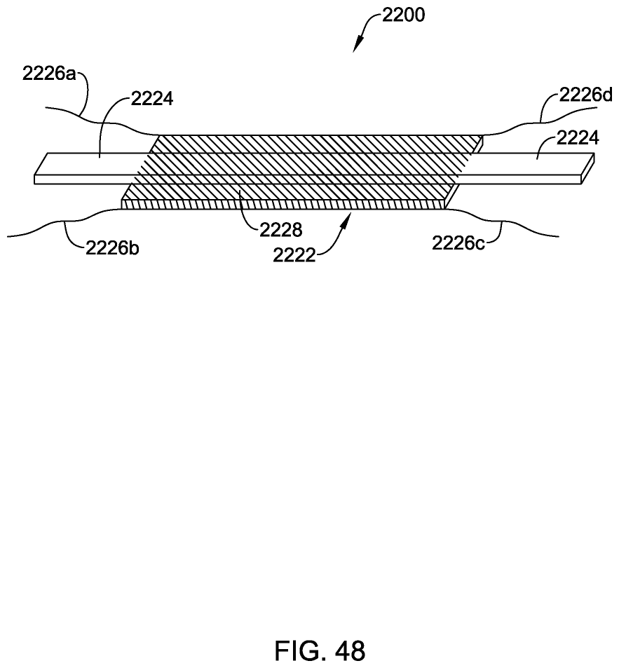

FIG. 48 illustrates that the suture tape 2224 may be attached to the felting patch 2222 via felting the suture tape 2224 to the felting patch 2222. It can be appreciated from FIG. 48 that the suture tape 2224 may be felted to the felting patch 2222 along the entire length of the felting patch 2222. It can be further appreciated that felting the suture tape 2224 to the felting patch 2222 may illustrate a manufacturing step in the construction of the repair implant 2200.

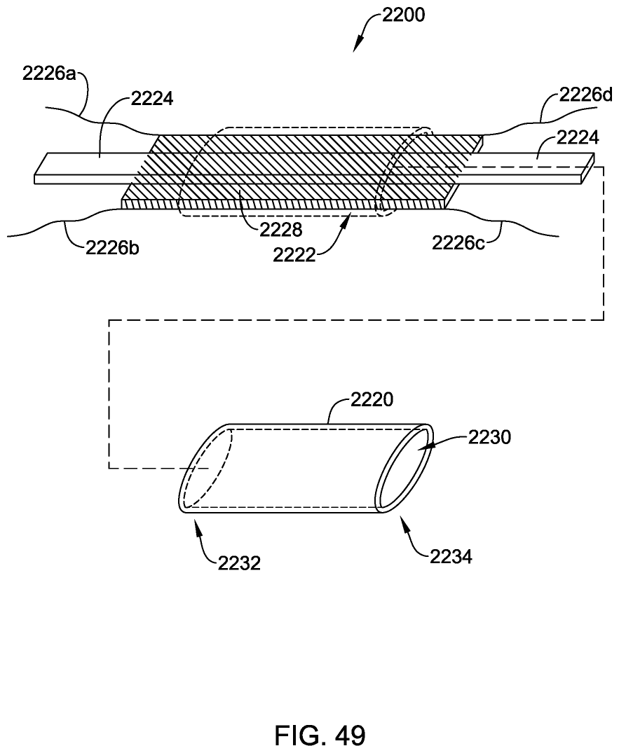

FIG. 49 illustrates a partial exploded view of the repair implant 2200 described herein, whereby FIG. 49 illustrates that the tubular biologic component 2220 may be attached to the felting patch 2222 and/or the suture tape 2224. It can be appreciated from FIG. 49 that, in some examples, the felting patch 2222 and suture tape 2224 assembly (shown in FIG. 48) may be inserted into a lumen 2230 of the biologic component 2220. Further, after being positioned within the lumen 2230 of the biologic component, the biologic component 2220 maybe be felted to the felting patch 2222 and/or suture tape 2224 assembly (shown in FIG. 48) to form the repair implant 2200 illustrated in FIG. 46.

The biologic component 2220 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 2220 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 2220. For example, a lattice structure and/or other structures described herein may be formed by molding which may be punched, woven, etc. to form the desired arrangement. The biologic component 2220 may be porous or included pathways to encourage tissue in-growth. In some embodiments, the biologic component 2220 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Referring to FIG. 46, it can be appreciated that both the felting patch 2222 and the suture tape 2224 may extend past a first end 2232 and a second end 2234 of the biologic component 2220. Accordingly, it can be further appreciated that the biologic component 2220 may be felted to the felting patch 2222 and/or suture tape 2224 along its entire length. Further, it can be appreciated that, in some examples, because the biologic component 2220 may extend circumferentially around both the felting patch 2222 and/or the suture tape 2224, any portion of the biologic construct 2220 may be felted to the felting patch 2222 and/or the suture tape 2224. It can be further appreciated that, in some examples, the suture tape 2224 and the biologic construct 2220 may be positioned relative to one another as shown in FIG. 46 and both the suture tape 2224 and the biologic construct 2220 may be felted to each other and the felting patch 2222.

It can be appreciated that the repair implant 2200 may be configured to provide a combination of structural features, properties and functions that may be used to treat partial or full thickness tears of soft tissues, including partial or full thickness tears of soft tissues. For example, the repair implant 2200 may be configured to treat partial or full thickness tears of an anterior cruciate ligament (ACL). The repair implant 2200 may encourage tissue ingrowth while also providing additional mechanical properties such as, but not limited to strength and stiffness. For example, the implant 2200 may provide a layer of collagen over injured tissue to facilitate healing.

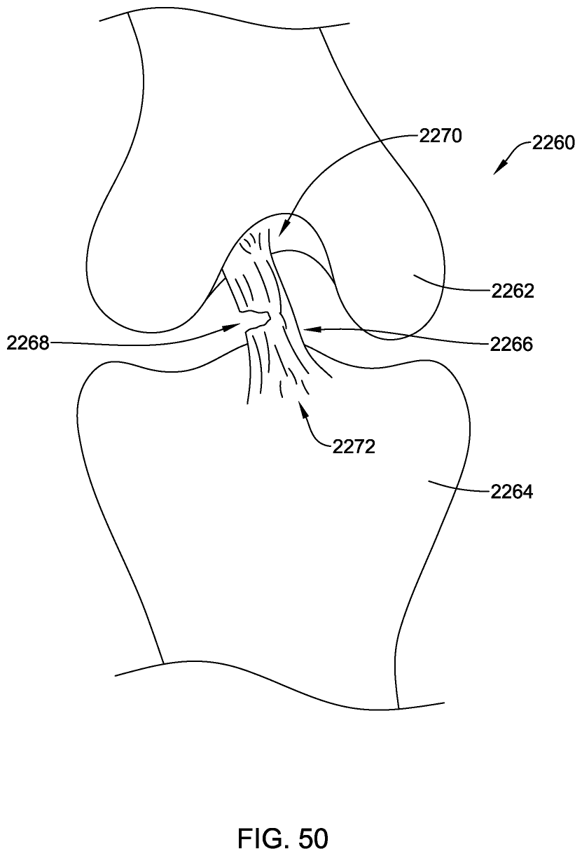

FIG. 50 illustrates a knee joint 2260 including the distal femur 2262 of and the tibia 2264. Additionally, FIG. 50 illustrates the anterior crucial ligament 2266 having a first end region attached to the distal femur 2262 at a femoral attachment region 2270 and second end region attached to the tibia 2264 at a tibial attachment region 2272. Additionally, FIG. 50 illustrates a tear 2268 of the anterior crucial ligament 2266.

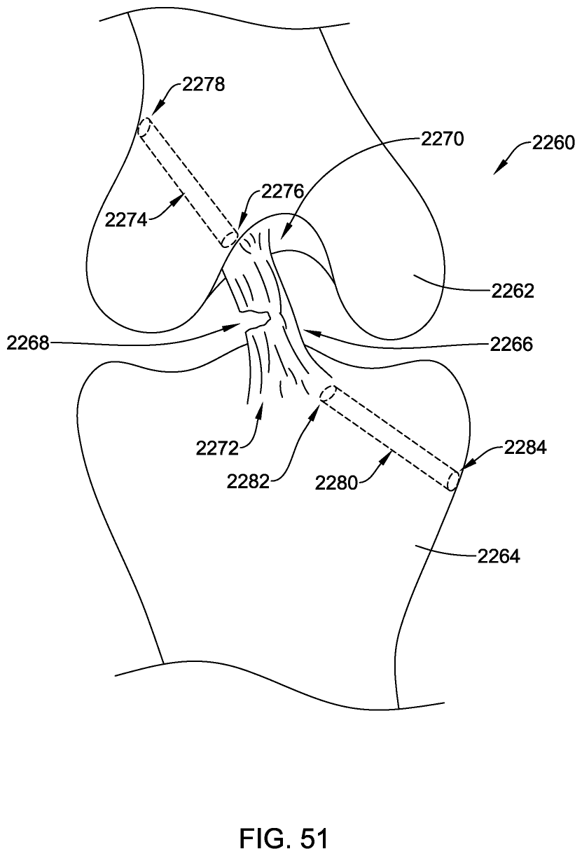

FIG. 51 illustrates a first step in using the repair implant 2200 to repair the tear 2268 in the anterior cruciate ligament 2266. FIG. 51 illustrates that prior to the placement of the repair implant 2200 relative to the tear 2268 of the in the anterior cruciate ligament 2266, a femoral bone tunnel 2274 may be drilled in the distal femur 2262 and a tibial bone tunnel 2280 may be drilled in the tibia 2264. The femoral bone tunnel 2274 may include a femoral entry opening 2276 and a femoral exit opening 2278. The tibial bone tunnel 2280 may include a tibial entry opening 2282 and a tibial exit opening 2284. The femoral entry opening 2276 may be positioned adjacent to the femoral attachment region 2270 of the anterior cruciate ligament 2266 and the tibial entry opening 2282 may be positioned adjacent to the tibial attachment region 2272 of the anterior cruciate ligament 2266.

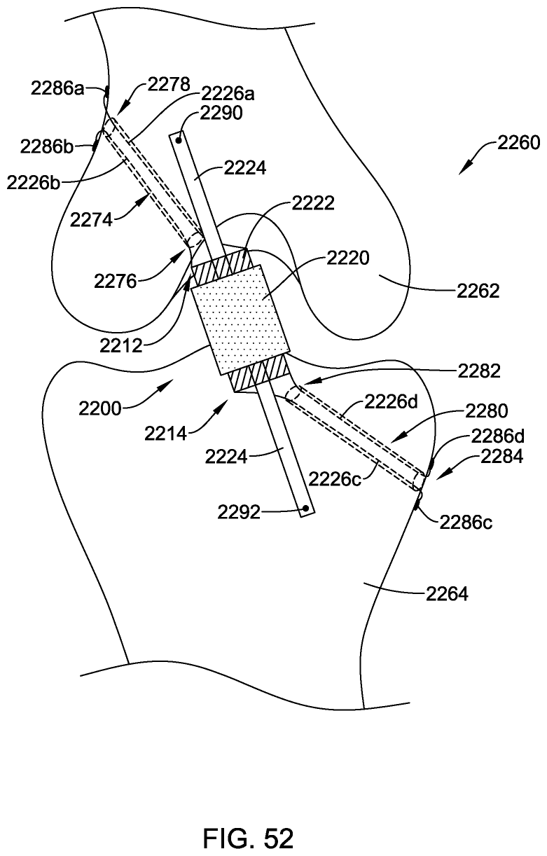

FIG. 52 illustrates the positioning of the repair implant 2200 relative to the tear 2268 of the anterior cruciate ligament 2266 described with respect to FIGS. 50-51. Placement of the repair implant 2200 relative to the tear 2268 of the anterior cruciate ligament 2266 may include passing the first suture 2226a and the second suture 2226b through the femoral entry opening 2276, through the femoral tunnel 2274, and through the femoral exit opening 2278. Alternatively, in other examples, only a single suture 2226a, 2226b may be passing through the femoral entry opening 2276, through the femoral tunnel 2274, and through the femoral exit opening 2278.

After passing the first suture 2226a and the second suture 2226b through the femoral exit opening 2278, the first suture 2226a may be attached to the distal femur 2262 via a bone anchor 2286a and the second suture 2226b may be attached to the distal femur 2262 via a bone anchor 2286b. After anchoring the first suture 2226a and the second suture 2226b to the distal femur 2262, placement of the repair implant 2200 relative to the tear 2268 of the anterior cruciate ligament 2266 may include passing the third suture 2226c and the fourth suture 2226d through the tibial entry opening 2282, through the tibial tunnel 2280, and through the tibial exit opening 2284. Alternatively, in other examples, only a single suture 2226c, 2226d may be passing through the tibial entry opening 2282, through the tibial tunnel 2280, and through the tibial exit opening 2284.

After passing the third suture 2226c and the fourth suture 2226d through the tibial exit opening 2284, the third suture 2226c may be attached to the tibia 2264 via a bone anchor 2286c and the fourth suture 2286d may be attached to the tibia 2264 via a bone anchor 2286d. It can be appreciated that placement and anchoring of the first suture 2226a, the second suture 2226b, the third suture 2226c, and the fourth suture 2226d may permit a clinician to apply an initial, primary tension force of the repair implant 2200 relative to the tear 2268 of the anterior cruciate ligament 2266.

After placement and anchoring of the first suture 2226a, second suture 2226b, third suture 2226c, and fourth suture 2226d, a clinician may attach one end of the suture tape 2274 to the distal femur 2262 using a bone anchor 2290 and the opposite end of the suture tape 2274 to the tibia 2264 using a bone anchor 2292. It can be appreciated that the placement and anchoring of the suture tape 2274 (after the placement and anchoring of the first suture 2226a, the second suture 2226b, the third suture 2226c, and the fourth suture 2226d) may permit a clinician to apply a tension to the repair implant 2200 while the knee joint 2260 is positioned in a desired flexion angle.

It can be further appreciated that after the placement and attachment of the first suture 2226a, the second suture 2226b, the third suture 2226c, the fourth suture 2226d, and the suture tape 2274 (to both the distal femur 2262 and the tibia 2264), the felting patch 2222 may be felted directly to portions of the anterior cruciate ligament 2266 (shown in FIGS. 50-51) adjacent to the tear 2268 of the anterior cruciate ligament 2266. For example, it can be appreciated that the first end region 2212 of the felting patch 2222 may be felted to a portion of the anterior cruciate ligament 2266 which is proximal to the tear 2268 and the second end region 2214 of the felting patch 2222 may be felted to a portion of the anterior cruciate ligament 2266 which is distal to the tear 2268 of the anterior cruciate ligament 2266. It can be further appreciated that felting the first end region 2212 of the felting patch 2222 to a portion of the anterior cruciate ligament 2266 which is proximal to the tear 2268 and felting the second end region 2214 of the felting patch 2222 to a portion of the anterior cruciate ligament 2266 which is distal to the tear 2268 of the anterior cruciate ligament 2266 may position the biologic component 2220 such that it contacts the tear 2268 of the anterior cruciate ligament 2266, thereby permitting the biologic component to encourage tissue ingrowth and facilitate healing of the injured tissue.

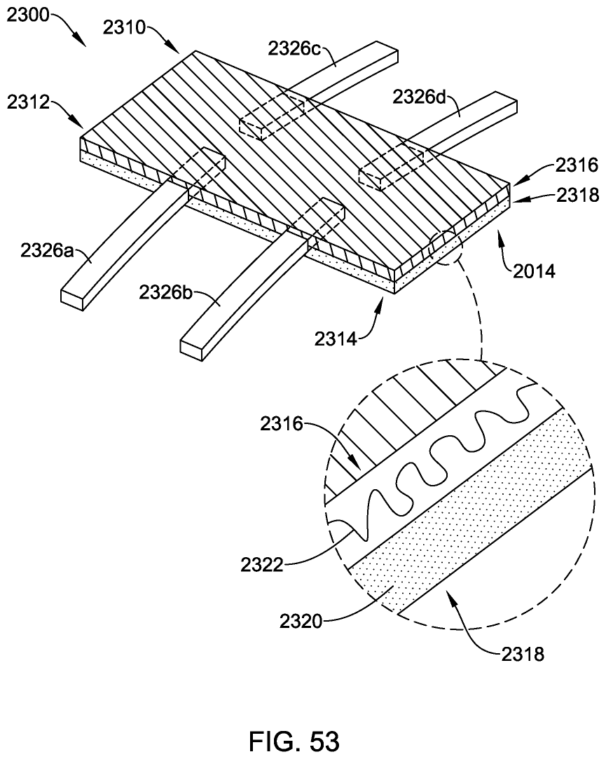

FIG. 53 illustrates a repair implant 2300. The repair implant 2300 may include a base component 2310 having a first end 2312 and a second end 2314. In some examples, the base component 2310 may include a rectangular shape. However, in other examples, the base component 2310 may include a circular, ovular, triangular, square, polygonal, or other geometric shape. Additionally, FIG. 53 further illustrates that the base component 2310 may include a first layer 2316 and a second layer 2318. For example, the detailed view of FIG. 53 illustrates the first layer 2316 positioned adjacent to (e.g., contacting, engaging, etc.) the second layer 2318.

Further, the detailed view of FIG. 53 illustrates that the first layer 2316 of the base component 2310 may include a feltable material 2322. For example, the detailed view of FIG. 53 illustrates the first later 2316 may include a plurality of feltable fibers 2322 which extend from the first end 2312 to the second end 2314 of the base component 2310.

In some examples, the plurality of individual feltable fibers 2322 forming the first layer 2316 may be matted, condensed, pressed, bonded, etc. together to form a non-woven structure or construct. In some examples, the individual feltable fibers 2322 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2322 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2322 may include combinations of any of the materials disclosed. For example, the feltable fibers 2322 may be formed from a combination of any of natural fibers, synthetic fibers, biodegradable fibers, collagen fibers, or the like. It can be further appreciated that the feltable fibers 2322 may be randomly aligned with one another to form a non-woven felted structure. In other examples, however, the feltable fibers 2322 may be aligned with one another to achieve unidirectional stiffness properties with the non-woven felted structure.

Further, the detailed view of FIG. 53 illustrates that the second layer 2318 of the base component 2310 may be formed from a biologic component 2320. For example, the second layer 2318 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the second layer 2318 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the second layer 2318 of the base component 2310. For example, a lattice structure and/or other structures described herein may be formed by molding which may be punched, woven, etc. to form the desired arrangement. The second layer 2318 of the base component 2310 may be porous or included pathways to encourage tissue in-growth. In some embodiments, the second layer 2318 of the base component 2310 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 53 further illustrates that the repair implant 2300 may also include one or more sutures attached to the base component 2310. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 53 illustrates that the repair implant 2300 may include sutures 2326a, 2326b, 2326c, 2326d each of which have a first end which may pass extend into the base component 2310. It can be appreciated that the first ends of the sutures 2326a, 2326b, 2326c, 2326d may be anchored within a portion of the base component 2310. FIG. 53 illustrates that the sutures 2326a, 2326b, 2326c, 2326d may be integrated with the first layer 2316 and/or the second layer 2318 of the base component 2310. For example, FIG. 53 illustrates that the sutures 2326a, 2326b, 2326c, 2326d may be positioned between the first layer 2316 and the second layer 2318 of the base component 2310. In some examples, one or more of the individual fibers 2322 of the first layer 2316 may be engaged with one or more of the sutures 2326a, 2326b, 2326c, 2326d. For example, one or more of the individual fibers 2322 of the first layer 2316 may be felted to the material used to form the sutures 2326a, 2326b, 2326c, 2326d, thereby securing the sutures 2326a, 2326b, 2326c, 2326d to a portion of the plurality of fibers 2322 defining the first layer 2316. Additionally, it can be appreciated that each of the sutures 2326a, 2326b, 2326c, 2326d may include a second end, opposite the first end, whereby the second end is configured to anchor into bone, etc.

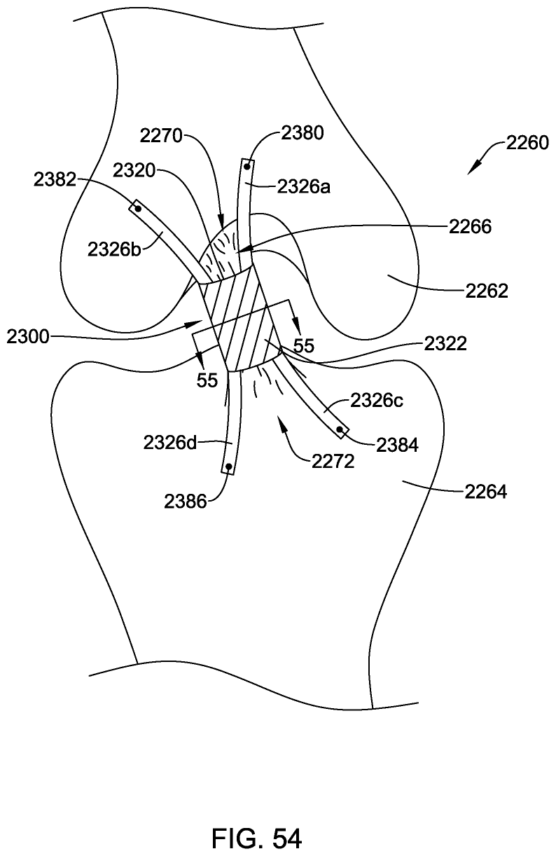

FIG. 54 illustrates the repair implant 2300 implanted along the anterior cruciate ligament 2266 of the example knee joint 2260 (the knee joint 2260 is also illustrated in FIG. 50). Like the knee joint illustrated in FIG. 50, the knee joint 2260 includes the distal femur 2262 of a femur and the tibia 2264. Additionally, FIG. 54 illustrates the anterior crucial ligament 2266 having a first end region attached to the distal femur 2262 at a femoral attachment region 2270 and second end region attached to the tibia 2264 at a tibial attachment region 2272. It can be appreciated that the example knee joint includes a tear 2268 (shown in FIG. 50) in the anterior cruciate ligament 2266.

FIG. 54 further illustrates that the initial placement of the repair implant 2300 along the tear of the 2268 (shown in FIG. 50) in the anterior cruciate ligament 2266 may include positioning and attaching the first suture 2326a and the second suture 2326b to the distal femur 2262. It can be appreciated that the first suture 2326a may be attached to the distal femur 2262 via a bone anchor 2380. It can be further appreciated that the second suture 2326a may be attached to the distal femur 2262 via a bone anchor 2382. Additionally, initial placement of the repair implant 2300 along the anterior cruciate ligament 2266 may include positioning and attaching the third suture 2326c and the fourth suture 2326d to the tibia 2264. It can be appreciated that the third suture 2326c may be attached to the tibia 2264 via a bone anchor 2384. It can be appreciated that the fourth suture 2326d may be attached to the tibia 2264 via a bone anchor 2386. It can be further appreciated that each of the sutures 2326a, 2326b, 2326c, 2326d may be individually tensioned (via the placement and attachment of the anchors 2380, 2382, 2384, 2386) to tailor the placement and engagement of the base component 2310 (including the biologic component 2320) relative to the tear 2368 of the anterior cruciate ligament 2266.

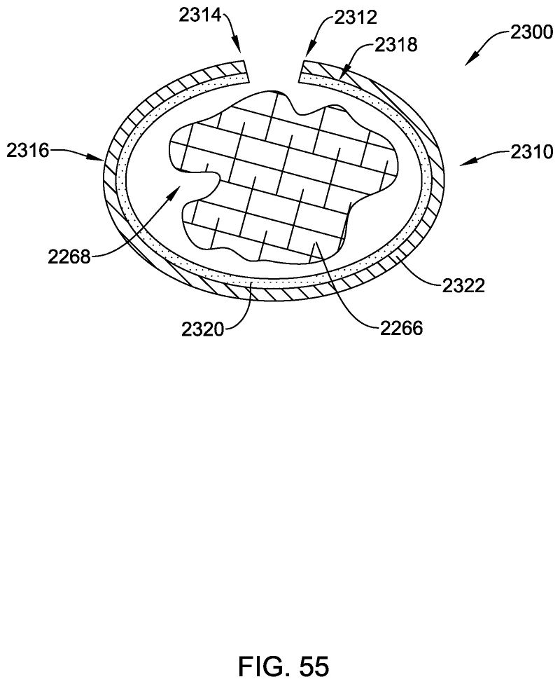

In addition to the placement and attachment of the anchors 2380, 2382, 2384, 2386 discussed herein, FIG. 54 further illustrates that positioning the repair implant 2300 along the injured tissue of the anterior cruciate ligament 2266 may include wrapping the base component 2310 around the anterior cruciate ligament 2266. For example, FIG. 55 illustrates a cross-sectional view taken along line 55-55 of FIG. 54. FIG. 55 illustrates the base component 2310 wrapped around the anterior cruciate ligament 2266 such that the first end 2312 is positioned near the second end 2314.

Further, FIG. 55 illustrates that the base component 2310 may be wrapped around the anterior cruciate ligament 2266 such that the biologic component 2320 of the second layer 2318 contacts the injured, target tissue (including the tear 2268) of the anterior cruciate ligament 2266. Additionally, it can be appreciated that positioning the base component 2310 around the anterior cruciate ligament 2266 such that the biologic component 2320 of the second layer 2318 contacts the injured, positions the first layer 2016 (including the feltable fibers 2316) facing outwardly relative to the anterior cruciate ligament 2266. Accordingly, it can be appreciated that this configuration may permit a clinician to attach a portion of the base component 2310 to the anterior cruciate ligament 2266 via felting the feltable fibers 2322 directly to the anterior cruciate ligament 2266. It can be further appreciated that the feltable fibers 2322 may be felted to a portion of the anterior cruciate ligament 2266 which is proximal to the tear 2268 and/or a portion of the anterior cruciate ligament 2266 which is distal to the tear 2268.

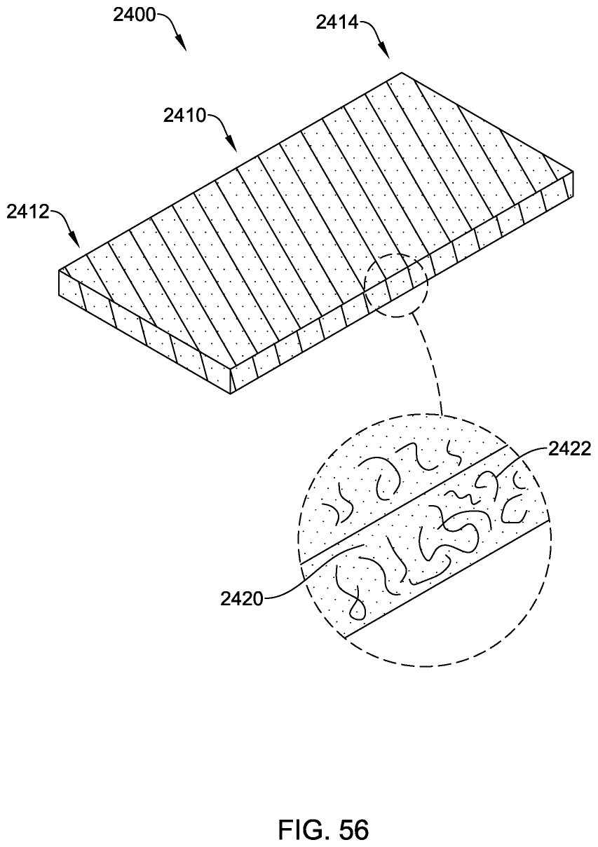

FIG. 56 illustrates repair implant 2400. The repair implant 2400 may include a base component 2410 having a first end region 2412 and a second end region 2414. In some examples, the base component 2410 may be constructed from a plurality of sutures and/or suture elements. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

Additionally, the detailed view of FIG. 56 illustrates that the repair implant 2400 may further include a plurality of feltable fibers 2422 integrated with the base component 2410. For example, it can be appreciated that the feltable fibers 2422 may be integrated with the suture elements which form the base component 2410. It can be appreciated that the individual feltable fibers 2422 may be matted, condensed, pressed, bonded, etc. together to form a non-woven structure or construct. In some examples, the individual feltable fibers 2422 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2422 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2422 may include combinations of any of the materials disclosed. For example, the feltable fibers 2422 may be formed from a combination of any of natural fibers, synthetic fibers, biodegradable fibers, collagen fibers, or the like. It can be further appreciated that the feltable fibers 2422 may be randomly aligned with one another to form a non-woven felted structure. In other examples, however, the feltable fibers 2422 may be aligned with one another to achieve unidirectional stiffness properties with the non-woven felted structure.

Additionally, FIG. 56 illustrates that the repair implant 2400 may further include a biologic component 2420 integrated with the base component 2410 and/or the feltable fibers 2422 described herein. For example, the biologic component 2420 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 2420 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 2420. For example, a lattice structure and/or other structures described herein may be formed by molding which may be punched, woven, etc. to form the desired arrangement. The biologic component 2420 may be porous or included pathways to encourage tissue in-growth. In some embodiments, the biologic component 2420 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

It can be appreciated that the repair implant 2400 may be configured to provide a combination of structural features, properties and functions that may be used to treat soft tissues. In some examples, the repair implant 2400 may be configured to reinforce and/or strengthen tissues. The repair implant 2400 may encourage tissue ingrowth while also providing additional mechanical properties such as, but not limited to, strength and stiffness. For example, the repair implant 2400 may be configured to provide additional rotational stability to the knee joint via augmenting the iliotibial band of the knee joint.

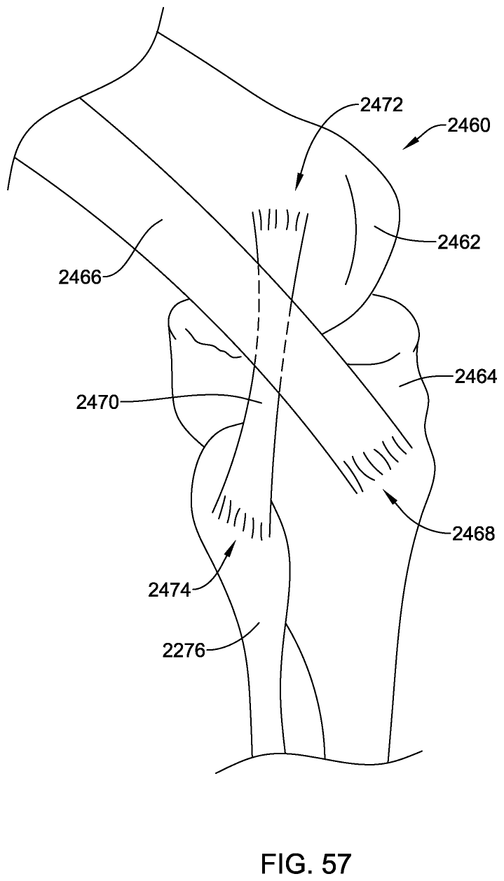

FIG. 57 illustrates a knee joint 2460 including a distal femur 2462, a tibia 2464 and a fibula 2476. Additionally, FIG. 57 illustrates the iliotibial band 2466 of the knee joint 2460 extending along the femur 2462 and attaching to the tibia 2464 at a tibial attachment region 2468. Further, FIG. 57 illustrates the lateral collateral ligament 2470 extending from a fibula attachment region 2474 along the fibula 2276 to a femur attachment region 2472 on the distal femur 2464. It can be appreciated from FIG. 57 that the lateral collateral ligament 2470 may extend underneath the iliotibial band 2466.

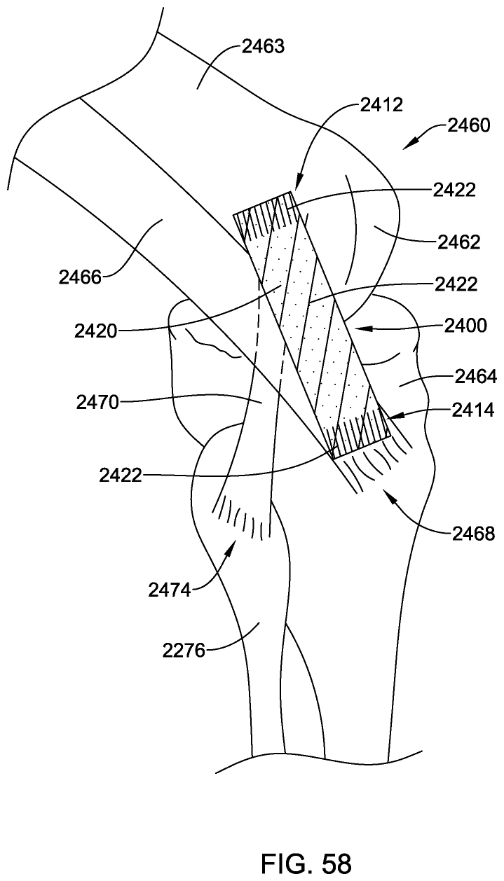

FIG. 58 illustrates the repair implant 2400 being utilized to provide additional rotational stability to the iliotibial band 2466 of the knee joint 2460. FIG. 58 illustrates that, in some examples, the first end region 2412 of the repair implant 2400 may be attached to the distal femur 2462 and the second end region 2414 of the repair implant 2400 may be attached along the iliotibial band 2466. Additionally, it can be appreciated that attaching the repair implant 2400 to the distal femur 2462 may include felting (or attaching using any suitable anchor described herein) the first end region 2412 of the repair implant 2400 directly to tissue along the femur 2462 and felting the second end region 2414 of the repair implant 2400 directly to the iliotibial band 2466.

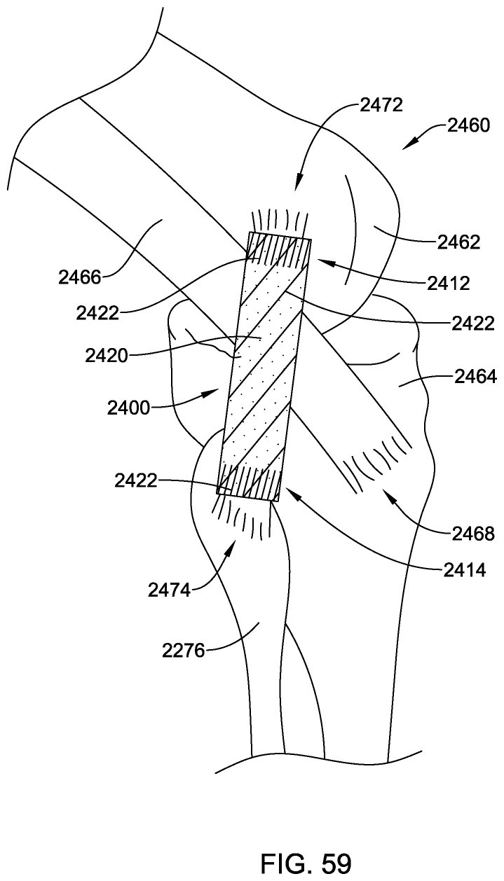

FIG. 59 illustrates another example in which the repair implant 2400 is being utilized to provide additional rotational stability to the iliotibial band 2466 of the knee joint 2460. FIG. 59 illustrates that, in some examples, the first end region 2412 of the repair implant 2400 may be attached to the lateral collateral ligament 2470 adjacent to the femur attachment region 2472 and the second end region 2414 of the repair implant 2400 may be attached to the lateral collateral ligament 2470 adjacent to the fibula attachment region 2474. Additionally, it can be appreciated that attaching the repair implant 2400 to the lateral collateral ligament 2470 may include felting the first end region 2412 and the second end region 2414 of the repair implant 2400 directly to the lateral collateral ligament 2470.

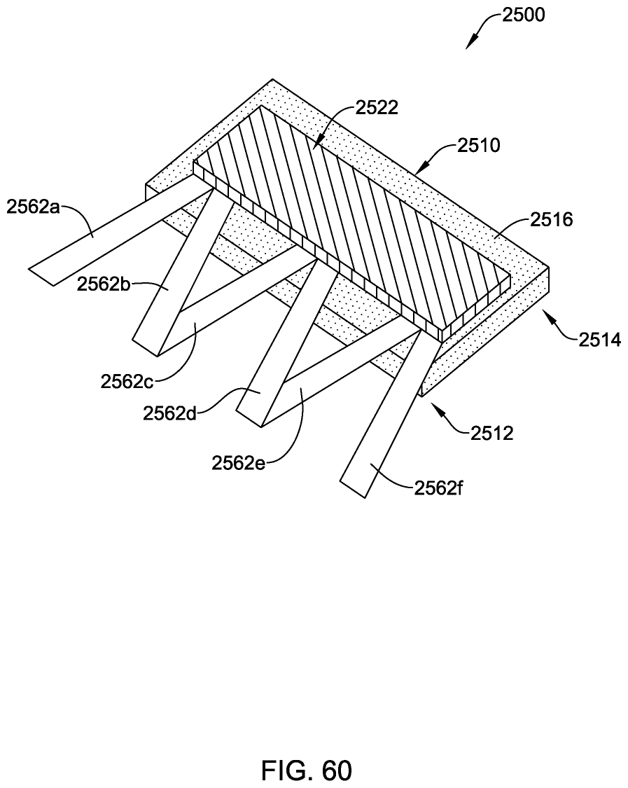

FIG. 60 illustrates a repair implant 2500. The repair implant 2500 may include a biologic component 2510 having a first end 2512 and a second end 2514. In some examples, the biologic component 2510 may include a rectangular shape. However, in other examples, the biologic component 2510 may include a circular, ovular, triangular, square, polygonal, or other geometric shape.

In some examples, the biologic component 2510 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 2510 may include a lattice structure that can be 3D printed in a flat 2-dimensional pattern or a 3-dimensional pattern depending on the 3D printing technology utilized. While 3D printing is one illustrative example, other suitable manufacturing techniques, including chemical bonding methods, adhesive bonding methods, etc. may be utilized to manufacture the biologic component 2510. For example, a lattice structure and/or other structures described herein may be formed by molding which may be punched, woven, etc. to form the desired arrangement. The biologic component 2510 may be porous or included pathways to encourage tissue in-growth. In some embodiments, biologic component 2510 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Further, FIG. 60 illustrates that the repair implant may include a plurality of feltable fibers 2522 positioned along a top surface 2516 of the biologic component 2510. In some examples, the feltable fibers 2522 may be formed into a rectangular-shaped, feltable strip of fibers 2522 extending along the upper surface 2516 of the biologic component 2510. In other examples, the feltable fibers 2522 may be formed into other shapes, including circular, ovular, triangular, square, polygonal, or other geometric shapes.

In some examples, the feltable fibers 2522 may be matted, condensed, pressed, bonded, etc. together to form a non-woven structure or construct. In some examples, the individual feltable fibers 2522 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2522 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2522 may include combinations of any of the materials disclosed. For example, the feltable fibers 2522 may be formed from a combination of any of natural fibers, synthetic fibers, biodegradable fibers, collagen fibers, or the like. It can be further appreciated that the feltable fibers 2522 may be randomly aligned with one another to form a non-woven felted structure. In other examples, however, the feltable fibers 2522 may be aligned with one another to achieve unidirectional stiffness properties with the non-woven felted structure.

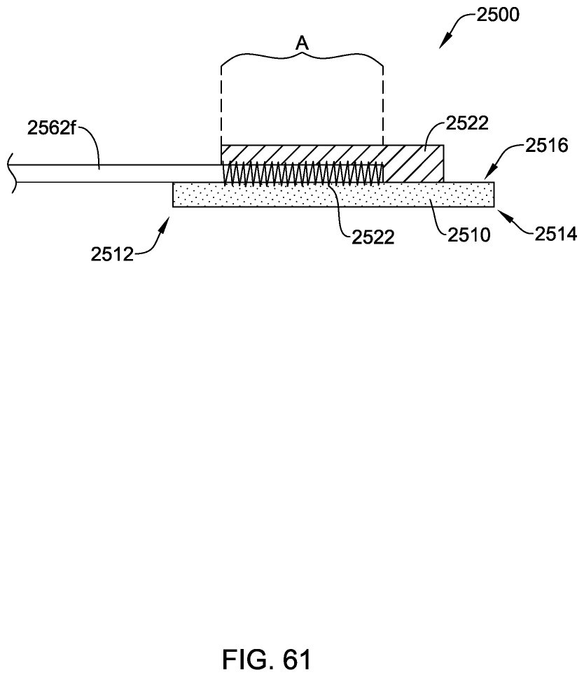

FIG. 60 further illustrates that the repair implant 2500 may also include one or more sutures attached to the biologic component 2510, the feltable fibers 2522 or both the biologic component 2510 and the feltable fibers 2522. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 60 illustrates that the repair implant 2500 may include sutures 2526a, 2526b, 2526c, 2526d, 2526e, 2526f. FIG. 60 illustrates that each of the sutures 2526a, 2526b, 2526c, 2526d, 2526e, 2526f may include a first end positioned on the upper surface 2516 of the biologic component 2510. Additionally, FIG. 60 illustrates that first ends of the sutures 2526a, 2526b, 2526c, 2526d, 2526e, 2526f may be positioned between the feltable fibers 2522 and the base component 2510. For example, FIG. 61 illustrates a side view of the repair implant 2500, whereby the suture 2526f is shown positioned along the upper surface 2516 of the base component 2510 and the feltable fibers 2522 are shown overlapping the first ends of the suture 2526f by a length “A” (it can be appreciated the sutures 2526a, 2526b, 2526c, 2526d, 2526e may also be overlapped by a length A of the feltable fibers 2522, but are hidden from view in FIG. 61).

Additionally, it can be appreciated that one or more of the feltable fibers 2522 may integrate with the first end of one or more of the sutures 2526a, 2526b, 2526c, 2526d, 2526e, 2526f to attach one or more of the sutures 2526a, 2526b, 2526c, 2526d, 2526e, 2526f to the base component 2510. For example, FIG. 61 illustrates one or more of the feltable fibers 2522 may be felted to both the material used to form the sutures 2526a, 2526b, 2526c, 2526d, 2526e, 2526f and the biologic component 2510, thereby securing the sutures 2526a, 2526b, 2526c, 2526d, 2526e, 2526f to the biologic component 2510. Additionally, it can be appreciated that each of the sutures 2526a, 2526b, 2526c, 2526d, 2526e, 2526f may include a second end, opposite the first end, whereby the second end of each suture 2526a, 2526b, 2526c, 2526d, 2526e, 2526f may be configured to anchor into bone, etc.

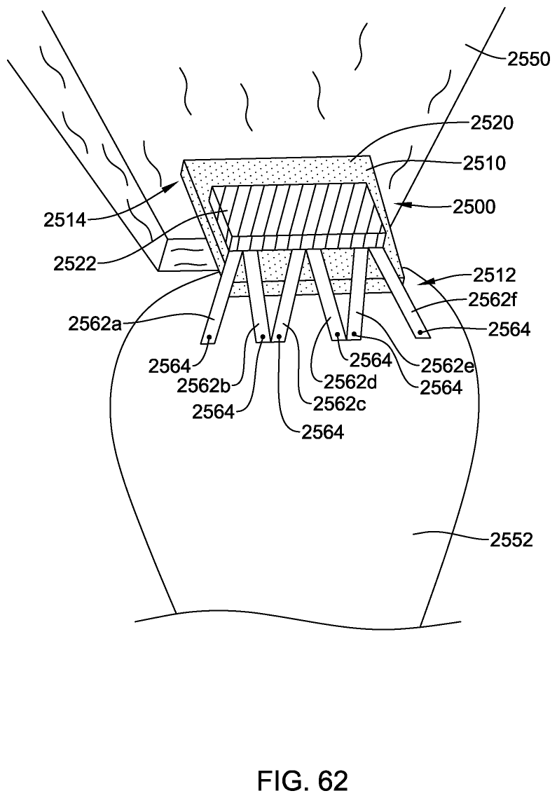

FIG. 62 illustrates a perspective view of the example tendon repair device 2500 implanted within a shoulder. It can be appreciated from FIG. 62 that the second end 2514 of the tendon repair device 2500 may be positioned adjacent to the rotator cuff tendon 2550 and the first end 2512 of the tendon repair device 2500 may be positioned adjacent to the footprint of the greater tuberosity 2552. Further, FIG. 62 illustrates the second end of each of the sutures 2526a, 2526b, 2526c, 2526d, 2526e, 2526f may be attached to the footprint of the greater tuberosity 2552 via a bone anchor 2564. It can be further appreciated that each of the sutures 2526a, 2526b, 2526c, 2526d, 2526e, 2526f may be individually tensioned (via the placement and attachment of the anchors 2564) to tailor the placement and engagement of the biologic component 2510 relative to the torn portion of the rotator cuff tendon 2150.

Additionally, it can be appreciated that, in some examples, after the repair implant 2500 has been initially positioned and attached using the sutures 2526a, 2526b, 2526c, 2526d, 2526e, 2526f as described herein, the repair implant 2500 may be further secured via felting portions of the repair implant 2500 to the rotator cuff tendon 2550 and/or the footprint of the greater tuberosity 2552. For example, the first end 2512 of the repair implant 2500 may be felted to the footprint of the greater tuberosity 2552 while the second end region 2514 of the repair implant 2500 may be felted to the rotator cuff tendon 2550.

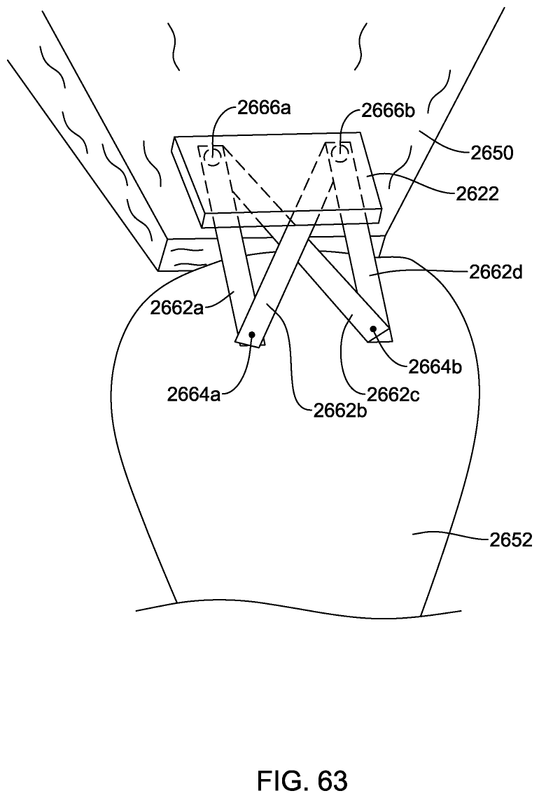

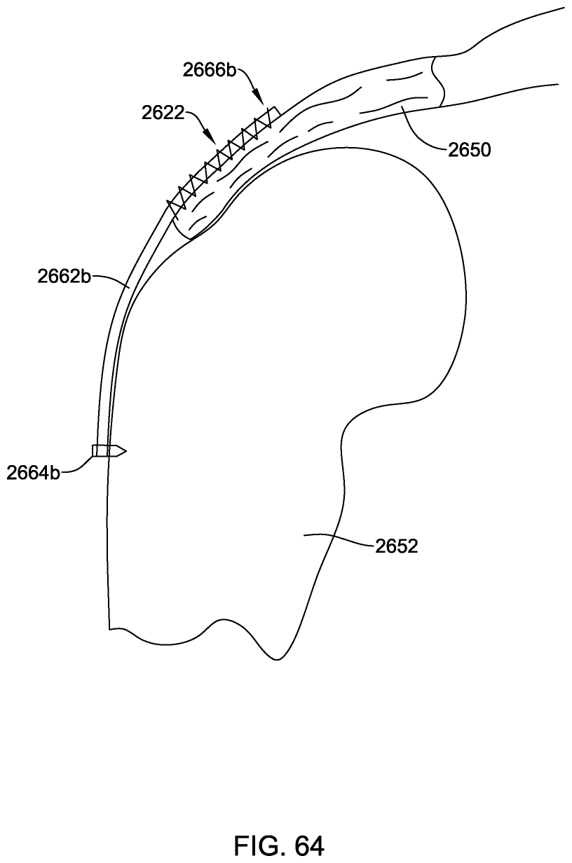

FIGS. 63-64 illustrate that a tendon repair may be performed using one or more sutures in combination (e.g., reinforced) with a feltable construct. For example, FIG. 63 illustrates a rotator cuff tendon repair technique using sutures 2662a, 2662b, 2662c, 2662d which may be reinforced by a feltable construct 2622. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

FIG. 63 illustrates that a first suture 2662a and a second suture 2662b may each include a first end which is anchored to the footprint of the greater tuberosity 2652 via a bone anchor 2664a. Additionally, FIG. 63 illustrates that a third suture 2662c and a fourth suture 2662d may each include a first end which is anchored to the footprint of the greater tuberosity 2652 via a bone anchor 2664b. Further, FIG. 63 illustrates that the first suture 2662a and the third suture 2662c may each include a second end which is anchored to the rotator cuff tendon 2650 via a bone anchor 2666a. Further yet, FIG. 63 illustrates that the second suture 2662b and the fourth suture 2662d may each include a second end which is anchored to the rotator cuff tendon 2650 via a bone anchor 2666b.

Additionally, FIG. 63 illustrates that after the positioning and anchoring of the sutures 2662a, 2662b, 2662c, 2662d, a feltable construct 2622 may be felted to the rotator cuff tendon 2650 over the sutures 2662a, 2662b, 2662c, 2662d. It can be appreciated that the feltable construct 2622 may include a plurality of the feltable fibers which may be matted, condensed, pressed, bonded, etc. together to form a non-woven structure or construct. In some examples, the individual feltable construct 2622 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable construct 2622 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable construct 2622 may include combinations of any of the materials disclosed. For example, the feltable construct 2622 may be formed from a combination of any of natural fibers, synthetic fibers, biodegradable fibers, collagen fibers, or the like. It can be further appreciated that feltable fibers forming the feltable construct 2622 may be randomly aligned with one another to form a non-woven felted structure. In other examples, however, the feltable fibers forming feltable construct 2622 may be aligned with one another to achieve unidirectional stiffness properties with the non-woven felted structure.

Additionally, FIG. 64 illustrates a side view of the suture 2662d extending along the footprint of the greater tuberosity 2652 and the rotator cuff tendon 2650, whereby FIG. 64 further illustrates the feltable construct 2622 felted over the suture 2662d and the bone anchor 2666b (it can be appreciated that the sutures 2662a, 2662b, 2662c are hidden from view in FIG. 64). It can be appreciated that felting the feltable construct 2622 over the sutures 2662a, 2662b, 2662c, 2662d may reinforce the attachment site of the sutures 2662a, 2662b, 2662c, 2662d along the rotator cuff tendon 2650 (including the bone anchors 2666a, 2666b), thereby preventing the sutures 2662a, 2662b, 2662c, 2662d from being torn out of the rotator cuff tendon.

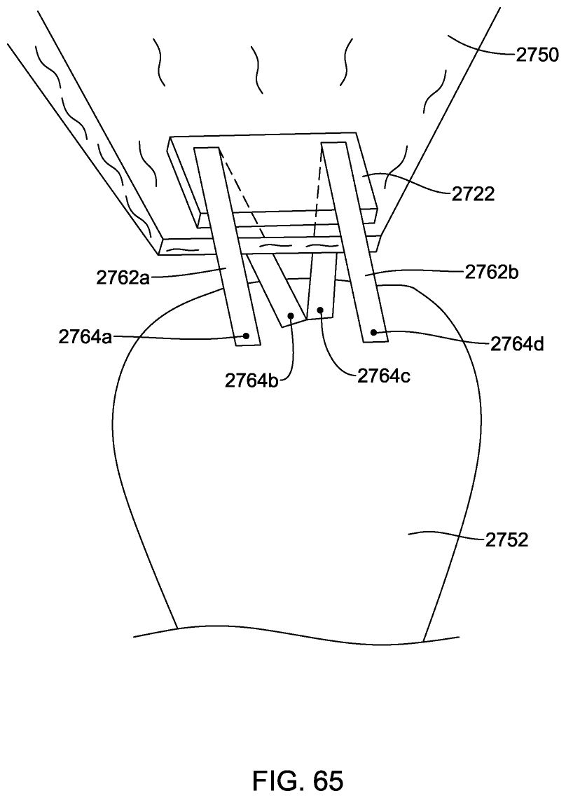

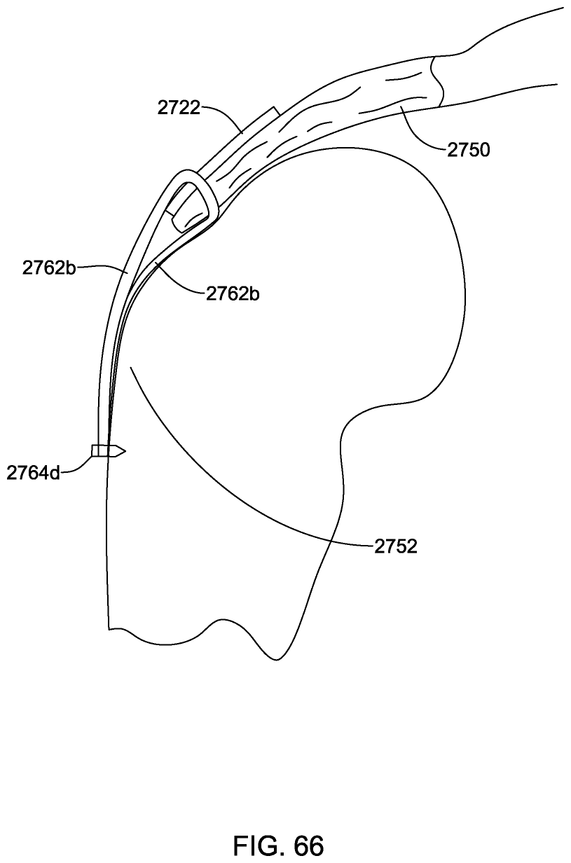

FIGS. 65-66 illustrate that a tendon repair may be performed using one or more sutures reinforced with a feltable construct. For example, FIG. 65 illustrates a rotator cuff tendon technique using sutures 2762a, 2762b, whereby each of the sutures 2762a, 2762b may be anchored to the footprint of the greater tuberosity 2752 and pass through a feltable construct 2722 and the rotator cuff tendon 2750. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. In some examples, the sutures 2762a, 2762b may be slidable sutures 2762a, 2762b configured to slide through one or more apertures in the feltable construct 2722.

In some examples, the feltable construct 2722 may include a plurality of feltable fibers which may be matted, condensed, pressed, bonded, etc. together to form a non-woven structure or construct. In some examples, the individual feltable construct 2722 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable construct 2722 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable construct 2722 may include combinations of any of the materials disclosed. For example, the feltable construct 2722 may be formed from a combination of any of natural fibers, synthetic fibers, biodegradable fibers, collagen fibers, or the like. It can be further appreciated that feltable fibers forming the feltable construct 2722 may be randomly aligned with one another to form a non-woven felted structure. In other examples, however, feltable fibers forming the feltable construct 2722 may be aligned with one another to achieve unidirectional stiffness properties with the non-woven felted structure.

FIG. 65 illustrates that a first suture 2762a may include a first end which is anchored to the footprint of the greater tuberosity 2752 via a bone anchor 2764a. Additionally, FIG. 65 illustrates that the first suture 2762a may pass through a feltable construct 2722 which has been felted to the rotator cuff tendon 2750. Further, after passing through the feltable construct 2722, FIG. 65 illustrates that the first suture 2762a may return to the footprint of the greater tuberosity 2752 whereby a second end of the suture 2762a may be anchored to the footprint of the greater tuberosity 2752 via a bone anchor 2764b. Similarly, FIG. 65 illustrates that a second suture 2762b may include a first end which is anchored to the footprint of the greater tuberosity 2752 via a bone anchor 2764c. Additionally, FIG. 65 illustrates that the second suture 2762b may pass through the feltable construct 2722 which has been felted to the rotator cuff tendon 2750. Further, after passing through the feltable construct 2722, FIG. 65 illustrates that the second suture 2762b may return to the footprint of the greater tuberosity 2752 whereby a second end of the suture 2762b may be anchored to the footprint of the greater tuberosity 2752 via a bone anchor 2762d.

Additionally, FIG. 66 illustrates a side view of the tendon repair shown in FIG. 65. FIG. 66 illustrates the suture 2762b extending from the bone anchor 2764d and along the footprint of the greater tuberosity 2752, whereby the suture 2762b passes through the feltable construct 2722 and the rotator cuff tendon 2750. It can be appreciated that the suture 2762a is hidden from view in FIG. 66, but as discussed herein, may function similarly to the suture 2762b. Further, as discussed herein, FIG. 66 illustrates the suture 2762b returning to the footprint of the greater tuberosity 2752 after passing through the feltable construct 2722 and the rotator cuff tendon 2750. Additionally, while hidden from view in FIG. 66, it can be appreciated from FIG. 65 that the second end of the suture 2762b may be attached to the footprint of the greater tuberosity 2752 via the bone anchor 2764c. It can be appreciated that felting the feltable construct 2722 to the rotator cuff tendon may reinforce the rotator cuff tendon 2750, thereby preventing the sutures 2762a, 2762b from tearing through the rotator cuff tendon along the portions of the rotator cuff tendon 2750 where the sutures 2762a, 2762b pass through the feltable construct 2722 and the rotator cuff tendon 2750.

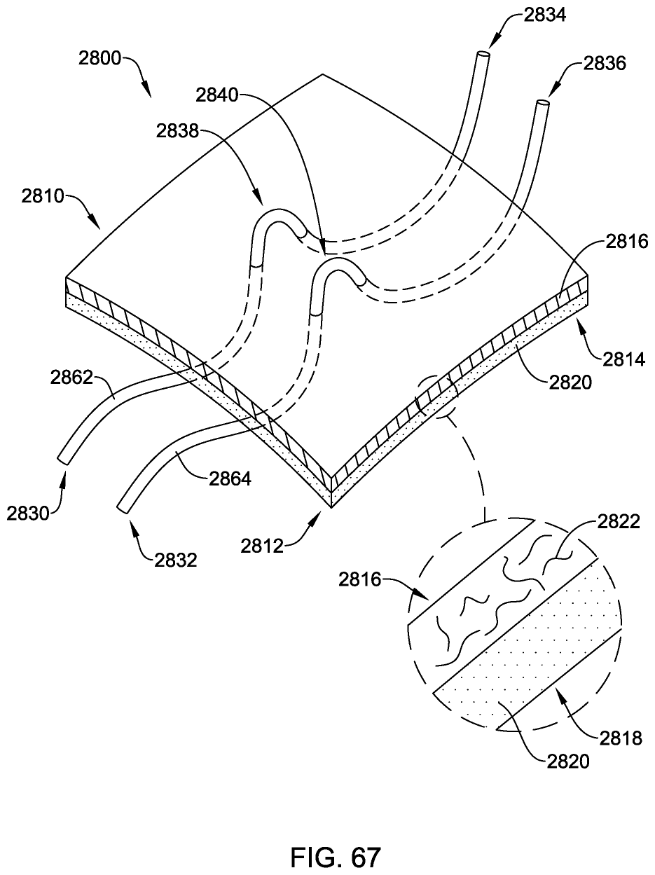

FIG. 67 illustrates a repair implant 2800. The repair implant 2800 may include a base component 2810 having a first end 2812 and a second end 2814. In some examples, the base component 2810 may include a rectangular shape. However, in other examples, the base component 2810 may include a circular, ovular, triangular, square, polygonal, or other geometric shape. Additionally, FIG. 67 further illustrates that the base component 2810 may include a first layer 2816 and a second layer 2818. For example, the detailed view of FIG. 67 illustrates the first layer 2816 positioned adjacent to (e.g., contacting, engaging, etc.) the second layer 2818.

Further, the detailed view of FIG. 67 illustrates that the first layer 2816 of the base component 2810 may include a feltable material 2822. For example, the detailed view of FIG. 67 illustrates the first layer 2816 may include a plurality of feltable fibers 2822 which extend from the first end 2812 to the second end 2814 of the base component 2810.

In some examples, the plurality of individual feltable fibers 2822 forming the first layer 2816 may be matted, condensed, pressed, bonded, etc. together to form a non-woven structure or construct. In some examples, the individual feltable fibers 2822 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2822 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2822 may include combinations of any of the materials disclosed. For example, the feltable fibers 2822 may be formed from a combination of any of natural fibers, synthetic fibers, biodegradable fibers, collagen fibers, or the like. It can be further appreciated that the feltable fibers 2822 may be randomly aligned with one another to form a non-woven felted structure. In other examples, however, the feltable fibers 2822 may be aligned with one another to achieve unidirectional stiffness properties with the non-woven felted structure.

Further, the detailed view of FIG. 67 illustrates that the second layer 2818 of the base component 2810 may be formed from a biologic component 2820. For example, the second layer 2818 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the second layer 2818 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the second layer 2818 of the base component 2810. For example, a lattice structure and/or other structures described herein may be formed by molding which may be punched, woven, etc. to form the desired arrangement. The second layer 2818 of the base component 2810 may be porous or included pathways to encourage tissue in-growth. In some embodiments, the second layer 2818 of the base component 2810 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 67 further illustrates that the repair implant 2800 may also include one or more sutures attached to the base component 2810. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 67 illustrates that the repair implant 2800 may include a first suture 2862 having a first end 2830, a second end 2834 and a medial region 2838. FIG. 67 further illustrates that both the first end 2830 and the second end 2834 of the first suture 2862 may pass through both the first layer 2816 and the second layer 2818, thereby positioning a portion of the medial region 2838 of the first suture 2862 along an outwardly facing surface of the first layer 2816. Additionally, FIG. 67 further illustrates that both the first end 2832 and the second end 2836 of the second suture 2864 may pass through both the first layer 2816 and the second layer 2818, thereby positioning a portion of the medial region 2840 of the second suture 2864 along an outwardly facing surface of the first layer 2816. In some examples, the sutures 2838, 2840 may be slidable sutures 2838, 2840 configured to slide through one or more apertures in the base component 2810.

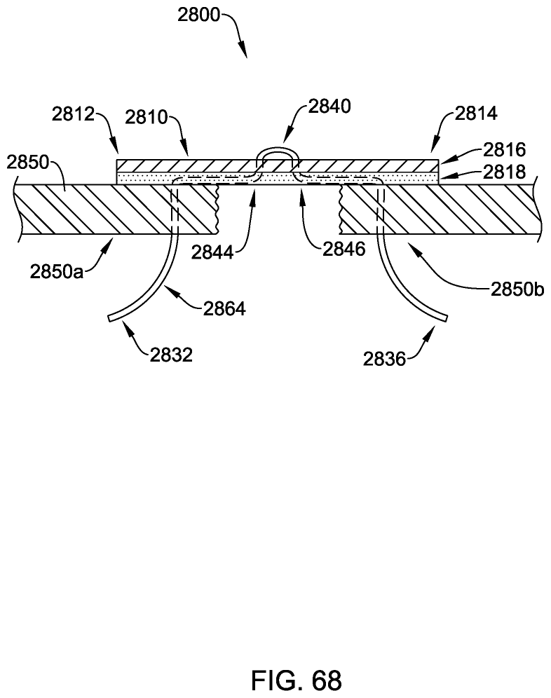

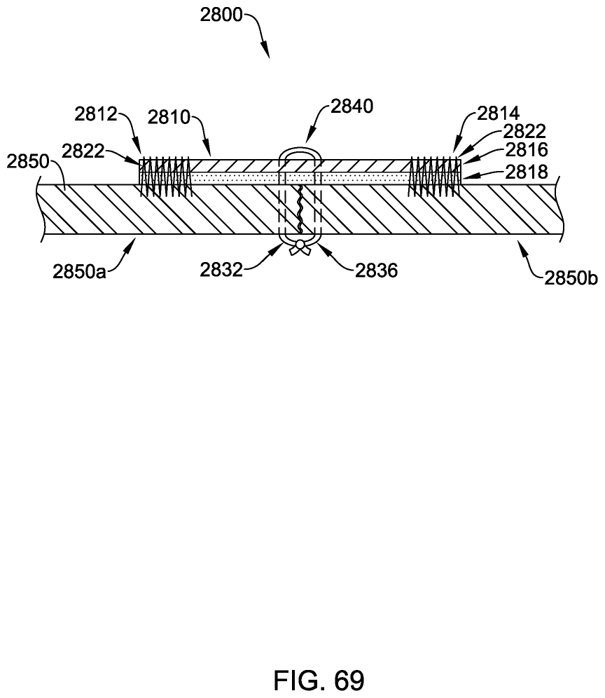

FIGS. 68-69 illustrates a process of utilizing the repair implant 2800 to repair a torn tendon 2850. FIG. 68 illustrates the first end 2812 of the repair implant 2800 being positioned adjacent to a first end region 2850a of the torn tendon 2850 and a second end 2816 of the repair implant 2800 being positioned adjacent to a second end region 2850b of the torn tendon 2850. Further, FIG. 68 illustrates that after the first end 2812 of the repair implant 2800 is positioned adjacent to the first end region 2850a of the torn tendon 2850, the first end 2832 of the second suture 2864 may be passed through the first end region 2850a of the tendon 2850. Additionally, FIG. 68 illustrates that after the second end 2814 of the repair implant 2800 is positioned adjacent to the second end region 2850b of the torn tendon 2850, the second end 2836 of the second suture 2864 may be passed through the second end region 2850b of the tendon 2850. Further, FIG. 68 illustrates that the second suture 2864 may extend along the second layer 2818 of the repair implant 2800 and pass through the second layer 2818 and the first layer 2816 via a first aperture 2844. Further, FIG. 68 illustrates that the second suture 2864 may pass back through the first layer 2816 and the second layer 2818 via a second aperture 2846. FIG. 68 further illustrates that the second suture 2864 may extend along the second layer 2818 of the repair implant 2800 before passing through the second end region 2850b of the torn tendon 2850. It can be appreciated that while the first suture 2862 (shown in FIG. 67) is hidden from view in FIG. 68, it may pass through first end region 2850a of the tendon 2850, through the base component 2810 (including the first layer 2816 and the second layer 2818) and the second end region 2850b of the tendon 2850 similarly to that described herein with respect to the second suture 2864.

FIG. 69 illustrates the first end 2832 and the second end 2836 of the second suture 2864 may be tightened such that the first end region 2850a and the second end region 2850b of the torn tendon 2850 are drawn in close proximity to one another, which may aid in the healing of the torn tendon 2850. Further, FIG. 69 illustrates that the first end 2832 and the second end 2836 of the second suture 2864 may be coupled (e.g., tied, fastened, etc.) together, which may maintain the proximity of the first end region 2850a to the second end region 2850b.

Additionally, FIGS. 68-69 illustrate that positioning the repair implant 2800 along the torn tendon 2850 as illustrated in FIGS. 68-69 may position the second layer 2818 directly against the tissue of the tendon 2850. It can be appreciated that in this configuration, the biologic component 2820 of the second layer 2818 may contact the injured tissue of the tendon 2850. Further, it can be appreciated that after the first end 2832 and the second end 2836 of the second suture 2864 are tightened such that the first end region 2850a and the second end region 2850b of the torn tendon 2850 are drawn in close proximity to one another, one or more portions of the first layer 2816 of the base component 2810 may be felted to the first end region 2850a and/or the second end region 2850b of the tendon 2850. For example, FIG. 69 illustrates that after the first end 2832 and the second end 2836 of the second suture 2864 are tightened such that the first end region 2850a and the second end region 2850b of the torn tendon 2850 are drawn in close proximity to one another, the feltable fibers 2822 of the first end 2812 of the base component 2810 may be felted to the first end region 2850a of the torn tendon 2850 and the feltable fibers 2822 of the second end 2814 of the base component 2810 may be felted to the second end region 2850b of the torn tendon 2850. It can be appreciated that felting the feltable fibers 2822 of the first layer 2816 to the tendon 2850 may position and engage the biologic component 2820 of the second layer 2018 with the injured tissue of the torn tendon 2850.

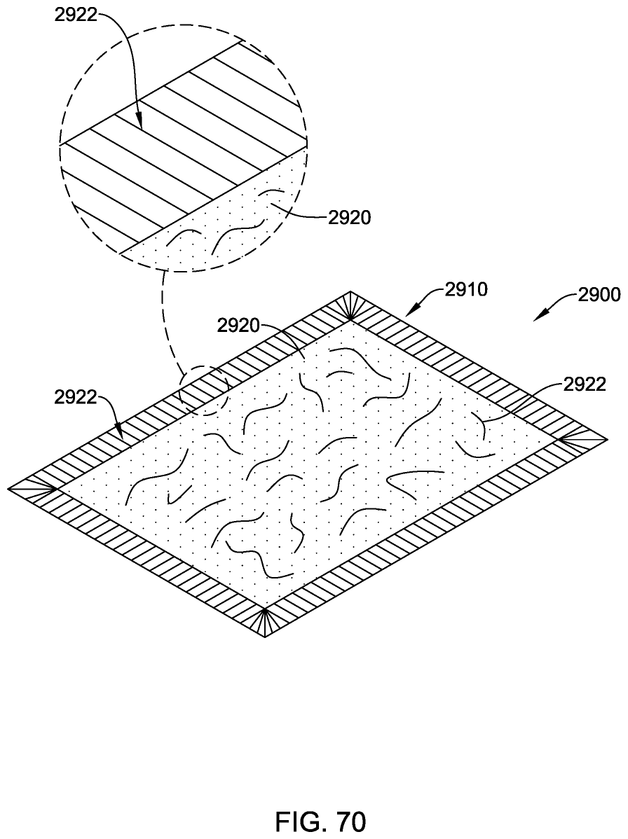

FIG. 70 illustrates repair implant 2900. The repair implant 2900 may include a square shape. However, it is contemplated that the repair implant 2900 may include a variety of shapes such as circular, rectangular, ovular, crescent-shaped, triangular, polygonal, etc.

Additionally, FIG. 70 illustrates that the repair implant 2900 may further include a plurality of feltable fibers 2922 integrated with a biologic component 2920. It can be appreciated that the individual feltable fibers 2922 may be matted, condensed, pressed, bonded, etc. together to form a non-woven structure or construct. In some examples, the individual feltable fibers 2922 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 2922 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 2922 may include combinations of any of the materials disclosed. For example, the feltable fibers 2922 may be formed from a combination of any of natural fibers, synthetic fibers, biodegradable fibers, collagen fibers, or the like. It can be further appreciated that the feltable fibers 2922 may be randomly aligned with one another to form a non-woven felted structure. In other examples, however, the feltable fibers 2922 may be aligned with one another to achieve unidirectional stiffness properties with the non-woven felted structure.

Additionally, the biologic component 2920 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 2920 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 2920. For example, a lattice structure and/or other structures described herein may be formed by molding which may be punched, woven, etc. to form the desired arrangement. The biologic component 2920 may be porous or included pathways to encourage tissue in-growth. In some embodiments, the biologic component 2920 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Additionally, the detailed view of FIG. 70 illustrates that the outer edges of the repair implant 2900 may be formed into a plurality of individual fibers 2922 which extend away from a central region of the repair implant 2900 for form a “feltable edge.” It can be appreciated that the individual fibers 2922 forming the feltable edge around the perimeter of the repair implant 2900 may be used to felt the repair implant 2900 at a target tissue site. In some examples, the individual fibers 2922 forming the perimeter may be formed by cutting the edges of the repair implant 2922 into individual fiber strands 2922 which may be used to felt the repair implant 2900 at a target tissue site.

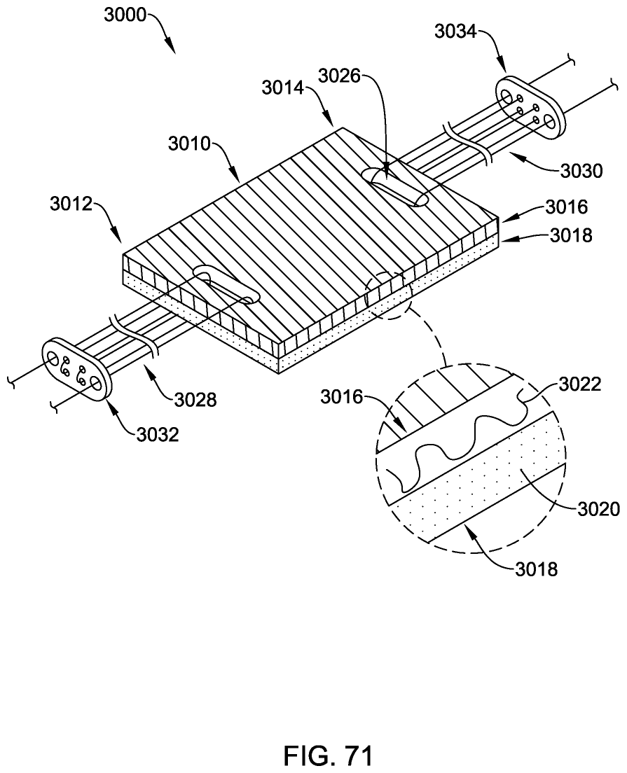

FIG. 71 illustrates a repair implant 3000. The repair implant 3000 may include a base component 3010 having a first end 3012 and a second end 3014. In some examples, the base component 3010 may include a rectangular shape. However, in other examples, the base component 3010 may include a circular, ovular, triangular, square, polygonal, or other geometric shape. Additionally, FIG. 71 further illustrates that the base component 3010 may include a first layer 3016 and a second layer 3018. For example, the detailed view of FIG. 71 illustrates the first layer 3016 contacting (e.g., engaging, etc.) the second layer 3018.

Further, the detailed view of FIG. 71 illustrates that the first layer 3016 of the base component 3010 may include a feltable material 3022. For example, the detailed view of FIG. 71 illustrates the first layer 3016 may include a plurality of feltable fibers 3022 which extend from the first end 3012 to the second end 3014 of the base component 3010.

In some examples, the plurality of individual feltable fibers 3022 forming the first layer 3016 may be matted, condensed, pressed, bonded, etc. together to form a non-woven structure or construct. In some examples, the individual feltable fibers 3022 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3022 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3022 may include combinations of any of the materials disclosed. For example, the feltable fibers 3022 may be formed from a combination of any of natural fibers, synthetic fibers, biodegradable fibers, collagen fibers, or the like. It can be further appreciated that the feltable fibers 3022 may be randomly aligned with one another to form a non-woven felted structure. In other examples, however, the feltable fibers 3022 may be aligned with one another to achieve unidirectional stiffness properties with the non-woven felted structure.

Further, the detailed view of FIG. 71 illustrates that the second layer 3018 of the base component 3010 may be formed from a biologic component 3020. For example, the second layer 3018 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the second layer 3018 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the second layer 3018 of the base component 3010. For example, a lattice structure and/or other structures described herein may be formed by molding which may be punched, woven, etc. to form the desired arrangement. The second layer 3018 of the base component 3010 may be porous or included pathways to encourage tissue in-growth. In some embodiments, the second layer 3018 of the base component 3010 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

In some examples, the base component 3010 may not include a first layer 3016 and a second layer 3018. Rather, in some examples, the base component 3010 may be formed from a biologic construct integrated with feltable material. The feltable material may be dispersed (e.g., distributed) throughout the biologic construct. For example, the base component 3010 may include feltable fibers (similar to the feltable 3022 described within) which are integrated with a collagen, a collagen element and/or a collagen-like material similar to other bioinductive collagen constructs described herein. The feltable fibers may be uniformed distributed thought the collagen or collagen-like material.

FIG. 71 further illustrates that the repair implant 3000 may further include a first plurality of tensioning members 3028 coupled to the base component 3010. For example, FIG. 71 illustrates that the first plurality of tensioning members 3028 may extend through a first aperture 3024 positioned along the first end 3012 of the base component 3010. It can be appreciated that the first aperture 3024 may extend through the first layer 3016 and the second layer 3018 of the base component 3010, thereby allowing the first plurality of tensioning member 3028 to pass through both the first layer 3016 and the second layer 3018 of the base component 3010. Additionally, FIG. 71 illustrates that the repair implant 3000 may further include a first anchor 3032 coupled to the first plurality of tensioning members 3028. FIG. 71 illustrates that the first plurality of tensioning members 3028 may extend through one or more apertures positioned in the first anchor 3032.

FIG. 71 further illustrates that the repair implant 3000 may further include a second plurality of tensioning members 3030 coupled to the base component 3010. For example, FIG. 71 illustrates that the second plurality of tensioning members 3030 may extend through an aperture 3026 positioned along the second end 3014 of the base component 3010. It can be appreciated that the second aperture 3026 may extend through the first layer 3016 and the second layer 3018 of the base component 3010, thereby allowing the second plurality of tensioning members 3030 to pass through both the first layer 3016 and the second layer 3018 of the base component 3010. Additionally, FIG. 71 illustrates that the repair implant 3000 may further include a second anchor 3034 coupled to the second plurality of tensioning members 3030. FIG. 71 illustrates that the second plurality of tensioning members 3030 may extend through one or more apertures positioned in the second anchor 3034. In some examples, the anchors 3032, 3034 may include buttons, knotless anchors, knotted anchors, bone anchors, combinations thereof or other similar anchors. In other examples, the repair implant 3000 may not include the anchors 3032, 3034, whereby the repair implant may be secured to a target site via attaching the tensioning members 3028, 3030 directing to an independent anchor (e.g., one or more anchor which are independent of the repair implant 3000 and not shown in FIG. 71).

Further, in some examples, the repair implant 3000 may include tensioning members on one side of the repair implant 3000. For example, the repair implant 3000 may only include the tensioning members 3028 and anchor 3032 coupled to the first end 3012 of the base component 3010 (e.g., the tensioning members 3030 and anchor 3034 would be omitted from the repair implant 3000 in this example). Similarly, the repair implant 3000 may only include the tensioning members 3030 and anchor 3034 coupled to the second end 3014 of the base component 3010 (e.g., the tensioning members 3028 and anchor 3032 would be omitted from the repair implant 3000 in this example).

It can be appreciated that the repair implant 3000 may be configured to provide a combination of structural features, properties and functions that may be used to treat partial or full thickness tears of soft tissues, including partial or full thickness tears of soft tissues. For example, the repair implant 3000 may be configured to treat partial or full thickness tears of an anterior cruciate ligament (ACL). The repair implant 3000 may encourage tissue ingrowth while also providing additional mechanical properties such as, but not limited to strength and stiffness. For example, the implant 3000 may provide a layer of collagen over injured tissue to facilitate healing.

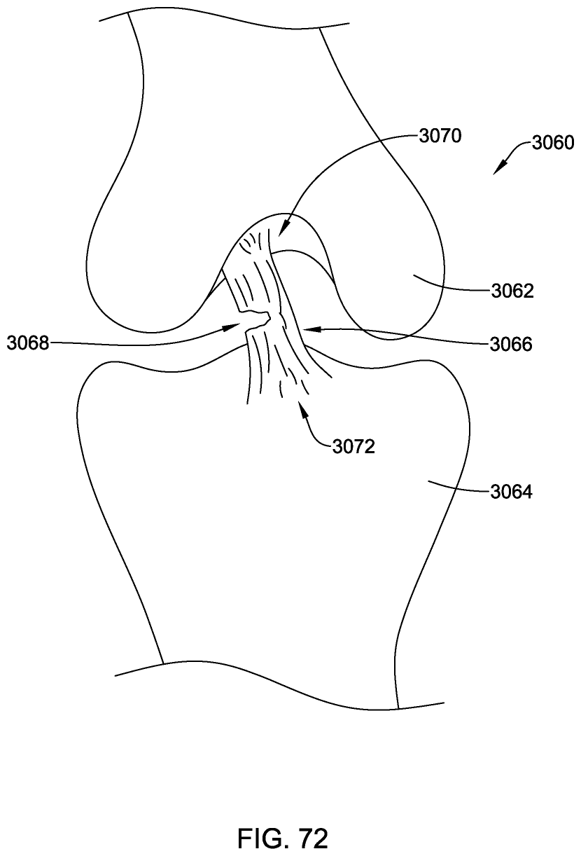

FIG. 72 illustrates a knee joint 3060 including the distal femur 3062 of and the tibia 3064. Additionally, FIG. 72 illustrates the anterior crucial ligament 3066 having a first end region attached to the distal femur 3062 at a femoral attachment region 3070 and second end region attached to the tibia 3064 at a tibial attachment region 3072. Additionally, FIG. 72 illustrates a tear 3068 of the anterior crucial ligament 3066.

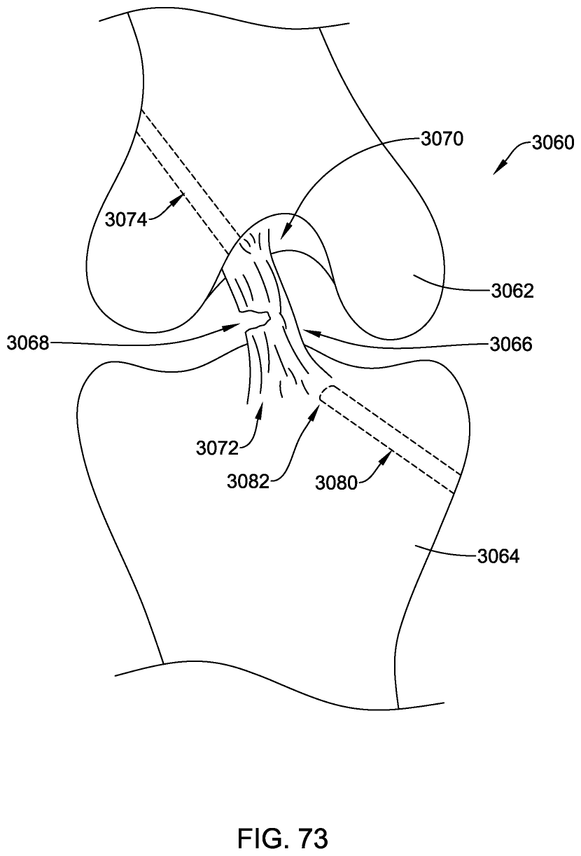

FIG. 73 illustrates a first step in using the repair implant 3000 to repair the tear 3068 in the anterior cruciate ligament 3066. FIG. 73 illustrates that prior to the placement of the repair implant 3000 relative to the tear 3068 of the in the anterior cruciate ligament 3066, a femoral bone tunnel 3074 may be drilled in the distal femur 3062 and a tibial bone tunnel 3080 may be drilled in the tibia 3064.

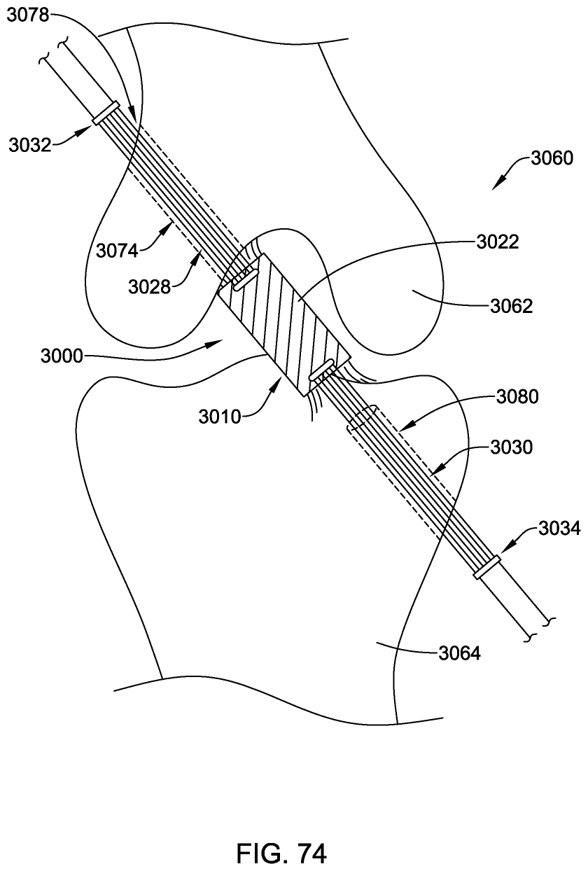

FIG. 74 illustrates the positioning of the repair implant 3000 relative to the tear 3068 of the anterior cruciate ligament 3066 described with respect to FIGS. 72-73. Placement of the repair implant 3000 relative to the tear 3068 of the anterior cruciate ligament 3066 may include passing the first anchor 3032 through the tibial bone tunnel 3080, along the anterior cruciate ligament 3066, and through the femoral bone tunnel 3074. FIG. 74 illustrates that after passing through the femoral bone tunnel 3074, the first anchor 3032 may be positioned adjacent to an outer surface of the femur and a portion of the first tensioning members 3028 may be positioned within the femoral bone tunnel 3074. Additionally, FIG. 74 illustrates that advancing (e.g., pulling) the first anchor 3032 and the first tensioning members 3028 through the femoral tunnel 3074 may also advance (e.g., pull) the base component 3010 through tibial tunnel 3080 to a position in which the base component 3010 is positioned along the tear 3068 of the anterior cruciate ligament 3066 (shown in FIGS. 72-73). It can be further appreciated from FIG. 74 that advancing (e.g., pulling) the first anchor 3032, the first plurality of tensioning members 3028 and the base component 3010 through the tibial tunnel 3080 may also advance (e.g., pull) the second plurality of tensioning members 3030 into the tibial tunnel 3080. Further, FIG. 74 illustrates that advancing (e.g., pulling) the second plurality of tensioning members 3030 into the tibial tunnel 3080 may pull the second anchor 3034 to a position adjacent an outer surface of the tibia 3064.

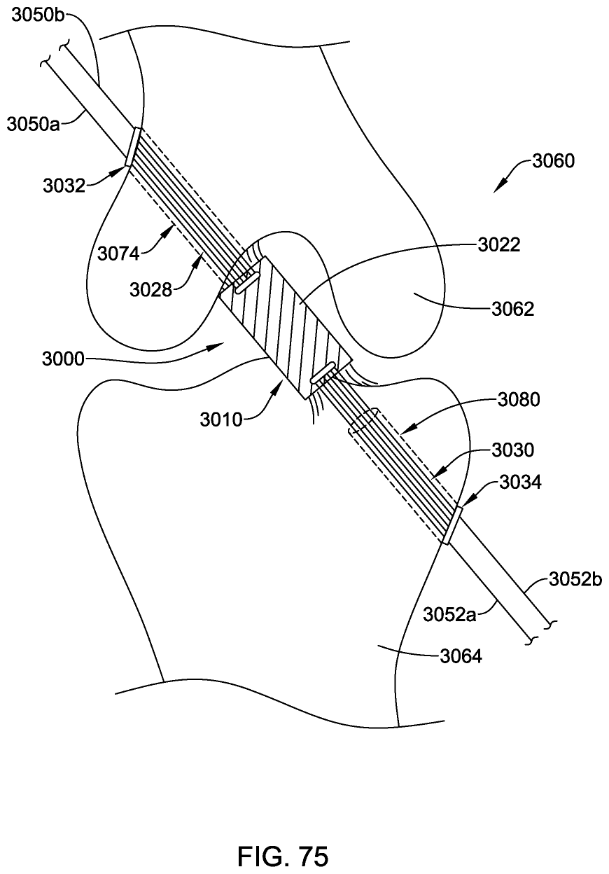

FIG. 75 illustrates that after the base component 3010 has been positioned along the tear 3068 of the anterior cruciate ligament 3066 (shown in FIGS. 72-73), the repair implant may be tensioned and attached to the femur 3062, the tibia and the anterior cruciate ligament 3066. For example, FIG. 75 illustrates that the first anchor 3032 may be attached to an outer surface of the femur 3062 via tensioning of a first tensioning member 3050a and a second tensioning member 3050b of the first plurality of tensioning members 3028. It can be appreciated that the first anchor 3032 may be sized such that, when flattened against the outer surface of the femur 3062, it cannot enter the femoral tunnel 3074, and therefore, a clinician may manipulate the first tensioning member 3050a and the second tensioning member 3050b of the first plurality of tensioning members 3028 to engage (e.g., compress against, tighten against, etc.) the first anchor 3032 along the outer surface of the femur 3062. Additionally, FIG. 75 illustrates that the second anchor 3034 may be attached to an outer surface of the tibia 3064 via tensioning of a third tensioning member 3052a and a fourth tensioning member 3052b of the second plurality of tensioning members 3030. It can be appreciated that the second anchor 3034 may be sized such that, when flattened against the outer surface of the tibia 3064, it cannot enter the tibial tunnel 3080, and therefore, a clinician may manipulate the third tensioning member 3052a and the fourth tensioning member 3052b of the second plurality of tensioning members 3030 to engage (e.g., compress against, tighten against, etc.) the second anchor 3034 along the outer surface of the tibia 3064. It can be further appreciated that third tensioning member 3052a and the fourth tensioning member 3052b may be individually tensioned to tailor the placement and engagement of the base component 3010 (including the biologic component 3020) relative to the tear 3068 of the anterior cruciate ligament 3066. It can be appreciated that after placement of first and second anchors 3032, 3034, the tensioning members 3050a, 3050b, 3052a, 3052b may be tied and trimmed flush to the outer surface of the first and second anchors 3032, 3034, respectively.

It can be further appreciated that after the placement of the base component 3010 along the tear 3068 of the anterior cruciate ligament 3066, a plurality of the fibers 3022 of the first layer 3016 of the base component 3010 may be felted to portions of the anterior cruciate ligament 3066 (shown in FIGS. 72-53) adjacent to the tear 3068. For example, it can be appreciated that a plurality of the fibers 3022 along the first end region 3012 of the base component 3010 may be felted to a portion of the anterior cruciate ligament 3066 which is proximal to the tear 3068 and a plurality of the fibers 3022 along the second end region 3014 of the base component 3010 may be felted to a portion of the anterior cruciate ligament 3066 which is distal to the tear 3068 of the anterior cruciate ligament 3066. It can be further appreciated that felting a plurality of the fibers 3022 to portions of the anterior cruciate ligament 3066 both proximal and distal to the tear 3068 may position the biologic component 3020 such that it contacts the tear 3068 of the anterior cruciate ligament 3066, thereby permitting the biologic component 3020 to encourage tissue ingrowth and facilitate healing of the injured tissue.

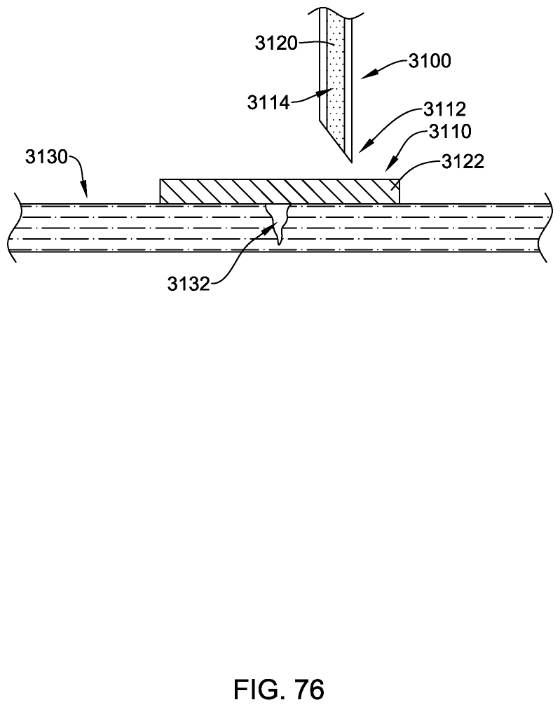

FIG. 76 illustrates a felting needle 3100. The felting needle 3100 may be attached to a felting drive mechanism 46 (shown in FIG. 3), whereby the felting drive mechanism 46 is configured to rapidly displace (e.g., oscillate, drive) the needle 3100 back and forth along the longitudinal axis of the needle 3100. It can be appreciated that the drive mechanism 46 may be driven by a motor (e.g., the drive mechanism may be coupled to a motorized system) or, alternatively, via pneumatic, magnetic, hydraulic or similar systems.

FIG. 76 further illustrates that the needle 3100 may include a distal end 3112 and a lumen 3114 extending therein. Further, FIG. 76 illustrates that the needle 3100 may include a biologic material 3120 disposed within a the lumen 3114. In some examples, the biologic material 3120 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well.

FIG. 76 further illustrates a feltable repair implant 3110 positioned along a tear 3132 in a tendon 3130. It can be appreciated that the feltable repair implant 3110 may be formed from a plurality of feltable fibers 3122. The individual feltable fibers 3122 may be matted, condensed, pressed, bonded, etc. together to form a non-woven structure or construct. In some examples, the individual feltable fibers 3122 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3122 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3122 may include combinations of any of the materials disclosed. For example, the feltable fibers 3122 may be formed from a combination of any of natural fibers, synthetic fibers, biodegradable fibers, collagen fibers, or the like. It can be further appreciated that the feltable fibers 3122 may be randomly aligned with one another to form a non-woven felted structure. In other examples, however, the feltable fibers 3122 may be aligned with one another to achieve unidirectional stiffness properties with the non-woven felted structure.

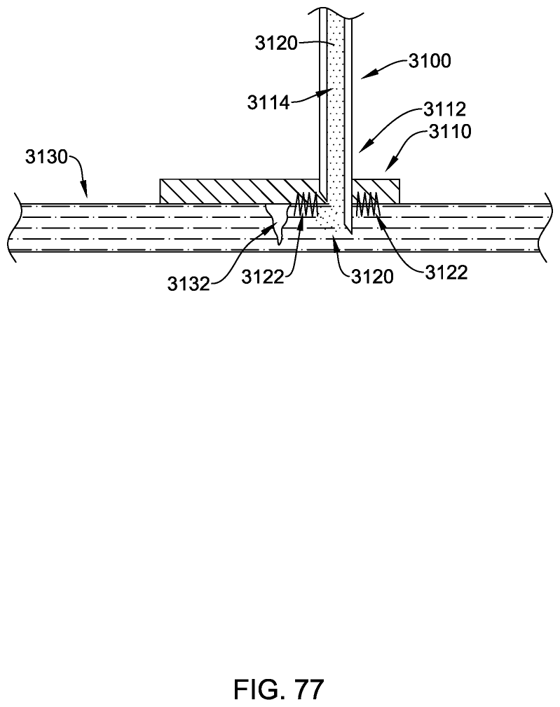

FIG. 77 illustrates the felting needle 3100 being used to felt the fibers 3122 of the feltable repair implant 3110 to a portion of the tendon 3130. For example, FIG. 77 illustrates the distal end 3112 of the needle 3100 being driven through the feltable repair implant 3110 such that some of the fibers 3122 of the feltable repair implant 3110 are pushed and/or pulled into the tendon 3130. Further, FIG. 77 illustrates that as the needle 3100 is pushed and/or pulled into the tendon 3130, some of the biologic material 3120 may flow out of the lumen 3114 and into the tissue of the tendon 3130 adjacent to the fibers 3122. It can be appreciated that as the needle 3100 continues to be pushed and/or pulled into the tendon 3130, the biologic material 3120 may continue to flow out of the lumen 3114 and into the tissue of the tendon 3130 which is adjacent the entire length of the feltable repair implant 3110. It can be further appreciated that the biologic material 3120 may aid in the healing of the torn tendon tissue.

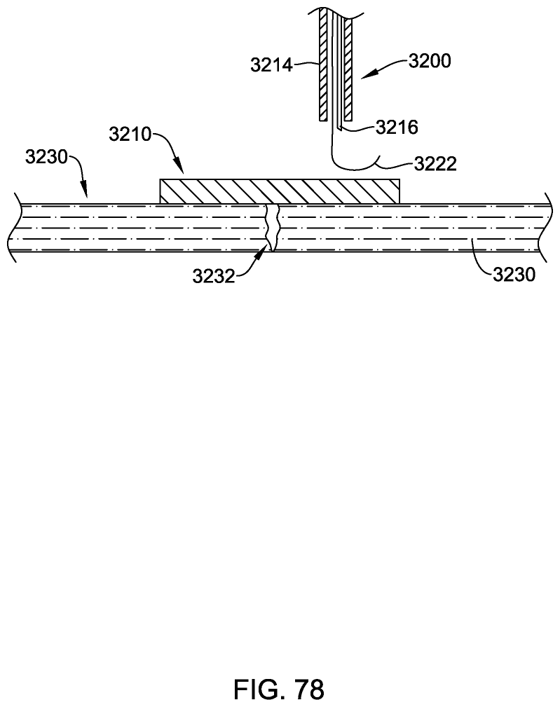

FIG. 78 illustrates a felting needle assembly 3200. The felting needle assembly 3200 may include a delivery shaft 3214. The delivery shaft 3214 may include a lumen 3218 extending therein. The felting needle assembly 3200 may further include a felting needle 3216 extending within the lumen 3218. The felting needle 3216 may be attached to a felting drive mechanism 46 (shown in FIG. 3), whereby the felting drive mechanism 46 is configured to rapidly displace (e.g., oscillate, drive) the needle 3216 back and forth along the longitudinal axis of the needle 3216. It can be appreciated that the drive mechanism 46 may be driven by a motor (e.g., the drive mechanism may be coupled to a motorized system) or, alternatively, via pneumatic, magnetic, hydraulic or similar systems.

FIG. 78 further illustrates that the felting needle assembly 3200 may further include a feltable material 3222 (e.g., feltable fiber) extending within the lumen 3218. It can be appreciated that the feltable material 3222 and the felting needle 3216 may coextend alongside one another in the lumen 3218 of the delivery shaft 3214. The feltable material 3222 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material 3222 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material 3222 may include combinations of any of the materials disclosed. For example, the feltable material 3222 may be formed from a combination of any of natural fibers, synthetic fibers, biodegradable fibers, collagen fibers, or the like.

FIG. 78 further illustrates a biologic repair implant 3210 positioned along a tear 3232 in a tendon 3230. It can be appreciated that the biologic repair implant 3210 may be formed from collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well.

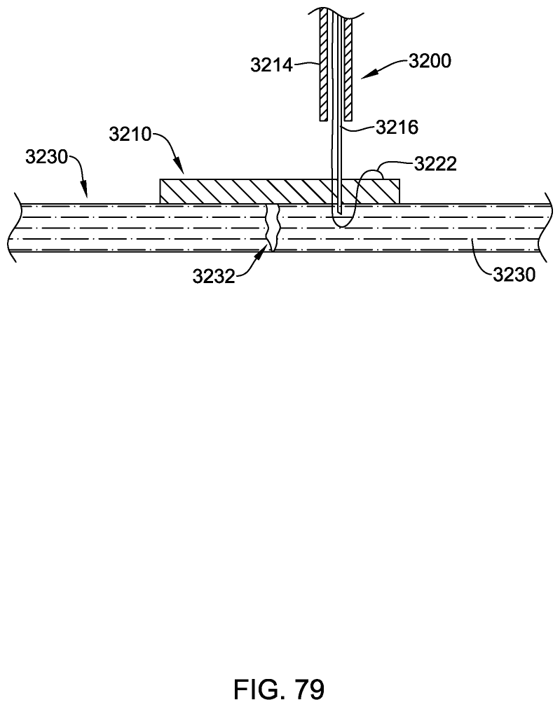

FIG. 79 illustrates the felting needle 3216 being used to felt the biologic repair implant 3210 to the tendon 3230. Further, FIG. 79 illustrates that the feltable material 3222 may be continuously fed through the lumen 3218 of the shaft 3214 to a position adjacent the biologic repair implant 3210. In other words, the feltable material 3222 may be continuously fed through the lumen 3218 of the shaft 3214 such that it contacts and rests along an outer-facing surface of the biologic repair implant 3210. Further, while the feltable material 3222 is being fed through the lumen 3218, the felting needle 3216 may push and/or pull the feltable material 3222 through the biologic repair implant 3210 and into the tendon 3230, thereby felting the biologic repair implant 3210 to the tendon 3230.

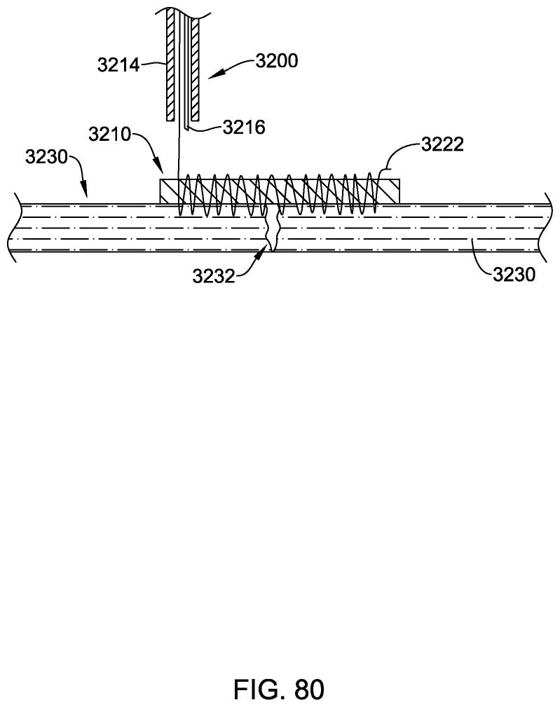

It can be appreciated from FIG. 80 that as the needle 3216 continues to be pushed and/or pulled into the tendon 3230, the feltable material 3222 may continue to be fed out of the lumen 3218, through the biologic repair implant 3210 and into the tissue of the tendon 3230 such that the entire length of the biologic repair implant 3210 is felted to the tendon 3230. It can be further appreciated that the biologic repair implant 3210 may aid in the healing of the tear 3230 in the tendon 3230.

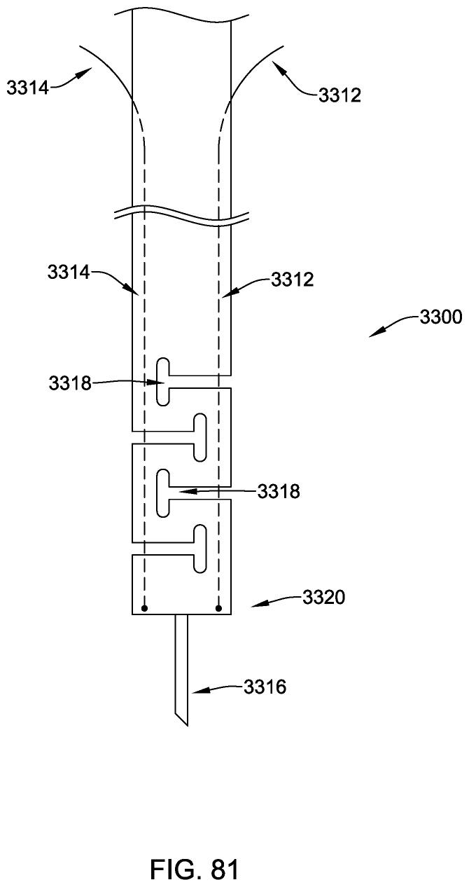

FIG. 81 illustrates a felting needle assembly 3300. The felting needle assembly 3300 may include a felting needle 3316 extending within a lumen of a delivery shaft 3324. FIG. 81 further illustrates that a portion of the delivery shaft 3324 may include one or more apertures 3318 (e.g., slots) which extend through the wall of the delivery shaft 3324. The apertures may be configured to permit the delivery shaft to flex (e.g., bend, curve, etc.) and may include a variety of shapes.

The felting needle 3316 may be attached to a felting drive mechanism 46 (shown in FIG. 3), whereby the felting drive mechanism 46 is configured to rapidly displace (e.g., oscillate, drive) the needle 3316 back and forth along the longitudinal axis of the needle 3316 and through the lumen of the delivery shaft 3324. It can be appreciated that the drive mechanism 46 may be driven by a motor (e.g., the drive mechanism may be coupled to a motorized system) or, alternatively, via pneumatic, magnetic, hydraulic or similar systems.

FIG. 81 further illustrates that the felting needle delivery shaft 3324 may further include a first pull wire 3312 extending within a wall of the delivery shaft 3324 and a second pull wire 3314 extending within a wall of the delivery shaft 3324. In other examples, the first pull wire 3312 and the second pull wire 3314 may extending along an inner surface of the delivery shaft 3324 (e.g., an inner surface of the delivery shaft which defines the lumen of the delivery shaft 3324). Additionally, it can be appreciated that the first pull wire 3312 may be offset approximately 180 degrees from the second pull wire 3314. Further, the first pull wire 3312 and the second pull wire 3314 may be attached at a distal end 3320 of the shaft 3324.

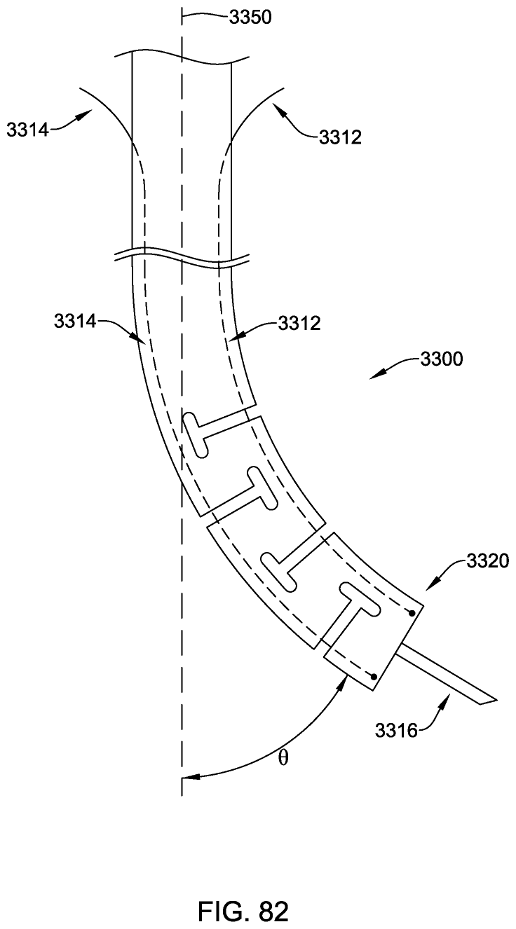

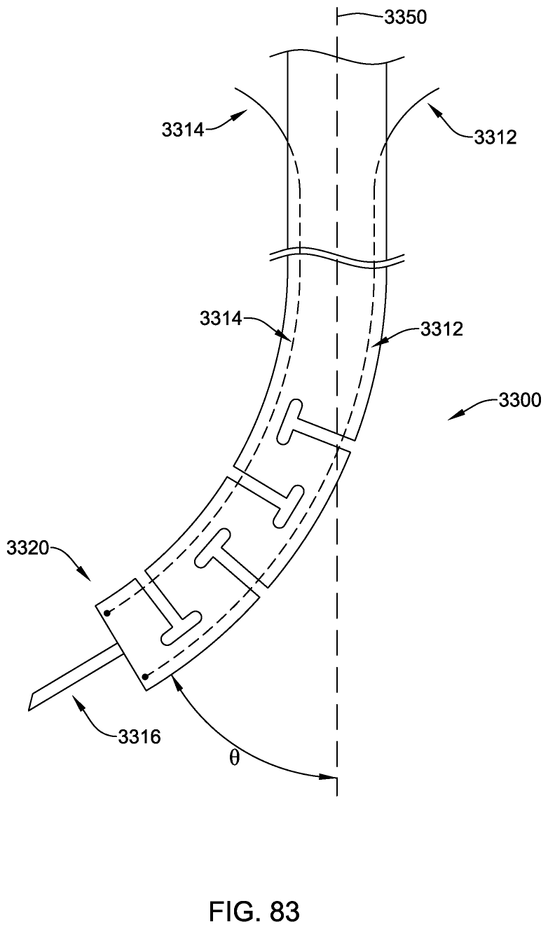

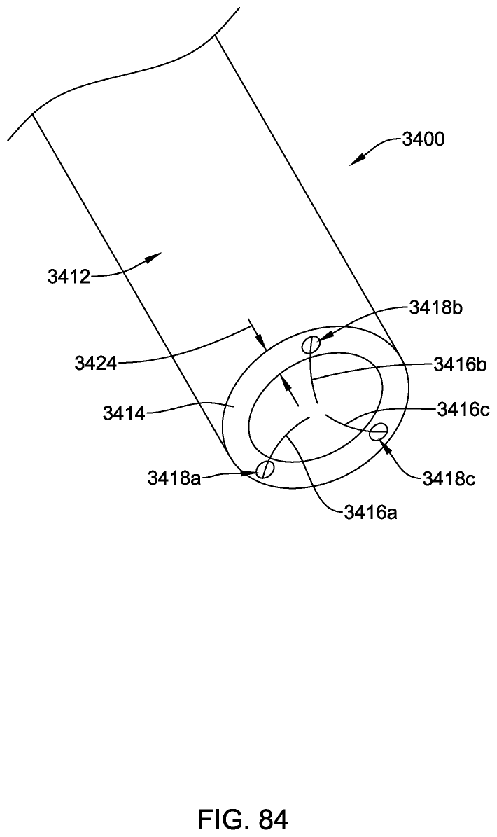

FIGS. 82-83 illustrate that the delivery shaft 3324 may be steered via the pulling of the first pull wire 3312 and the second pull wire 3314. For example, FIG. 82 illustrates that the delivery shaft 3324 may be steered (e.g., curved, bent, etc.) a first direction via the pulling of the first pull wire 3312 which is attached to the distal end region 3320 of the delivery shaft 3324. Similarly, FIG. 83 illustrates that the delivery shaft 3324 may be steered (e.g., curved, bent, etc.) a second direction opposite the first direction via the pulling of the second pull wire 3314 which is attached to the distal end region 3320 of the delivery shaft 3324. It can be further appreciated that the felting drive mechanism 46 may be configured to rapidly displace (e.g., oscillate, drive) the needle 3316 back and forth along the longitudinal axis of the needle 3316 and through the lumen of the delivery shaft 3324 when the delivery shaft 3324 is steered in any direction.

FIGS. 82-83 illustrate that the delivery shaft 3324 shown in FIG. 82-83 may configured to bend (in either direction) approximately about 10 to 90 degrees (depicted by the angle θ extending between the axis 3350 and the delivery shaft 3324) relative to the position of the delivery shaft 3324 shown in FIG. 81, or about 5 to 70 degrees relative to the position of the delivery shaft 3324 shown in FIG. 81, or about 10 to 60 degrees relative to the position of the delivery shaft 3324 shown in FIG. 81, or about 10 to 50 degrees relative to the position of the delivery shaft 3324 shown in FIG. 81, or about 15 to 50 degrees relative to the position of the delivery shaft 3324 shown in FIG. 81, or about 30 to 50 degrees relative to the position of the delivery shaft 3324 shown in FIG. 81, or about 45 degrees relative to the position of the delivery shaft 3324 shown in FIG. 81.

In some examples, the delivery shaft 3324 may be configured to be held in a fixed position (at a given angle) whereby the felting drive mechanism 46 may be configured to rapidly displace (e.g., oscillate, drive) the needle 3316 back and forth along the longitudinal axis of the needle 3316 and through the lumen of the delivery shaft 3324.

FIG. 84 illustrates a felting needle assembly 3400. The felting needle assembly 3400 may include a shaft 3412 having a wall 3424. The shaft 3412 may further include a distal face 3414. FIG. 84 further illustrates that the felting needle assembly 3400 may also include a plurality of felting needles extending within the wall 3424 of the shaft 3412. For example, FIG. 84 illustrates that the felting needle assembly 3400 may include a first felting needle 3416a extending through a lumen 3418a in the wall of the shaft 3412, a second felting needle 3416b extending through a lumen 3418b in the wall of the shaft 3412 and a third felting needle 3416c extending through a lumen 3418c in the wall of the shaft 3412. Additionally, FIG. 84 illustrates that each of the felting needles 3416a, 3416b, 3416c may be configured to curved toward a central, longitudinal axis of the shaft 3412.

It can be appreciated that each of the felting needles 3416a, 3416b, 3416c may be attached to a felting drive mechanism 46 (shown in FIG. 3), whereby the felting drive mechanism 46 is configured to rapidly displace (e.g., oscillate, drive) the needle 3316 back and forth through the lumens 3418a, 3418b, 3418c, respectively. It can be appreciated that the drive mechanism 46 may be driven by a motor (e.g., the drive mechanism may be coupled to a motorized system) or, alternatively, via pneumatic, magnetic, hydraulic or similar systems.

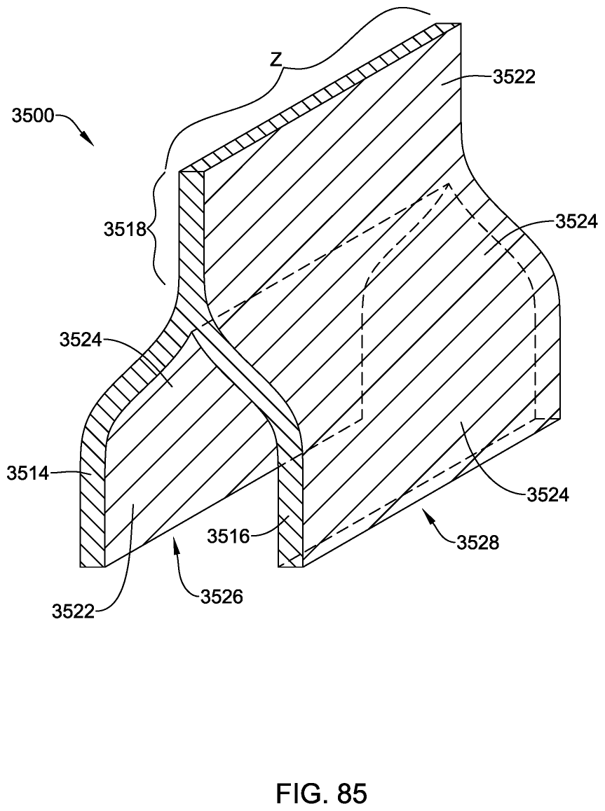

FIG. 85 illustrates a repair implant 3500. The repair implant 3500 may include a first flap 3514, a second flap 3516 and tab 3518. FIG. 85 illustrates that the first flap 3514 may include a first free edge 3526 and the second flap may include a second free edge 3528. It some examples, the first flap 3514 and the second flap 3516 may be able to flex (e.g., spread) away from one another while joining with one another along a connection region at the tab 3518. Further, FIG. 85 illustrates that the repair implant 3500 may have a width “Z”. In some examples, the width Z may be about 1 mm to about 30 mm, or about 2 mm to about 20 mm, about 4 mm to about 15 mm, about 10 mm to about 15 mm, about 5 mm to about 20 mm.

Further, the repair implant 3500 (including the first flap 3514, the second flap 3516 and the tab 3518) may include a base material 3524 formed from a biologic component. The base material 3524 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 3500 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 3500. For example, a lattice structure and/or other structures described herein may be formed by molding which may be punched, woven, etc. to form the desired arrangement. The repair implant 3500 may be porous or included pathways to encourage tissue in-growth. In some embodiments, the repair implant 3500 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 85 further illustrates that the repair implant 3500 may further include a feltable material 3522 integrated (e.g., interwoven, interlinked, interlaced, knitted, crisscrossed, netted, randomly-dispersed, uniformly-dispersed, arranged, etc.) within the base material 3524. For example, FIG. 85 illustrates the repair implant 3500 may include a plurality of feltable fibers 3522 integrated within a collagen base material 3524. It can be appreciated that the feltable fibers 3522 may be distributed within the collagen base material 3524 forming the first flap 3514, a second flap 3516 and the tab 3518 of the repair implant 3500.

In some examples, the plurality of individual feltable fibers 3522 may be matted, condensed, pressed, bonded, etc. together to form a non-woven structure or construct integrated with the collagen base material 3524. In some examples, the individual feltable fibers 3522 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3522 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3522 may include combinations of any of the materials disclosed. For example, the feltable fibers 3522 may be formed from a combination of any of natural fibers, synthetic fibers, biodegradable fibers, collagen fibers, or the like. It can be further appreciated that the feltable fibers 3522 may be randomly aligned with one another to form a non-woven felted structure. In other examples, however, the feltable fibers 3522 may be aligned with one another to achieve unidirectional stiffness properties with the non-woven felted structure.

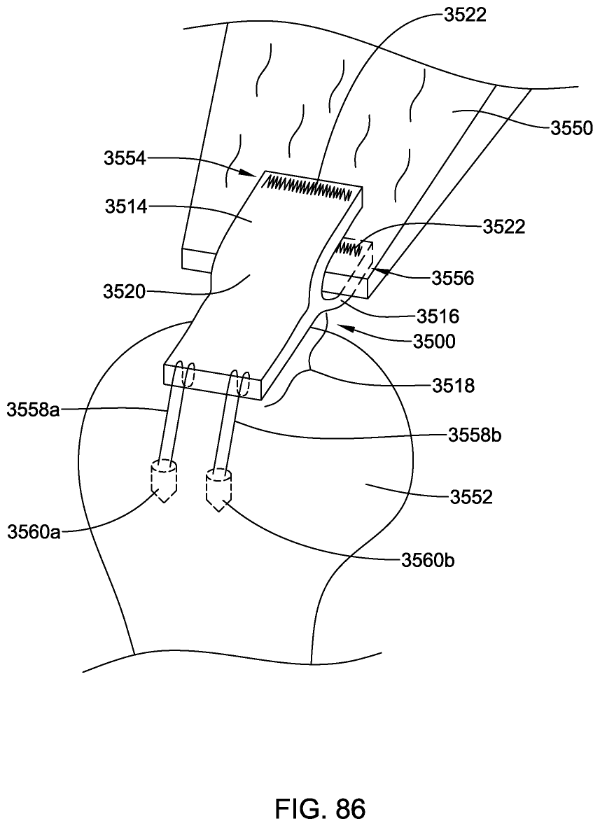

FIG. 86 illustrates a perspective view of the example tendon repair device 3500 implanted within a shoulder. It can be appreciated from FIG. 86 that the first flap 3514 and the second flap 3516 may be positioned adjacent to a rotator cuff tendon 3550 whereby the first flap 3514 may be positioned along a superior surface of the rotator cuff tendon 3550 and the second flap 3516 may be positioned along the inferior surface of the rotator cuff tendon 3550. In other words, together the first flap 3514 and the second flap 3516 may sandwich a portion of the rotator cuff tendon 3550. Additionally, FIG. 86 illustrates that the first flap 3514 may be attached to the rotator cuff tendon 3550 along a feltable region 3554. It can be appreciated that the first flap 3514 may be attached to the rotator cuff tendon 3550 along the feltable region 3554 via felting the feltable fibers 3522 to the rotator cuff tendon 3550. Further, FIG. 86 illustrates that the second flap 3516 may be attached to the rotator cuff tendon 3550 along a feltable region 3556. It can be appreciated that the second flap 3516 may be attached to the rotator cuff tendon 3550 along the feltable region 3556 via felting the feltable fibers 3522 to the rotator cuff tendon 3550.

FIG. 86 further illustrates that the tab 3518 of the tendon repair device 3500 may be attached to the footprint of the greater tuberosity 3552 via a combination of sutures and anchors. For example, FIG. 86 illustrates that a first suture 3558a and a second suture 3558b may each be woven through a portion of the tab 3518 of the repair implant 3550. Further, it can be appreciated that the ends of each of the sutures 3558a, 3558b may be positioned along the footprint of the greater tuberosity 3552 such that an appropriate amount of tension is imparted to the rotator cuff tendon 3550 via a pull force applied to the biologic component 3500. After applying tension to the repair implant 3550, the ends of the first suture 3558a may be attached to the footprint of the greater tuberosity 3552 via an anchor 3560a (e.g., bone anchor, knotless bone anchor, etc.) and the ends of the second suture 3558b may be attached to the footprint of the greater tuberosity 3552 via an anchor 3560b (e.g., bone anchor, knotless anchor, etc.). It can be further appreciated that each of the sutures 3558a, 3558b may be individually tensioned (via the placement and attachment of the anchors 3560a, 3560b) to tailor the placement and engagement of the biologic component 3520 of the repair implant 3500 relative to the torn portion of the rotator cuff tendon 3550.

For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

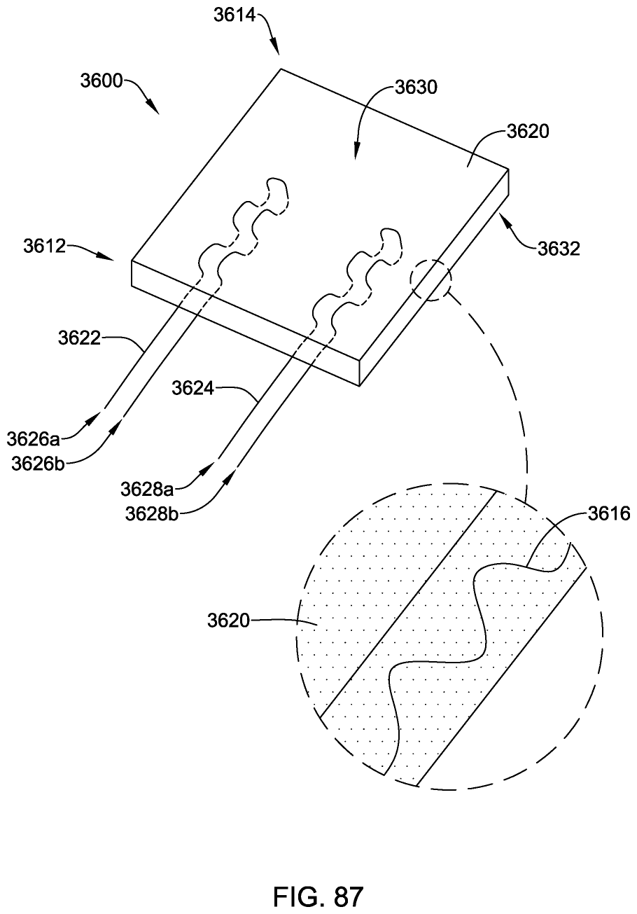

FIG. 87 illustrates a repair implant 3600. The repair implant 3600 may include a biologic component 3620 having a first end region 3612 and a second end region 3614. The biologic component 3620 may include collagen, a collagen element and/or a collagen-like material similar to other bioinductive collagen constructs described herein. In some instances, the biologic component 3620 may be described as a collagen implant.

Additionally, the detailed view of FIG. 87 illustrates that the repair implant 3600 may also include a plurality of feltable fibers 3616 integrated into the biologic component 3620. In some examples, the feltable fibers 3616 may be uniformly dispersed within the biologic component 3620 from the first end region 3612 to the second end region 3614. In other examples, the feltable fibers 3616 may be non-uniformly dispersed within the biologic component 3620. In yet other examples, the feltable fibers 3616 may be described as a “felting mesh” that is integrated within the biologic component 3620.

The feltable fibers 3616 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3616 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3616 may include combinations of any of the materials disclosed. For example, the feltable fibers 3616 may be formed from a combination of any of natural fibers, synthetic fibers, biodegradable fibers, collagen fibers, or the like.

FIG. 87 further illustrates that the repair implant 3600 may also include one or more sutures attached to the biologic component 3620. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 87 illustrates that the repair implant 3600 may include sutures 3622, 3624. While FIG. 87 illustrates the repair implant 3600 include two sutures 3622, 3624, it is contemplated that the repair implant 3600 may include 1, 2, 3, 4, 5, 6 or more sutures.

Further, FIG. 87 illustrates that each of the sutures 3622, 3624 may pass through the body of the biologic component 3620 from a first face 3630 of the biologic component 3620 to a second face 3632 of the biologic component 3620. In some instances, each of the sutures 3622, 3624 may take the form of the a slidable suture which weaves in a substantially wave pattern within the body of the biologic component 3620. In other words, each of the sutures 3622, 3624 may pass in and out of the body biologic component 3620, whereby a portion of each of the sutures 3622, 3624 extends from the first face 3630 to the second face 3632 of the biologic component 3620 in a wave-like pattern. Further, it can be appreciated that the suture 3622 may include a first end 3626a and a second end 3626b and the suture 3624 may include a first end 3628a and a second end 3628b. The ends 3626a, 3626b of the suture 3622 and the ends 3628a, 3628b of the suture 3624 may extend away from the biologic component 3620 of the repair implant 3600.

As discussed herein, the repair implant 3600 may be configured such that each of the sutures 3626a, 3626b may freely “slide” through the body of the biologic component 3620 from a first face 3630 to a second face 3620 of the biologic component 3620. In other words, the repair implant 3600 may be configured such that pulling on the first end 3626a of the suture 3622 may permit the suture 3622 to slide (e.g., move, pass through, etc.) through the apertures (e.g., holes, openings, etc.) through which the suture 3622 passes from the first face 3630 of the biologic component 3620 to the second face 3632 biologic component 3620. Accordingly, it can be appreciated that pulling first end 3626a of the suture 3622 may space the first end 3626a farther away from the biologic component 3620 while moving the second end 3626b closer to the biologic component 3620. Similarly, pulling on the first end 3628a of the suture 3624 may permit the suture 3624 to slide (e.g., move, pass through, etc.) through the apertures (e.g., holes, openings, etc.) through which the suture 3624 passes from the first face 3630 of the biologic component 3620 to the second face 3632 of the biologic component 3620. Accordingly, it can be appreciated that pulling first end 3628a of the suture 3624 may space the first end 3628a farther away from the biologic component 3620 while moving the second end 3628b closer to the biologic component 3620.

It can be appreciated that the sutures 3622, 3624 may be utilized to secure the repair implant 3620 at a target site. For example, the ends 3626a, 3626b, 3628a, 3628b of the sutures 3622, 3624 may be utilized to secure and tension the repair implant 3620 to a target site (e.g., footprint of the greater tuberosity or similar site) using one or more of a variety of anchors (e.g., bone anchors, knotless anchors, etc.).

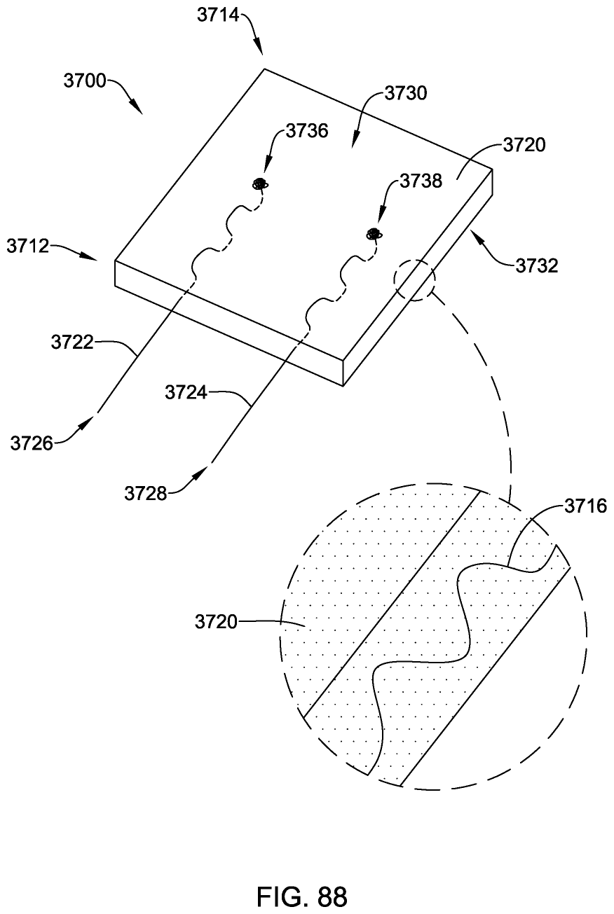

FIG. 88 illustrates a repair implant 3700. The repair implant 3700 may include a biologic component 3720 having a first end region 3712 and a second end region 3714. The biologic component 3720 may include collagen, a collagen element and/or a collagen-like material similar to other bioinductive collagen constructs described herein. In some instances, the biologic component 3720 may be described as a collagen implant.

Additionally, the detailed view of FIG. 88 illustrates that the repair implant 3700 may also include a plurality of feltable fibers 3716 integrated into the biologic component 3720. In some examples, the feltable fibers 3716 may be uniformly dispersed within the biologic component 3720 from the first end region 3712 to the second end region 3714. In other examples, the feltable fibers 3716 may be non-uniformly dispersed within the biologic component 3720. In yet other examples, the feltable fibers 3716 may be described as a “felting mesh” that is integrated within the biologic component 3720.

The feltable fibers 3716 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3716 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3716 may include combinations of any of the materials disclosed. For example, the feltable fibers 3716 may be formed from a combination of any of natural fibers, synthetic fibers, biodegradable fibers, collagen fibers, or the like.

FIG. 88 further illustrates that the repair implant 3700 may also include one or more sutures attached to the biologic component 3720. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 88 illustrates that the repair implant 3700 may include sutures 3722, 3724. While FIG. 88 illustrates the repair implant 3700 include two sutures 3722, 3724, it is contemplated that the repair implant 3700 may include 1, 2, 3, 4, 5, 6 or more sutures.

Further, FIG. 88 illustrates that each of the sutures 3722, 3724 may pass through the body of the biologic component 3720 from a first face 3730 of the biologic component 3720 to a second face 3732 of the biologic component 3720. In some instances, each of the sutures 3722, 3724 may take the form of the a slidable suture which weaves in a substantially wave-like pattern within the body of the biologic component 3720. In other words, each of the sutures 3722, 3724 may pass in and out of the body biologic component 3720, whereby a portion of each of the sutures 3722, 3724 extends from the first face 3730 to the second face 3732 of the biologic component 3720 in a wave-like pattern. Further, it can be appreciated that the suture 3722 may include a first end 3726 and the suture 3724 may include a first end 3728. The end 3726 of the suture 3722 and the end 3728 of the suture 3724 may extend away from the biologic component 3720 of the repair implant 3700.

Additionally, FIG. 88 illustrates that the suture 3722 may include a second end 3736 which may be fixedly attached to the biologic component 3720. For example, the suture 3722 may include a second end 3736 which forms knot along the biologic component 3720. In some examples, the second end 3736 of the suture 3722 may engage one or more of the feltable fibers 3716 of the biologic component, thereby fixedly attaching the second end 3736 to the biologic component 3720. Similarly, the suture 3724 may include a second end 3738 which forms knot along the biologic component 3720. In some examples, the second end 3738 of the suture 3724 may engage one or more of the feltable fibers 3716 of the biologic component, thereby fixedly attaching the second end 3736 to the biologic component 3720.

As discussed herein, the repair implant 3700 may be configured such that a portion of each of the sutures 3722, 3724 may freely “slide” through the body of the biologic component 3720 from a first face 3730 to a second face 3732 of the biologic component 3720. In other words, the repair implant 3700 may be configured such that pulling on the first end 3726 of the suture 3722 may permit a portion of the suture 3722 to slide (e.g., move, pass through, etc.) through the apertures (e.g., holes, openings, etc.) through which the suture 3722 passes from the first face 3730 of the biologic component 3720 to the second face 3732 of the biologic component 3720. Accordingly, because the second end 3736 of the suture 3722 is fixedly attached to the biologic component 3720, it can be appreciated that pulling first end 3726 of the suture 3722 may impart a tension on the biologic component 3720 while also permitting the portion the suture 3722 extending through the biologic component 3720 to slide freely through the biologic component 3720. Similarly, because the second end 3738 of the suture 3724 is fixedly attached to the biologic component 3720, it can be appreciated that pulling first end 3728 of the suture 3724 may impart a tension on the biologic component 3720 while also permitting the portion the suture 3724 extending through the biologic component 3720 to slide freely through the biologic component 3720.

It can be appreciated that the sutures 3722, 3724 may be utilized to secure the repair implant 3720 at a target site. For example, the ends 3726, 3728 of the sutures 3722, 3724 may be utilized to secure and tension the repair implant 3720 to a target site (e.g., footprint of the greater tuberosity or similar site) using one or more of a variety of anchors (e.g., bone anchors, knotless anchors, etc.).

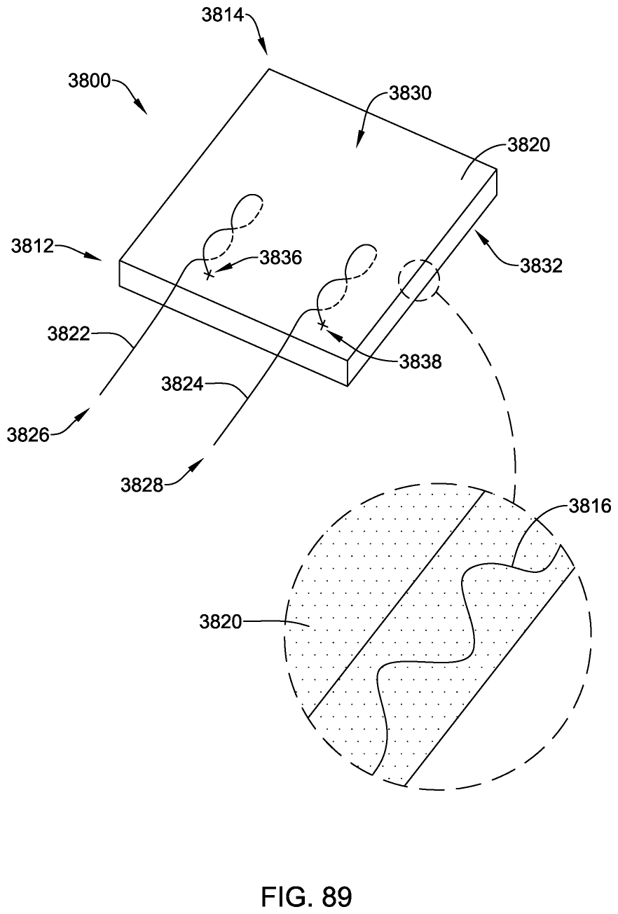

FIG. 89 illustrates a repair implant 3800. The repair implant 3800 may include a biologic component 3820 having a first end region 3812 and a second end region 3814. The biologic component 3820 may include collagen, a collagen element and/or a collagen-like material similar to other bioinductive collagen constructs described herein. In some instances, the biologic component 3820 may be described as a collagen implant.

Additionally, the detailed view of FIG. 89 illustrates that the repair implant 3800 may also include a plurality of feltable fibers 3816 integrated into the biologic component 3820. In some examples, the feltable fibers 3816 may be uniformly dispersed within the biologic component 3820 from the first end region 3812 to the second end region 3814. In other examples, the feltable fibers 3816 may be non-uniformly dispersed within the biologic component 3820. In yet other examples, the feltable fibers 3816 may be described as a “felting mesh” that is integrated within the biologic component 3820.

The feltable fibers 3816 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3816 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3816 may include combinations of any of the materials disclosed. For example, the feltable fibers 3816 may be formed from a combination of any of natural fibers, synthetic fibers, biodegradable fibers, collagen fibers, or the like.

FIG. 89 further illustrates that the repair implant 3800 may also include one or more sutures attached to the biologic component 3820. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof. For example, FIG. 89 illustrates that the repair implant 3800 may include sutures 3822, 3824. While FIG. 89 illustrates the repair implant 3800 include two sutures 3822, 3824, it is contemplated that the repair implant 3800 may include 1, 2, 3, 4, 5, 6 or more sutures.

Further, FIG. 89 illustrates that each of the sutures 3822, 3824 may pass through the body of the biologic component 3820 from a first face 3830 of the biologic component 3820 to a second face 3832 of the biologic component 3820. In some instances, each of the sutures 3822, 3824 may take the form of the a slidable suture which weaves in a substantially wave-like pattern within the body of the biologic component 3820. In other words, each of the sutures 3822, 3824 may pass in and out of the body biologic component 3820, whereby a portion of each of the sutures 3822, 3824 extends from the first face 3830 to the second face 3832 of the biologic component 3820 in a wave-like pattern. Further, it can be appreciated that the suture 3822 may include a first end 3826 and the suture 3824 may include a first end 3828. The end 3786 of the suture 3822 and the end 3828 of the suture 3824 may extend away from the biologic component 3820 of the repair implant 3800.

FIG. 89 further illustrates that a portion of each of the sutures 3822, 3824 may take the form of a “figure eight” pattern along the biologic component 3820. It can be appreciated that the a portion of each of the sutures 3822, 3824 may take the form of other patterns along the biologic component 3820. For example, a portion of each of the sutures 3822, 3824 may take the form of a star pattern, a chevron pattern, a spiral pattern, a zig-zag pattern, or any other suitable, similar patterns.

Additionally, FIG. 89 illustrates that the suture 3822 may include a second end 3836 which may be fixedly attached to the biologic component 3820. For example, the suture 3822 may include a second end 3836 which forms knot along the biologic component 3820. In some examples, the second end 3836 of the suture 3822 may engage one or more of the feltable fibers 3816 of the biologic component, thereby fixedly attaching the second end 3836 to the biologic component 3820. Similarly, the suture 3824 may include a second end 3838 which forms knot along the biologic component 3820. In some examples, the second end 3838 of the suture 3824 may engage one or more of the feltable fibers 3816 of the biologic component, thereby fixedly attaching the second end 3836 to the biologic component 3820.

As discussed herein, the repair implant 3800 may be configured such that a portion of each of the sutures 3822, 3824 may freely “slide” through the body of the biologic component 3820 from a first face 3830 to a second face 3832 of the biologic component 3820. In other words, the repair implant 3800 may be configured such that pulling on the first end 3826 of the suture 3822 may permit a portion of the suture 3822 to slide (e.g., move, pass through, etc.) through the apertures (e.g., holes, openings, etc.) through which the suture 3822 passes from the first face 3830 of the biologic component 3820 to the second face of the 3832 biologic component 3820. Accordingly, because the second end 3836 of the suture 3822 is fixedly attached to the biologic component 3820, it can be appreciated that pulling first end 3826 of the suture 3822 may impart a tension on the biologic component 3820 while also permitting the portion the suture 3822 extending through the biologic component 3820 to slide freely through the biologic component 3820. Similarly, because the second end 3838 of the suture 3824 is fixedly attached to the biologic component 3820, it can be appreciated that pulling first end 3828 of the suture 3824 may impart a tension on the biologic component 3820 while also permitting the portion the suture 3824 extending through the biologic component 3820 to slide freely through the biologic component 3820.

It can be appreciated that the sutures 3822, 3824 may be utilized to secure the repair implant 3820 at a target site. For example, the ends 3826, 3828 of the sutures 3822, 3824 may be utilized to secure and tension the repair implant 3820 to a target site (e.g., footprint of the greater tuberosity or similar site) using one or more of a variety of anchors (e.g., bone anchors, knotless anchors, etc.).

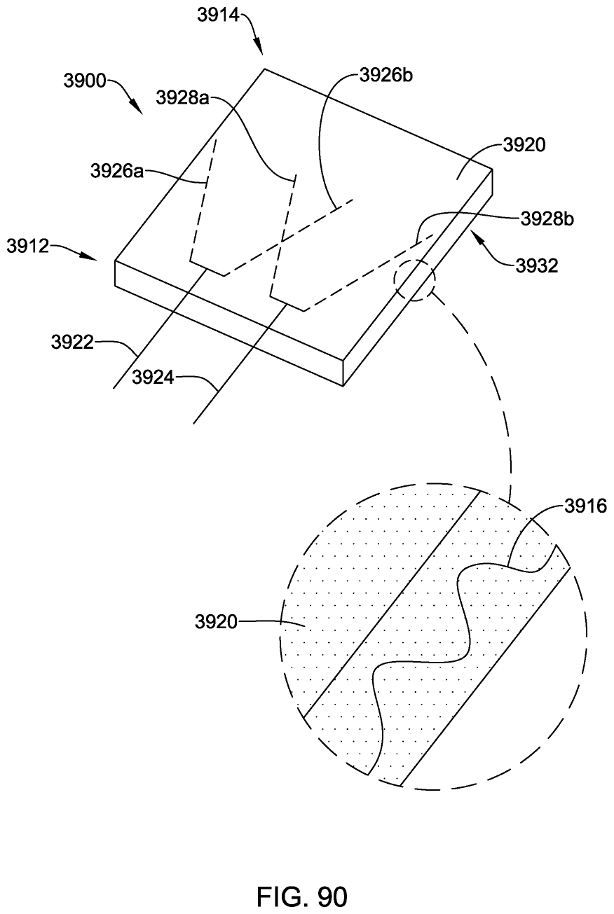

FIG. 90 illustrates a repair implant 3900. The repair implant 3900 may include a biologic component 3920 having a first end region 3912 and a second end region 3914. The biologic component 3920 may include collagen, a collagen element and/or a collagen-like material similar to other bioinductive collagen constructs described herein. In some instances, the biologic component 3920 may be described as a collagen implant.

Additionally, the detailed view of FIG. 90 illustrates that the repair implant 3900 may also include a plurality of feltable fibers 3916 integrated into the biologic component 3920. In some examples, the feltable fibers 3916 may be uniformly dispersed within the biologic component 3920 from the first end region 3912 to the second end region 3914. In other examples, the feltable fibers 3916 may be non-uniformly dispersed within the biologic component 3920. In yet other examples, the feltable fibers 3916 may be described as a “felting mesh” that is integrated within the biologic component 3920.

The feltable fibers 3916 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 3916 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 3916 may include combinations of any of the materials disclosed. For example, the feltable fibers 3916 may be formed from a combination of any of natural fibers, synthetic fibers, biodegradable fibers, collagen fibers, or the like.

FIG. 90 further illustrates that the repair implant 3900 may also include one or more sutures 3922, 3924 attached to the biologic component 3920. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

Further, FIG. 90 illustrates that the suture 3922 may be configured such that it includes a first arm 3926a and a second arm 3926b. In some examples, the first arm 3926a and a second arm 3926b may be separate components (e.g., separate reinforcing members that are attached to the suture 3922). In other examples, the first arm 3926a and a second arm 3926b may be an extension of the suture 3922. Each of the first arm 3926a and the second arm 3926b may be attached to the suture 3922. In other examples, each of the first arm 3926a and the second arm 3926b may be unattached to the suture 3922. Additionally, it can be appreciated from FIG. 90 that the first arm 3926a and the second arm 3926b may extend into the biologic component 3920. For example, all or a portion of the first arm 3926a and/or the second arm 3926b may extend into and anchor within a portion of the body of the biologic component 3920. Further, in some examples, the first arm 3926a and/or the second arm 3926b may engage the feltable fibers 3916, thereby anchoring the sutures first arm 3926a and/or the second arm 3926b within the feltable mesh formed by the feltable fibers 3916.

Similarly, the suture 3924 may be configured such that it includes a first arm 3928a and a second arm 3928b. In some examples, the first arm 3928a and a second arm 3928b may be separate components (e.g., separate reinforcing members that are attached to the suture 3924). In other examples, the first arm 3928a and a second arm 3928b may be an extension of the suture 3924. Each of the first arm 3928a and the second arm 3928b may be attached to the suture 3924. In other examples, each of the first arm 3928a and the second arm 3928b may be unattached to the suture 3924. Additionally, it can be appreciated from FIG. 90 that the first arm 3928a and the second arm 3928b may extend into the biologic component 3920. For example, all or a portion of the first arm 3928a and/or the second arm 3928b may extend into and anchor within a portion of the body of the biologic component 3920. Further, in some examples, the first arm 3928a and/or the second arm 3928b may engage the feltable fibers 3916, thereby anchoring the sutures first arm 3928a and/or the second arm 3928b within the feltable mesh formed by the feltable fibers 3916. It can be further appreciated that the arms 3926a, 3926b, 3928a, 3928b may be configured to reinforce and strengthen the biologic component 3920.

It can be appreciated that the sutures 3922, 3924 may be utilized to secure the repair implant 3920 at a target site. For example, the sutures 3922, 3924 may be utilized to secure and tension the repair implant 3920 to a target site (e.g., footprint of the greater tuberosity or similar site) using one or more of a variety of anchors (e.g., bone anchors, knotless anchors, etc.). For example, the biologic component 3920 may be attached (e.g., felted) to the supraspinatus tendon using felting techniques described herein and the sutures 3922, 3924 may be attached to the footprint of the greater tuberosity using one or more of a variety of anchors (e.g., bone anchors, knotless anchors, etc.).

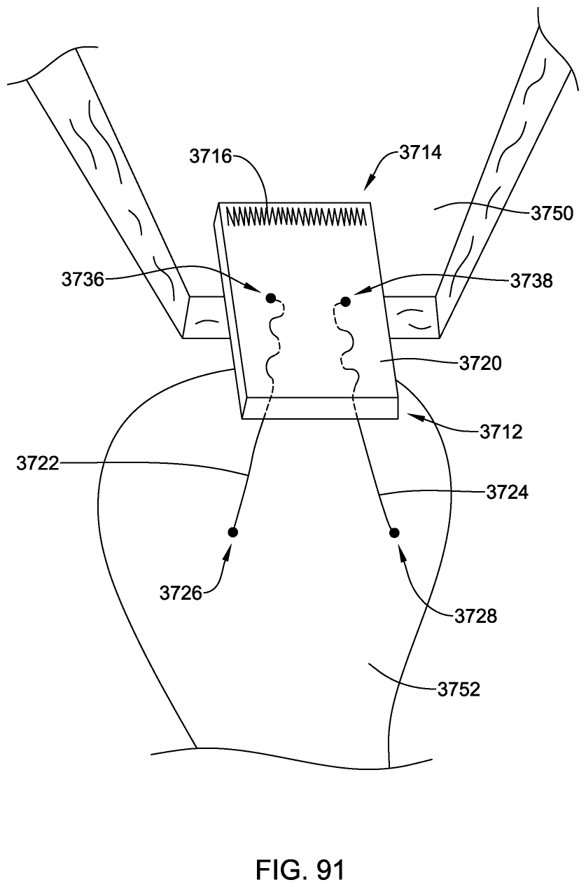

FIG. 91 illustrates a perspective view of the repair implant 3700 (shown in FIG. 88) implanted within a shoulder. It can be appreciated from FIG. 91 that the biologic component 3720 may be attached to the rotator cuff tendon 3750 along a portion of the second end region 3714. For example, it can be appreciated that the biologic component 3720 may be attached to the rotator cuff tendon 3750 along the second end region 3714 via felting the feltable fibers 3716 to the rotator cuff tendon 3750.

FIG. 91 further illustrates that repair implant 3700 may be attached to the footprint of the greater tuberosity 3752 via a combination of slidable sutures and anchors. For example, it can be appreciated that a portion of the slidable sutures 3722, 3724 may be positioned along the footprint of the greater tuberosity 3752 such that an appropriate amount of tension is imparted to the rotator cuff tendon 3750 via a pull force applied to the biologic component 3720. After applying tension to the repair implant 3750, the end 3726 of the first slidable suture 3722 may be attached to the footprint of the greater tuberosity 3752 via an anchor 3726 (e.g., bone anchor, knotless bone anchor, etc.) and the end of the second slidable suture 3724 may be attached to the footprint of the greater tuberosity 3552 via an anchor 3728 (e.g., bone anchor, knotless bone anchor, etc.). It can be further appreciated that each of the slidable sutures 3722, 3724 may be individually tensioned (via the placement and attachment of the anchors 3726, 3728) to tailor the placement and engagement of the biologic component 3720 of the repair implant 3700 relative to the torn portion of the rotator cuff tendon 3750. It can be appreciated that the repair implants 3600, 3800, 3900 may be attached to the rotator cuff tendon 3750 and the attached to the footprint of the greater tuberosity 3752 similarly to that described herein with respect to the repair implant 3700 and illustrated in FIG. 91.

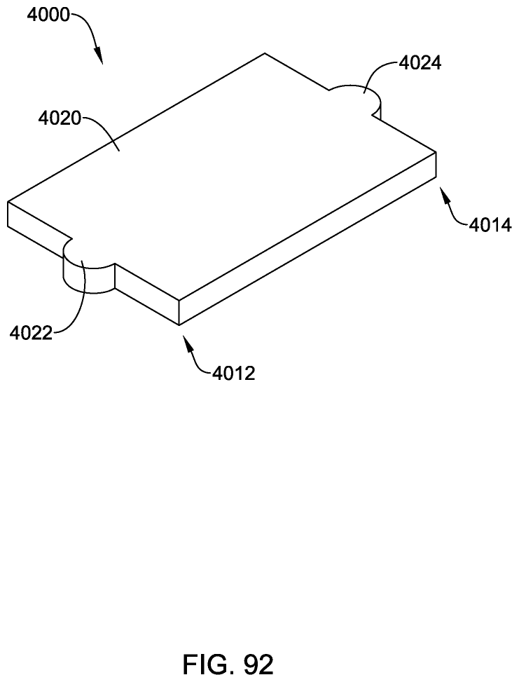

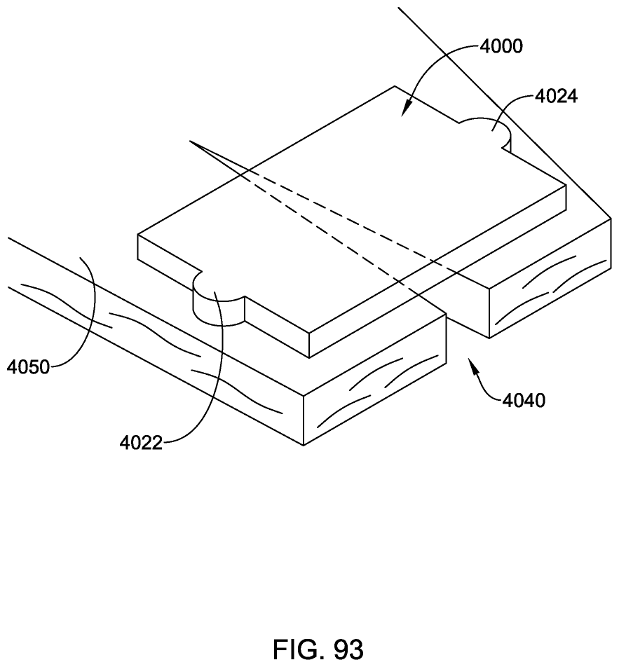

FIG. 92 illustrates a repair implant 4000. The repair implant 4000 may include a first end region 4012 and a second end region 4014 positioned opposite the first end region 4012.

In some examples, the repair implant 4000 may be formed from a plurality of individual feltable fibers which are matted, condensed, pressed, bonded, etc. together to form a non-woven structure. In some examples, the individual feltable fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers may include combinations of any of the materials disclosed. For example, the feltable fibers may be formed from a combination of any of natural fibers, synthetic fibers, biodegradable fibers, collagen fibers, or the like. It can be further appreciated that the feltable fibers may be randomly aligned with one another to form a non-woven felted structure. In other examples, however, the feltable fibers may be aligned with one another to achieve unidirectional stiffness properties with the non-woven felted structure.

Further, in some examples, the repair implant 4000 may include a collagen base material integrated (e.g., interwoven, interlinked, interlaced, knitted, crisscrossed, netted, randomly-dispersed, uniformly-dispersed, arranged, etc.) with the feltable fibers. The collagen base material may include collagen, a collagen element and/or a collagen-like material similar to the bioinductive collagen constructs described herein. It can be appreciated that the collagen base material may be integrated within the feltable fibers from the first end region 4012 to the second end region 4014 of the repair implant 4000.

FIG. 92 further illustrates that the repair implant 4000 may include one or more tabs 4022, 4024 projecting from the first end region 4012 and/or the second end region 4014. While FIG. 92 illustrates the repair implant including two tabs 4022, 4024, it is contemplated that the repair implant 4000 may include 1, 2, 3, 4, 5, 6, or more tabs 4022, 4024. It can be appreciated that the tabs 4022, 4024 may be configured to provide the repair implant 4000 with a region (e.g., portion, area, etc.) which may be fixed to a target tissue site prior to the felting of the repair implant 4000 to the target tissue site.

For example, FIG. 93 illustrates the repair implant 4000 positioned on a target tissue site 4050. Further, FIG. 93 illustrates the repair implant 4000 positioned adjacent to a tear 4040 of a target tissue site 4050. In some examples, the target tissue site 4050 may include a meniscus (such as the torn meniscus 1838 described with respect to FIG. 37), rotator cuff tissue, rotator cuff tendons, Achilles tendon, anterior cruciate ligament, or any other similar tissue, tendon, etc. that may utilize a repair implant.

As described herein, FIG. 93 illustrates that the repair implant 4000 may be positioned across the tear 4040 and initially attached to the target tissue site 4050 by attaching the tabs 4022, 4024 to the target tissue 4050. In some examples, the tabs 4022, 4024 may be attached to the target tissue 4050 via one or more staples, a suture anchor, knotless anchor, or other similar fixation methodology. Attachment of the tabs 4022, 4024 may stabilize the repair implant 4000 along the target tissue 4050 or to add stability to an injured tissue site before felting action starts. It can be appreciated that after the repair implant 4000 is initially attached to the target tissue 4050 along the tabs 4022, 4024, the remainder of the repair implant 4000, or a portion thereof, may be felted to the target tissue 4050 using any of the felting techniques described herein.

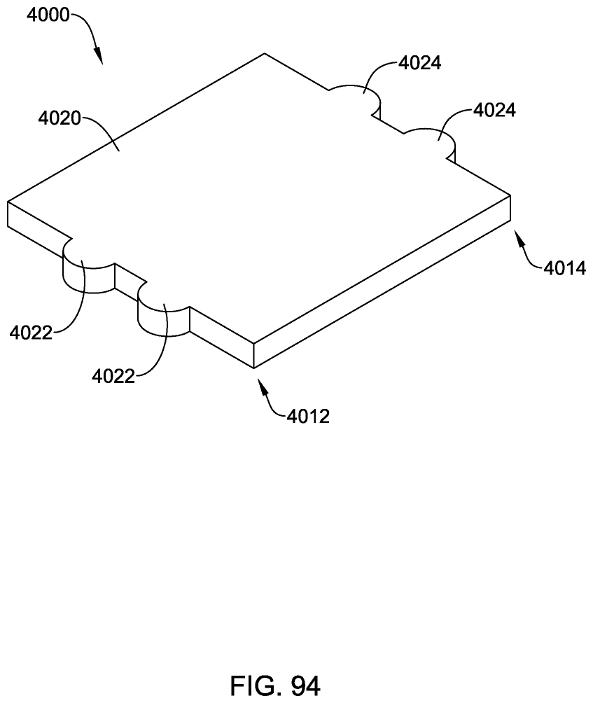

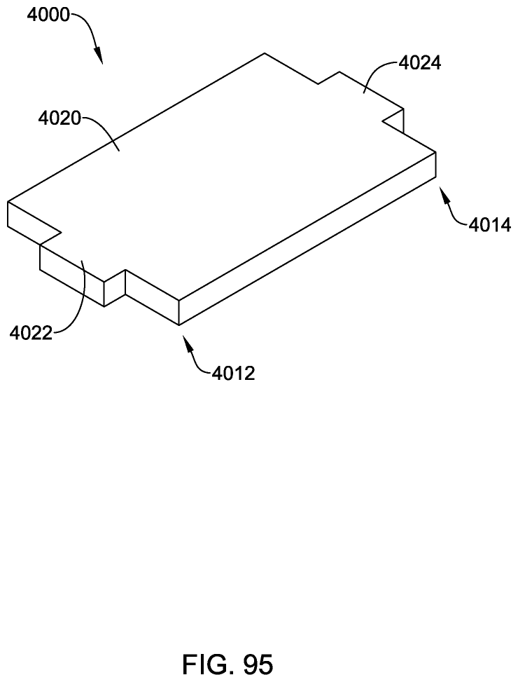

FIGS. 94-95 schematically depict various example arrangements of the felt patch 4000. These examples are in addition to the example arrangement of the felt patch 4000 including the tabs 4022, 4024 described with respect to FIGS. 92-93. The example arrangements of the felt patch 4000 including the tabs 4022, 4024 shown in FIGS. 94-95 are schematic representations that depict alternative arrangements. The edges/boundaries shown in FIGS. 94-95 are intended to mimic the edge/boundaries of the pattern/arrangement shown in FIG. 92, or a variation thereof, and can be utilized in any of the devices disclosed as alternatives of the felt patch 4000.

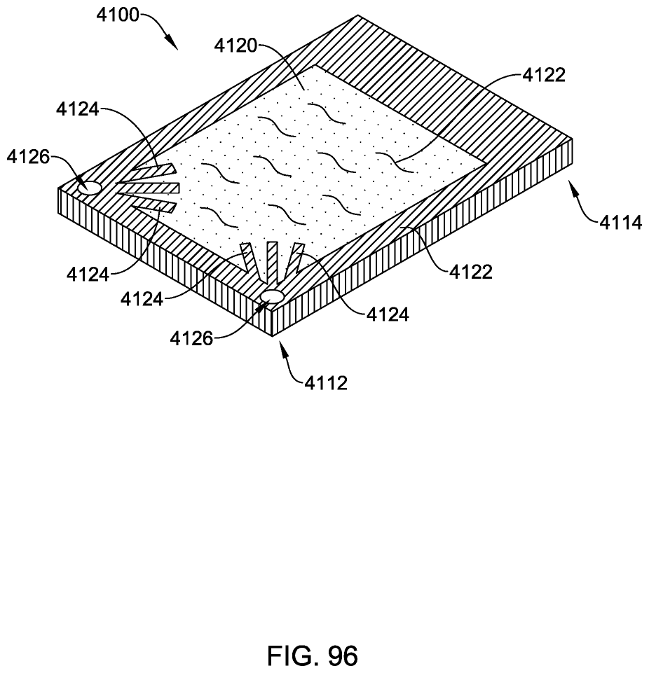

FIG. 96 illustrates repair implant 4100. The repair implant may include a first end region 4112 and a second end region 4114. The repair implant 4100 may include a square shape. However, it is contemplated that the repair implant 4100 may include a variety of shapes such as circular, rectangular, ovular, crescent-shaped, triangular, polygonal, etc.

Additionally, FIG. 96 illustrates that, in some examples, the repair implant 4100 may further include a plurality of feltable fibers 4122 integrated with a biologic component 4120. It can be appreciated that the individual feltable fibers 4122 may be matted, condensed, pressed, bonded, etc. together to form a non-woven structure or construct. In some examples, the individual feltable fibers 4122 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 4122 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 4122 may include combinations of any of the materials disclosed. For example, the feltable fibers 4122 may be formed from a combination of any of natural fibers, synthetic fibers, biodegradable fibers, collagen fibers, or the like. It can be further appreciated that the feltable fibers 4122 may be randomly aligned with one another to form a non-woven felted structure. In other examples, however, the feltable fibers 4122 may be aligned with one another to achieve unidirectional stiffness properties with the non-woven felted structure.

Additionally, the biologic component 4120 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 4120 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 4120. For example, a lattice structure and/or other structures described herein may be formed by molding which may be punched, woven, etc. to form the desired arrangement. The biologic component 4120 may be porous or included pathways to encourage tissue in-growth. In some embodiments, the biologic component 4120 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Additionally, the detailed view of FIG. 96 illustrates that the edges of the repair implant 4100 may be formed from a plurality of individual feltable fibers 4122. It can be appreciated that the individual fibers 4122 of the repair implant 4100 may be used to felt the repair implant 4100 along a target tissue site. It can be appreciated that, in some examples, the repair implant 4100 may include a biologic component free of feltable fibers 4122, yet include edges formed from a plurality of individual feltable fibers 4122. In yet other examples, the edges of the repair implant 4100 may be formed from materials other than the feltable fibers 4122.

Additionally, FIG. 96 illustrates that the repair implant 4100 may include a plurality of reinforcing members 4124 (e.g., reinforcement legs, fingers, etc.) which may extend from the perimeter of the repair implant 4100 toward a central region of the repair implant 4100. It can be appreciated that, in some examples, the reinforcement members 4124 may be formed from the feltable fibers 4122. In other examples, the reinforcement members 4124 may be formed from materials other than the feltable fibers 4122.

FIG. 96 further illustrates that the repair implant 4100 may include one or more apertures 4126 which extend through the repair implant 4100. The apertures 4126 may be positioned in opposite corners, along the first end region 4112, of the repair implant 4100. It can be appreciated that each aperture 4126 may be configured to permit one or more sutures to extend therethrough. Sutures that extend through the apertures 4126 may be utilized to tension and secure the first end region 4112 of the repair implant 4100 to a target tissue site (e.g., tissue, tendon, bone, etc.). It can further be appreciated that the reinforcement members 4124 may be configured to distribute forces imparted onto the biologic component 4120 when the sutures are tensioned and secured. In other words, the reinforcement members 4124 may be configured to distribute loading forces which are concentrated at the apertures 4126 when the sutures are tensioned and secured.

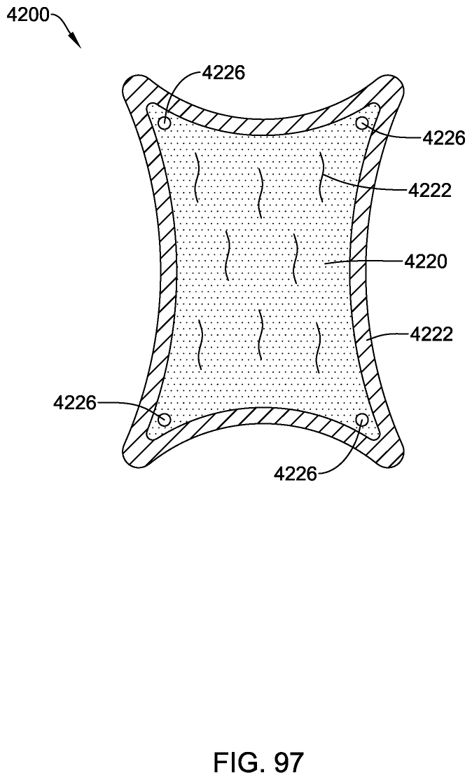

FIG. 97 illustrates repair implant 4200. The repair implant 4200 may include a plurality of feltable fibers 4222 integrated with a biologic component 4220. It can be appreciated that the individual feltable fibers 4222 may be matted, condensed, pressed, bonded, etc. together to form a non-woven structure or construct. In some examples, the individual feltable fibers 4222 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 4222 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 4222 may include combinations of any of the materials disclosed. For example, the feltable fibers 4222 may be formed from a combination of any of natural fibers, synthetic fibers, biodegradable fibers, collagen fibers, or the like. It can be further appreciated that the feltable fibers 4222 may be randomly aligned with one another to form a non-woven felted structure. In other examples, however, the feltable fibers 4222 may be aligned with one another to achieve unidirectional stiffness properties with the non-woven felted structure.

Additionally, the biologic component 4220 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 4220 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 4220. For example, a lattice structure and/or other structures described herein may be formed by molding which may be punched, woven, etc. to form the desired arrangement. The biologic component 4220 may be porous or included pathways to encourage tissue in-growth. In some embodiments, the biologic component 4220 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Additionally, the detailed view of FIG. 97 illustrates that the edges of the repair implant 4200 may be formed from a plurality of individual feltable fibers 4222. It can be appreciated that the individual fibers 4222 of the repair implant 4200 may be used to felt the repair implant 4200 along a target tissue site. It can be appreciated that, in some examples, the repair implant 4200 may include a biologic component 4220 free of feltable fibers 4222, yet include edges formed from a plurality of individual feltable fibers 4222. In yet other examples, the edges of the repair implant 4200 may be formed from materials other than the feltable fibers 4222.

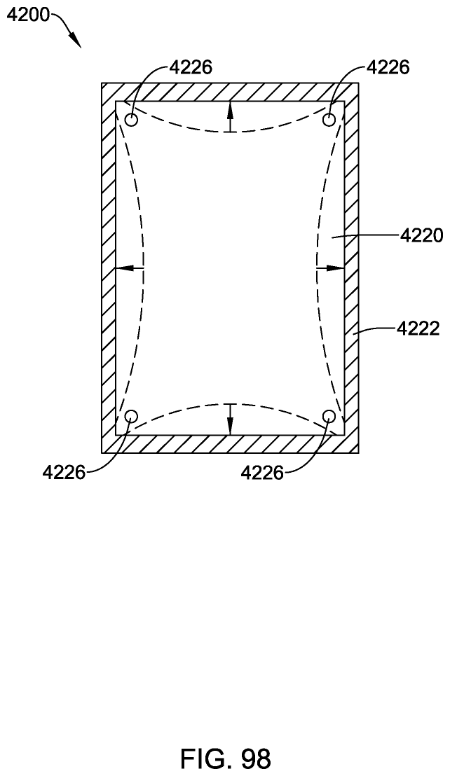

FIG. 97 further illustrates that the repair implant 4200 may include one or more apertures 4226 which extend through the repair implant 4200. The apertures 4226 may be positioned in the corners of the repair implant 4200. It can be appreciated that each aperture 4226 may be configured to permit one or more sutures to extend therethrough. Sutures that extend through the apertures 4226 may be utilized to tension and secure the repair implant 4200 to a target tissue site (e.g., tissue, tendon, bone, etc.). For example, FIG. 98 illustrates the relative shape the repair implant 4200 may form as tension is applied (e.g., via sutures extending through the apertures 4226) to the four corners of the repair implant 4200. Comparing the shape of the repair implant 4200 shown in FIG. 97 to the shape of the repair implant 4200 shown in FIG. 98 illustrates that as the curved edges (illustrated in FIG. 97) of the repair implant 4200 are pulled tight, they may attempt to straighten out, thereby pulling the center of the biologic component 4220 outward and increasing the surface area of the biologic component 4220 which may contact target tissue.

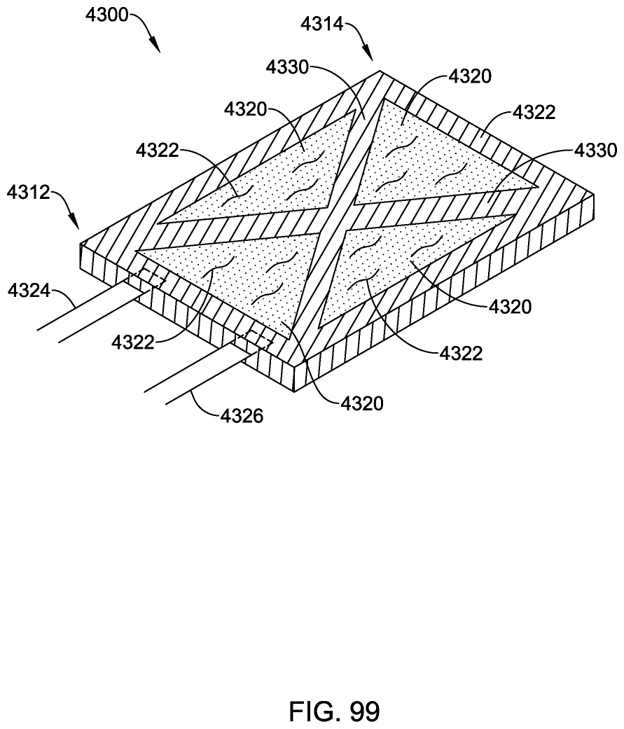

FIG. 99 illustrates repair implant 4300. The repair implant 4300 may include a first end region 4312 and a second end region 4314. The repair implant 4300 may include a square shape. However, it is contemplated that the repair implant 4300 may include a variety of shapes such as circular, rectangular, ovular, crescent-shaped, triangular, polygonal, etc.

Additionally, FIG. 99 illustrates that, in some examples, the repair implant 4300 may further include a plurality of feltable fibers 4322 integrated with a biologic component 4320. It can be appreciated that the individual feltable fibers 4322 may be matted, condensed, pressed, bonded, etc. together to form a non-woven structure or construct. In some examples, the individual feltable fibers 4322 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 4322 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 4322 may include combinations of any of the materials disclosed. For example, the feltable fibers 4322 may be formed from a combination of any of natural fibers, synthetic fibers, biodegradable fibers, collagen fibers, or the like. It can be further appreciated that the feltable fibers 4322 may be randomly aligned with one another to form a non-woven felted structure. In other examples, however, the feltable fibers 4322 may be aligned with one another to achieve unidirectional stiffness properties with the non-woven felted structure.

Additionally, the biologic component 4320 may include collagen, a collagen element and/or a collagen-like material similar to other bioinductive collagen constructs described herein. The collagen component may include reconstituted collagen manufactured from highly purified type 1 collagen from bovine tendons. However, other sources of collagen may be used as well. In some embodiments, the biologic component 4320 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the biologic component 4320. For example, a lattice structure and/or other structures described herein may be formed by molding which may be punched, woven, etc. to form the desired arrangement. The biologic component 4120 may be porous or included pathways to encourage tissue in-growth. In some embodiments, the biologic component 4320 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

Additionally, the detailed view of FIG. 99 illustrates that the edges of the repair implant 4300 may be formed from a plurality of individual feltable fibers 4322. It can be appreciated that the individual fibers 4322 of the repair implant 4300 may be used to felt the repair implant 4300 along a target tissue site. It can be appreciated that, in some examples, the repair implant 4300 may include a biologic component free of feltable fibers 4322, yet include edges formed from a plurality of individual feltable fibers 4322. In yet other examples, the edges of the repair implant 4300 may be formed from materials other than the feltable fibers 4322.

Additionally, FIG. 99 illustrates that the repair implant 4300 may include a plurality of reinforcing ribs 4330 which may extend from corner-to-corner, from the first end region 4312 to the second end region 4314, of the repair implant 4300, thereby forming an “X” shape in a central region of the repair implant 4300. It can be appreciated that, in some examples, the reinforcement ribs 4330 may be formed from the feltable fibers 4322. In other examples, the reinforcement ribs 4330 may be formed from materials other than the feltable fibers 4322.

FIG. 99 further illustrates that the repair implant 4300 may include one or more sutures 4324, 4326 extending from the first end region 4312 of the repair implant 4300. It can be appreciated that the sutures 4324, 4326 may be utilized to tension and secure the first end region 4312 of the repair implant 4300 to a target tissue site (e.g., tissue, tendon, bone, etc.). Further, the second end region 4314 of the repair implant 4300 may be secured to a target tissue site via felting the feltable fibers 4322 positioned along the first end region 4314 to the target tissue site using any of the felting techniques described herein. It can further be appreciated that the reinforcement ribs 4330 may be configured to distribute forces imparted onto the biologic component 4330 when the sutures are tensioned and secured. In other words, the reinforcement ribs 4330 may create a “load path” along which forces imparted onto the biologic component 4330 may be distributed.

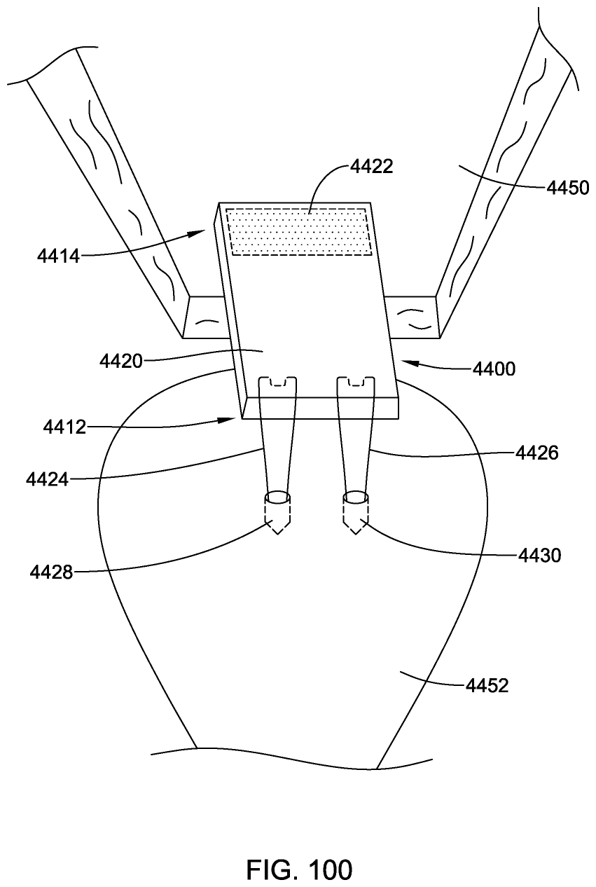

FIG. 100 is a perspective view of a repair implant 4400 implanted within a shoulder. The repair implant 4400 may include a biologic component 4420 having a first end region 4412 and a second end region 4414. FIG. 100 illustrates the first end region 4412 positioned adjacent to a footprint of the greater tuberosity 4452 and the second end region 4414 positioned adjacent to a rotator cuff tendon 4450. The biologic component 4420 may include collagen, a collagen element, a reinforced collagen element, a non-absorbable implant, a collagen-like material similar to other bioinductive collagen constructs described herein and/or combinations of collagen or collagen-like materials described herein. In some instances, the biologic component 4420 may be described as a collagen implant.

Additionally, the repair implant 4400 may also include a plurality of feltable fibers 4422 positioned along the second end region 4414 of the biologic component 4420. The feltable fibers 4422 may be integrated with the biologic component 4420. In some examples, the feltable fibers 4422 may be uniformly dispersed within the biologic component 4420 along the second end region 4414. In other examples, the feltable fibers 4422 may be non-uniformly dispersed within the biologic component 4420. In yet other examples, the feltable fibers 4422 may be described as a “felting mesh” that is integrated within the biologic component 4420.

The feltable fibers 4422 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 4422 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 4422 may include combinations of any of the materials disclosed. For example, the feltable fibers 4422 may be formed from a combination of any of natural fibers, synthetic fibers, biodegradable fibers, collagen fibers, or the like.

FIG. 100 illustrates that the second end region 4414 of the biologic component 4420 of the tendon repair device 4400 may be secured to the rotator cuff tendon 4450 via felting the feltable fibers 4422 to the rotator cuff tendon 4450 tissue using any of the felting techniques described herein.

FIG. 100 further illustrates that the first end region 4412 of the biologic component 4420 of the tendon repair device 4400 may be secured to the footprint of the greater tuberosity 4452 via a combination of sutures and anchors. For example, FIG. 100 illustrates that a first suture 4424 and a second suture 4426 may each be woven through a portion of the first end region 4412 of the repair implant 4450. In some examples, each of the sutures 4424, 4426 may be slidable (e.g., slidably attached) relative to the biologic component 4420 or may be fixed to the implant. The sutures 4424, 4426 may be integrated into the implant itself during manufacturing, or placed there during the procedure with the use of a suture passer or threading needle. Further, it can be appreciated that the ends of each of the sutures 4424, 4426 may be positioned along the footprint of the greater tuberosity 4452 such that an appropriate amount of tension is imparted to the rotator cuff tendon 4450 via a pull force applied to the biologic component 4400. After applying tension to the repair implant 4450, the ends of the first suture 4424 may be attached to the footprint of the greater tuberosity 4452 via an anchor 4428 (e.g., bone anchor, knotless bone anchor, etc.) and the ends of the second suture 4426 may be attached to the footprint of the greater tuberosity 4452 via an anchor 4430 (e.g., bone anchor, knotless anchor, etc.). It can be further appreciated that each of the sutures 4424, 44236 may be individually tensioned (via the placement and attachment of the anchors 4428, 4430) to tailor the placement and engagement of the biologic component 4420 of the repair implant 4400 relative to the torn portion of the rotator cuff tendon 4450.

For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

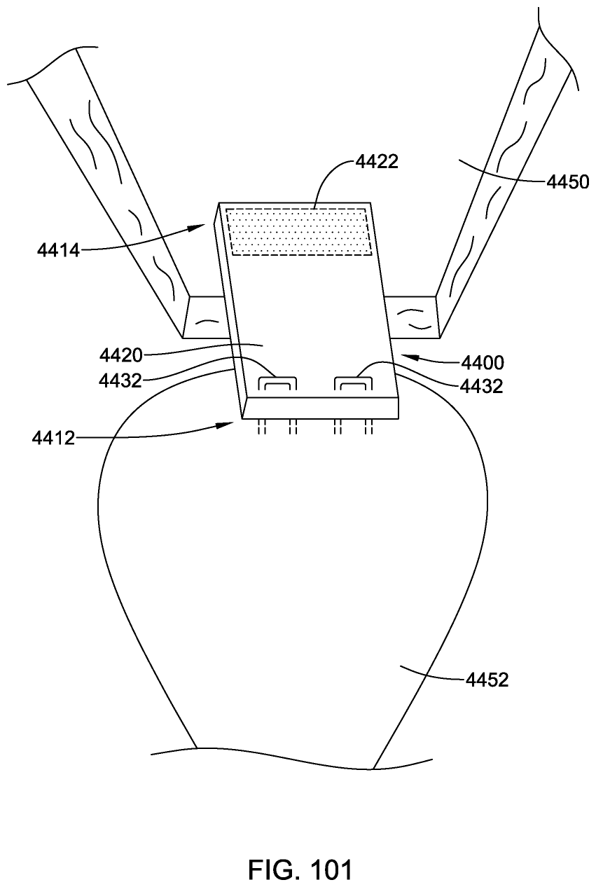

FIG. 101 illustrates the example repair implant 4400 described with respect to FIG. 100. As described with respect to FIG. 100, the repair implant 4400 may include a biologic component 4420 having a first end region 4412 and a second end region 4414. As described herein, FIG. 101 illustrates that the second end region 4414 of the biologic component 4420 of the tendon repair device 4400 may be secured to the rotator cuff tendon 4450 via felting the feltable fibers 4422 to the rotator cuff tendon 4452 tissue using any of the felting techniques described herein. Further, FIG. 101 illustrates that the first end region 4412 of the biologic component 4420 of the tendon repair device 4400 may be secured to the footprint of the greater tuberosity 4452 via one or more staples 4432. Alternatively, the staples 4432 may be used in conjunction with a bone anchor and one or more sutures.

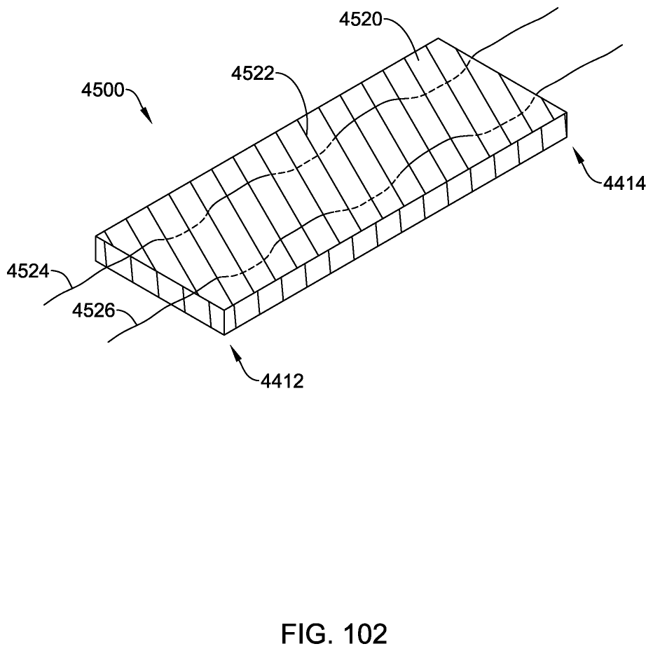

FIG. 102 illustrates a repair implant 4500. The repair implant 4500 may include a first end region 4512 and a second end region 4514 positioned opposite the first end region 4512. FIG. 102 illustrates that the repair implant 4500 may include a rectangular configuration extending from the first end region 4512 to the second end region 4514. However, it is contemplated that the repair implant 4500 may include a variety of shapes such as square, rectangular, ovular, crescent-shaped, triangular, polygonal, etc.

Further, the repair implant 4500 may include a base material 4520 formed from a biologic component. The base material 4520 may include collagen, a collagen element and/or a collagen-like material similar to the bioinductive collagen constructs described herein. The collagen component may include reconstituted collagen manufactured from highly purified type 1 collagen from bovine tendons. However, other sources of collagen may be used as well. In some embodiments, the repair implant 4500 may include a lattice structure that can be 3D printed in a flat 2-dimensional pattern or a 3-dimensional pattern depending on the 3D printing technology utilized. While 3D printing is one illustrative example, other suitable manufacturing techniques may be utilized to manufacture the repair implant 4500. For example, a lattice structure and/or other structures described herein may be formed by molding which may be punched, woven, etc. to form the desired arrangement. The repair implant 4500 may be porous or included pathways to encourage tissue in-growth. In some embodiments, the repair implant 4500 may include a material defining a plurality of pores that encourage tissue growth therein. In some embodiments, the size and/or spacing of the pores and/or pathways may be varied or changed based on the lattice structure, if so provided. In some cases, the size, spacing, and/or direction of the pores and/or pathways may be selected to encourage tissue growth in a particular orientation. It can be appreciated that the porosity and tissue in-growth allows for new collagen to integrate with collagen of the native soft tissue (e.g., tendon) for functional load carrying. Examples of pore defining structures may include, but not be limited to open cell foam structures, mesh structures, micromachined layered structures and structures comprising a plurality of fibers. In some embodiments, fibers may be interlinked with one another. Various processes may be used to interlink the fibers with one another. Examples of processes that may be suitable in some applications include weaving, knitting, crocheting, and braiding.

FIG. 102 further illustrates that the repair implant 4500 may further include a feltable material 4522 integrated (e.g., interwoven, interlinked, interlaced, knitted, crisscrossed, netted, randomly-dispersed, uniformly-dispersed, arranged, etc.) within the base material 4524. For example, FIG. 102 illustrates the repair implant 4500 may include a plurality of feltable fibers 4522 integrated within a collagen base material 4524. It can be appreciated that the feltable fibers 4522 may be distributed within the collagen base material 4524 from the first end region 4512 to the second end region 4514 of the repair implant 4500.

In some examples, the plurality of individual feltable fibers 4522 may be matted, condensed, pressed, bonded, etc. together to form a non-woven structure or construct integrated with the collagen base material 4524. In some examples, the individual feltable fibers 4522 may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers 4522 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers 4522 may include combinations of any of the materials disclosed. For example, the feltable fibers 4522 may be formed from a combination of any of natural fibers, synthetic fibers, biodegradable fibers, collagen fibers, or the like. It can be further appreciated that the feltable fibers 4522 may be randomly aligned with one another to form a non-woven felted structure. In other examples, however, the feltable fibers 4522 may be aligned with one another to achieve unidirectional stiffness properties with the non-woven felted structure.

FIG. 102 further illustrates that the repair implant 4500 may include sutures 4524, 4526 extending within the base material 4520. FIG. 102 illustrates the repair implant 4500 including two sutures 4524, 4526, however, it can be appreciated that the repair implant 4500 may include 1, 2, 3, 4, 5, 6 or more sutures. Further, in some instances, each of the sutures 4524, 4526 may take the form of the a slidable suture which weaves in a substantially wave pattern within the base material 4520. It can be appreciated from FIG. 102 that the ends of the sutures 4524, 4526 may extend away from the base material 4520 of the repair implant 4500.

As discussed herein, the repair implant 4500 may be configured such that when an end of a suture 4524, 4526 is pulled, the suture 4524, 4526 may freely slide through the base material 4520. In other words, pulling one end of a suture 4524, 4526 away from the base material 4520 may draw the opposite end of the suture 4524, 4526 closer to the base material 4520. It can be appreciated that the sutures 4524, 4526 may be configured to tension and secure the base material to a target tissue site (e.g., tissue, tendon, bone, etc.).

For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

In some embodiments, the repair implant 4500 may be configured to provide a combination of structural features, properties and functions that may be used to treat partial or full thickness tears of soft tissues, including partial or full thickness tears of the rotator cuff tendons, ACL repair, LCL repair, the gluteal tendon, meniscus tears, tendons/ligaments of the hip joint capsule, Achilles tendon, etc. The repair implant 4500 may encourage tissue ingrowth while also providing additional mechanical properties such as, but not limited to strength and stiffness. For example, the implant 4500 may provide a layer of collagen over injured tissue to facilitate healing.

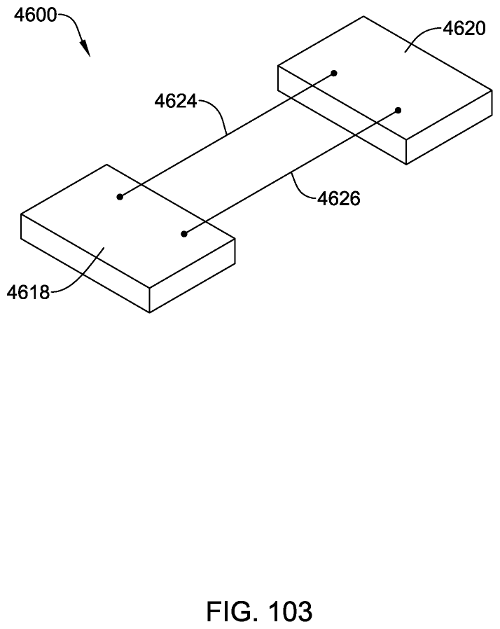

FIG. 103 illustrates a repair implant 4600. The repair implant 4600 may include a first feltable implant 4618 (e.g., feltable scaffold) and a second feltable implant 4620 (e.g., feltable scaffold). Further, FIG. 103 illustrates that the first feltable implant 4618 may be attached to the second feltable implant 4620 by a first suture 4624 and a second suture 4626. FIG. 103 illustrates the repair implant 4600 including two sutures 4624, 4626, however, it can be appreciated that the repair implant 4600 may include 1, 2, 3, 4, 5, 6 or more sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

The first feltable implant 4618 and the second feltable implant 4620 may be formed from a plurality of individual feltable fibers which are matted, condensed, pressed, bonded, etc. together to form a non-woven structure. In some examples, the individual feltable fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers may include combinations of any of the materials disclosed. For example, the feltable fibers may be formed from a combination of any of natural fibers, synthetic fibers, biodegradable fibers, collagen fibers, or the like. It can be further appreciated that the feltable fibers may be randomly aligned with one another to form a non-woven felted structure. In other examples, however, the feltable fibers may be aligned with one another to achieve unidirectional stiffness properties with the non-woven felted structure.

Further, in some examples, the first feltable implant 4618 and the second feltable implant 4620 may include a collagen base material integrated (e.g., interwoven, interlinked, interlaced, knitted, crisscrossed, netted, randomly-dispersed, uniformly-dispersed, arranged, etc.) with the feltable fibers. The collagen base material may include collagen, a collagen element and/or a collagen-like material similar to the bioinductive collagen constructs described herein. It can be appreciated that the collagen base material may be integrated within the feltable fibers forming the first feltable implant 4618 and the second feltable implant 4620.

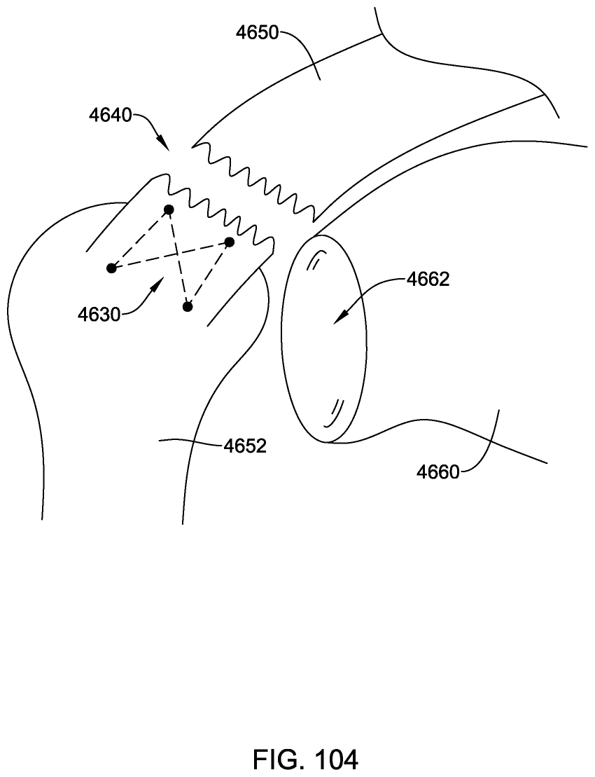

FIG. 104 illustrates a footprint of the greater tuberosity 4652 and the glenoid fossa 4662 of the scapula 4660. The glenoid fossa 4662 comprises a shallow depression in the scapula 4660. A distal portion of the supraspinatus tendon 4650 is also shown. FIG. 104 further illustrates that the tendon 4650 includes a tear 4640 extending through the tendon 4650. The tear 4640 may be described as a full or a partial thickness tear. Further, the tear illustrated in FIG. 104 may take the form of a Type 2 rotator cuff retear whereby a tear occurs in the medial cuff of the rotator cuff, close to the musculotendinous junction (MTJ), after a previous rotator cuff repair (the primary repair implant is depicted in FIG. 104 as reference numeral 4630). The tear may also be a Type 1 retear (retear of the primary repair of the tendon).

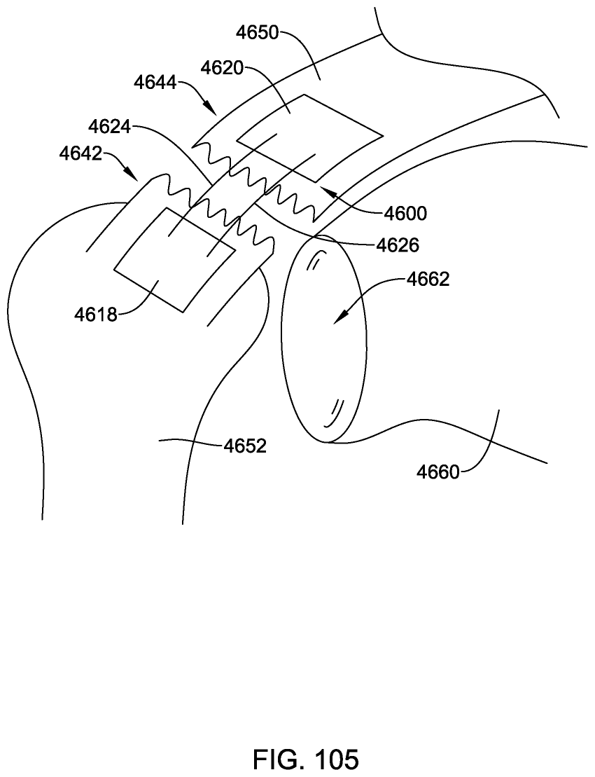

It can be appreciated that the repair implant 4600 may be utilized in a revision surgery to repair a tendon retear, such as the tendon retear illustrated and described with respect to FIG. 104. For example, FIG. 105 illustrates the second feltable implant 4620 attached to a portion 4644 of the torn supraspinatus tendon 4650. It can be appreciated that the second feltable implant 4620 may be attached to the portion 4644 of the supraspinatus tendon 4650 via felting the feltable fibers of the second feltable implant 4620 to the portion 4644 of the supraspinatus tendon 4650 using any of the felting techniques described herein. It can be appreciated that a similar technique may be completed with a longer, single feltable implant where the muscle is felted first, then pulled laterally to the native tendon, and then the second more lateral portion of the implant is felted to the tendon. Alternatively, the medial portion of the implant may be felted, and then the lateral portion could be fixed to bone without felting of the lateral aspect of the implant.

Further, after the attachment of the second feltable implant 4620 to the portion 4644 of the supraspinatus tendon 4650, the repair implant 4600 may bridge the tear in the torn tendon 4650 and be tensioned via positioning and securing the first feltable implant 4618 along a portion 4642 of the torn tendon 4650 which remains fixed to the footprint of the greater tuberosity (e.g., a portion of the tendon which remains in place adjacent to the humeral tuberosity). It can be appreciated that tensioning the repair implant 4600 as described herein may reposition the portion 4642 of the torn tendon 4650 relative to the portion 4644 of the torn tendon 4650, thereby reapproximating the desired position of the tendon 4650 prior to the retear of the tendon 4650. It can be appreciated that, after the first feltable implant 4618 is positioned along the portion 4642 of the supraspinatus tendon 4650, it may be attached to the portion 4642 of the supraspinatus tendon 4650 via felting the feltable fibers of the first feltable implant 4618 to the portion 4642 of the supraspinatus tendon 4650 using any of the felting techniques described herein. It can be further appreciated that the primary repair 4630 illustrated in FIG. 104 is hidden from view by the first feltable implant 4618, which has been positioned overtop of the primary repair 4630 in FIG. 105.

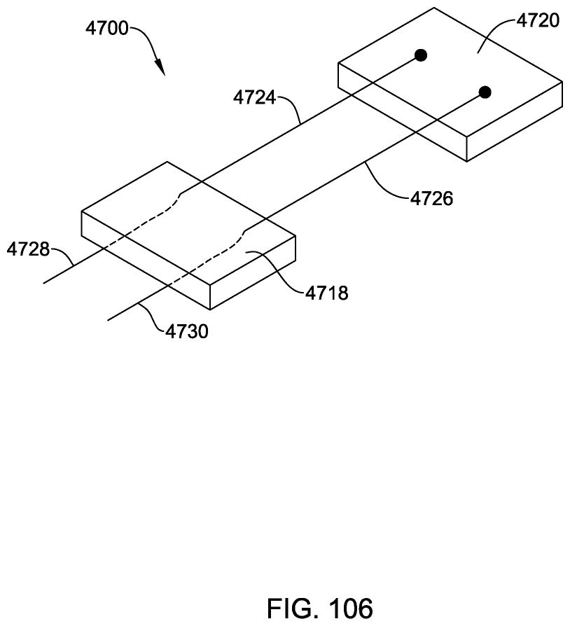

FIG. 106 illustrates a repair implant 4700 which may be similar in form and function to the repair implant 4600 described herein. The repair implant 4700 may include a first feltable implant 4718 (e.g., feltable scaffold) and a second feltable implant 4720 (e.g., feltable scaffold). Further, FIG. 106 illustrates the first feltable implant 4718 may be attached to the second feltable implant 4720 by a first suture 4724 and a second suture 4726. FIG. 106 illustrates the repair implant 4700 including two sutures 4724, 4726, however, it can be appreciated that the repair implant 4700 may include 1, 2, 3, 4, 5, 6 or more sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

It can be further appreciated that each of the sutures 4724, 4726 may take the form of the a slidable suture extending through the first feltable implant 4718. Slidable sutures may be beneficial in that they may provide the ability to tension both feltable sections 4718, 4720 of the implant 4700 post implantation. Each of the sutures 4724, 4726 may be configured to extend along a top face of the first feltable implant 4718, pass through the body of the first feltable implant 4718 and exit a bottom face of the first feltable implant 4718, whereby each suture 4724, 4726 is free to slide through the body of the first feltable implant 4718. Further, it can be appreciated from FIG. 106 that the ends of the sutures 4724, 4726 may extend away from the first feltable implant 4718 of the repair implant 4700. It can be appreciated that the ends of the sutures 4724, 4726 may be fixedly attached (e.g., knotted, tied, felted, etc.) to the second feltable implant 4720, whereby the sutures 4724, 4726 are not slidable relative to the second feltable implant 4720.

Additionally, the first feltable implant 4718 and the second feltable implant 4720 may be formed from a plurality of individual feltable fibers which are matted, condensed, pressed, bonded, etc. together to form a non-woven structure. In some examples, the individual feltable fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable fibers may include combinations of any of the materials disclosed. For example, the feltable fibers may be formed from a combination of any of natural fibers, synthetic fibers, biodegradable fibers, collagen fibers, or the like. It can be further appreciated that the feltable fibers may be randomly aligned with one another to form a non-woven felted structure. In other examples, however, the feltable fibers may be aligned with one another to achieve unidirectional stiffness properties with the non-woven felted structure.

Further, in some examples, the first feltable implant 4718 and the second feltable implant 4720 may include a collagen base material integrated (e.g., interwoven, interlinked, interlaced, knitted, crisscrossed, netted, randomly-dispersed, uniformly-dispersed, arranged, etc.) with the feltable fibers. The collagen base material may include collagen, a collagen element and/or a collagen-like material similar to the bioinductive collagen constructs described herein. It can be appreciated that the collagen base material may be integrated within the feltable fibers forming the first feltable implant 4718 and the second feltable implant 4720.

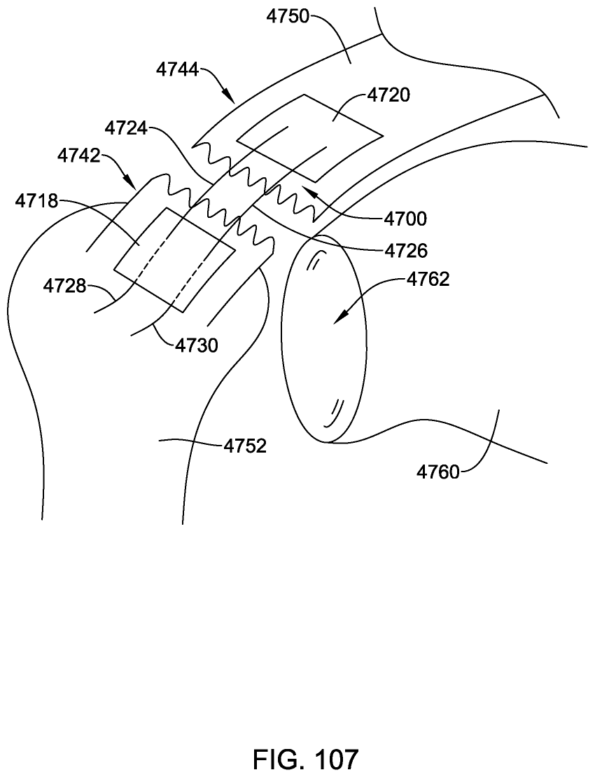

Similar to that described herein with respect to FIGS. 103-105, it can be appreciated that the repair implant 4700 may be utilized in a revision surgery to repair a tendon retear, such as the tendon retear illustrated and described with respect to FIG. 104. For example, FIG. 107 illustrates the second feltable implant 4720 attached to a portion 4744 of the torn supraspinatus tendon 4750. It can be appreciated that the second feltable implant 4720 may be attached to the portion 4744 of the supraspinatus tendon 4750 via felting the feltable fibers of the second feltable implant 4720 to the portion 4744 of the supraspinatus tendon 4750 using any of the felting techniques described herein.

Further, after the attachment of the second feltable implant 4720 to the portion 4744 of the supraspinatus tendon 4750, the repair implant 4700 may bridge the tear in the torn tendon 4750, whereby the first feltable implant 4718 may be positioned and attached to the portion 4742 of the supraspinatus tendon 4750 via felting the feltable fibers of the first feltable implant 4718 to the portion 4742 of the supraspinatus tendon 4750 using any of the felting techniques described herein. Further, after attaching the first feltable implant 4718 to the portion 4742 of the supraspinatus tendon 4750, each of the slidable sutures 4724, 4726 may be tensioned via pulling and sliding the sutures 4724, 4726 through the first feltable implant 4718. After the tensioning the sutures 4724, 4726, the slidable sutures 4724, 4726 may be fixedly attached (e.g., knotted, tied, felted, etc.) to the first feltable implant 4718, after which the sutures 4724, 4726 would not be slidable relative to the first feltable implant 4718 or the second feltable implant 4720.

It can be appreciated that tensioning the repair implant 4700 as described herein may reposition the portion 4742 of the torn tendon 4750 relative to the portion 4744 of the torn tendon 4750, thereby reapproximating the desired position of the tendon 4750 prior to the retear of the tendon 4750.

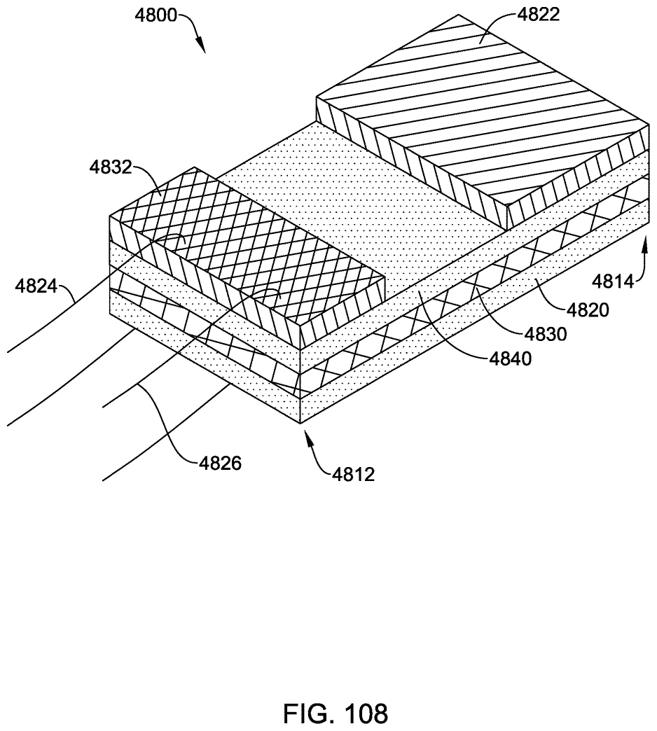

FIG. 108 illustrates a repair implant 4800. The repair implant 4800 may include a first end region 4812 and a second end region 4814. FIG. 108 illustrates that the repair implant 4800 may include a square shape. However, it is contemplated that the repair implant 4800 may include a variety of shapes such as circular, rectangular, ovular, crescent-shaped, triangular, polygonal, etc.

FIG. 108 illustrates that the repair implant 4800 may include a bottom layer 4820 formed from a biologic component. The biologic component may include collagen, a collagen element and/or a collagen-like material similar to other bioinductive collagen constructs described herein.

FIG. 108 further illustrates that the repair implant 4800 may include a layer 4830 of reinforcing material (e.g., reinforcing fibers, reinforcing mesh, etc.) positioned on top of the bottom layer 4820. The reinforcing fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the reinforcing fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the reinforcing fibers may include combinations of any of the materials disclosed. For example, the reinforcing fibers may be formed from a combination of any of natural fibers, synthetic fibers, biodegradable fibers, collagen fibers, or the like.

FIG. 108 further illustrates that the repair implant 4800 may include another layer 4840 of biologic material positioned on top of the layer 4830. The layer 4840 may be formed from a biologic component including collagen, a collagen element and/or a collagen-like material similar to other bioinductive collagen constructs described herein.

Additionally, FIG. 108 illustrates that the repair implant 108 may include a feltable material 4822 positioned on top of the layer 4840. The feltable material 4822 may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material 4822 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material 22, 24 may include combinations of any of the materials disclosed. For example, the feltable material 4822 may be formed from a combination of any of natural fibers, synthetic fibers, biodegradable fibers, collagen fibers, or the like. It can be further appreciated that the individual fibers used to construct the feltable material 4822 may be randomly aligned with one another to form the non-woven felted structure. In other examples, however, the individual fibers used to construct the feltable material 4822 may be aligned with one another to achieve unidirectional stiffness properties with the non-woven felted structure. It can be appreciated that the second end region 4814 may be attached to a target tissue site via felting the feltable material 4822 to the target tissue using any of the felting techniques described herein.

FIG. 108 further illustrates that the repair implant 4800 may further include a reinforcing material 4832 positioned on top of the layer 4840. It can be appreciated that the reinforcing material 4832 may be similar in form and function to the reinforcing material described herein with respect to the layer 4830. It can be further appreciated that the reinforcing material 4832 may be configured to reinforce the insertion points at which a first suture 4824 and a second suture 4826 pass into the repair implant 4800. As illustrated in FIG. 108, the sutures 4824, 4826 may pass from the reinforcement layer 4830 to the layer 4820, whereby the sutures 4824, 4826 may exit the layer 4820. As described herein, the sutures 4824, 4826 may be utilized to secure the repair implant 4800 at a target site. Tension may be applied to the sutures 4824, 4826 when securing the repair implant 4800 at a target site. Accordingly, the reinforcing material 4832 may be configured to prevent “tear out” of the sutures 4824, 4826 from the layers 4820, 4830, 4840, 4832. when tension is applied.

For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

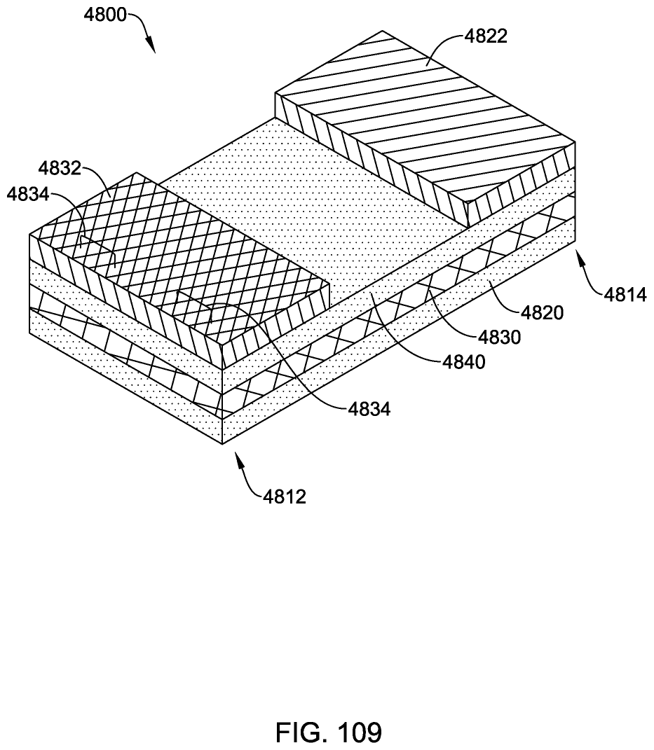

FIG. 109 illustrates the example repair implant 4800 described with respect to FIG. 108. As described with respect to FIG. 108, the repair implant 4800 may include a layer of biologic material 4820, a layer of reinforcing material 4830, a layer of biologic material 4840, feltable material 4822 and reinforcing material 4832. As described herein, FIG. 109 illustrates that the second end region 4814 of the repair implant 4800 may be secured to target tissue via felting the feltable material 4822 to the target tissue using any of the felting techniques described herein. Further, FIG. 109 illustrates that the first end region 4812 of the repair implant 4800 may be secured to a target site via one or more staples 4834.

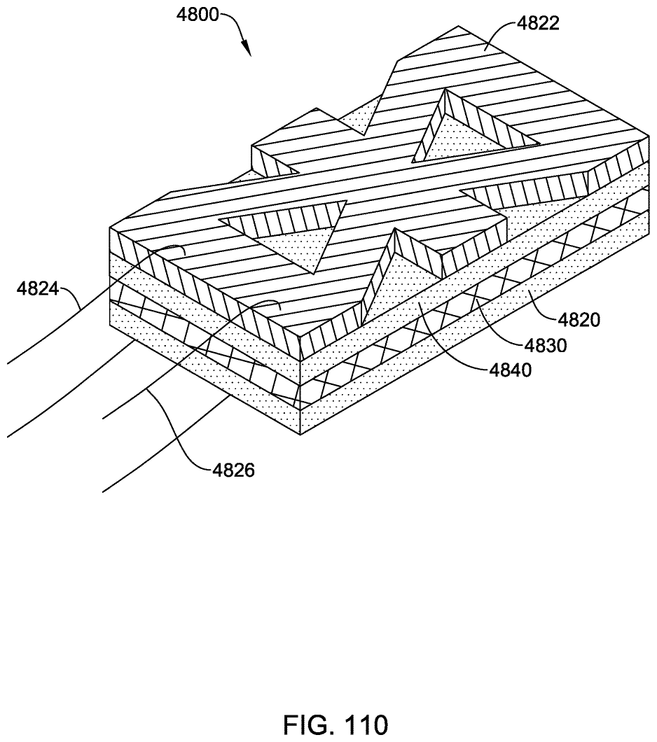

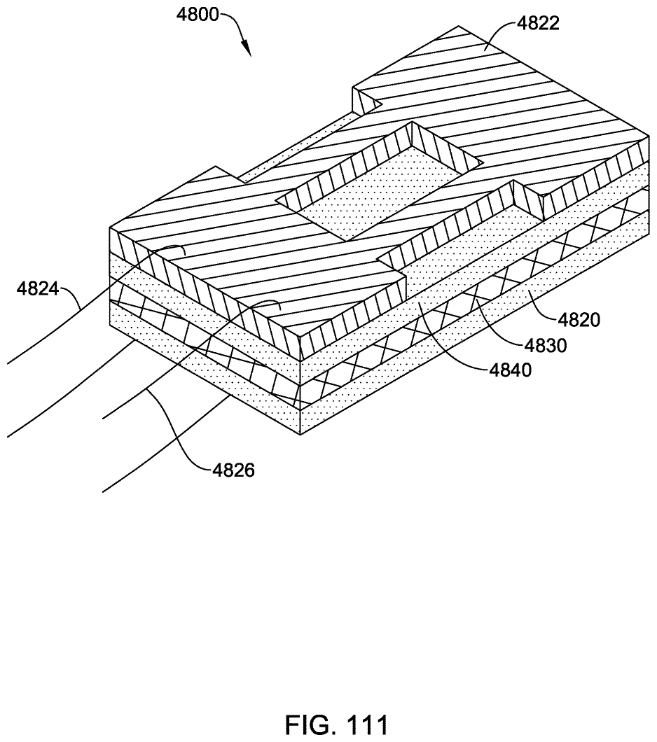

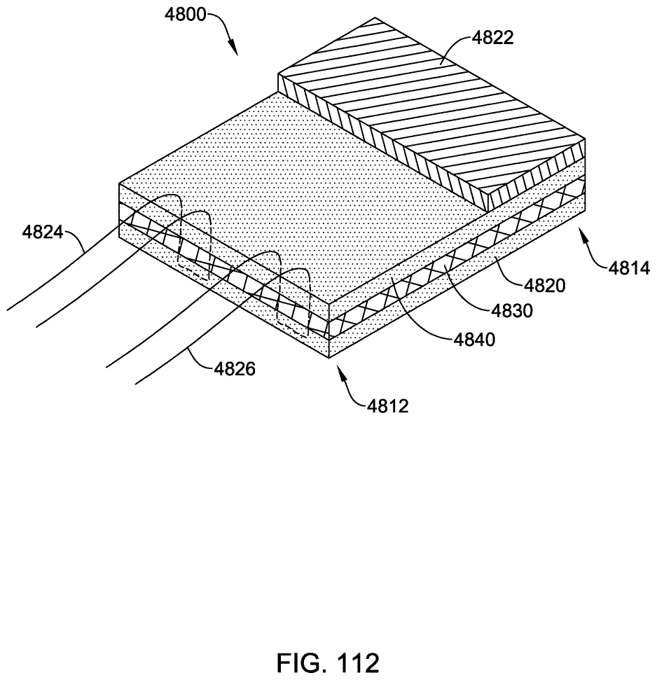

FIGS. 110-112 schematically depict various example arrangements of the layer of biologic material 4820, a layer of reinforcing material 4830, a layer of biologic material 4840 and feltable material 4822 of the implant 4800. These examples are in addition to the example arrangement of the biologic material 4820, a layer of reinforcing material 4830, a layer of biologic material 4840, feltable material 4822 and reinforcing material 4832 described with respect to FIG. 108. The example arrangements of the biologic material 4820, a layer of reinforcing material 4830, a layer of biologic material 4840, feltable material 4822 and reinforcing material 4832 shown in FIGS. 110-111 are schematic representations that depict alternative arrangements. The edges/boundaries shown in FIGS. 110-111 are intended to mimic the edge/boundaries of the pattern/arrangement shown in FIG. 108, or a variation thereof, and can be utilized in any of the devices disclosed as alternatives of the base/top layer. Further, similar to the reinforcement material 4832 described herein, it can be appreciated that the feltable material 4822 illustrated in FIGS. 110-111 may be configured to reinforce the repair implant 4800 prevent “tear out” of the sutures 4824, 4826 from the layers 4820, 4830, 4840, 4822. Further yet, the example shown in FIG. 112 illustrates an alternative method in which the sutures 4824, 4826 may be coupled (e.g., woven, stitched, engaged, etc.) to the layers 4820, 4830, 4840.

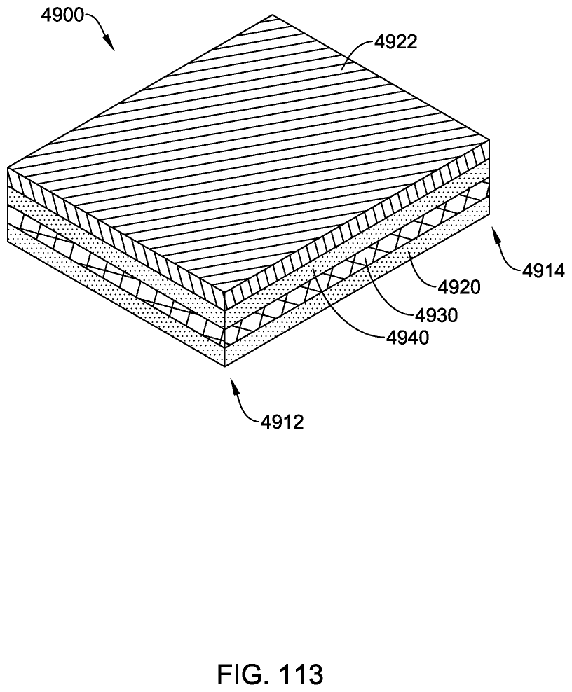

FIG. 113 illustrates a repair implant 4900. The repair implant 4900 may include a first end region 4912 and a second end region 4914. FIG. 113 illustrates that the repair implant 4900 may include a square shape. However, it is contemplated that the repair implant 4900 may include a variety of shapes such as circular, rectangular, ovular, crescent-shaped, triangular, polygonal, etc.

FIG. 113 illustrates that the repair implant 4900 may include a bottom layer 4920 formed from a biologic component. The biologic component may include collagen, a collagen element and/or a collagen-like material similar to other bioinductive collagen constructs described herein.

FIG. 113 further illustrates that the repair implant 4900 may include a layer 4930 of reinforcing material (e.g., reinforcing fibers, reinforcing mesh, etc.) positioned on top of the bottom layer 4920. The reinforcing fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the reinforcing fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the reinforcing fibers may include combinations of any of the materials disclosed. For example, the reinforcing fibers may be formed from a combination of any of natural fibers, synthetic fibers, biodegradable fibers, collagen fibers, or the like.

FIG. 113 further illustrates that the repair implant 4900 may include a layer 4940 of biologic material positioned on top of the layer 4930. The layer 4940 may be formed from a biologic component including collagen, a collagen element and/or a collagen-like material similar to other bioinductive collagen constructs described herein.

Additionally, FIG. 113 illustrates that the repair implant 4900 may include a feltable material 4922 positioned on top of the layer 4940. The feltable material 4922 may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material 4922 may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material 4922 may include combinations of any of the materials disclosed. For example, the feltable material 4922 may be formed from a combination of any of natural fibers, synthetic fibers, biodegradable fibers, collagen fibers, or the like. It can be further appreciated that the individual fibers used to construct the feltable material 4922 may be randomly aligned with one another to form the non-woven felted structure. In other examples, however, the individual fibers used to construct the feltable material 4922 may be aligned with one another to achieve unidirectional stiffness properties with the non-woven felted structure. It can be appreciated that the repair implant 4900 may be attached to a target tissue site via felting the feltable material 4922 to the target tissue using any of the felting techniques described herein.

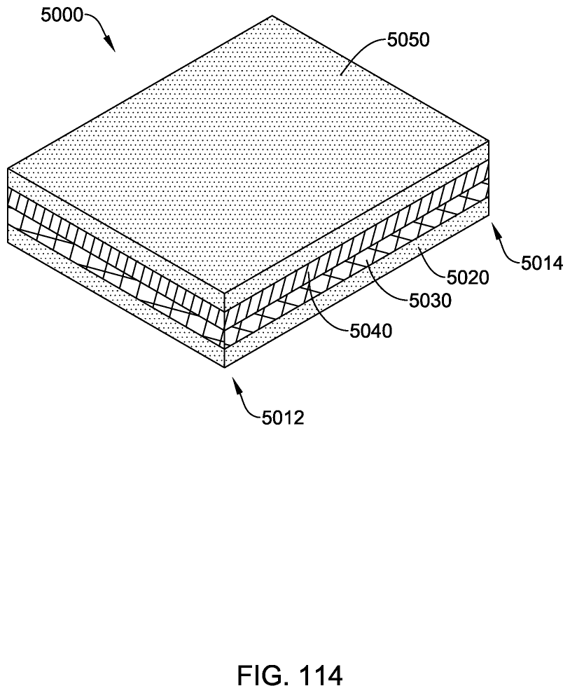

FIG. 114 illustrates a repair implant 5000. The repair implant 5000 may include a first end region 5012 and a second end region 5014. FIG. 114 illustrates that the repair implant 5000 may include a square shape. However, it is contemplated that the repair implant 5000 may include a variety of shapes such as circular, rectangular, ovular, crescent-shaped, triangular, polygonal, etc.

FIG. 114 illustrates that the repair implant 5000 may include a bottom layer 5020 formed from a biologic component. The biologic component may include collagen, a collagen element and/or a collagen-like material similar to other bioinductive collagen constructs described herein.

FIG. 114 further illustrates that the repair implant 5000 may include a layer 4930 of reinforcing material (e.g., reinforcing fibers, reinforcing mesh, etc.) positioned on top of the bottom layer 5020. The reinforcing fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the reinforcing fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the reinforcing fibers may include combinations of any of the materials disclosed. For example, the reinforcing fibers may be formed from a combination of any of natural fibers, synthetic fibers, biodegradable fibers, collagen fibers, or the like.

Additionally, FIG. 114 illustrates that the repair implant 5000 may include a layer 5040 of feltable material positioned on top of the layer 5030. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. It can be appreciated that the repair implant 5000 may be attached to a target tissue site via felting the feltable material to the target tissue using any of the felting techniques described herein.

FIG. 114 further illustrates that the repair implant 5000 may include a layer 5050 of biologic material positioned on top of the layer 5040. The layer 5040 of biologic material may be formed from collagen, a collagen element and/or a collagen-like material similar to other bioinductive collagen constructs described herein.

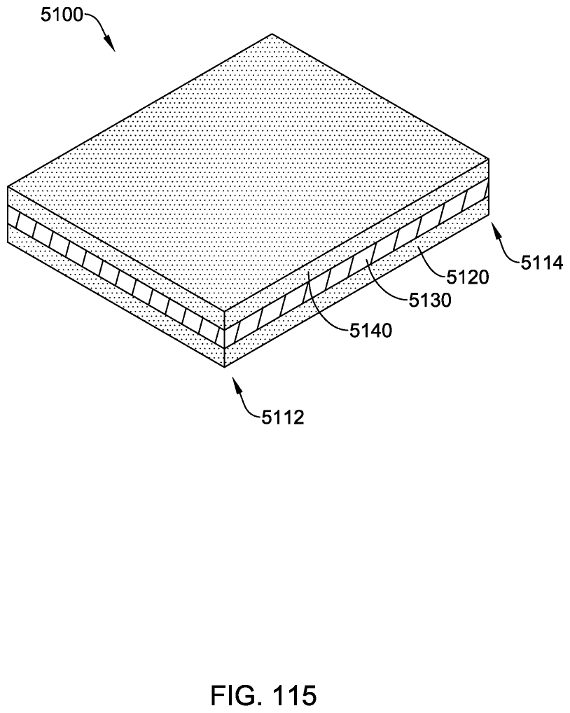

FIG. 115 illustrates a repair implant 5100. The repair implant 5100 may include a first end region 5112 and a second end region 5114. FIG. 115 illustrates that the repair implant 5100 may include a square shape. However, it is contemplated that the repair implant 5100 may include a variety of shapes such as circular, rectangular, ovular, crescent-shaped, triangular, polygonal, etc.

FIG. 115 illustrates that the repair implant 5100 may include a bottom layer 5120 formed from a biologic component. The biologic component may include collagen, a collagen element and/or a collagen-like material similar to other bioinductive collagen constructs described herein.

Additionally, FIG. 115 illustrates that the repair implant 5100 may include a layer 5130 of feltable material positioned on top of the layer 5120. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 5130 of feltable material illustrated in FIG. 115 may be configured to reinforce the repair implant 5100. Further, it can be appreciated that the repair implant 5100 may be attached to a target tissue site via felting the layer 5130 feltable material to the target tissue using any of the felting techniques described herein.

FIG. 115 further illustrates that the repair implant 5100 may include a layer 5140 of biologic material positioned on top of the layer 5130. The layer 5140 may be formed from a biologic component including collagen, a collagen element and/or a collagen-like material similar to other bioinductive collagen constructs described herein.

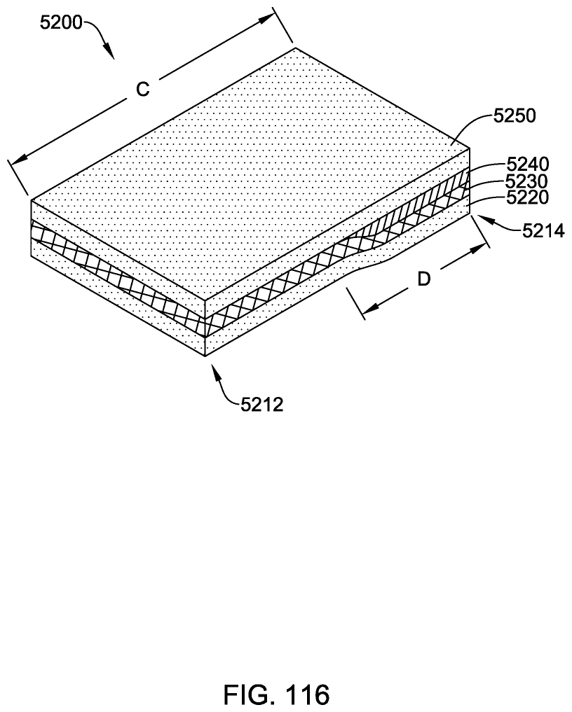

FIG. 116 illustrates a repair implant 5200. The repair implant 5200 may include a first end region 5212 and a second end region 5214. FIG. 116 illustrates that the repair implant 5200 may include a square shape. However, it is contemplated that the repair implant 5200 may include a variety of shapes such as circular, rectangular, ovular, crescent-shaped, triangular, polygonal, etc.

FIG. 116 illustrates that the repair implant 5200 may include a bottom layer 5220 formed from a biologic component. The biologic component may include collagen, a collagen element and/or a collagen-like material similar to other bioinductive collagen constructs described herein.

FIG. 116 further illustrates that the repair implant 5200 may include a layer 5230 of reinforcing material (e.g., reinforcing fibers, reinforcing mesh, etc.) positioned on top of the bottom layer 5220. The reinforcing fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the reinforcing fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the reinforcing fibers may include combinations of any of the materials disclosed. For example, the reinforcing fibers may be formed from a combination of any of natural fibers, synthetic fibers, biodegradable fibers, collagen fibers, or the like.

Additionally, FIG. 116 illustrates that the repair implant 5200 may include a layer 5240 of feltable material positioned along the second end region 5214. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. It can be appreciated that the repair implant 5200 may be attached to a target tissue site via felting the layer 5240 of feltable material to the target tissue using any of the felting techniques described herein.

FIG. 116 further illustrates that the repair implant 5200 may have a length “C”. Additionally, FIG. 116 illustrate that the layer 5240 of the repair implant 5200 may have a length “D”. In some examples, the length D may be about 20% to about 80% of the length C, or about 30% to about 70% of the length C, or about 40% to about 60% of the length C, or about 15% to about 50% of the length C, or about 20% to about 40% of the length C, or about 10% to about 30% of the length C, or about 5% to about 30% of the length C, or about 2% to about 15% of the length C, or about 1% to about 10% of the length C. Further, it can be appreciated from FIG. 116 that the repair implant may taper in thickness from the second end region 5214 to the first end region 5212. Accordingly, it can be appreciated that the second end region 5214 may be thicker than the first end region 5212 because the layer 5240 of feltable material may only extend along a portion of the length C of the repair implant 5200. In other words, the repair implant 5200 may include four layers 5220, 5230, 5240, 5250 along the second end region and three layers 5220, 5230, 5250 along the first end region 5212 whereby the layer 5030 tapers and discontinues along the length C of the repair implant 5200 from the second end region 5214 to the first end region 5212.

FIG. 116 further illustrates that the repair implant 5200 may include a layer 5250 of biologic material positioned on top of the layer 5240. The layer 5240 of biologic material may be formed from collagen, a collagen element and/or a collagen-like material similar to other bioinductive collagen constructs described herein.

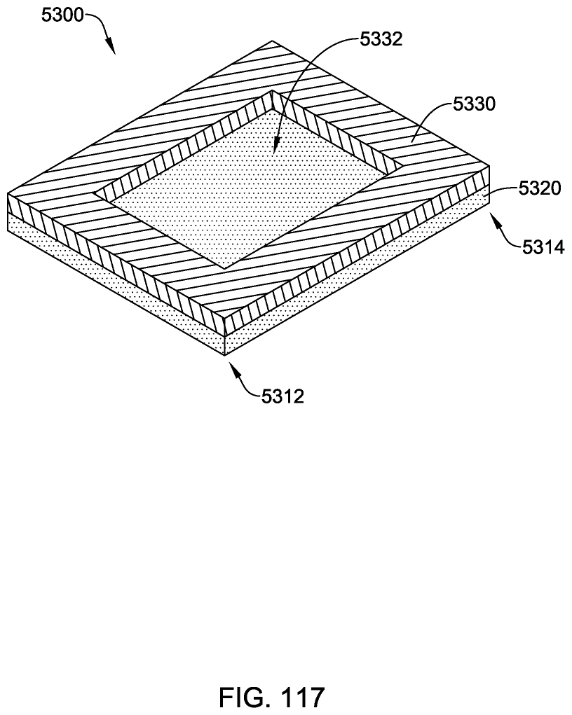

FIG. 117 illustrates a repair implant 5300. The repair implant 5300 may include a first end region 5312 and a second end region 5314. FIG. 117 illustrates that the repair implant 5300 may include a square shape. However, it is contemplated that the repair implant 5300 may include a variety of shapes such as circular, rectangular, ovular, crescent-shaped, triangular, polygonal, etc.

FIG. 117 illustrates that the repair implant 5300 may include a bottom layer 5320 formed from a biologic component. The biologic component may include collagen, a collagen element and/or a collagen-like material similar to other bioinductive collagen constructs described herein.

Additionally, FIG. 117 illustrates that the repair implant 5300 may include a layer 5330 of feltable material positioned on top of the layer 5320. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 5330 of feltable material illustrated in FIG. 117 may be configured to reinforce the repair implant 5300. Further, it can be appreciated that the repair implant 5300 may be attached to a target tissue site via felting the layer 5330 feltable material to the target tissue using any of the felting techniques described herein.

FIG. 117 further illustrates that the layer 5330 of feltable material may extend around the perimeter and/or edges of the bottom layer 5320. Accordingly, it can be appreciated that a central region 5332 of the repair implant 5300 may be free of feltable material.

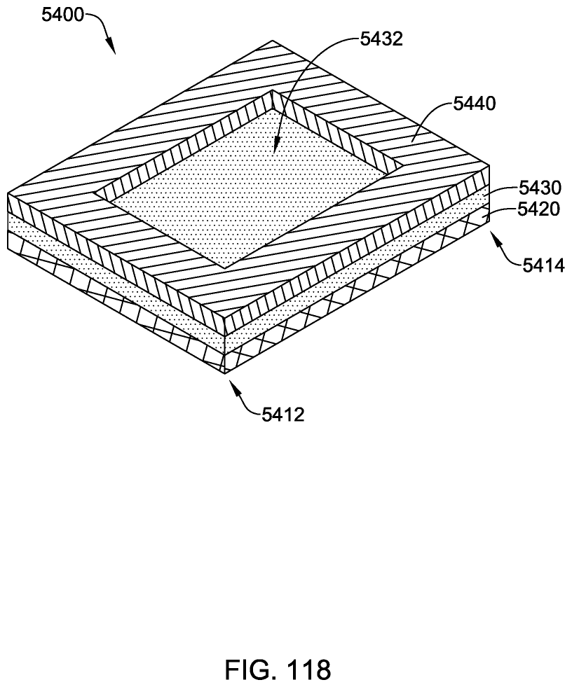

FIG. 118 illustrates a repair implant 5400. The repair implant 5400 may include a first end region 5412 and a second end region 5414. FIG. 118 illustrates that the repair implant 5400 may include a square shape. However, it is contemplated that the repair implant 5400 may include a variety of shapes such as circular, rectangular, ovular, crescent-shaped, triangular, polygonal, etc.

FIG. 118 illustrates that the repair implant 5400 may include a bottom layer 5420 of reinforcing material (e.g., reinforcing fibers, reinforcing mesh, etc.). The reinforcing fibers may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the reinforcing fibers may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the reinforcing fibers may include combinations of any of the materials disclosed. For example, the reinforcing fibers may be formed from a combination of any of natural fibers, synthetic fibers, biodegradable fibers, collagen fibers, or the like.

FIG. 118 further illustrates that the repair implant 5400 may include a layer 5430 formed from a biologic component. The biologic component may include collagen, a collagen element and/or a collagen-like material similar to other bioinductive collagen constructs described herein.

Additionally, FIG. 118 illustrates that the repair implant 5400 may include a layer 5440 of feltable material positioned on top of the layer 5430. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 5440 of feltable material illustrated in FIG. 118 may be configured to reinforce the repair implant 5400. Further, it can be appreciated that the repair implant 5400 may be attached to a target tissue site via felting the layer 5440 feltable material to the target tissue using any of the felting techniques described herein.

FIG. 118 further illustrates that the layer 5440 of feltable material may extend around the perimeter and/or edges of the layer 5430. Accordingly, it can be appreciated that a central region 5432 of the repair implant 5400 may be free of feltable material.

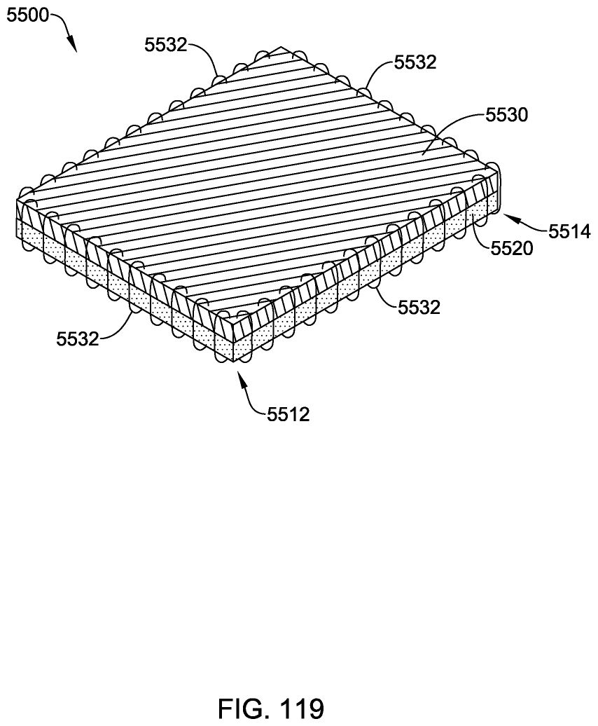

FIG. 119 illustrates a repair implant 5500. The repair implant 5500 may include a first end region 5512 and a second end region 5514. FIG. 119 illustrates that the repair implant 5500 may include a square shape. However, it is contemplated that the repair implant 5500 may include a variety of shapes such as circular, rectangular, ovular, crescent-shaped, triangular, polygonal, etc.

FIG. 119 illustrates that the repair implant 5500 may include a bottom layer 5520 formed from a biologic component. The biologic component may include collagen, a collagen element and/or a collagen-like material similar to other bioinductive collagen constructs described herein.

Additionally, FIG. 119 illustrates that the repair implant 5500 may include a layer 5530 of feltable material positioned on top of the layer 5520. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 5530 of feltable material illustrated in FIG. 119 may be configured to reinforce the repair implant 5500. Further, it can be appreciated that the repair implant 5500 may be attached to a target tissue site via felting the layer 5530 feltable material to the target tissue using any of the felting techniques described herein.

FIG. 119 further illustrates that the layer 5520 of biologic material and the layer 5530 of feltable material of the repair implant 5500 may be attached to each other via stitching 5532 which may extend around the perimeter of the repair implant 5500. The stitching 5532 may be formed using a single, continuous stitch or a plurality of stitches arranged in any configuration around the edges of the repair implant 5500. In some examples, the stitching 5532 may take the form of a “whip stitch” which extends around the perimeter of the repair implant 5500. The stitching 5532 may take the form of a wire, fiber, thread, filament, string, suture, bioabsorbable thread, suture-wire, suture tape, fiber wire, braided sutures, woven sutures, non-absorbable sutures, absorbable sutures, synthetic sutures, natural sutures, multifilament sutures, monofilament sutures and any combinations thereof. Further, the stitching 5532 may also cross longitudinally, horizontally, diagonally, in a curved or non-linear fashion, or in any combination across the implant 5500 to reinforce and/or attach the layers 5520, 5530 together.

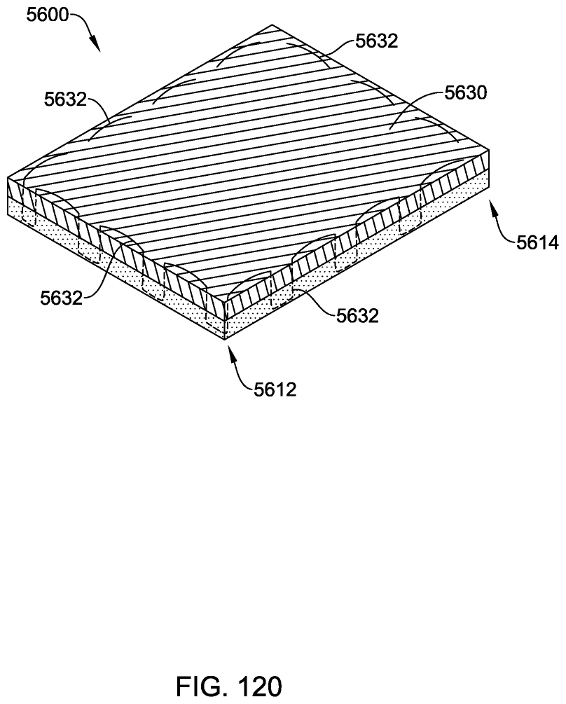

FIG. 120 illustrates a repair implant 5600. The repair implant 5600 may include a first end region 5612 and a second end region 5614. FIG. 120 illustrates that the repair implant 5600 may include a square shape. However, it is contemplated that the repair implant 5600 may include a variety of shapes such as circular, rectangular, ovular, crescent-shaped, triangular, polygonal, etc.

FIG. 120 illustrates that the repair implant 5600 may include a bottom layer 5620 formed from a biologic component. The biologic component may include collagen, a collagen element and/or a collagen-like material similar to other bioinductive collagen constructs described herein.

Additionally, FIG. 120 illustrates that the repair implant 5600 may include a layer 5630 of feltable material positioned on top of the layer 5620. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 5630 of feltable material illustrated in FIG. 120 may be configured to reinforce the repair implant 5600. Further, it can be appreciated that the repair implant 5600 may be attached to a target tissue site via felting the layer 5630 feltable material to the target tissue using any of the felting techniques described herein.

FIG. 120 further illustrates that the layer 5620 of biologic material and the layer 5630 of feltable material of the repair implant 5600 may be attached to each other via stitching 5632 which may extend around the perimeter of the repair implant 5600. The stitching 5632 may weave in a substantially wave pattern along the edges of the repair implant 5600. In other words, the stitching may pass in and out of the layers 5620, 5630, whereby a portion of each of the stitching extends from the layer 5630 to the layer 5620 of the repair implant 5600 in a wave-like pattern. The stitching 5632 may take the form of a wire, fiber, thread, filament, string, suture, bioabsorbable thread, suture-wire, suture tape, fiber wire, braided sutures, woven sutures, non-absorbable sutures, absorbable sutures, synthetic sutures, natural sutures, multifilament sutures, monofilament sutures and any combinations thereof. Further, the stitching 5632 may also be non-continuous, but in selected spots around the implant 5600.

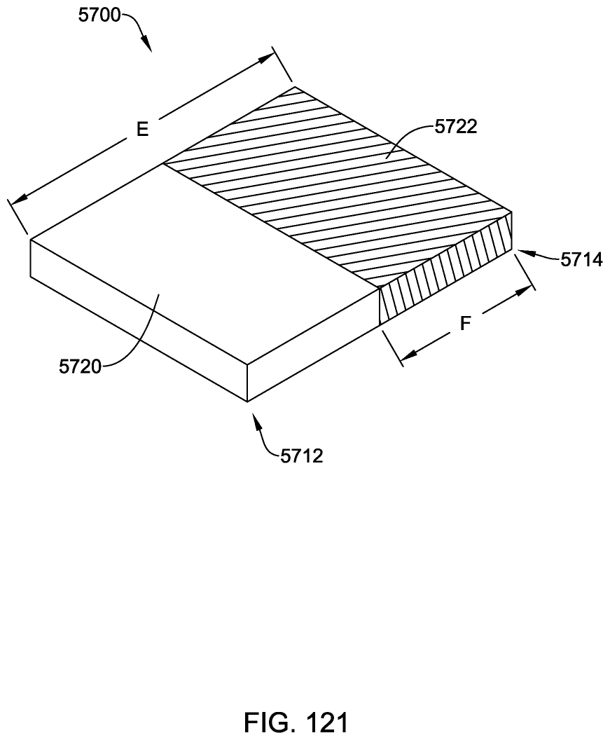

FIG. 121 illustrates a repair implant 5700. The repair implant 5700 may include a first end region 5712 and a second end region 5714. FIG. 121 illustrates that the repair implant 5700 may include a square shape. However, it is contemplated that the repair implant 5700 may include a variety of shapes such as circular, rectangular, ovular, crescent-shaped, triangular, polygonal, etc.

FIG. 121 illustrates that the repair implant 5700 may include a portion 5720 formed from a biologic component. The biologic component may include collagen, a collagen element and/or a collagen-like material similar to other bioinductive collagen constructs described herein.

Additionally, FIG. 121 illustrates that the repair implant 5700 may include a portion 5722 of feltable material positioned adjacent to the portion 5720 of biologic material. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Further, it can be appreciated that the repair implant 5700 may be attached to a target tissue site via felting the portion 5722 of feltable material to the target tissue using any of the felting techniques described herein.

FIG. 121 further illustrates that the repair implant 5700 may have a length “E”. Additionally, FIG. 121 illustrate that the portion 5722 of the repair implant 5700 may have a length “F”. In some examples, the length F may be about 20% to about 80% of the length E, or about 30% to about 70% of the length E, or about 40% to about 60% of the length E, or about 15% to about 50% of the length E, or about 20% to about 40% of the length E, or about 10% to about 30% of the length E, or about 5% to about 30% of the length E, or about 2% to about 15% of the length E, or about 1% to about 10% of the length E.

It can be appreciated that any of the layered or non-layered repair implant embodiments described herein (e.g., the repair implant examples shown in FIGS. 108-121) may be constructed using an electrospinning method such as that described herein with respect to FIG. 20. It can also be appreciated that, in other examples, any of the layered or non-layered repair implant embodiments described herein (e.g., the examples shown in FIGS. 108-121) may be constructed using chemical bonding methods and/or techniques. Further, any of the embodiments disclosed herein may be constructed using manufacturing methods which include bonding of different layers, including chemical bonding, adhesive bonding, or similar bonding methods of different layers or any components of any layered or non-layered repair implant or components thereof. In some examples, chemical bonding may include adhesive bonding of materials and/or components.

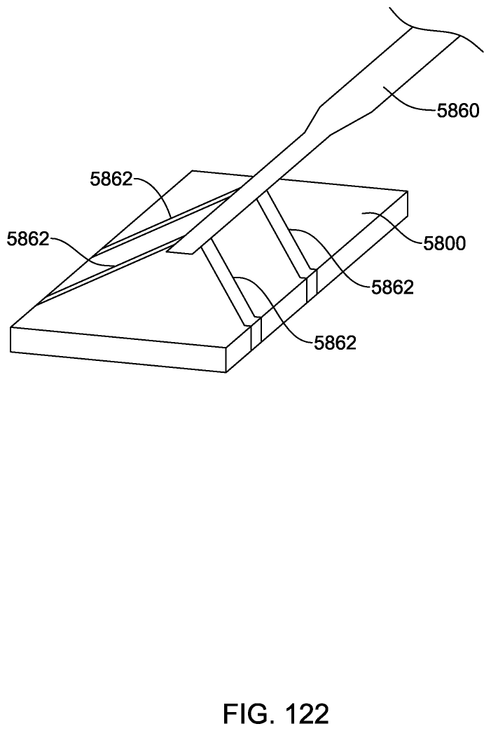

FIG. 122 illustrates a delivery device 5860 coupled to a repair implant 5800. The delivery system 5860 may be configured to position the repair implant 5800 at a position at a target tissue site within the body. FIG. 122 further illustrates the delivery device 5860 may include a plurality of attachment arms 5862 configured to grip (e.g., hold, etc.) the repair implant 5800. In some examples, the attachment arms 5862 may be directed at an angle relative to the implant 5800. In other examples, the attachment arms 5862 may be directed horizontal relative to the implant 5800.

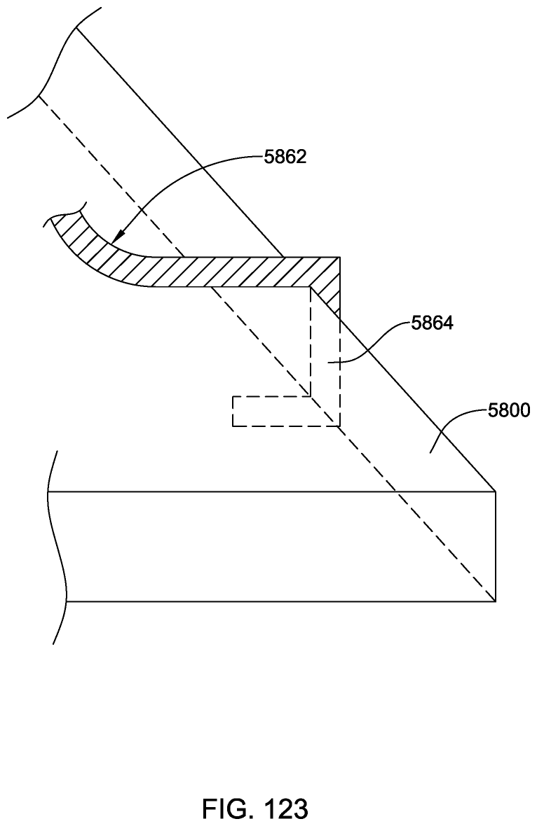

FIG. 123 illustrates a perspective view of an attachment arm 5862 of the delivery device 5860 (shown in FIG. 122). It can be appreciated that each of the attachment arms 5862 may include an attachment portion 5864 which is configured to wrap around a portion of the repair implant 5800. For example, FIG. 123 illustrates that the attachment portion 5864 may wrap around or through the repair implant 5800 from a top surface of the repair implant 5800 to a bottom surface of the repair implant 5800. In other examples, the attachment portion 5864 may fully pierce or partially pierce the repair implant 5800 for additional strength. It can be appreciated that the attachment portion 5864 of each of the attachment arms 5862 may be configured to grip the repair implant 5800 such that the repair implant 5800 may be manipulated by the delivery device 5860 yet also may be released from each of the attachment arms 5862 after the repair implant 5800 is secured to a target tissue site.

While FIG. 123 illustrates that the attachment portion 5864 may wrap around the repair implant 5800 from a top surface of the repair implant 5800 to a bottom surface of the repair implant 5800, in other examples, the attachment portion 5864 of each attachment arm 5862 may weave into and out of the body of the repair implant 5800 to secure the repair implant 5800 to each attachment arm 5862 of the delivery device 5800.

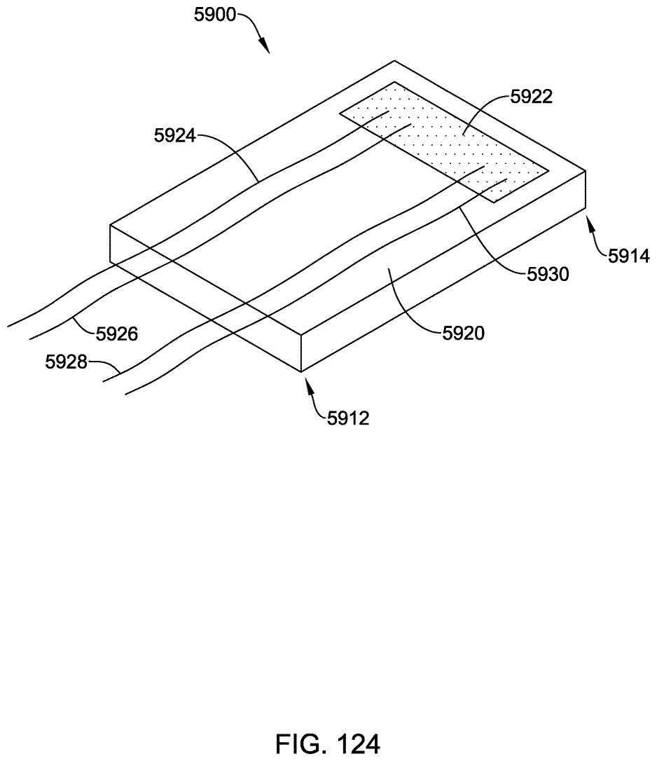

FIG. 124 illustrates repair implant 5900. The repair implant 5900 may include a first end region 5912 and a second end region 5914. FIG. 124 illustrates that the repair implant 5900 may include a rectangular shape. However, it is contemplated that the repair implant 5900 may include a variety of shapes such as circular, square, ovular, crescent-shaped, triangular, polygonal, etc.

FIG. 124 illustrates that the repair implant 5900 may include a base material 5920 formed from a biologic component. The biologic component may include collagen, a collagen element and/or a collagen-like material similar to other bioinductive collagen constructs described herein.

Additionally, FIG. 124 illustrates that the second end region 5914 of the repair implant 5900 may include a portion of feltable material 5922. The feltable material 5922 may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Further, it can be appreciated that the repair implant 5900 may be attached to a target tissue site via felting the portion 5922 of feltable material to the target tissue using any of the felting techniques described herein.

FIG. 124 further illustrates the repair implant 5900 may include four sutures 5924, 5926, 5928, 5930, fixedly attached to the feltable material 5922. FIG. 5900 illustrates the repair implant include four sutures, however, it can be appreciated that the repair implant 5900 may include 1, 2, 3, 4, 5, 6 or more sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

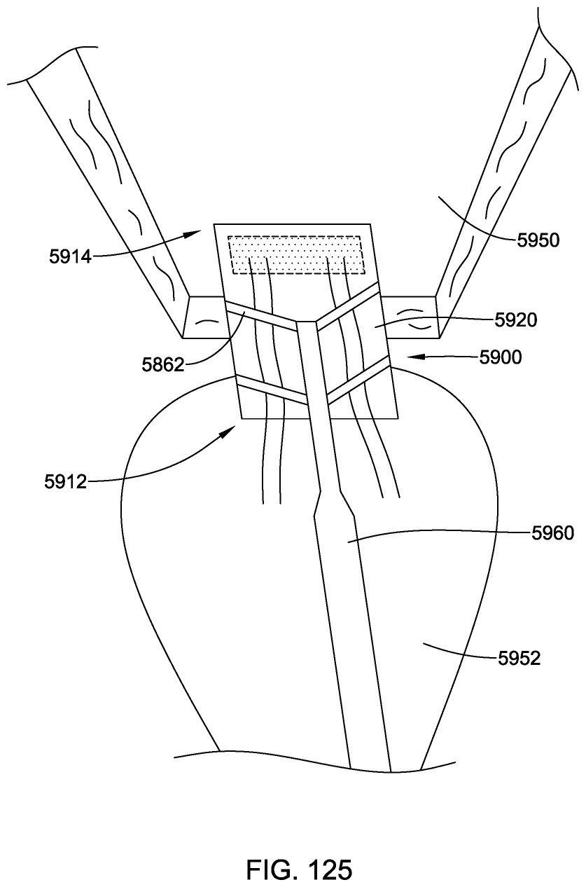

FIG. 125 illustrates the repair device 5960 (similar in form and function to the repair device 5860 described herein) being utilized to position the repair implant 5900 within the shoulder. For example, FIG. 125 illustrates the repair device 5960 positioning the repair implant 5900 within the shoulder such that the second end region 5914 of the repair implant 5900 is positioned along the rotator cuff tendon 5950 and the first end region 5912 of the repair implant 5900 is positioned along the footprint of the greater tuberosity 5952. It can be appreciated that the biologic material 5920 is positioned along both the rotator cuff tendon 5950 and the footprint of the greater tuberosity 5952. As discussed herein, FIG. 125 illustrates the attachment arms 5862 of the repair device 5960 gripping the repair implant 5900.

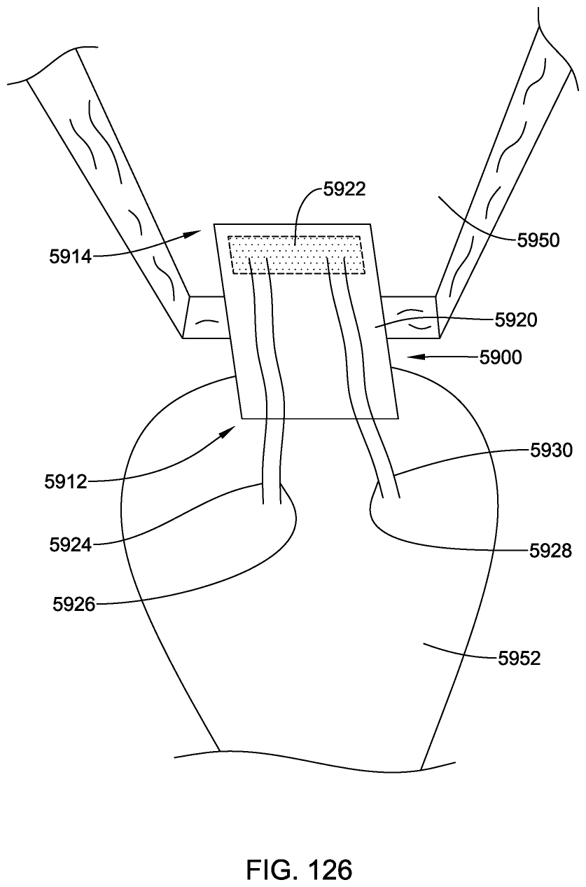

FIG. 126 illustrates a first step in securing the repair implant 5900 within the shoulder. FIG. 126 illustrates the repair device 5960 positioning the repair implant 5900 within the shoulder such that the second end region 5914 of the repair implant 5900 is positioned along the rotator cuff tendon 5950 and the first end region 5912 of the repair implant 5900 is positioned along the footprint of the greater tuberosity 5952. It can be appreciated that the biologic material 5920 is positioned along both the rotator cuff tendon 5950 and the footprint of the greater tuberosity 5952. FIG. 126 illustrates that a first step in securing the repair implant 5900 within the shoulder may include felting the feltable material 5922 to the tendon 5950 using any of the felting techniques described herein. FIG. 126 further illustrates the four sutures 5924, 5926, 5928, 5930, fixedly attached to the feltable material 5922 and extending away therefrom.

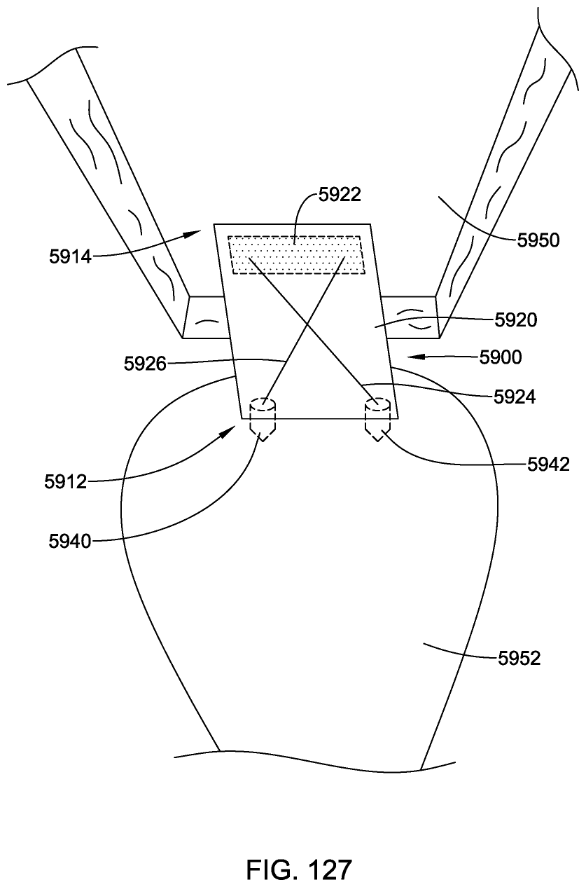

FIG. 127 illustrates a second step in securing the repair implant 5900 within the shoulder. FIG. 127 illustrates that a second step in securing the repair implant 5900 within the shoulder may include tensioning and securing the suture 5924 to an anchor 5942 (e.g., bone anchor, knotless anchor, etc.) and securing the suture 5926 to an anchor 5940 (e.g., bone anchor, knotless anchor, etc.). In this example, the repair implant 5900 may only include the sutures 5940, 5942. Accordingly, FIG. 127 omits the sutures 5928, 5930. It can be appreciated that the second end region 5914 biologic material 5920 is positioned along the rotator cuff tendon 5950 and the first end region 5912 biologic material 5920 is positioned along the footprint of the greater tuberosity 5952.

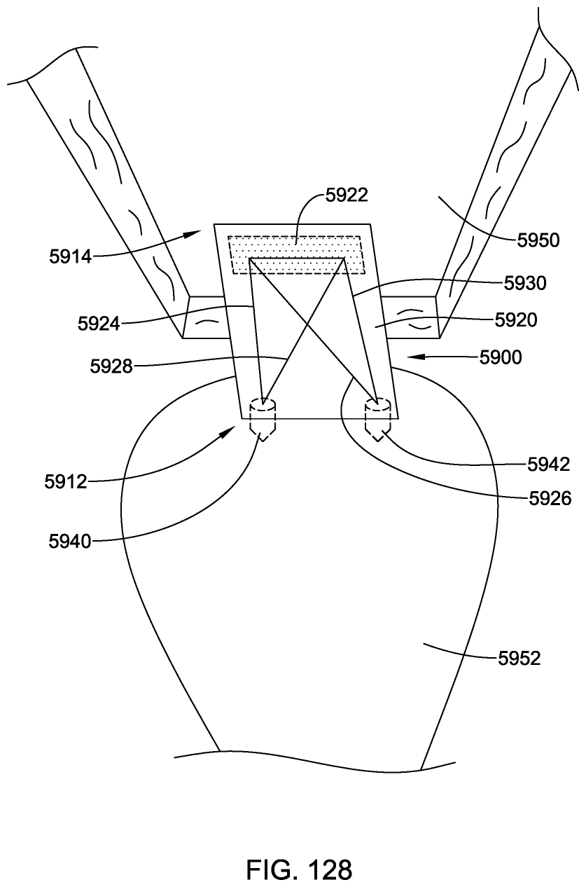

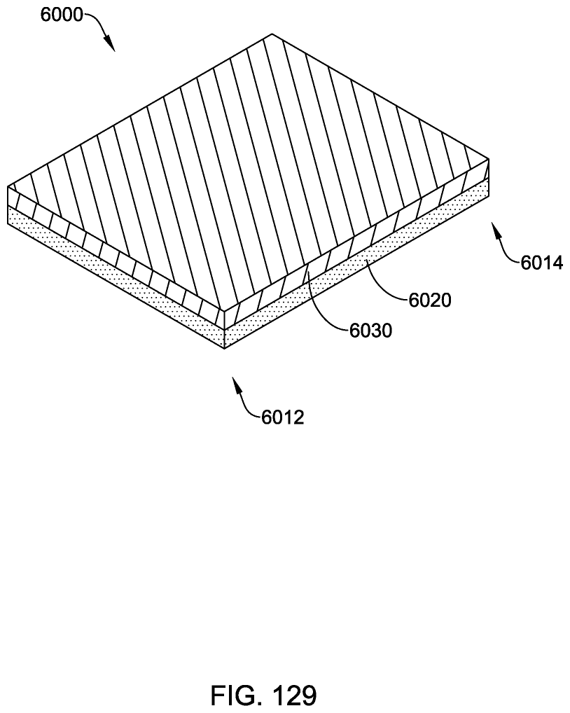

FIG. 128 illustrates an alternate second step in securing the repair implant 5900 within the shoulder. FIG. 128 illustrates that a second step in securing the repair implant 5900 within the shoulder may include tensioning and securing the sutures 5924, 5928 to an anchor 5940 (e.g., bone anchor, knotless anchor, etc.) and securing the sutures 5926, 5930 to an anchor 5942 (e.g., bone anchor, knotless anchor, etc.). It can be appreciated that the second end region 5914 biologic material 5920 is positioned along the rotator cuff tendon 5950 and the first end region 5912 biologic material 5920 is positioned along the footprint of the greater tuberosity 5952.

It can be appreciated that the example repair implants 5900 illustrated in FIGS. 127-128 may be implanted over a primary implant or repair (such as a primary implant illustrated and described with respect to FIG. 104). It can be appreciated that the repair implants 5900 may be positioned over top of a primary repair (such as a primary implant illustrated and described with respect to FIG. 104). It can be appreciated that in these instances, the anchors 5940, 5942 shown in FIGS. 127-128 may form a “third row” row of anchors which are positioned more laterally of the anchors used for the primary repair. For example, the anchors 5940, 5942 shown in FIGS. 127-128 may form a “third row” row of anchors which are positioned more laterally of knotless anchors which may have been utilized for the primary repair.

FIG. 129 illustrates a repair implant 6000. The repair implant 6000 may include a first end region 6012 and a second end region 6014. FIG. 129 illustrates that the repair implant 6000 may include a square shape. However, it is contemplated that the repair implant 6000 may include a variety of shapes such as circular, rectangular, ovular, crescent-shaped, triangular, polygonal, etc.

FIG. 129 illustrates that the repair implant 6000 may include a bottom layer 6020 formed from a biologic component. The biologic component may include collagen, a collagen element and/or a collagen-like material similar to other bioinductive collagen constructs described herein.

Additionally, FIG. 129 illustrates that the repair implant 6000 may include a layer 6030 of feltable material positioned on top of the layer 6020. The feltable material may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure. Additionally, like other reinforcement materials described herein, it can be appreciated that the layer 6030 of feltable material illustrated in FIG. 129 may be configured to reinforce the repair implant 6000. Further, it can be appreciated that the repair implant 6000 may be attached to a target tissue site via felting the layer 6030 of feltable material to the target tissue using any of the felting techniques described herein.

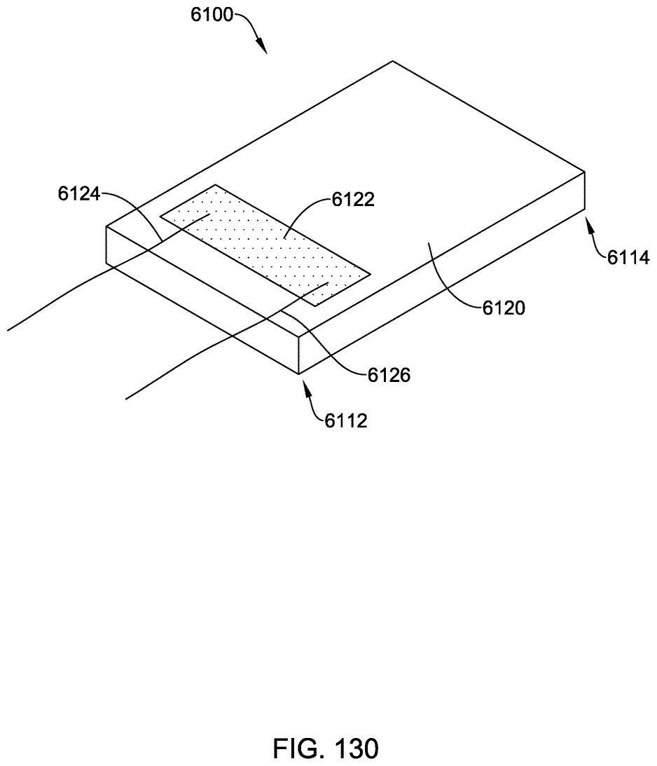

FIG. 130 illustrates a repair implant 6100. The repair implant 6100 may include a first end region 6112 and a second end region 6114. FIG. 130 illustrates that the repair implant 6100 may include a rectangular shape. However, it is contemplated that the repair implant 6100 may include a variety of shapes such as circular, square, ovular, crescent-shaped, triangular, polygonal, etc.

FIG. 130 illustrates that the repair implant 6100 may include a base material 6120 formed from a biologic component. The biologic component may include collagen, a collagen element and/or a collagen-like material similar to other bioinductive collagen constructs described herein.

Additionally, FIG. 130 illustrates that the first end region 6112 of the repair implant 6100 may include a portion of feltable material 6122. The feltable material 6122 may be constructed from a plurality of individual fibers which are matted, condensed, pressed, bonded, etc. together to form a non-woven structure or construct. In some examples, the individual fibers forming feltable material described herein may include natural fibers (e.g., cotton, silk, wool, etc.), synthetic fibers (e.g., polypropylene, polyamide, polytetrafluoroethylene, polyethylene, ultra-high-molecular-weight polyethylene, polyethylene terephthalate, or other high-strength fibers, etc.), metallic (e.g., stainless steel wool, titanium wool, magnesium wool, etc.), collagen fibers, or any combination thereof. Additionally, the feltable material may be formed from fibers which include a biodegradable material (e.g., poly-L-lactic acid, poly-glycolic-acid, polycaprolactone, poly-4-hydroxybutyrate, degradable polyurethanes, degradable polythiolurethanes, polytrimethylene carbonate, etc.). In some examples, the feltable material may include combinations of any of the materials disclosed. For example, the feltable material may be formed from a combination of any of natural fibers, synthetic fibers, biodegradable fibers, collagen fibers, or the like. It can be further appreciated that the individual fibers used to construct the feltable material may be randomly aligned with one another to form the non-woven felted structure. In other examples, however, the individual fibers used to construct the feltable material may be aligned with one another to achieve unidirectional stiffness properties with the non-woven felted structure.

Additionally, like other reinforcement materials described herein, it can be appreciated that the feltable material 6122 illustrated in FIG. 130 may be configured to reinforce the repair implant 6100. Further, it can be appreciated that the repair implant 6100 may be attached to a target tissue site via felting the feltable material 6122 to a target tissue using any of the felting techniques described herein. For example, the feltable material 6122 of the repair implant 6100 may be felted to a portion of a torn tendon which remains fixed to the footprint of the greater tuberosity.

FIG. 130 further illustrates the repair implant 6100 may include sutures 6124, 6126 fixedly attached to the feltable material 6122 and/or the base material 6120. FIG. 130 illustrates the repair implant 6100 may include two sutures, however, it can be appreciated that the repair implant 6100 may include 1, 2, 3, 4, 5, 6 or more sutures. For purposes of discussion herein, “sutures” may include suture-wire, suture tape, fiber wire, braided sutures, woven sutures, non-absorbable sutures, absorbable sutures, synthetic sutures, natural sutures, multifilament sutures, monofilament sutures and any combinations thereof.

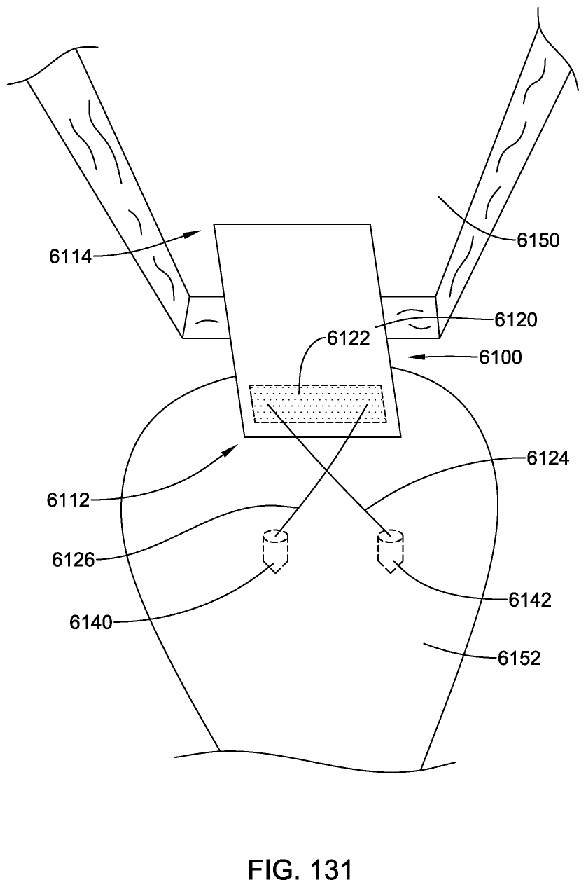

FIG. 131 illustrates the repair implant 6100 positioned and secured within the shoulder. It can be appreciated that the second end region 6114 of the repair implant 6100 may be positioned along the rotator cuff tendon 6150 and the first end region 6112 may be positioned along the footprint of the greater tuberosity 6152. The second end region 6114 may be secured to the rotator cuff tendon 6150 via one or more sutures, staples, anchors, or combinations thereof. Further, FIG. 131 illustrates that the suture 6124 may be attached to an anchor 6142 (e.g., bone anchor, knotless anchor, etc.) and that the suture 6126 to an anchor 6140 (e.g., bone anchor, knotless anchor, etc.), thereby securing the first end region 6112 of the repair implant 6100 along the footprint of the greater tuberosity 6152. It can be appreciated that the second end region 6114 of the biologic material 6120 is positioned along the rotator cuff tendon 6150 and the first end region 6112 of the biologic material 6120 is positioned along the footprint of the greater tuberosity 6152. Additionally, it can be appreciated that the feltable material 6122 may reinforce the sutures 6124, 6126 along the portion of the repair implant 6100 to which they attach. Further, in some examples, the feltable material 6122 of the repair implant 6100 may be felted to a portion of a torn tendon which remains fixed to the footprint of the greater tuberosity in addition to the suture 6124 being attached to the anchor 6142 and the suture 6126 attached to the anchor 6140.

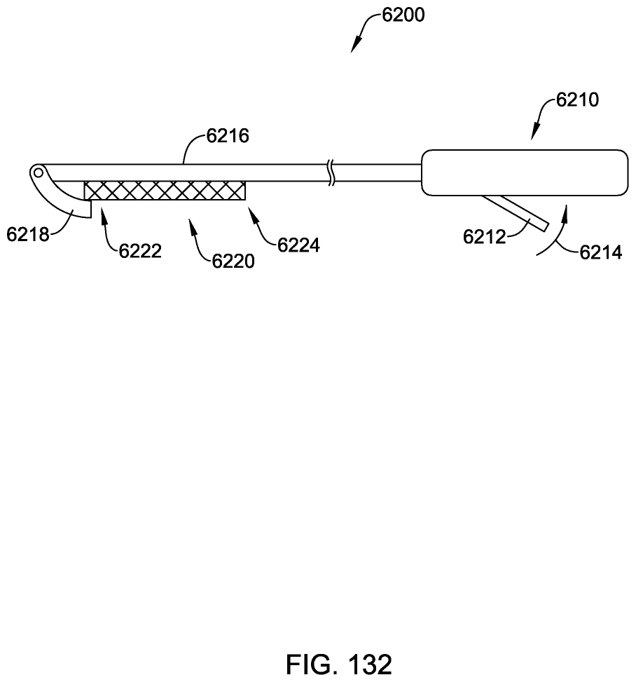

FIG. 132 illustrates an implant delivery device 6200. The delivery device 6200 may include an actuation shaft 6216 having a proximal end attached to a handle 6210. The handle 6210 may include a lever 6212. Additionally, the distal end region of the actuation shaft 6216 may be coupled to a gripping member 6218 (e.g., clamp, jaw, etc.).

FIG. 132 further illustrates that actuation of the lever 6212 may actuate the gripping member 6218. For example, it can be appreciated that actuation of the lever 6212 may shift the gripping member 6218 from an open configuration to a closed configuration. It can further be appreciated from FIG. 132 that squeezing the lever 6212 from a position away from the body of the handle 6210 to a position closer to the body of the handle 6210, as depicted by the arrow 6214, may shift the gripping member 6218 from an open configuration to a closed configuration.

FIG. 132 further illustrates that actuation of the gripping member 6218 may permit the delivery device 6200 to grasp (e.g., grip, clamp, grab, constrain, compress, etc.) a medical implant 6220 (or other medical element) between the gripping member 6218 and the actuation shaft 6216. FIG. 132 illustrates that the medical implant 6220 may include a proximal region 6224 and a distal region 6222. It can be appreciated that the configuration of the delivery device 6200 illustrated in FIG. 132 may permit the delivery device 6200 to grasp the distal region 6222 of a medical implant 6220, thereby permitting the delivery device to “pull” and advance the medical implant 6220 to a tissue repair site. Once positioned along the tissue repair site, the lever 6212 may be released, whereby the gripping member 6218 releases the medical implant 6220 by shifting from the closed position to an open position. Once the implant is released, the delivery device 6200 may be removed from the patient.

For purposes of discussion herein, any of the “anchors” described herein may include bone anchors, knotless anchors, knotted anchors, suture anchors, metal anchors, bioabsorbable anchors, biocomposite anchors, biostable anchors, screw-in anchors, bone fixation anchors, staples, combinations thereof or any other suitable anchor.

For purposes of discussion herein, any of the materials, constructs, components, feltable constructs and/or feltable components of any of the examples described herein may include collagen, a collagen element, a collagen-like material similar to other bioinductive collagen constructs described herein, polymers, polymers including an absorbable material such as PGA, PLGA, HA, or other suitable absorbable polymers, non-absorbable materials, or any combination of collagen, absorbable polymers, and nonabsorbable polymers, biologic materials, non-biologic materials, biologic components, non-biologic components, variations thereof or combinations thereof.

For purposes of discussion herein, any of the materials, constructs, components, feltable constructs and/or feltable components of any of the examples described herein may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as VESTAMID®, GRILAMID® available from EMS American Grilon, and/or the like), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP).

For purposes of discussion herein, any of the example sutures described herein may be fixed to implant, or slidably attached. Sutures may be integrated into an implant itself during manufacturing, or placed there during the procedure with the use of a suture passer or threading needle.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A medical implant, comprising:
a first base material having a first end region and a second end region; and
a feltable material adjacent to the first base material;
wherein the feltable material includes a plurality of feltable fibers configured to attach the implant to a target tissue.

2. The medical implant of claim 1, wherein at least a portion of the first base material is formed from collagen.

3. The medical implant of claim 1, wherein at least a portion of the first base material is formed from a polymer.

4. The medical implant of claim 1, wherein the feltable material is dispersed within the first base material.

5. The medical implant of claim 1, wherein the feltable material is chemically bonded to the first base material.

6. The medical implant of claim 1, wherein the feltable material is positioned on top of the first base material.

7. The medical implant of claim 6, wherein the feltable material is positioned along the perimeter of the first base material, and wherein a central portion of the first base material is free of feltable material.

8. The medical implant of claim 1, further comprising a second base material, wherein the feltable material is positioned between the first base material and the second base material.

9. The medical implant of claim 8, wherein at least a portion of the first base material and the second base material are formed from collagen.

10. The medical implant of claim 8, further comprising a reinforcing material, wherein the reinforcing material is positioned between the feltable material and the second base material, and wherein the feltable material is positioned between the first base material and the reinforcing material.

11. The medical implant of claim 8, wherein the medical implant includes a length extending from the first end region of the base material to the second end region of the base material, and wherein the feltable material extends along only a portion of the length.

12. The medical implant of claim 8, wherein the first end region is devoid of the feltable material.

13. The medical implant of claim 8, wherein the first end region has a first thickness and the second end region has a second thickness greater than the first thickness.

14. The medical implant of claim 9, further comprising a reinforcing material, wherein the reinforcing material is positioned between the first base material and the second base material, and wherein the first base material is positioned between the feltable material and the reinforcing material.

15. The medical implant of claim 14, wherein the feltable material is positioned along the second end region of the medical implant and wherein the first end region is devoid of the feltable material.

16. The medical implant of claim 15, further comprising a reinforcing material.

17. The medical implant of claim 16, wherein the first base material is positioned between the reinforcing material and the feltable material.

18. The medical implant of claim 1, wherein the feltable material is positioned along a perimeter of the first base material, and wherein a central portion of the first base material is free of the feltable material.

19. A medical implant, comprising:
a first base material having a first end region and a second end region;
a feltable material coupled to the first base material; and
a suture coupled to the first end region;
wherein the feltable material is configured to anchor the second end region to a first target tissue;
wherein the suture is configured to anchor the first end region to a bone anchor.

20. A medical implant, comprising:
a base component including a first end region, a first base material, and a feltable material;
a first suture coupled to a first end region;
a second suture coupled to a first end region;
a first bone anchor coupled to the first suture; and
a second bone anchor coupled to the second suture.
